(19) United States
(12) Patent Application Publication
(10) Pub. No.: US 2012/0077802 A1
(43) Pub. Date: Mar. 29, 2012

(54) HISTAMINE H3 INVERSE AGONISTS AND ANTAGONISTS AND METHODS OF USE THEREOF

(75) Inventors: Milan Chytil, Clinton, MA (US); Sharon R. Engel, Hudson, MA (US); Qun Kevin Fang, Wellesley, MA (US)

(73) Assignee: SUNOVION PHARMACEUTICALS INC., Marlborough, MA (US)

(21) Appl. No.: 13/376,368

(22) PCT Filed: Jun. 9, 2010
(86) PCT No.: PCT/US10/37964

§ 371 Date: Dec. 5, 2011

Related U.S. Application Data
(60) Provisional application No. 61/185,936, filed on Jun. 10, 2009.

(51) Int. Cl. A61K 31/55; C07D 491/107; A61K 31/438; A61K 31/5377; A61K 31/513; A61K 31/497; A61K 31/496; A61K 31/4545; A61P 25/18; A61P 25/28; A61P 25/16; A61P 25/00; A61P 1/08; A61P 25/08; A61P 3/04; A61P 3/10; A61P 1/00; A61P 25/14; A61P 25/22; A61P 3/00; A61P 25/04; A61P 25/24; C07D 491/20

(52) U.S. Cl. 514/212.02; 546/18; 544/70; 546/17; 540/543; 544/230; 514/278; 514/233.2; 514/269; 514/255.05; 514/253.11; 514/254.04

(57) ABSTRACT

Provided herein are spiro-cyclic compounds, methods of synthesis, and methods of use thereof. The compounds provided herein are useful for the treatment, prevention, and/or management of various disorders, including, e.g., neurological disorders and metabolic disorders. Compounds provided herein inhibit the activity of histamine H3 receptors and modulate the release of various neurotransmitters, such as, e.g., histamine, acetylcholine, norepinephrine, and dopamine (e.g. at the synapse). Pharmaceutical compositions containing the compounds and their methods of use are also provided herein.

This application claims priority to U.S. Provisional Patent Application No. 61/185,936, filed on Jun. 10, 2009, the content of which is hereby incorporated by reference herein in its entirety.

### I. FIELD

Provided herein are compounds useful as histamine H3 receptor inverse agonists or antagonists, compositions comprising the compounds, and methods of their use.

### II. BACKGROUND

Histamine producing cells locate in the tuberomammillary nucleus (TMN) and project throughout the brain and the spinal cord to form a histamine neurotransmitter system. Four histamine receptors, histamine H1, H2, H3, and H4 receptors, have been identified to date. The human H3 receptor was cloned in 1999. See, e.g., Lovenberg et al., Mol. Pharmacol. 55(6): 1101-07 (1999).

Histamine H3 receptors (also referred to as H3 receptors or H3 herein) are expressed on neurons throughout the CNS, particularly the forebrain. H3 receptors are primarily localized at the pre-synaptic site of the neurons and act as auto-receptors to regulate neurotransmitter release. H3 receptor is a G-protein coupled receptor (GPCR) that signals primarily through the Gi/o pathway. Activation of the pre-synaptic H3 receptors located on histaminergic neurons leads to a decrease in histamine release; whereas inhibition of H3 receptors with an antagonist or inverse agonist leads to an increase in histamine at the synapse. Thus H3 receptor ligands are capable of modifying histaminergic neurotransmission in the brain: agonists decrease it, and antagonists or inverse agonists increase it. H3 receptors from the brain have significant constitutive activity in the absence of agonists. Consequently, inverse agonists will reduce receptor activity, increase histamine release, and activate histaminergic neurons. See, e.g., Goodman & Gilman's Pharmacological Basis of Therapeutics, 629 (11^{th} ed. 2006).

H3 receptors are also found on the terminals of other neurotransmitter producing neurons, where they serve as pre-synaptic hetero-receptors to regulate the release of other neurotransmitters. H3 receptor antagonists have been shown to increase acetylcholine, norepinephrine, and dopamine in the extra-cellular fluid. The ability for H3 receptors to modulate the release of a variety of neurotransmitters suggests a wide range of therapeutic indications for H3 antagonists and inverse agonists.

H3 receptor antagonists or inverse agonists that cross the blood-brain barrier have a range of central effects through the activation of histaminergic neurons. For example, in animal experiments, H3 antagonists or inverse agonists induced marked arousal and wakefulness, improved attention and learning, and demonstrated beneficial effects in animal models of convulsions. Thus these compounds may be used to treat conditions such as cognitive impairment, pathological diurnal somnolence, and epilepsy without sedative side effects. The ability of these compounds to improve wakefulness could also lead to an improved sleep pattern, and therefore H3 antagonists or inverse agonists may also be useful in treating sleeping disorders, such as insomnia.

Preclinical research with H3 antagonists and inverse agonists suggests that this class of ligands may offer novel treatments for a variety of disorders, including but not limited to, cognitive impairments (such as those associated with Alzheimer's and Parkinson's diseases), schizophrenia, attention deficit hyperactivity disorder (ADHD), pain, and obesity. Additionally, these ligands have been shown to possess wake-promoting properties in both pre-clinical and clinical studies and may be useful in disorders associated with excessive daytime sleepiness. Additional uses of H3 ligands include, but are not limited to, disorders of the mood such as anxiety and depression, seizures, vertigo, movement disorders, and gastrointestinal (GI) motility disorders.

In addition, it is reported that H3 receptors may be associated with other various neurological disorders. Therefore, there is a great need for effective H3 inverse agonists and antagonists as therapeutics for treatment of various disorders, such as neurological disorders.

### III. SUMMARY

Provided herein are compounds of formula (I), or pharmaceutically acceptable salt or stereoisomer thereof:

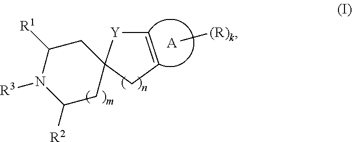

wherein R¹, R², R³, R, Y, ring A, k, m, and n are defined herein elsewhere. The compounds are useful as histamine H3 receptor inverse agonists or antagonists.

Also provided herein are compositions and dosage forms comprising compounds provided herein. Compositions and dosage forms provided herein may comprise one or more additional active ingredients.

Also provided herein are methods for the treatment, prevention, and/or management of one or more disorder(s) using the compounds provided herein. Also provided herein are methods for the treatment, prevention, and/or management of one or more disorder(s) using the compositions provided herein. Also provided herein are methods for the treatment, prevention, and/or management of one or more symptoms of a disorder provided herein comprising administering a compound provided herein. Also provided herein are methods for the treatment, prevention, and/or management of one or more symptoms of a disorder provided herein comprising administering a composition provided herein. Also provided herein are uses of the compounds provided herein in the manufacture of a medicament for the treatment, prevention, and/or management of one or more disorder(s) provided herein. Also provided herein are uses of the compositions provided herein in the manufacture of a medicament for the treatment, prevention, and/or management of one or more disorder(s) provided herein. Also provided herein are compounds for use in the treatment, prevention, and/or management of one or more disorder(s) provided herein. Also provided herein are compositions for use in the treatment, prevention, and/or management of one or more disorder(s) provided herein. Disorders that may be treated, prevented, and/or managed include, but are not limited to, neurological disorders; neurodegenerative diseases; schizophrenia; Alzheimer's disease; Parkinson's disease; affective disorders; attention deficit hyperactivity disorder (ADHD); psychosis; convulsion; seizures; vertigo; epilepsy; narcolepsy; pain (e.g. neuropathic pain); sensitization that accompanies many neuropathic pain disorders; mood disorders such as depression and anxiety; excessive daytime sleepiness such as that seen in narcolepsy, Parkinson's disease, multiple sclerosis, shift workers, and jet lag, or as a relief of side effects of other medications; insomnia; substance abuse; cognitive impairments, impairments of learning, impairments of memory, impairments of attention, vigilance or speed of response, such as those associated with Alzheimer's disease, Parkinson's disease, schizophrenia, mild cognitive impairment (MCI), and ADHD; metabolic disorders such as diabetes and obesity; disorders related to satiety and gastric activity, or as a side effects of other medications; diseases affecting the enteric system, such as acid secretion, digestion, and gut motility; and movement disorders such as Parkinson's disease, restless leg syndrome (RLS), Huntington's disease; and any other neurological disorders described herein elsewhere.

In another embodiment, provided herein is a method of inhibiting or reducing the activity of histamine H3 receptors. The method comprises contacting the H3 receptor with a compound provided herein.

Also provided herein is a method of regulating the release of neurotransmitters, including but not limited to, histamine, acetylcholine, norepinephrine, and dopamine, at the synapse. The method comprises contacting the cell with a compound provided herein. In an exemplary embodiment, the cell is a brain cell, such as, for example, a neuronal cell or a glial cell.

### IV. DETAILED DESCRIPTION

Unless defined otherwise, all technical and scientific terms used herein have the same meaning as those commonly understood by one of ordinary skill in the art. All publications and patents referred to herein are incorporated by reference herein in their entireties.

### A. DEFINITIONS

As used herein, and unless otherwise indicated, the term “alkyl” refers to a linear or branched saturated monovalent hydrocarbon radical, wherein the alkyl may optionally be substituted with one or more substituents. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 12 (C₁₋₁₂), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

As used herein, and unless otherwise specified, the term “alkenyl” refers to a linear or branched monovalent hydrocarbon radical, which contains one or more, in one embodiment, one to five, carbon-carbon double bonds. The alkenyl may be optionally substituted one or more substituents. The term “alkenyl” also encompasses radicals having “cis” and “trans” configurations, or alternatively, “E” and “Z” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

As used herein, and unless otherwise specified, the term “alkynyl” refers to a linear or branched monovalent hydrocarbon radical, which contains one or more, in one embodiment, one to five, carbon-carbon triple bonds. The alkynyl may be optionally substituted one or more substituents. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀, or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

As used herein, and unless otherwise specified, the term “cycloalkyl” refers to a cyclic saturated bridged and/or non-bridged monovalent hydrocarbon radical, which may be optionally substituted one or more substituents as described herein. In certain embodiments, the cycloalkyl has from 3 to 20 (C₃₋₂₀), from 3 to 15 (C₃₋₁₅), from 3 to 12 (C₃₋₁₂), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, decalinyl, and adamantyl.

As used herein, and unless otherwise specified, the term “heteroalkyl” refers to a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms are optionally oxidized and the nitrogen heteroatom can optionally be quaternized. The heteroatom(s) O, N and S may be placed at any interior position of the heteroalkyl group. The heteroatom Si can be placed at any position of the heteroalkyl group, including the position at which the heteroalkyl group is attached to the remainder of the molecule. In one embodiment, the heteroatom(s) O, N, and S can be placed at the external position distal to where the heteroalkyl group is attached to the remainder of the molecule. In one embodiment, the heteroatom(s) O, N, and S cannot be placed at the position at which the heteroalkyl group is attached to the remainder of the molecule. In one embodiment, the heteroatom(s) O, N, and S can be placed at the position at which the heteroalkyl group is attached to the remainder of the molecule. Examples include —O—CH₃, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms can be consecutive, such as, for example, —CH2-NH—OCH₃ and —CH₂—O—Si(CH₃)₃. Also included in the term “heteroalkyl” are those radicals described as “heteroalkylene” and “heterocycloalkyl.” The term “heteroalkylene” by itself or as part of another substituent means a divalent radical derived from heteroalkyl, as exemplified by —CH₂—CH₂—S—CH₂—CH₂— and —CH₂—S—CH₂—CH₂—NH—CH₂—. In one embodiment, for heteroalkylene groups, heteroatoms can also occupy either or both of the chain termini. In one embodiment, for heteroalkylene linking groups, as well as all other linking group provided herein, no orientation of the linking group is implied.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may also be optionally substituted with one or more substituents.

As used herein, and unless otherwise specified, the term “arylalkyl” or “aralkyl” refers to a monovalent alkyl group substituted with aryl. In certain embodiments, both alkyl and aryl may be optionally substituted with one or more substituents.

As used herein, and unless otherwise specified, the term “heteroaryl” refers to a monocyclic aromatic group and/or multicyclic aromatic group that contain at least one aromatic ring, wherein at least one aromatic ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may be optionally substituted with one or more substituents.

As used herein, and unless otherwise specified, the term “heterocycloalkyl,” “heterocyclyl,” or “heterocyclic” refers to a monocyclic non-aromatic ring system and/or multicyclic ring system that contains at least one non-aromatic ring, wherein at least one non-aromatic ring contains one or more heteroatoms independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocyclic may be optionally substituted with one or more substituents.

As used herein, and unless otherwise specified, the term “halogen”, “halide” or “halo” refers to fluorine, chlorine, bromine, and/or iodine.

As used herein, and unless otherwise specified, “isotopic composition” refers to the amount of each isotope present for a given atom; “natural isotopic composition” refers to the naturally occurring isotopic composition or abundance for a given atom. In one embodiment, as used herein, and unless otherwise specified, the term “hydrogen” encompasses proton (¹H), deuterium (²H), tritium (³H), and/or mixtures thereof. In one embodiment, the hydrogen in a given position of the compounds provided herein may have a natural isotopic composition or an isotopic composition enriched with one or more isotope(s) (e.g., proton, deuterium, and/or tritium). Unless otherwise designated, the atoms of the compounds recited herein are meant to represent any known isotope of that atom or an isotopic composition thereof, including, without limitation, ¹²C, ¹³C and/or ¹⁴C; ³²S, ³³S, ³⁴S, and/or ³⁶S; ¹⁴N and/or ¹⁵N; and ¹⁶O, ¹⁷O and/or ¹⁸O).

As used herein, and unless otherwise specified, the term “optionally substituted” is intended to mean that a group, such as an alkyl, alkenyl, alkynyl, cycloalkyl, heteroalkyl, aryl, aralkyl, heteroaryl, or heterocyclyl, may be substituted with one or more substituents independently selected from, e.g., (a) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl, each optionally substituted with one or more, in one embodiment, one, two, three, or four, substituents Q¹; and (b) halo, cyano (—CN), nitro (—NO₂), —C(O)R^{a}, —C(O)OR^{a}, —C(O)NR^{b}R^{c}, —C(NR^{a})NR^{b}R^{c}, —OR^{a}, —OC(O) R^{a}, —OC(O)OR^{a}, —OC(O)NR^{b}R^{c}, —OC(═NR^{a})NR^{b}R^{c}, —OS(O) R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each optionally substituted with one or more, in one embodiment, one, two, three, or four, substituents Q¹; or (iii) R^{b} and R^{c} together with the N atom to which they are attached form heteroaryl or heterocyclyl, optionally substituted with one or more, in one embodiment, one, two, three, or four, substituents Q¹. As used herein, all groups that can be substituted are “optionally substituted,” unless otherwise specified.

In one embodiment, each Q¹ is independently selected from the group consisting of (a) cyano, halo, and nitro; and (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)R^{h}, —NR^{e}C(O)NR^{f}R^{g}, —NR^{e}C(═NR^{h})NR^{f}R^{g}, —NR^{e}S(O) R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{f} and R^{g} together with the N atom to which they are attached form heteroaryl or heterocyclyl.

As used herein, and unless otherwise specified, the term “pharmaceutically acceptable salts” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. In some embodiments, the salt is formed from hydrochloric, hydrobromic, phosphoric, or sulfuric acid. In one embodiment, the salt is formed from hydrochloride salt.

As used herein, and unless otherwise specified, the term “solvate” refers to a compound provided herein or a salt thereof, which further includes a stoichiometric or non-stoichiometric amount of solvent bound by non-covalent intermolecular forces. Where the solvent is water, the solvate is a hydrate.

As used herein, and unless otherwise specified, the term “stereoisomer” encompasses all enantiomerically/stereomerically pure and enantiomerically/stereomerically enriched compounds provided herein. In certain embodiments, the term “stereoisomer” encompasses a single enantiomer or a single diastereomer. In certain embodiments, the term “stereoisomer” encompasses a mixture of two or more enantiomers and/or diastereomers.

As used herein and unless otherwise specified, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

As used herein and unless otherwise indicated, the term “stereomerically enriched” means a composition that comprises greater than about 55% by weight of one stereoisomer of a compound, greater than about 60% by weight of one stereoisomer of a compound, greater than about 70% by weight, or greater than about 80% by weight of one stereoisomer of a compound.

As used herein, and unless otherwise indicated, the term “enantiomerically pure” means a stereomerically pure composition of a compound having one chiral center. Similarly, the term “enantiomerically enriched” means a stereomerically enriched composition of a compound having one chiral center.

In certain embodiments, as used herein, and unless otherwise specified, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) or (−), is not related to the absolute configuration of the molecule, R or S.

As used herein, and unless otherwise specified, the term “compound” referred to herein, such as, e.g., a compound of formula (I), (II), (III), (IVa), (IVb), (IVc), (Va), (Vb), (VIa), (VIb), (VIIa), (VIIb), (VIIIa), or (VIIIb), is intended to encompass one or more of the following: a free base of the compound or a salt thereof, or a stereoisomer, a mixture of two or more stereoisomers, a solid form (e.g., a crystal form or an amorphous form), a mixture of two or more solid forms, a solvate (e.g., a hydrate), a cocrystal, a complex, or a prodrug thereof. In certain embodiments, the term “compound” referred to herein is intended to encompass a pharmaceutical acceptable form of the compound, such as, e.g., a free base of the compound or a pharmaceutically acceptable salt thereof, or a stereoisomer, a mixture of two or more stereoisomers, a solid form (e.g., a crystal form or an amorphous form), a mixture of two or more solid forms, a solvate (e.g., a hydrate), a cocrystal, a complex, or a prodrug thereof. In certain embodiments, the term “compound” referred to herein is intended to encompass a free base of the compound or a salt thereof, or a stereoisomer, a mixture of two or more stereoisomers, a solid form (e.g., a crystal form or an amorphous form), a mixture of two or more solid forms, or a solvate (e.g., a hydrate) thereof. In certain embodiments, the term “compound” referred to herein is intended to encompass a solid form (e.g., a crystal form or an amorphous form) or a mixture of two or more solid forms of a free base of the compound or a salt thereof. In certain embodiments, the term “compound” referred to herein is intended to encompass a solvate (e.g., a hydrate) of a free base of the compound or a salt thereof. In one embodiment, a salt of the compound provided herein contains a suitable acid as provided herein as the counterion of the compound to form the salt. In one embodiment, the salt is a pharmaceutically acceptable salt as described herein elsewhere.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, and unless otherwise specified, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, 2nd Edition, Gibson ed., CRC Press LLC: Boca Raton, Fla., 2009.

As used herein, and unless otherwise specified, the terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer of a compound described herein.

As used herein, and unless otherwise specified, the terms “drug” and “therapeutic agent” refer to a compound, or a pharmaceutical composition thereof, which is administered to a subject for treating, preventing, managing, or ameliorating one or more symptoms of a condition, disorder, or disease.

As used herein, and unless otherwise indicated, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound provided herein, with or without other additional active agent, after the onset of symptoms of the particular disease.

As used herein, and unless otherwise indicated, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of disease or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

As used herein, and unless otherwise specified, the terms “manage,” “managing,” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder. In this regard, the term “managing” encompasses treating a patient who had suffered from the particular disease in an attempt to prevent or minimize the recurrence of the disease.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice and the like. In specific embodiments, the subject is a human.

As used herein, and unless otherwise specified, the term “histamine receptor ligand” refers to any compound, which binds to a histamine receptor. Unless otherwise specified, the histamine receptor includes, but is not limited to, histamine H3 receptor. Ligands include endogenous ligands for a given histamine receptor as well as drug molecules and other compounds, such as synthetic molecules known to bind to a particular histamine receptor. In one example, the ligands include those labeled with one or more radioisotopes, such as tritium, or otherwise (e.g., fluorescently) labeled. It is within the abilities of the skilled person to select an appropriate ligand for a given histamine receptor. For example, known ligands for the histamine receptor include histamine, R-γ-Me-histamine, imetit, thioperamide, clobenpropit, and the like.

As used herein, and unless otherwise specified, the term “neurological disorder” refers to any condition of the central or peripheral nervous system of a mammal. The term “neurological disorder” includes, but is not limited to, neurodegenerative diseases (e.g., Alzheimer's disease, Parkinson's disease and amyotrophic lateral sclerosis), neuropsychiatric diseases (e.g., schizophrenia and anxieties, such as general anxiety disorder), and affective disorders (e.g., depression and attention deficit disorder). Exemplary neurological disorders include, but are not limited to, MLS (cerebellar ataxia), Huntington's disease, Down syndrome, multi-infarct dementia, status epilecticus, contusive injuries (e.g., spinal cord injury and head injury), viral infection induced neurodegeneration, (e.g., AIDS, encephalopathies), epilepsy, benign forgetfulness, closed head injury, sleep disorders, depression (e.g., bipolar disorder), dementias, movement disorders, psychoses, alcoholism, post-traumatic stress disorder and the like. “Neurological disorder” also includes any condition associated with the disorder. In one embodiment, a method of treating a neurodegenerative disorder includes methods of treating loss of memory and/or loss of cognition associated with a neurodegenerative disorder. In one embodiment, a method of treating a neurodegenerative disorder includes methods of treating cognitive function, memory performance, learning performance, speed of reaction, and/or time to respond associated with a neurodegenerative disorder. An exemplary method would also include treating or preventing loss of neuronal function characteristic of neurodegenerative disorder. “Neurological disorder” also includes any disease or condition that is implicated, at least in part, in monoamine (e.g., norepinephrine) signaling pathways (e.g., cardiovascular disease).

As used herein, and unless otherwise specified, the term “affective disorder” includes depression, attention deficit disorder, attention deficit disorder with hyperactivity, bipolar and manic conditions, and the like. The terms “attention deficit disorder” (ADD) and “attention deficit disorder with hyperactivity” (ADDH), or attention deficit/hyperactivity disorder (AD/HD), are used herein in accordance with the accepted meanings as found in the Diagnostic and Statistical Manual of Mental Disorders, 4^{th} ed., American Psychiatric Association (1997) (DSM-IV™).

As used herein, and unless otherwise specified, the term “depression” includes all forms of depression including, but not limited to, major depressive disorder (MDD), bipolar disorder, seasonal affective disorder (SAD) and dysthymia. “Major depressive disorder” is used herein interchangeably with “unipolar depression” and “major depression.” “Depression” may also includes any condition commonly associated with depression, such as all forms of fatigue (e.g., chronic fatigue syndrome) and cognitive deficits.

As used herein, and unless otherwise specified, the terms “obsessive-compulsive disorder,” “substance abuse,” “pre-menstrual syndrome,” “anxiety,” “eating disorders” and “migraine” are used herein in a manner consistent with their accepted meanings in the art. See, e.g., DSM-IV™. For example, the term “eating disorder,” as used herein, refers to abnormal compulsions to avoid eating or uncontrollable impulses to consume abnormally large amounts of food. These disorders may affect not only the social well-being, but also the physical well-being of sufferers. Examples of eating disorders include, but are not limited to, anorexia nervosa, bulimia, and binge eating.

As used herein, and unless otherwise specified, the term “pain” refers to an unpleasant sensory and emotional experience. The term “pain,” as used herein, refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic peripheral neuropathic pain (See, e.g., Harrison's Principles of Internal Medicine, pp. 93-98 (Wilson et al., eds., 12th ed. 1991); Williams et al., J. of Med. Chem. 42: 1481-1485 (1999), herein each incorporated by reference in their entirety). “Pain” is also meant to include mixed etiology pain, dual mechanism pain, allodynia, causalgia, central pain, hyperesthesia, hyperpathia, dysesthesia, and hyperalgesia. In addition, The term “pain” includes pain resulting from dysfunction of the nervous system: organic pain states that share clinical features of neuropathic pain and possible common pathophysiology mechanisms, but are not initiated by an identifiable lesion in any part of the nervous system.

The term “somatic pain,” as used herein, refers to a normal nerve response to a noxious stimulus such as injury or illness, e.g., trauma, burn, infection, inflammation, or disease process such as cancer, and includes both cutaneous pain (e.g., skin, muscle or joint derived) and visceral pain (e.g., organ derived).

The term “neuropathic pain,” as used herein, refers to a heterogeneous group of neurological conditions that result from damage to the nervous system. The term also refers to pain resulting from injury to or dysfunctions of peripheral and/or central sensory pathways, and from dysfunctions of the nervous system, where the pain often occurs or persists without an obvious noxious input. This includes pain related to peripheral neuropathies as well as central neuropathic pain. Common types of peripheral neuropathic pain include diabetic neuropathy (also called diabetic peripheral neuropathic pain, or DN, DPN, or DPNP), post-herpetic neuralgia (PHN), and trigeminal neuralgia (TGN). Central neuropathic pain, involving damage to the brain or spinal cord, can occur following stroke, spinal cord injury, and as a result of multiple sclerosis, and is also encompassed by the term. Other types of pain that are meant to be included in the definition of neuropathic pain include, but are not limited to, pain from neuropathic cancer pain, HIV/AIDS induced pain, phantom limb pain, and complex regional pain syndrome.

The term also encompasses the common clinical features of neuropathic pain including, but not limited to, sensory loss, allodynia (non-noxious stimuli produce pain), hyperalgesia and hyperpathia (delayed perception, summation, and painful after sensation). Pain is often a combination of nociceptive and neuropathic types, for example, mechanical spinal pain and radiculopathy or myelopathy.

As used herein, and unless otherwise specified, the term “acute pain” refers to the normal, predicted physiological response to a noxious chemical, thermal or mechanical stimulus typically associated with invasive procedures, trauma and disease. It is generally time-limited, and may be viewed as an appropriate response to a stimulus that threatens and/or produces tissue injury. The term also refers to pain which is marked by short duration or sudden onset.

As used herein, and unless otherwise specified, the term “chronic pain” encompasses the pain occurring in a wide range of disorders, for example, trauma, malignancies and chronic inflammatory diseases such as rheumatoid arthritis. Chronic pain may last more than about six months. In addition, the intensity of chronic pain may be disproportionate to the intensity of the noxious stimulus or underlying process. The term also refers to pain associated with a chronic disorder, or pain that persists beyond resolution of an underlying disorder or healing of an injury, and that is often more intense than the underlying process would predict. It may be subject to frequent recurrence.

As used herein, and unless otherwise specified, the term “inflammatory pain” is pain in response to tissue injury and the resulting inflammatory process. Inflammatory pain is adaptive in that it elicits physiologic responses that promote healing. However, inflammation may also affect neuronal function. Inflammatory mediators, including PGE₂ induced by the COX2 enzyme, bradykinins, and other substances, bind to receptors on pain-transmitting neurons and alter their function, increasing their excitability and thus increasing pain sensation. Much chronic pain has an inflammatory component. The term also refers to pain which is produced as a symptom or a result of inflammation or an immune system disorder.

As used herein, and unless otherwise specified, the term “visceral pain” refers to pain which is located in an internal organ.

As used herein, and unless otherwise specified, the term “mixed etiology pain” refers to pain that contains both inflammatory and neuropathic components.

As used herein, and unless otherwise specified, the term “dual mechanism pain” refers to pain that is amplified and maintained by both peripheral and central sensitization.

As used herein, and unless otherwise specified, the term “causalgia” refers to a syndrome of sustained burning, allodynia, and hyperpathia after a traumatic nerve lesion, often combined with vasomotor and sudomotor dysfunction and later trophic changes. As used herein, and unless otherwise specified, the term “central pain” refers to pain initiated by a primary lesion or dysfunction in the central nervous system.

As used herein, and unless otherwise specified, the term “hyperesthesia” refers to increased sensitivity to stimulation, excluding the special senses.

As used herein, and unless otherwise specified, the term “hyperpathia” refers to a painful syndrome characterized by an abnormally painful reaction to a stimulus, especially a repetitive stimulus, as well as an increased threshold. It may occur with allodynia, hyperesthesia, hyperalgesia, or dysesthesia.

As used herein, and unless otherwise specified, the term “dysesthesia” refers to an unpleasant abnormal sensation, whether spontaneous or evoked. In certain embodiments, dysesthesia include hyperalgesia and allodynia.

As used herein, and unless otherwise specified, the term “hyperalgesia” refers to an increased response to a stimulus that is normally painful. It reflects increased pain on suprathreshold stimulation.

As used herein, and unless otherwise specified, the term “allodynia” refers to pain due to a stimulus that does not normally provoke pain.

As used herein, and unless otherwise specified, the term “Diabetic Peripheral Neuropathic Pain” (DPNP), also called diabetic neuropathy, DN or diabetic peripheral neuropathy), refers to chronic pain caused by neuropathy associated with diabetes mellitus. The classic presentation of DPNP is pain or tingling in the feet that can be described not only as “burning” or “shooting” but also as severe aching pain. Less commonly, patients may describe the pain as itching, tearing, or like a toothache. The pain may be accompanied by allodynia and hyperalgesia and an absence of symptoms, such as numbness.

As used herein, and unless otherwise specified, the term “Post-Herpetic Neuralgia”, also called “Postherpetic Neuralgia (PHN)”, refers to a painful condition affecting nerve fibers and skin. Without being limited by a particular theory, it is a complication of shingles, a second outbreak of the varicella zoster virus (VZV), which initially causes chickenpox.

As used herein, and unless otherwise specified, the term “neuropathic cancer pain” refers to peripheral neuropathic pain as a result of cancer, and can be caused directly by infiltration or compression of a nerve by a tumor, or indirectly by cancer treatments such as radiation therapy and chemotherapy (chemotherapy-induced neuropathy).

As used herein, and unless otherwise specified, the term “HIV/AIDS peripheral neuropathy” or “HIV/AIDS related neuropathy” refers to peripheral neuropathy caused by HIV/AIDS, such as acute or chronic inflammatory demyelinating neuropathy (AIDP and CIDP, respectively), as well as peripheral neuropathy resulting as a side effect of drugs used to treat HIV/AIDS.

As used herein, and unless otherwise specified, the term “Phantom Limb Pain” refers to pain appearing to come from where an amputated limb used to be. Phantom limb pain can also occur in limbs following paralysis (e.g., following spinal cord injury). “Phantom Limb Pain” is usually chronic in nature.

As used herein, and unless otherwise specified, the term “Trigeminal Neuralgia (TN)” refers to a disorder of the fifth cranial (trigeminal) nerve that causes episodes of intense, stabbing, electric-shock-like pain in the areas of the face where the branches of the nerve are distributed (lips, eyes, nose, scalp, forehead, upper jaw, and lower jaw). It is also known as the “suicide disease”.

As used herein, and unless otherwise specified, the term “Complex Regional Pain Syndrome (CRPS),” formerly known as Reflex Sympathetic Dystrophy (RSD), refers to a chronic pain condition whose key symptom is continuous, intense pain out of proportion to the severity of the injury, which gets worse rather than better over time. The term encompasses type 1 CRPS, which includes conditions caused by tissue injury other than peripheral nerve, and type 2 CRPS, in which the syndrome is provoked by major nerve injury, and is sometimes called causalgia.

As used herein, and unless otherwise specified, the term “fibromyalgia” refers to a chronic condition characterized by diffuse or specific muscle, joint, or bone pain, along with fatigue and a range of other symptoms. Previously, fibromyalgia was known by other names such as fibrositis, chronic muscle pain syndrome, psychogenic rheumatism and tension myalgias.

As used herein, and unless otherwise specified, the term “convulsion” refers to a neurological disorder and is used interchangeably with “seizure,” although there are many types of seizure, some of which have subtle or mild symptoms instead of convulsions. Seizures of all types may be caused by disorganized and sudden electrical activity in the brain. In some embodiments, convulsions are a rapid and uncontrollable shaking during which the muscles contract and relax repeatedly.

### B. COMPOUNDS

In one embodiment, provided herein is a compound of formula (I):

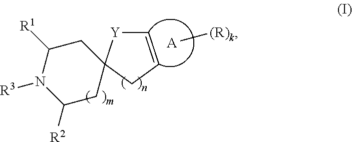

or a pharmaceutically acceptable salt or stereoisomer thereof, wherein

ring A is optionally substituted 5- or 6-membered aryl or heteroaryl;

Y is O, S, NH, or CH₂;

k is 0, 1, 2, 3, or 4;

m is 0, 1, 2, 3, or 4;

n is 0, 1, 2, 3, or 4;

R¹, R², and R³ are independently hydrogen, ═O, (C₁-C₁₀)alkyl, (C₂-C₁₀)alkenyl, (C₃-C₁₀)cycloalkyl, (C₆-C₁₀)aralkyl, (C₁-C₁₀)heteroalkyl, (3 to 10 membered) heterocyclyl, (6 to 10 membered)aryl, or (5 to 10 membered)heteroaryl, each of which may be optionally substituted with one or more R¹⁰; optionally R¹ and R², or R¹ and R³, or R² and R³ together with the atoms to which they are attached form an optionally substituted 3 to 10 membered cycloalkyl or heterocyclyl ring;

each occurrence of R is independently hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹⁰; optionally two adjacent R substituents together with the atoms to which they are attached form an optionally substituted 3 to 10 membered cycloalkyl, heterocyclyl, aryl, or heteroaryl ring;

each occurrence of R¹⁰ is independently a bond, hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered) heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹¹; optionally two germinal or vicinal R¹⁰ substituents together with the atom(s) to which they are attached form an optionally substituted 3 to 10 membered ring;

each occurrence of R¹¹ is independently hydrogen, halo, cyano, (C₁-C₁₀)alkyl optionally substituted with one or more R¹², (C₂-C₁₀)alkenyl optionally substituted with one or more R¹², (C₃-C₁₀)cycloalkyl optionally substituted with one or more R¹², (C₁-C₁₀)heteroalkyl optionally substituted with one or more R¹², (3 to 10 membered) heterocyclyl optionally substituted with one or more R¹², (C₆-C₁₂)aralkyl optionally substituted with one or more R¹², (6 to 10 membered)aryl optionally substituted with one or more R¹², (5 to 10 membered)heteroaryl optionally substituted with one or more R¹², ═O, —R¹³, —OR¹³, —NR¹³R¹⁴, —N(R¹³)C(O)R¹⁴, C(O)NR¹³R¹⁴, —C(O)R¹³, C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹¹ substituents together with the atom(s) to which they are attached form an optionally substituted 3 to 10 membered ring;

each occurrence of R¹² is independently hydrogen, halo, cyano, (C₁-C₆)alkyl optionally substituted with one or more R¹³, (C₂-C₆)alkenyl optionally substituted with one or more R¹³, (C₃-C₇)cycloalkyl optionally substituted with one or more R¹³, (3 to 8 membered)heterocyclyl optionally substituted with one or more R¹³, (6 to 10 membered)aryl optionally substituted with one or more R¹³, (5 to 10 membered) heteroaryl optionally substituted with one or more R¹³, ═O, —R¹³, —OR¹³, —NR¹³R¹⁴, —N(R¹³)C(O)R¹⁴, —C(O)NR¹³R¹⁴, K C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵, optionally two germinal or vicinal R¹² substituents together with the atom(s) to which they are attached form an optionally substituted 3 to 10 membered ring; and

R¹³, R¹⁴, and R¹⁵ are independently hydrogen, halo, cyano, (C₁-C₆)alkyl, (C₂-C₆)alkenyl, (C₃-C₇)cycloalkyl, (C₇-C₁₀)aralkyl; (C₁-C₆)heteroalkyl, (3 to 8 membered) heterocyclyl, (6 to 10 membered)aryl, or (5 to 10 membered)heteroaryl; optionally two germinal or vicinal R¹³ substituents together with the atom(s) to which they are attached form an optionally substituted 3 to 10 membered ring; optionally R¹³ and R¹⁴, or R¹⁴ and R¹⁵ together with the atom(s) to which they are attached form an optionally substituted 3 to 10 membered ring.

In one embodiment, provided herein is a compound of formula (I):

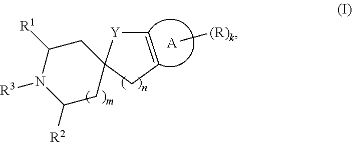

or a pharmaceutically acceptable salt or stereoisomer thereof, wherein

ring A is optionally substituted 5- or 6-membered aryl or heteroaryl;

Y is O, S, NH, or CH₂;

k is 0, 1, 2, 3, or 4;

m is 0, 1, 2, 3, or 4;

n is 1, 2, or 3;

(i) R¹ and R³ together with the atoms to which they are attached form a 3 to 10 membered heterocyclyl optionally substituted with one or more R¹⁰; and R² is hydrogen, ═O, (C₁-C₁₀)alkyl, (C₂-C₁₀)alkenyl, (C₃-C₁₀)cycloalkyl, (C₆-C₁₀)aralkyl, (C₁-C₁₀) heteroalkyl, (3 to 10 membered) heterocyclyl, (6 to 10 membered)aryl, or (5 to 10 membered)heteroaryl, each of which may be optionally substituted with one or more R¹⁰; or (ii) R² and R³ together with the atoms to which they are attached form a 3 to 10 membered heterocyclyl optionally substituted with one or more R¹⁰; and R¹ is hydrogen, ═O, (C₁-C₁₀)alkyl, (C₂-C₁₀)alkenyl, (C₃-C₁₀)cycloalkyl, (C₆-C₁₀)aralkyl, (C₁-C₁₀) heteroalkyl, (3 to 10 membered) heterocyclyl, (6 to 10 membered)aryl, or (5 to 10 membered)heteroaryl, each of which may be optionally substituted with one or more R¹⁰;

each occurrence of R is independently hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹⁰; optionally two adjacent R substituents together with the atoms to which they are attached form an optionally substituted 3 to 10 membered cycloalkyl, heterocyclyl, aryl, or heteroaryl ring;

each occurrence of R¹⁰ is independently a bond, hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered) heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹¹; optionally two germinal or vicinal R¹⁰ substituents together with the atom(s) to which they are attached form an optionally substituted 3 to 10 membered ring;

each occurrence of R¹¹ is independently hydrogen, halo, cyano, (C₁-C₁₀)alkyl optionally substituted with one or more R¹², (C₂-C₁₀)alkenyl optionally substituted with one or more R¹², (C₃-C₁₀)cycloalkyl optionally substituted with one or more R¹², (C₁-C₁₀)heteroalkyl optionally substituted with one or more R¹², (3 to 10 membered) heterocyclyl optionally substituted with one or more R¹², (C₆-C₁₂)aralkyl optionally substituted with one or more R¹², (6 to 10 membered)aryl optionally substituted with one or more R¹², (5 to 10 membered)heteroaryl optionally substituted with one or more R¹², ═O, —R¹³, —OR¹³, —NR¹³R¹⁴, —N(R¹³)C(O)R¹⁴, —C(O)NR¹³R¹⁴, —C(O)R¹³, C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹¹ substituents together with the atom(s) to which they are attached form an optionally substituted 3 to 10 membered ring;

each occurrence of R¹² is independently hydrogen, halo, cyano, (C₁-C₆)alkyl optionally substituted with one or more R¹³, (C₂-C₆)alkenyl optionally substituted with one or more R¹³, (C₃-C₇)cycloalkyl optionally substituted with one or more R¹³, (3 to 8 membered)heterocyclyl optionally substituted with one or more R¹³, (6 to 10 membered)aryl optionally substituted with one or more R¹³, (5 to 10 membered) heteroaryl optionally substituted with one or more R¹³, ═O, —R¹³, —OR¹³, —NR¹³R¹⁴, —N(R¹³)C(O)R¹⁴, —C(O)NR¹³R¹⁴, K C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹² substituents together with the atom(s) to which they are attached form an optionally substituted 3 to 10 membered ring; and

R¹³, R¹⁴, and R¹⁵ are independently hydrogen, halo, cyano, (C₁-C₆)alkyl, (C₂-C₆)alkenyl, (C₃-C₇)cycloalkyl, (C₇-C₁₀)aralkyl; (C₁-C₆)heteroalkyl, (3 to 8 membered) heterocyclyl, (6 to 10 membered)aryl, or (5 to 10 membered)heteroaryl; optionally two germinal or vicinal R¹³ substituents together with the atom(s) to which they are attached form an optionally substituted 3 to 10 membered ring; optionally R¹³ and R¹⁴, or R¹⁴ and R¹⁵ together with the atom(s) to which they are attached form an optionally substituted 3 to 10 membered ring.

In one embodiment, ring A is optionally substituted 6-membered aryl. In another embodiment, ring A is optionally substituted 5-membered heteroaryl. In another embodiment, ring A is optionally substituted 6-membered heteroaryl. Ring A is optionally substituted with one, two, three, or four R substituents.

In one embodiment, ring A is optionally substituted phenyl. In another embodiment, ring A is optionally substituted pyridyl. In another embodiment, ring A is optionally substituted pyrimidinyl. In another embodiment, ring A is optionally substituted pyrazinyl. In another embodiment, ring A is optionally substituted pyridazinyl. In another embodiment, ring A is optionally substituted pyridonyl. In another embodiment, ring A is optionally substituted furanyl. In another embodiment, ring A is optionally substituted thienyl. In another embodiment, ring A is optionally substituted pyrrolyl. In another embodiment, ring A is optionally substituted imidazolyl. In another embodiment, ring A is optionally substituted pyrazolyl. In another embodiment, ring A is optionally substituted oxazolyl. In another embodiment, ring A is optionally substituted thiazolyl.

In one embodiment, Y is O. In another embodiment, Y is S. In another embodiment, Y is NH. In another embodiment, Y is CH₂.

In one embodiment, k is 0. In another embodiment, k is 1. In another embodiment, k is 2. In another embodiment, k is 3. In another embodiment, k is 4.

In one embodiment, m is 0. In another embodiment, m is 1. In another embodiment, m is 2. In another embodiment, m is 3. In another embodiment, m is 4.

In one embodiment, n is 0. In another embodiment, n is 1. In another embodiment, n is 2. In another embodiment, n is 3. In another embodiment, n is 4. In one embodiment, n is 1, 2, or 3.

In one embodiment, R¹ is hydrogen. In another embodiment, R¹ is ═O. In another embodiment, R¹ is (C₁-C₁₀)alkyl optionally substituted with one or more R¹⁰. In another embodiment, R¹ is (C₂-C₁₀)alkenyl optionally substituted with one or more R¹⁰. In another embodiment, R¹ is (C₃-C₁₀)cycloalkyl optionally substituted with one or more R¹⁰. In another embodiment, R¹ is (C₆-C₁₀)aralkyl optionally substituted with one or more R¹⁰. In another embodiment, R¹ is (C₁-C₁₀)heteroalkyl optionally substituted with one or more R¹⁰. In another embodiment, R¹ is (3 to 10 membered)heterocyclyl optionally substituted with one or more R¹⁰. In another embodiment, R¹ is (6 to 10 membered)aryl optionally substituted with one or more R¹⁰. In another embodiment, R¹ is (5 to 10 membered)heteroaryl optionally substituted with one or more R¹⁰.

In one embodiment, R² is hydrogen. In another embodiment, R² is ═O. In another embodiment, R² is (C₁-C₁₀)alkyl optionally substituted with one or more R¹⁰. In another embodiment, R² is (C₂-C₁₀)alkenyl optionally substituted with one or more R¹⁰. In another embodiment, R² is (C₃-C₁₀)cycloalkyl optionally substituted with one or more R¹⁰. In another embodiment, R² is (C₆-C₁₀)aralkyl optionally substituted with one or more R¹⁰. In another embodiment, R² is (C₁-C₁₀)heteroalkyl optionally substituted with one or more R¹⁰. In another embodiment, R² is (3 to 10 membered)heterocyclyl optionally substituted with one or more R¹⁰. In another embodiment, R² is (6 to 10 membered)aryl optionally substituted with one or more R¹⁰. In another embodiment, R² is (5 to 10 membered)heteroaryl optionally substituted with one or more R¹⁰.

In one embodiment, R³ is hydrogen. In one embodiment, R³ is ═O. In another embodiment, R³ is (C₁-C₁₀)alkyl optionally substituted with one or more R¹⁰. In another embodiment, R³ is (C₂-C₁₀)alkenyl optionally substituted with one or more R¹⁰. In another embodiment, R³ is (C₃-C₁₀)cycloalkyl optionally substituted with one or more R¹⁰. In another embodiment, R³ is (C₆-C₁₀)aralkyl optionally substituted with one or more R¹⁰. In another embodiment, R³ is (C₁-C₁₀)heteroalkyl optionally substituted with one or more R¹⁰. In another embodiment, R³ is (3 to 10 membered)heterocyclyl optionally substituted with one or more R¹⁰. In another embodiment, R³ is (6 to 10 membered)aryl optionally substituted with one or more R¹⁰. In another embodiment, R³ is (5 to 10 membered) heteroaryl optionally substituted with one or more R¹⁰.

In one embodiment, R¹ and R² together with the atoms to which they are attached form an optionally substituted 3- to 10-membered cycloalkyl ring. In another embodiment, R¹ and R² together with the atoms to which they are attached form an optionally substituted 3- to 10-membered heterocyclyl ring.

In one embodiment, R¹ and R³ together with the atoms to which they are attached form an optionally substituted 3- to 10-membered cycloalkyl ring. In another embodiment, R¹ and R³ together with the atoms to which they are attached form an optionally substituted 3- to 10-membered heterocyclyl ring. In one embodiment, R¹ and R³ together with the atoms to which they are attached form a 3- to 10-membered heterocyclyl optionally substituted with one or more R¹⁰. In one embodiment, R¹ and R³ together with the atoms to which they are attached form a 4- to 7-membered heterocyclyl optionally substituted with one or more R¹⁰. In one embodiment, R¹ and R³ together with the atoms to which they are attached form a 5- to 6-membered heterocyclyl (e.g., a pyrrolidinyl, piperidinyl, morpholinyl, and piperazinyl ring) optionally substituted with one or more R¹⁰. In one embodiment, R¹ and R³ together with the atoms to which they are attached form a pyrrolidinyl ring optionally substituted with one or more R¹⁰. In one embodiment, R¹ and R³ together with the atoms to which they are attached form a piperidinyl ring optionally substituted with one or more R¹⁰.

In one embodiment, R² and R³ together with the atoms to which they are attached form an optionally substituted 3 to 10 membered cycloalkyl ring. In another embodiment, R² and R³ together with the atoms to which they are attached form an optionally substituted 3 to 10 membered heterocyclyl ring. In one embodiment, R¹ and R³ together with the atoms to which they are attached form a 3- to 10-membered heterocyclyl optionally substituted with one or more R¹⁰. In one embodiment, R² and R³ together with the atoms to which they are attached form a 4- to 7-membered heterocyclyl optionally substituted with one or more R¹⁰. In one embodiment, R² and R³ together with the atoms to which they are attached form a 5- to 6-membered heterocyclyl (e.g., a pyrrolidinyl, piperidinyl, morpholinyl, and piperazinyl ring) optionally substituted with one or more R¹⁰. In one embodiment, R² and R³ together with the atoms to which they are attached form a pyrrolidinyl ring optionally substituted with one or more R¹⁰. In one embodiment, R² and R³ together with the atoms to which they are attached form a piperidinyl ring optionally substituted with one or more R¹⁰.

In one embodiment, R³ is (C₁-C₆)alkyl optionally substituted with one or more R¹⁰. In another embodiment, R³ is (C₁-C₄)alkyl optionally substituted with one or more R¹⁰. In another embodiment, R³ is (C₃-C₆)alkyl optionally substituted with one or more R¹⁰. In another embodiment, R³ is (C₂-C₄)alkyl optionally substituted with one or more R¹⁰. In another embodiment, R³ is (C₁-C₂)alkyl optionally substituted with one or more R¹⁰. In another embodiment, R³ is (C₁-C₃)alkyl optionally substituted with one or more R¹⁰. In another embodiment, R³ is (C₁)alkyl optionally substituted with one, two, or three R¹⁰.

In one embodiment, R³ is (C₂-C₆)alkenyl optionally substituted with one or more R¹⁰. In another embodiment, R³ is (C₃-C₆)alkenyl optionally substituted with one or more R¹⁰. In another embodiment, R³ is (C₂-C₄)alkenyl optionally substituted with one or more R¹⁰.

In one embodiment, R³ is (C₃-C₇)cycloalkyl optionally substituted with one or more R¹⁰. In another embodiment, R³ is cyclopropyl optionally substituted with one or more R¹⁰. In another embodiment, R³ is cyclobutyl optionally substituted with one or more R¹⁰. In another embodiment, R³ is cyclopentyl optionally substituted with one or more R¹⁰. In another embodiment, R³ is cyclohexyl optionally substituted with one or more R¹⁰. In another embodiment, R³ is cycloheptyl optionally substituted with one or more R¹⁰. In one embodiment, R³ is (C₈)cycloalkyl optionally substituted with one or more R¹⁰. In one embodiment, R³ is (C₉)cycloalkyl optionally substituted with one or more R¹⁰. In one embodiment, R³ is (C₁₀)cycloalkyl optionally substituted with one or more R¹⁰.

In one embodiment, R³ is (C₆-C₈)aralkyl optionally substituted with one or more R¹⁰. In another embodiment, R³ is benzyl optionally substituted with one or more R¹⁰. In another embodiment, R³ is phenethyl optionally substituted with one or more R¹⁰.

In one embodiment, R³ is (C₁-C₆)heteroalkyl optionally substituted with one or more R¹⁰. In another embodiment, R³ is (C₁-C₄)heteroalkyl optionally substituted with one or more R¹⁰. In another embodiment, R³ is (C₃-C₆)heteroalkyl optionally substituted with one or more R¹⁰. In another embodiment, R³ is (C₂-C₄)heteroalkyl optionally substituted with one or more R¹⁰. In another embodiment, R³ is (C₁-C₂) heteroalkyl optionally substituted with one or more R¹⁰. In another embodiment, R³ is (C₁-C₃)heteroalkyl optionally substituted with one or more R¹⁰.

In one embodiment, R³ is (3 to 8 membered)heterocyclyl optionally substituted with one or more R¹⁰. In another embodiment, R³ is (3 to 6 membered) heterocyclyl optionally substituted with one or more R¹⁰. In another embodiment, R³ is (4 to 6 membered)heterocyclyl optionally substituted with one or more R¹⁰. In another embodiment, R³ is 3-membered heterocyclyl optionally substituted with one or more R¹⁰. In another embodiment, R³ is 4-membered heterocyclyl optionally substituted with one or more R¹⁰. In another embodiment, R³ is 5-membered heterocyclyl optionally substituted with one or more R¹⁰. In another embodiment, R³ is 6-membered heterocyclyl optionally substituted with one or more R¹⁰. In another embodiment, R³ is 7-membered heterocyclyl optionally substituted with one or more R¹⁰. In another embodiment, R³ is 8-membered heterocyclyl optionally substituted with one or more R¹⁰. In another embodiment, R³ is 9-membered heterocyclyl optionally substituted with one or more R¹⁰. In another embodiment, R³ is 10-membered heterocyclyl optionally substituted with one or more R¹⁰.

In one embodiment, R³ is 6-membered aryl optionally substituted with one or more R¹⁰. In another embodiment, R³ is 10-membered aryl optionally substituted with one or more R¹⁰.

In one embodiment, R³ is 5-membered heteroaryl optionally substituted with one or more R¹⁰. In another embodiment, R³ is 6-membered heteroaryl optionally substituted with one or more R¹⁰. In another embodiment, R³ is 9-membered heteroaryl optionally substituted with one or more R¹⁰. In another embodiment, R³ is 10-membered heteroaryl optionally substituted with one or more R¹⁰.

In one embodiment, R is hydrogen. In another embodiment, R is halo. In another embodiment, R is cyano. In another embodiment, R is (C₁-C₁₀)alkyl optionally substituted with one or more R¹⁰. In another embodiment, R is (C₂-C₁₀)alkenyl optionally substituted with one or more R¹⁰. In another embodiment, R is (C₃-C₁₀) cycloalkyl optionally substituted with one or more R¹⁰. In another embodiment, R is (C₁-C₁₀)heteroalkyl optionally substituted with one or more R¹⁰. In another embodiment, R is (3 to 10 membered)heterocyclyl optionally substituted with one or more R¹⁰. In another embodiment, R is (6 to 10 membered)aryl optionally substituted with one or more R¹⁰. In another embodiment, R is (5 to 10 membered)heteroaryl optionally substituted with one or more R¹⁰. In another embodiment, R is alkoxyl optionally substituted with one or more R¹⁰. In another embodiment, R is aminoalkyl optionally substituted with one or more R¹⁰. In another embodiment, R is hydroxyl optionally substituted with one or more R¹⁰. In another embodiment, R is amino optionally substituted with one or more R¹⁰. In another embodiment, R is imino optionally substituted with one or more R¹⁰. In another embodiment, R is amido optionally substituted with one or more R¹⁰. In another embodiment, R is carbonyl optionally substituted with one or more R¹⁰. In another embodiment, R is thiol optionally substituted with one or more R¹⁰. In another embodiment, R is sulfinyl optionally substituted with one or more R¹⁰. In another embodiment, R is sulfonyl optionally substituted with one or more R¹⁰.

In some embodiments, two adjacent R substituents together with the atoms to which they are attached form an optionally substituted 3 to 10 membered cycloalkyl. In other embodiments, two adjacent R substituents together with the atoms to which they are attached form an optionally substituted 3 to 10 membered heterocyclyl. In other embodiments, two adjacent R substituents together with the atoms to which they are attached form an optionally substituted 6 to 10 membered aryl. In other embodiments, two adjacent R substituents together with the atoms to which they are attached form an optionally substituted 5 to 10 membered heteroaryl.

In one embodiment, R is (C₁-C₆)alkyl optionally substituted with one or more R¹⁰. In another embodiment, R is (C₁-C₄)alkyl optionally substituted with one or more R¹⁰. In another embodiment, R is (C₃-C₆)alkyl optionally substituted with one or more R¹⁰. In another embodiment, R is (C₂-C₄)alkyl optionally substituted with one or more R¹⁰. In another embodiment, R is (C₁-C₂)alkyl optionally substituted with one or more R¹⁰. In another embodiment, R is (C₁-C₃)alkyl optionally substituted with one or more R¹⁰. In another embodiment, R is (C₁)alkyl optionally substituted with one, two, or three R¹⁰.

In one embodiment, R is (C₂-C₆)alkenyl optionally substituted with one or more R¹⁰. In another embodiment, R is (C₃-C₆)alkenyl optionally substituted with one or more R¹⁰. In another embodiment, R is (C₂-C₄)alkenyl optionally substituted with one or more R¹⁰.

In one embodiment, R is (C₃-C₇)cycloalkyl optionally substituted with one or more R¹⁰. In another embodiment, R is cyclopropyl optionally substituted with one or more R¹⁰. In another embodiment, R is cyclobutyl optionally substituted with one or more R¹⁰. In another embodiment, R is cyclopentyl optionally substituted with one or more R¹⁰. In another embodiment, R is cyclohexyl optionally substituted with one or more R¹⁰. In another embodiment, R is cycloheptyl optionally substituted with one or more R¹⁰. In one embodiment, R is (C₈)cycloalkyl optionally substituted with one or more R¹⁰. In one embodiment, R is (C₉)cycloalkyl optionally substituted with one or more R¹⁰. In one embodiment, R is (C₁₀)cycloalkyl optionally substituted with one or more R¹⁰.

In one embodiment, R is (C₁-C₆)heteroalkyl optionally substituted with one or more R¹⁰. In another embodiment, R is (C₁-C₄)heteroalkyl optionally substituted with one or more R¹⁰. In another embodiment, R is (C₃-C₆)heteroalkyl optionally substituted with one or more R¹⁰. In another embodiment, R is (C₂-C₄)heteroalkyl optionally substituted with one or more R¹⁰. In another embodiment, R is (C₁-C₂)heteroalkyl optionally substituted with one or more R¹⁰. In another embodiment, R is (C₁-C₃)heteroalkyl optionally substituted with one or more R¹⁰.

In one embodiment, R is (3 to 8 membered)heterocyclyl optionally substituted with one or more R¹⁰. In another embodiment, R is (3 to 6 membered) heterocyclyl optionally substituted with one or more R¹⁰. In another embodiment, R is (4 to 6 membered)heterocyclyl optionally substituted with one or more R¹⁰. In another embodiment, R is 3-membered heterocyclyl optionally substituted with one or more R¹⁰. In another embodiment, R is 4-membered heterocyclyl optionally substituted with one or more R¹⁰. In another embodiment, R is 5-membered heterocyclyl optionally substituted with one or more R¹⁰. In another embodiment, R is 6-membered heterocyclyl optionally substituted with one or more R¹⁰. In another embodiment, R is 7-membered heterocyclyl optionally substituted with one or more R¹⁰. In another embodiment, R is 8-membered heterocyclyl optionally substituted with one or more R¹⁰. In another embodiment, R is 9-membered heterocyclyl optionally substituted with one or more R¹⁰. In another embodiment, R is 10-membered heterocyclyl optionally substituted with one or more R¹⁰.

In one embodiment, R is 6-membered aryl optionally substituted with one or more R¹⁰. In another embodiment, R is 10-membered aryl optionally substituted with one or more R¹⁰.

In one embodiment, R is 5-membered heteroaryl optionally substituted with one or more R¹⁰. In another embodiment, R is 6-membered heteroaryl optionally substituted with one or more R¹⁰. In another embodiment, R is 9-membered heteroaryl optionally substituted with one or more R¹⁰. In another embodiment, R is 10-membered heteroaryl optionally substituted with one or more R¹⁰.

In one embodiment, R is —OR¹⁰. In another embodiment, R is —N(R¹⁰)₂. In another embodiment, R is —C(O)R¹⁰. In another embodiment, R is —C(O)N(R¹⁰)₂. In another embodiment, R is —NR¹⁰C(O)R¹⁰. In another embodiment, R is —SR¹⁰. In another embodiment, R is —S(O)R¹⁰. In another embodiment, R is —S(O)₂R¹⁰.

In one embodiment, R¹⁰ is a bond. In one embodiment, R¹⁰ is a bond substituted with R¹¹ (i.e., R¹⁰ is R¹¹). In another embodiment, R¹⁰ is hydrogen. In another embodiment, R¹⁰ is halo. In another embodiment, R¹⁰ is cyano. In another embodiment, R¹⁰ is (C₁-C₁₀)alkyl optionally substituted with one or more R¹¹. In another embodiment, R¹⁰ is (C₂-C₁₀)alkenyl optionally substituted with one or more R¹¹. In another embodiment, R¹⁰ is (C₃-C₁₀)cycloalkyl optionally substituted with one or more R¹¹. In another embodiment, R¹⁰ is (C₁-C₁₀)heteroalkyl optionally substituted with one or more R¹¹. In another embodiment, R¹⁰ is (3 to 10 membered)heterocyclyl optionally substituted with one or more R¹¹. In another embodiment, R¹⁰ is (6 to 10 membered)aryl optionally substituted with one or more R¹¹. In another embodiment, R¹⁰ is (5 to 10 membered)heteroaryl optionally substituted with one or more R¹¹. In another embodiment, R¹⁰ is alkoxyl optionally substituted with one or more R¹¹. In another embodiment, R¹⁰ is aminoalkyl optionally substituted with one or more R¹¹. In another embodiment, R¹⁰ is hydroxyl optionally substituted with one or more R¹¹. In another embodiment, R¹⁰ is amino optionally substituted with one or more R¹¹. In another embodiment, R¹⁰ is imino optionally substituted with one or more R¹¹. In another embodiment, R¹⁰ is amido optionally substituted with one or more R¹¹. In another embodiment, R¹⁰ is carbonyl optionally substituted with one or more R¹¹. In another embodiment, R¹⁰ is thiol optionally substituted with one or more R¹¹. In another embodiment, R¹⁰ is sulfinyl optionally substituted with one or more R¹¹. In another embodiment, R¹⁰ is sulfonyl optionally substituted with one or more R¹¹.

In some embodiments, two germinal or vicinal R¹⁰ substituents together with the atom(s) to which they are attached form an optionally substituted 3 to 10 membered ring, including, but not limited to, cycloalkyl, heterocyclyl, aryl, and heteroaryl rings.

In one embodiment, R¹⁰ is (C₁-C₆)alkyl optionally substituted with one or more R¹¹. In another embodiment, R¹⁰ is (C₁-C₄)alkyl optionally substituted with one or more R¹¹. In another embodiment, R¹⁰ is (C₃-C₆)alkyl optionally substituted with one or more R¹¹. In another embodiment, R¹⁰ is (C₂-C₄)alkyl optionally substituted with one or more R¹¹. In another embodiment, R¹⁰ is (C₁-C₂)alkyl optionally substituted with one or more R¹¹. In another embodiment, R¹⁰ is (C₁-C₃)alkyl optionally substituted with one or more R¹¹. In another embodiment, R¹⁰ is (C₁)alkyl optionally substituted with one, two, or three R¹¹.

In one embodiment, R¹⁰ is (C₂-C₆)alkenyl optionally substituted with one or more R¹¹. In another embodiment, R¹⁰ is (C₃-C₆)alkenyl optionally substituted with one or more R¹¹. In another embodiment, R¹⁰ is (C₂-C₄)alkenyl optionally substituted with one or more R¹¹.

In one embodiment, R¹⁰ is (C₃-C₇)cycloalkyl optionally substituted with one or more R¹¹. In another embodiment, R¹⁰ is cyclopropyl optionally substituted with one or more R¹¹. In another embodiment, R¹⁰ is cyclobutyl optionally substituted with one or more R¹¹. In another embodiment, R¹⁰ is cyclopentyl optionally substituted with one or more R¹¹. In another embodiment, R¹⁰ is cyclohexyl optionally substituted with one or more R¹¹. In another embodiment, R¹⁰ is cycloheptyl optionally substituted with one or more R¹¹. In one embodiment, R¹⁰ is (C₈)cycloalkyl optionally substituted with one or more R¹¹. In one embodiment, R¹⁰ is (C₉)cycloalkyl optionally substituted with one or more R¹¹. In one embodiment, R¹⁰ is (C₁₀)cycloalkyl optionally substituted with one or more R¹¹.

In one embodiment, R¹⁰ is (C₁-C₆)heteroalkyl optionally substituted with one or more R¹¹. In another embodiment, R¹⁰ is (C₁-C₄)heteroalkyl optionally substituted with one or more R¹¹. In another embodiment, R¹⁰ is (C₃-C₆)heteroalkyl optionally substituted with one or more R¹¹. In another embodiment, R¹⁰ is (C₂-C₄)heteroalkyl optionally substituted with one or more R¹¹. In another embodiment, R¹⁰ is (C₁-C₂) heteroalkyl optionally substituted with one or more R¹¹. In another embodiment, R¹⁰ is (C₁-C₃)heteroalkyl optionally substituted with one or more R¹¹.

In one embodiment, R¹⁰ is (3 to 8 membered)heterocyclyl optionally substituted with one or more R¹¹. In another embodiment, R¹⁰ is (3 to 6 membered) heterocyclyl optionally substituted with one or more R¹¹. In another embodiment, R¹⁰ is (4 to 6 membered)heterocyclyl optionally substituted with one or more R¹¹. In another embodiment, R¹⁰ is 3-membered heterocyclyl optionally substituted with one or more R¹¹. In another embodiment, R¹⁰ is 4-membered heterocyclyl optionally substituted with one or more R¹¹. In another embodiment, R¹⁰ is 5-membered heterocyclyl optionally substituted with one or more R¹¹. In another embodiment, R¹⁰ is 6-membered heterocyclyl optionally substituted with one or more R¹¹. In another embodiment, R¹⁰ is 7-membered heterocyclyl optionally substituted with one or more R¹¹. In another embodiment, R¹⁰ is 8-membered heterocyclyl optionally substituted with one or more R¹¹. In another embodiment, R¹⁰ is 9-membered heterocyclyl optionally substituted with one or more R¹¹. In another embodiment, R¹⁰ is 10-membered heterocyclyl optionally substituted with one or more R¹¹.

In one embodiment, R¹⁰ is 6-membered aryl optionally substituted with one or more R¹¹. In another embodiment, R¹⁰ is 10-membered aryl optionally substituted with one or more R¹¹.

In one embodiment, R¹⁰ is 5-membered heteroaryl optionally substituted with one or more R¹¹. In another embodiment, R¹⁰ is 6-membered heteroaryl optionally substituted with one or more R¹¹. In another embodiment, R¹⁰ is 9-membered heteroaryl optionally substituted with one or more R¹¹. In another embodiment, R¹⁰ is 10-membered heteroaryl optionally substituted with one or more R¹¹.

In one embodiment, R¹⁰ is —OR¹¹. In another embodiment, R¹⁰ is —N(R¹¹)₂. In another embodiment, R¹⁰ is C(O)R¹¹. In another embodiment, R¹⁰ is —C(O)N(R¹¹)₂. In another embodiment, R¹⁰ is —NR¹¹C(O)R¹¹. In another embodiment, R¹⁰ is —SR¹¹. In another embodiment, R¹⁰ is —S(O)R¹¹. In another embodiment, R¹⁰ is —S(O)₂R¹¹.

In one embodiment, R¹¹ is hydrogen. In another embodiment, R¹¹ is halo. In another embodiment, R¹¹ is cyano. In another embodiment, R¹¹ is (C₁-C₁₀)alkyl optionally substituted with one or more R¹². In another embodiment, R¹¹ is (C₂-C₁₀)alkenyl optionally substituted with one or more R¹². In another embodiment, R¹¹ is (C₃-C₁₀)cycloalkyl optionally substituted with one or more R¹². In another embodiment, R¹¹ is (C₁-C₁₀)heteroalkyl optionally substituted with one or more R¹². In another embodiment, R¹¹ is (3 to 10 membered)heterocyclyl optionally substituted with one or more R¹². In another embodiment, R¹¹ is (C₆-C₁₂)aralkyl optionally substituted with one or more R¹². In another embodiment, R¹¹ is (6 to 10 membered)aryl optionally substituted with one or more R¹². In another embodiment, R¹¹ is (5 to 10 membered) heteroaryl optionally substituted with one or more R¹². In another embodiment, R¹¹ is ═O. In another embodiment, R¹¹ is —R¹³. In another embodiment, R¹¹ is —OR¹³. In another embodiment, R¹¹ is —NR¹³R¹⁴. In another embodiment, R¹¹ is —N(R¹³)C(O)R¹⁴ In another embodiment, R¹¹ is —C(O)NR¹³R¹⁴. In another embodiment, R¹¹ is —C(O)R¹³. In another embodiment, R¹¹ is —C(O)OR¹³. In another embodiment, R¹¹ is —OC(O)R¹³. In another embodiment, R¹¹ is —OC(O)NR¹³R¹⁴. In another embodiment, R¹¹ is —NR¹³C(O)OR¹⁴. In another embodiment, R¹¹ is —SR¹³. In another embodiment, R¹¹ is —S(O)R¹³. In another embodiment, R¹¹ is —S(O)₂R¹³. In another embodiment, R¹¹ is —S(O)₂NR¹³R¹⁴. In another embodiment, R¹¹ is —NR¹³S(O)₂R¹⁴. In another embodiment, R¹¹ is —NR¹³C(O)NR¹⁴R¹⁵.

In some embodiments, two germinal or vicinal R¹¹ substituents together with the atom(s) to which they are attached form an optionally substituted 3 to 10 membered ring, including, but not limited to, cycloalkyl, heterocyclyl, aryl, and heteroaryl rings.

In one embodiment, R¹¹ is (C₁-C₆)alkyl optionally substituted with one or more R¹². In another embodiment, R¹¹ is (C₁-C₄)alkyl optionally substituted with one or more R¹². In another embodiment, R¹¹ is (C₃-C₆)alkyl optionally substituted with one or more R¹². In another embodiment, R¹¹ is (C₂-C₄)alkyl optionally substituted with one or more R¹². In another embodiment, R¹¹ is (C₁-C₂)alkyl optionally substituted with one or more R¹². In another embodiment, R¹¹ is (C₁-C₃)alkyl optionally substituted with one or more R¹². In another embodiment, R¹¹ is (C₁)alkyl optionally substituted with one, two, or three R¹².

In one embodiment, R¹¹ is (C₂-C₆)alkenyl optionally substituted with one or more R¹². In another embodiment, R¹¹ is (C₃-C₆)alkenyl optionally substituted with one or more R¹². In another embodiment, R¹¹ is (C₂-C₄)alkenyl optionally substituted with one or more R¹².

In one embodiment, R¹¹ is (C₃-C₇)cycloalkyl optionally substituted with one or more R¹². In another embodiment, R¹¹ is cyclopropyl optionally substituted with one or more R¹². In another embodiment, R¹¹ is cyclobutyl optionally substituted with one or more R¹². In another embodiment, R¹¹ is cyclopentyl optionally substituted with one or more R¹². In another embodiment, R¹¹ is cyclohexyl optionally substituted with one or more R¹². In another embodiment, R¹¹ is cycloheptyl optionally substituted with one or more R¹². In one embodiment, R¹¹ is (C₈)cycloalkyl optionally substituted with one or more R¹². In one embodiment, R¹¹ is (C₉)cycloalkyl optionally substituted with one or more R¹². In one embodiment, R¹¹ is (C₁₀)cycloalkyl optionally substituted with one or more R¹².

In one embodiment, R¹¹ is (C₁-C₆)heteroalkyl optionally substituted with one or more R¹². In another embodiment, R¹¹ is (C₁-C₄)heteroalkyl optionally substituted with one or more R¹². In another embodiment, R¹¹ is (C₃-C₆)heteroalkyl optionally substituted with one or more R¹². In another embodiment, R¹¹ is (C₂-C₄)heteroalkyl optionally substituted with one or more R¹². In another embodiment, R¹¹ is (C₁-C₂) heteroalkyl optionally substituted with one or more R¹². In another embodiment, R¹¹ is (C₁-C₃)heteroalkyl optionally substituted with one or more R¹².

In one embodiment, R¹¹ is (3 to 8 membered)heterocyclyl optionally substituted with one or more R¹². In another embodiment, R¹¹ is (3 to 6 membered) heterocyclyl optionally substituted with one or more R¹². In another embodiment, R¹¹ is (4 to 6 membered)heterocyclyl optionally substituted with one or more R¹². In another embodiment, R¹¹ is 3-membered heterocyclyl optionally substituted with one or more R¹². In another embodiment, R¹¹ is 4-membered heterocyclyl optionally substituted with one or more R¹². In another embodiment, R¹¹ is 5-membered heterocyclyl optionally substituted with one or more R¹². In another embodiment, R¹¹ is 6-membered heterocyclyl optionally substituted with one or more R¹². In another embodiment, R¹¹ is 7-membered heterocyclyl optionally substituted with one or more R¹². In another embodiment, R¹¹ is 8-membered heterocyclyl optionally substituted with one or more R¹². In another embodiment, R¹¹ is 9-membered heterocyclyl optionally substituted with one or more R¹². In another embodiment, R¹¹ is 10-membered heterocyclyl optionally substituted with one or more R¹².

In one embodiment, R¹¹ is (C₆-C₁₀)aralkyl optionally substituted with one or more R¹². In another embodiment, R¹¹ is (C₆-C₈)aralkyl optionally substituted with one or more R¹². In another embodiment, R¹¹ is benzyl optionally substituted with one or more R¹². In another embodiment, R¹¹ is phenethyl optionally substituted with one or more R¹².

In one embodiment, R¹¹ is 6-membered aryl optionally substituted with one or more R¹². In another embodiment, R¹¹ is 10-membered aryl optionally substituted with one or more R¹².

In one embodiment, R¹¹ is 5-membered heteroaryl optionally substituted with one or more R¹². In another embodiment, R¹¹ is 6-membered heteroaryl optionally substituted with one or more R¹². In another embodiment, R¹¹ is 9-membered heteroaryl optionally substituted with one or more R¹². In another embodiment, R¹¹ is 10-membered heteroaryl optionally substituted with one or more R¹².

In one embodiment, R¹² is hydrogen. In another embodiment, R¹² is halo. In another embodiment, R¹² is cyano. In another embodiment, R¹² is (C₁-C₆)alkyl optionally substituted with one or more R¹³. In another embodiment, R¹² is (C₂-C₆)alkenyl optionally substituted with one or more R¹³. In another embodiment, R¹² is (C₃-C₇)cycloalkyl optionally substituted with one or more R¹³. In another embodiment, R¹² is (3 to 8 membered)heterocyclyl optionally substituted with one or more R¹³. In another embodiment, R¹² is (6 to 10 membered)aryl optionally substituted with one or more R¹³. In another embodiment, R¹² is (5 to 10 membered)heteroaryl optionally substituted with one or more R¹³. In another embodiment, R¹² is ═O. In another embodiment, R¹² is —R¹³. In another embodiment, R¹² is —OR¹³. In another embodiment, R¹² is —NR¹³R¹⁴. In another embodiment, R¹² is —N(R¹³)C(O)R¹⁴. In another embodiment, R¹² is —C(O)NR¹³R¹⁴. In another embodiment, R¹² is —C(O)R¹³. In another embodiment, R¹² is —C(O)OR¹³. In another embodiment, R¹² is —OC(O)R¹³. In another embodiment, R¹² is —OC(O)NR¹³R¹⁴. In another embodiment, R¹² is —NR¹³C(O)OR¹⁴. In another embodiment, R¹² is —SR¹³. In another embodiment, R¹² is —S(O)R¹³. In another embodiment, R¹² is —S(O)₂R¹³. In another embodiment, R¹² is —S(O)₂NR¹³R¹⁴. In another embodiment, R¹² is —NR¹³S(O)₂R¹⁴. In another embodiment, R¹² is —NR¹³C(O)NR¹⁴R¹⁵.

In some embodiments, two germinal or vicinal R¹² substituents together with the atom(s) to which they are attached form an optionally substituted 3 to 10 membered ring, including, but not limited to, cycloalkyl, heterocyclyl, aryl, and heteroaryl rings.

In one embodiment, R¹² is (C₁-C₄)alkyl optionally substituted with one or more R¹³. In another embodiment, R¹² is (C₃-C₆)alkyl optionally substituted with one or more R¹³. In another embodiment, R¹² is (C₂-C₄)alkyl optionally substituted with one or more R¹³. In another embodiment, R¹² is (C₁-C₂)alkyl optionally substituted with one or more R¹³. In another embodiment, R¹² is (C₁-C₃)alkyl optionally substituted with one or more R¹³. In another embodiment, R¹² is (C₁)alkyl optionally substituted with one, two, or three R¹³.

In one embodiment, R¹² is (C₃-C₆)alkenyl optionally substituted with one or more R¹³. In another embodiment, R¹² is (C₂-C₄)alkenyl optionally substituted with one or more R¹³.

In one embodiment, R¹² is cyclopropyl optionally substituted with one or more R¹³. In another embodiment, R¹² is cyclobutyl optionally substituted with one or more R¹³. In another embodiment, R¹² is cyclopentyl optionally substituted with one or more R¹³. In another embodiment, R¹² is cyclohexyl optionally substituted with one or more R¹³. In another embodiment, R¹² is cycloheptyl optionally substituted with one or more R¹³.

In one embodiment, R¹² is (3 to 6 membered)heterocyclyl optionally substituted with one or more R¹³. In another embodiment, R¹² is (4 to 6 membered) heterocyclyl optionally substituted with one or more R¹³. In another embodiment, R¹² is 3-membered heterocyclyl optionally substituted with one or more R¹³. In another embodiment, R¹² is 4-membered heterocyclyl optionally substituted with one or more R¹³. In another embodiment, R¹² is 5-membered heterocyclyl optionally substituted with one or more R¹³. In another embodiment, R¹² is 6-membered heterocyclyl optionally substituted with one or more R¹³. In another embodiment, R¹² is 7-membered heterocyclyl optionally substituted with one or more R¹³. In another embodiment, R¹² is 8-membered heterocyclyl optionally substituted with one or more R¹³.

In one embodiment, R¹² is 6-membered aryl optionally substituted with one or more R¹³. In another embodiment, R¹² is 10-membered aryl optionally substituted with one or more R¹³.

In one embodiment, R¹² is 5-membered heteroaryl optionally substituted with one or more R¹³. In another embodiment, R¹² is 6-membered heteroaryl optionally substituted with one or more R¹³. In another embodiment, R¹² is 9-membered heteroaryl optionally substituted with one or more R¹³. In another embodiment, R¹² is 10-membered heteroaryl optionally substituted with one or more R¹³.

In one embodiment, R¹³ is hydrogen. In another embodiment, R¹³ is halo. In another embodiment, R¹³ is cyano. In another embodiment, R¹³ is (C₁-C₆)alkyl. In another embodiment, R¹³ is (C₂-C₆)alkenyl. In another embodiment, R¹³ is (C₃-C₇)cycloalkyl. In another embodiment, R¹³ is (C₇-C₁₀)aralkyl. In another embodiment, R¹³ is (C₁-C₆)heteroalkyl. In another embodiment, R¹³ is (3 to 8 membered) heterocyclyl. In another embodiment, R¹³ is (6 to 10 membered)aryl. In another embodiment, R¹³ is (5 to 10 membered)heteroaryl.

In one embodiment, R¹⁴ is hydrogen. In another embodiment, R¹⁴ is halo. In another embodiment, R¹⁴ is cyano. In another embodiment, R¹⁴ is (C₁-C₆)alkyl. In another embodiment, R¹⁴ is (C₂-C₆)alkenyl. In another embodiment, R¹⁴ is (C₃-C₇)cycloalkyl. In another embodiment, R¹⁴ is (C₇-C₁₀)aralkyl. In another embodiment, R¹⁴ is (C₁-C₆)heteroalkyl. In another embodiment, R¹⁴ is (3 to 8 membered) heterocyclyl. In another embodiment, R¹⁴ is (6 to 10 membered)aryl. In another embodiment, R¹⁴ is (5 to 10 membered)heteroaryl.

In one embodiment, R¹⁵ is hydrogen. In another embodiment, R¹⁵ is halo. In another embodiment, R¹⁵ is cyano. In another embodiment, R¹⁵ is (C₁-C₆)alkyl. In another embodiment, R¹⁵ is (C₂-C₆)alkenyl. In another embodiment, R¹⁵ is (C₃-C₇)cycloalkyl. In another embodiment, R¹⁵ is (C₇-C₁₀)aralkyl. In another embodiment, R¹⁵ is (C₁-C₆)heteroalkyl. In another embodiment, R¹⁵ is (3 to 8 membered) heterocyclyl. In another embodiment, R¹⁵ is (6 to 10 membered)aryl. In another embodiment, R¹⁵ is (5 to 10 membered)heteroaryl.

In some embodiments, two germinal or vicinal R¹³ substituents together with the atom(s) to which they are attached form an optionally substituted 3 to 10 membered ring. In other embodiments, R¹³ and R¹⁴ together with the atom(s) to which they are attached form an optionally substituted 3 to 10 membered ring. In other embodiments, R¹⁴ and R¹⁵ together with the atom(s) to which they are attached form an optionally substituted 3 to 10 membered ring. The 3 to 10 membered ring includes, but is not limited to, cycloalkyl, heterocyclyl, aryl, and heteroaryl rings.

In one embodiment, provided herein is a compound of formula (II):

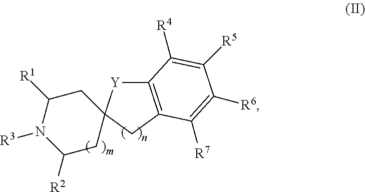

or a pharmaceutically acceptable salt or stereoisomer thereof, wherein

R⁴, R⁵, R⁶, and R⁷ are each independently R; optionally R⁴ and R⁵, or R⁵ and R⁶, or R⁶ and R⁷ together with the atoms to which they are attached form an optionally substituted 3 to 10 membered cycloalkyl, heterocyclyl, aryl, or heteroaryl ring; and

R¹, R², R³, R, Y, m, and n are defined herein elsewhere.

In one embodiment, R⁴, R⁵, and R⁷ are hydrogen, and R⁶ is as defined herein elsewhere. In one embodiment, R⁴, R⁶, and R⁷ are hydrogen, and R⁵ is as defined herein elsewhere. In one embodiment, R⁴ and R⁷ are hydrogen, and R⁵ and R⁶ are as defined herein elsewhere.

In one embodiment, provided herein is a compound of formula (III):

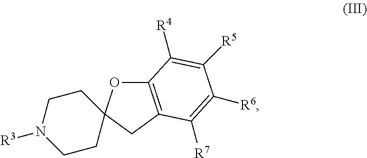

or a pharmaceutically acceptable salt or stereoisomer thereof, wherein

R³, R⁴, R⁵, R⁶, and R⁷ are defined herein elsewhere.

In one embodiment, R³ is hydrogen. In another embodiment, R³ is (C₃-C₇)cycloalkyl optionally substituted with one or more R¹⁰. In another embodiment, R³ is (3 to 8 membered)heterocyclyl optionally substituted with one or more R¹⁰. In another embodiment, R³ is —CR¹⁶R¹⁷R¹⁸, wherein R¹⁶, R¹⁷, and R¹⁸ are independently R¹⁰.

In one embodiment, R³ is (C₃-C₇)cycloalkyl optionally substituted with one or more R¹⁰; R⁴ and R⁷ are hydrogen; and R⁵ and R⁶ are independently hydrogen, halo, cyano, (C₁-C₆)alkyl optionally substituted with one or more R¹⁰, (C₃-C₇)cycloalkyl optionally substituted with one or more R¹⁰, (3 to 10 membered)heterocyclyl optionally substituted with one or more R¹⁰, (6 to 10 membered)aryl optionally substituted with one or more R¹⁰, (5 to 10 membered)heteroaryl optionally substituted with one or more R¹⁰, —OR¹⁰, —N(R¹⁰)₂, —C(O)R¹⁰, NR¹⁰C(O)R¹⁰, or —C(O)N(R¹⁰)₂; wherein R¹⁰ is defined herein elsewhere.

In one embodiment, R³ is optionally substituted cyclobutyl. In another embodiment, R³ is optionally substituted cyclopentyl. In another embodiment, R³ is optionally substituted cyclohexyl. In another embodiment, R³ is unsubstituted cyclobutyl. In another embodiment, R³ is unsubstituted cyclopentyl. In another embodiment, R³ is unsubstituted cyclohexyl. In another embodiment, R³ is cyclobutyl, cyclopentyl, or cyclohexyl, each of which is optionally substituted with one or more hydrogen, halo, cyano, or (C₁-C₄)alkyl optionally substituted with one or more halo.

In one embodiment, R⁴ and R⁷ are hydrogen; and R⁵ and R⁶ are each independently hydrogen, halo, cyano, (C₁-C₆)alkyl optionally substituted with one or more R¹⁰, (C₃-C₇)cycloalkyl optionally substituted with one or more R¹⁰, (3 to 10 membered)heterocyclyl optionally substituted with one or more R¹⁰, (6 to 10 membered) aryl optionally substituted with one or more R¹⁰, (5 to 10 membered)heteroaryl optionally substituted with one or more R¹⁰, —OR¹⁰, —N(R¹⁰)₂, —C(O)R¹⁰, —NR¹⁰C(O)R¹⁰, or —C(O)N(R¹⁰)₂.

In one embodiment, R⁴ and R⁷ is hydrogen; one of R⁵ and R⁶ is hydrogen, and the other is hydrogen, halo, cyano, (C₁-C₆)alkyl optionally substituted with one or more R¹⁰, (C₃-C₇)cycloalkyl optionally substituted with one or more R¹⁰, (3 to 10 membered)heterocyclyl optionally substituted with one or more R¹⁰, (6 to 10 membered)aryl optionally substituted with one or more R¹⁰, (5 to 10 membered) heteroaryl optionally substituted with one or more R¹⁰, —OR¹⁰, —N(R¹⁰)₂, —C(O)R¹⁰, —NR¹⁰C(O)R¹⁰, —NR¹⁰C(O)R¹⁰, or —C(O)N(R¹⁰)₂; wherein R¹⁰ is defined herein elsewhere. In one embodiment, R⁵ is hydrogen, and R⁶ is defined herein elsewhere. In one embodiment, R⁶ is hydrogen, and R⁵ is defined herein elsewhere.

In one embodiment, R⁴, R⁵, and R⁷ are hydrogen; and R⁶ is hydrogen, halo, optionally substituted 9-membered heteroaryl, optionally substituted phenyl, optionally substituted pyrazolyl, optionally substituted imidazolyl, optionally substituted pyrimidinyl, optionally substituted pyrazinyl, optionally substituted pyridyl, optionally substituted indolyl, optionally substituted benzimidazolyl, optionally substituted imidazopyridinyl, optionally substituted piperidinyl, optionally substituted piperazinyl, or —CH₂R¹⁰. In one embodiment, R⁴, R⁵, and R⁷ are hydrogen; and R⁶ is hydrogen, halo, optionally substituted phenyl, optionally substituted pyrazolyl, optionally substituted imidazolyl, optionally substituted pyrimidinyl, optionally substituted pyrazinyl, optionally substituted pyridyl, optionally substituted indolyl, optionally substituted benzimidazolyl, optionally substituted imidazopyridinyl, optionally substituted piperidinyl, optionally substituted piperazinyl, or —CH₂R¹⁰. In one embodiment, R⁴, R⁵, and R⁷ are hydrogen; and R⁶ is (5 to 10 membered) heteroaryl optionally substituted with one or more R¹⁰. In one embodiment, R⁴, R⁵, and R⁷ are hydrogen; and R⁶ is 9-membered heteroaryl optionally substituted with one or more R¹⁰. In one embodiment, each occurrence of R¹⁰ is hydrogen, halo, cyano, optionally substituted with (C₁-C₄)alkyl, optionally substituted aryl, optionally substituted heteroaryl, optionally substituted heterocyclyl, optionally substituted amino, optionally substituted amido, or optionally substituted alkoxyl. In some embodiment, R¹⁰ is amino optionally substituted with (C₁-C₄)alkyl. In one embodiment, R¹⁰ is dimethylamino. In some embodiments, R¹⁰ is optionally substituted heterocyclyl, including, but not limited to, pyrrolidinyl and morpholinyl. In some embodiments, R¹⁰ is amino optionally substituted with one or more optionally substituted aryl. In one embodiment, R¹⁰ is amino optionally substituted with 4-cyanophenyl. In some embodiments, R⁶ is phenyl optionally substituted with one or more R¹⁰. In some embodiments, R¹⁰ is halo, cyano, optionally substituted amido, optionally substituted aminoalkyl, or optionally substituted (C₁-C₄)alkyl. In one embodiment, R¹⁰ is aminomethyl. In one embodiment, R¹⁰ is acetamide. In some embodiments, R⁶ is phenyl optionally substituted with one or more cyano. In some embodiments, R⁶ is pyrimidinyl optionally substituted with one or more alkoxyl. In one embodiment, R⁶ is pyrimidinyl optionally substituted with one or more methoxy. In some embodiments, R⁶ is piperazinyl optionally substituted with (C₁-C₄)alkyl.

In one embodiment, R⁴, R⁶, and R⁷ are hydrogen; and R⁵ is hydrogen, halo, optionally substituted 9-membered heteroaryl, optionally substituted phenyl, optionally substituted pyrimidinyl, optionally substituted pyrazinyl, optionally substituted pyridyl, optionally substituted piperidinyl, optionally substituted piperazinyl, or —CH₂R¹⁰. In one embodiment, R⁴, R⁶, and R⁷ are hydrogen; and R⁵ is hydrogen, halo, optionally substituted phenyl, optionally substituted pyrimidinyl, optionally substituted pyrazinyl, optionally substituted pyridyl, optionally substituted piperidinyl, optionally substituted piperazinyl, or —CH₂R¹⁰. In some embodiment, R¹⁰ is amino optionally substituted with (C₁-C₄)alkyl. In one embodiment, R¹⁰ is dimethylamino. In some embodiments, R¹⁰ is optionally substituted heterocyclyl, including, but not limited to, pyrrolidinyl and morpholinyl. In some embodiments, R¹⁰ is amino optionally substituted with one or more optionally substituted aryl. In one embodiment, R¹⁰ is amino optionally substituted with 4-cyanophenyl. In some embodiments, R⁵ is phenyl optionally substituted with one or more R¹⁰. In some embodiments, R¹⁰ is halo, cyano, optionally substituted amido, optionally substituted aminoalkyl, or optionally substituted (C₁-C₄)alkyl. In one embodiment, R¹⁰ is aminomethyl. In one embodiment, R¹⁰ is acetamide. In some embodiments, R⁵ is phenyl optionally substituted with one or more cyano. In some embodiments, R⁵ is pyrimidinyl optionally substituted with one or more alkoxyl. In one embodiment, R⁵ is pyrimidinyl optionally substituted with one or more methoxy. In some embodiments, R⁵ is piperazinyl optionally substituted with (C₁-C₄)alkyl.

In one embodiment, specific examples include, but are not limited to, the following compounds:

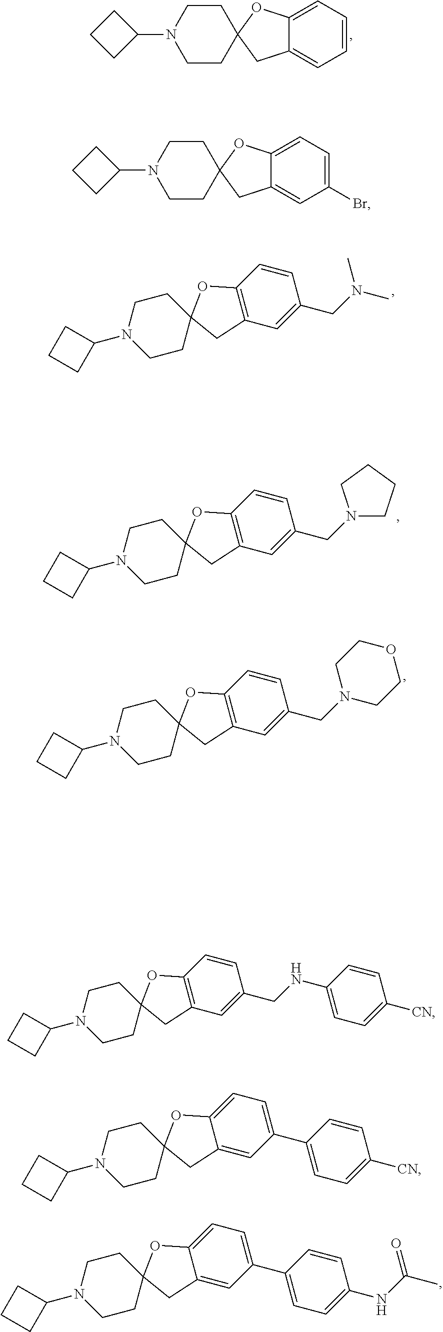

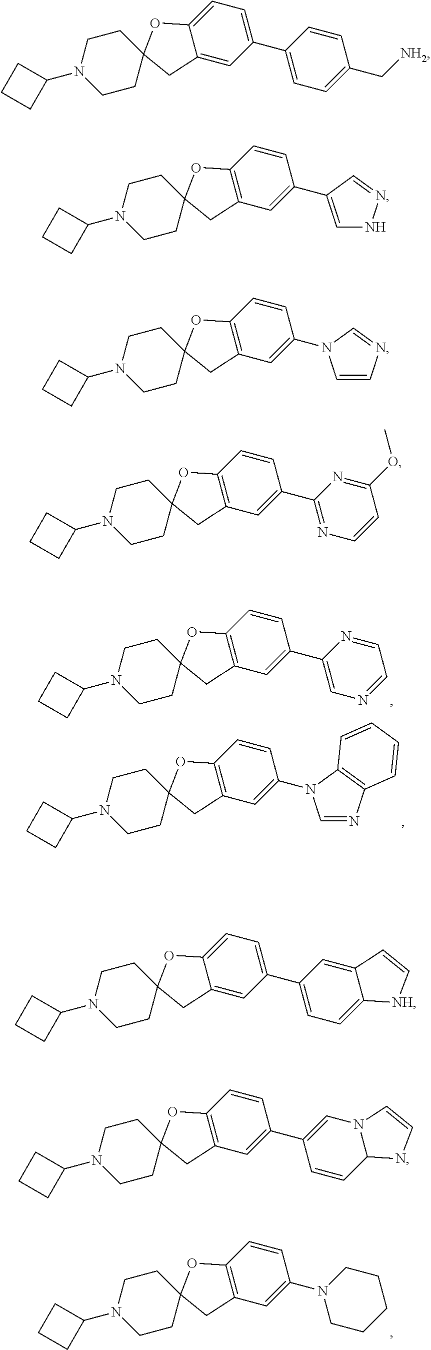

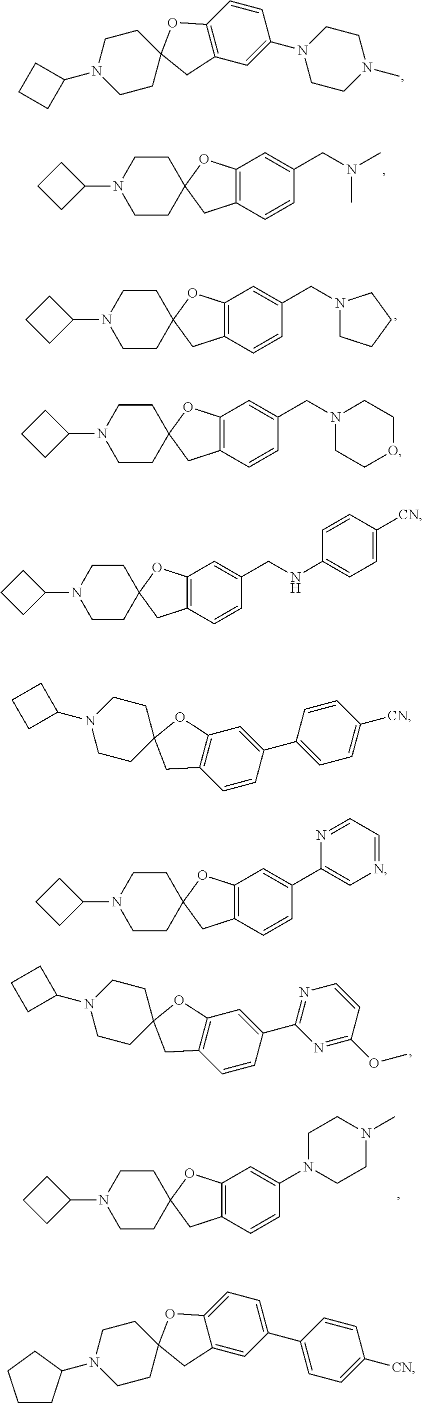

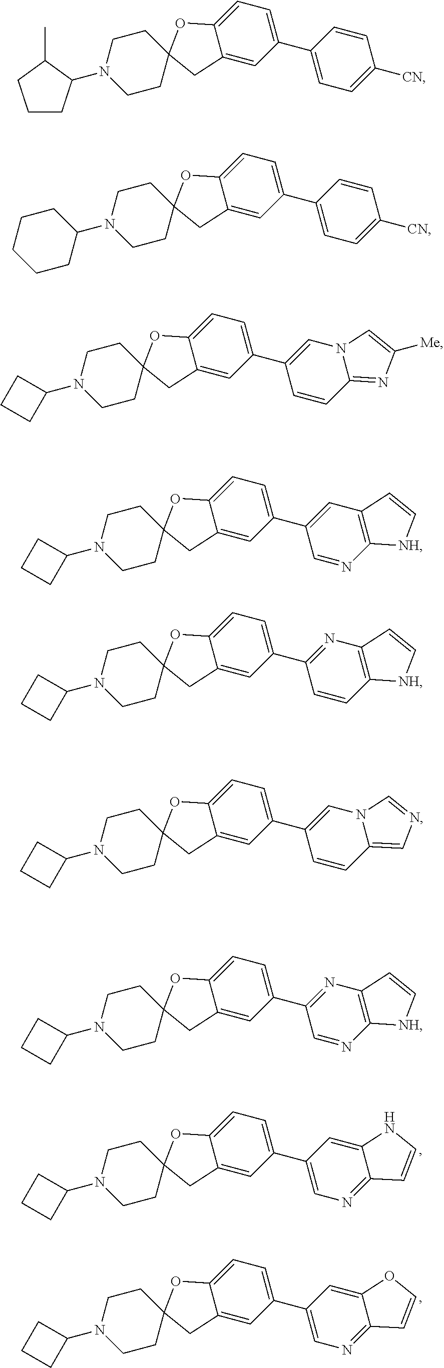

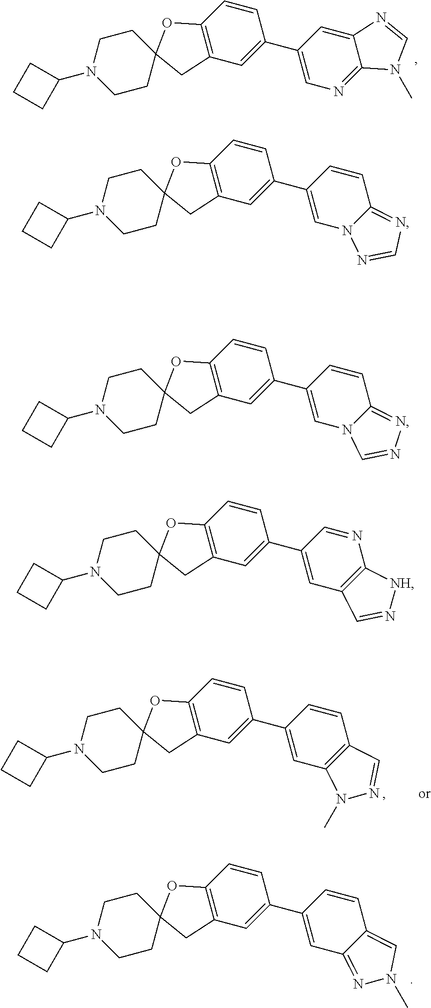

In one embodiment, R³ is (3 to 8 membered)heterocyclyl optionally substituted with one or more R¹⁰; R⁴ and R⁷ are hydrogen; R⁵ and R⁶ are independently hydrogen, halo, cyano, (C₁-C₆)alkyl optionally substituted with one or more R¹⁰, (C₃-C₇)cycloalkyl optionally substituted with one or more R¹⁰, (3 to 10 membered) heterocyclyl optionally substituted with one or more R¹⁰, (6 to 10 membered)aryl optionally substituted with one or more R¹⁰, (5 to 10 membered)heteroaryl optionally substituted with one or more R¹⁰, —OR¹⁰, —N(R¹⁰)₂, C(O)R¹⁰, NR¹⁰C(O)R¹⁰, or —C(O)N(R¹⁰)₂; wherein R¹⁰ is defined herein elsewhere.

In one embodiment, R³ is optionally substituted piperidinyl. In some embodiments, R³ is piperidinyl optionally substituted with one or more (C₁-C₄)alkyl optionally substituted with one or more halo. In one embodiment, R³ is 4-methyl-piperidinyl. In another embodiment, R³ is optionally substituted tetrahydro-2H-pyranyl. In some embodiments, R³ is unsubstituted tetrahydro-2H-pyranyl.

In one embodiment, R⁴ and R⁷ is hydrogen; one of R⁵ and R⁶ is hydrogen, and the other is hydrogen, halo, optionally substituted indolyl, or optionally substituted phenyl. In another embodiment, R⁴, R⁵, and R⁷ are hydrogen; and R⁶ is hydrogen, halo, indolyl optionally substituted with one or more R¹⁰, or phenyl optionally substituted with one or more R¹⁰. In some embodiments, R¹⁰ is halo, cyano, optionally substituted amido, optionally substituted aminoalkyl, or optionally substituted (C₁-C₄)alkyl.

In one embodiment, specific examples include, but are not limited to, the following compounds:

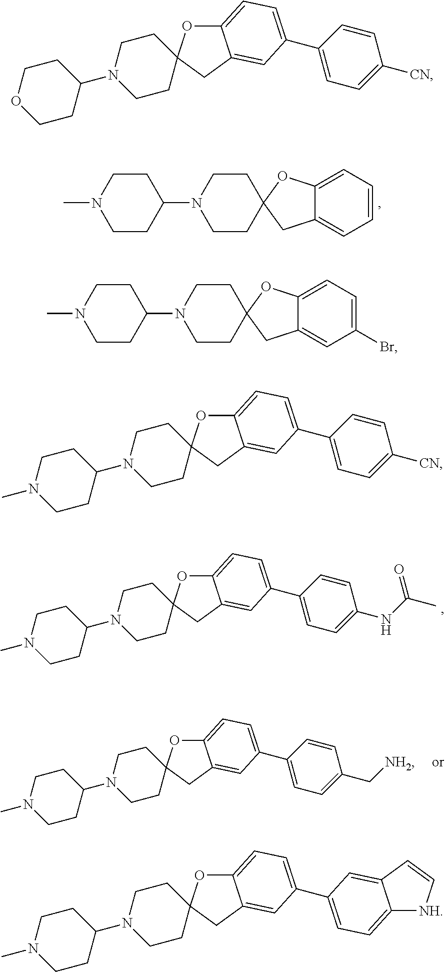

In one embodiment, R³ is —CR¹⁶R¹⁷R¹⁸, wherein R¹⁶, R¹⁷ and R¹⁸ are each independently R¹⁰; R⁴ and R⁷ are hydrogen; and R⁵ and R⁶ are independently hydrogen, halo, cyano, (C₁-C₆)alkyl optionally substituted with one or more R¹⁰, (C₃-C₇)cycloalkyl optionally substituted with one or more R¹⁰, (3 to 10 membered)heterocyclyl optionally substituted with one or more R¹⁰, (6 to 10 membered)aryl optionally substituted with one or more R¹⁰, (5 to 10 membered)heteroaryl optionally substituted with one or more R¹⁰, —OR¹⁰, —N(R¹⁰)₂, —C(O)R¹⁰, —NR¹⁰C(O)R¹⁰, or —C(O)N(R¹⁰)₂; wherein R¹⁰ is defined herein elsewhere.

In one embodiment, R³ is —CR¹⁶R¹⁷R¹⁸, wherein R¹⁶, R¹⁷, and R¹⁸ are each independently hydrogen, optionally substituted (C₁-C₆)alkyl, optionally substituted (C₃-C₇)cycloalkyl, or optionally substituted heteroaryl. In some embodiments, R³ is —CR¹⁶R¹⁷R¹⁸, wherein R¹⁶, R¹⁷, and R¹⁸ are each independently hydrogen, methyl, cyclopentyl, or imidazolyl. In another embodiment, R³ is —CR¹⁶R¹⁷R¹⁸, wherein R¹⁶ is hydrogen, R¹⁷ is hydrogen or methyl, R¹⁸ is methyl, optionally substituted (C₃-C₇)cycloalkyl, or optionally substituted heteroaryl. In another embodiment, R³ is —CR¹⁶R¹⁷R¹⁸, wherein R¹⁶ is hydrogen, R¹⁷ is hydrogen or methyl, R¹⁸ is methyl, cyclobutyl, or imidazolyl. In another embodiment, R³ is isopropyl, cyclobutylmethyl, 1-cyclobutylethyl, or imidazolylmethyl.

In one embodiment, R⁴ and R⁷ is hydrogen; one of R⁵ and R⁶ is hydrogen, and the other is hydrogen, halo, optionally substituted indolyl, or optionally substituted phenyl. In another embodiment, R⁴, R⁵, and R⁷ are hydrogen; and R⁶ is hydrogen, halo, indolyl optionally substituted with one or more R¹⁰, or phenyl optionally substituted with one or more R¹⁰. In some embodiments, R¹⁰ is halo, cyano, optionally substituted amido, optionally substituted aminoalkyl, or optionally substituted (C₁-C₄)alkyl.

In one embodiment, specific examples include, but are not limited to, the following compounds:

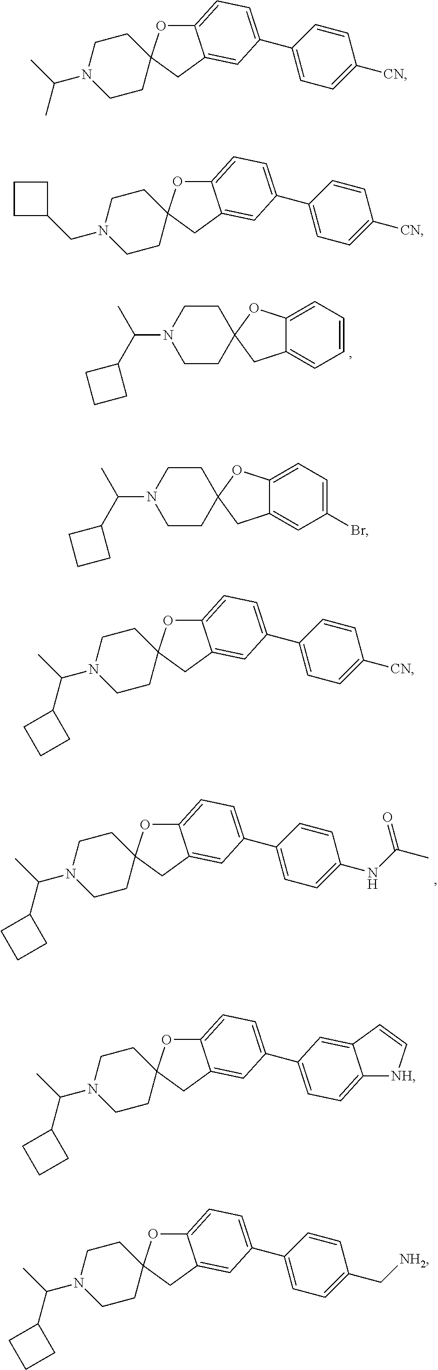

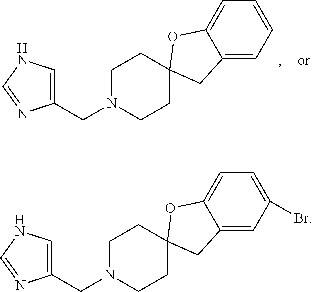

In one embodiment, provided herein is a compound of formula (III):

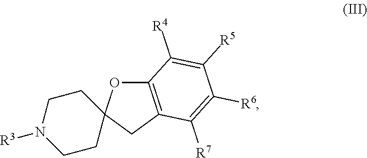

or a pharmaceutically acceptable salt or stereoisomer thereof, wherein

R³ is hydrogen, ═O, (C₁-C₁₀)alkyl, (C₂-C₁₀)alkenyl, (C₃-C₁₀)cycloalkyl, (C₆-C₁₀)aralkyl, (C₁-C₁₀)heteroalkyl, (3 to 10 membered)heterocyclyl, (6 to 10 membered) aryl, or (5 to 10 membered)heteroaryl, each of which may be optionally substituted with one or more R¹⁰;

R⁴, R⁵, and R⁷ are each independently hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹⁰;

R⁶ is (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, thiol, sulfinyl, or sulfonyl, each of which may be optionally substituted with one or more R¹⁰; with the proviso that (i) R⁶ is not 6-membered heteroaryl substituted with oxo, hydroxyl, or halo; and (ii) R⁶ is not phenyl substituted with amido or sulfonyl;

optionally R⁴ and R⁵, R⁵ and R⁶, or R⁶ and R⁷ together with the atoms to which they are attached form an optionally substituted 3 to 10 membered cycloalkyl, heterocyclyl, aryl, or heteroaryl ring;

each occurrence of R¹⁰ is independently a bond, hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered) heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹¹; optionally two germinal or vicinal R¹⁰ substituents together with the atom(s) to which they are attached form an optionally substituted 3 to 10 membered ring;

each occurrence of R¹¹ is independently hydrogen, halo, cyano, (C₁-C₁₀)alkyl optionally substituted with one or more R¹², (C₂-C₁₀)alkenyl optionally substituted with one or more R¹², (C₃-C₁₀)cycloalkyl optionally substituted with one or more R¹², (C₁-C₁₀)heteroalkyl optionally substituted with one or more R¹², (3 to 10 membered) heterocyclyl optionally substituted with one or more R¹², (C₆-C₁₂)aralkyl optionally substituted with one or more R¹², (6 to 10 membered)aryl optionally substituted with one or more R¹², (5 to 10 membered)heteroaryl optionally substituted with one or more R¹², ═O, —R¹³, —OR¹³, —NR¹³R¹⁴, —N(R¹³)C(O)R¹⁴, C(O)NR¹³R¹⁴, C(O)R¹³, C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹¹ substituents together with the atom(s) to which they are attached form an optionally substituted 3 to 10 membered ring;

each occurrence of R¹² is independently hydrogen, halo, cyano, (C₁-C₆)alkyl optionally substituted with one or more R¹³, (C₂-C₆)alkenyl optionally substituted with one or more R¹³, (C₃-C₇)cycloalkyl optionally substituted with one or more R¹³, (3 to 8 membered)heterocyclyl optionally substituted with one or more R¹³, (6 to 10 membered)aryl optionally substituted with one or more R¹³, (5 to 10 membered), heteroaryl optionally substituted with one or more R¹³, ═O, R¹³, OR¹³, —NR¹³R¹⁴, —N(R¹³)C(O)R¹⁴, —C(O)NR¹³R¹⁴, —C(O)R¹³, C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹² substituents together with the atom(s) to which they are attached form an optionally substituted 3 to 10 membered ring; and

R¹³, R¹⁴, and R¹⁵ are independently hydrogen, halo, cyano, (C₁-C₆)alkyl, (C₂-C₆)alkenyl, (C₃-C₇)cycloalkyl, (C₇-C₁₀)aralkyl; (C₁-C₆)heteroalkyl, (3 to 8 membered) heterocyclyl, (6 to 10 membered)aryl, or (5 to 10 membered)heteroaryl; optionally two germinal or vicinal R¹³ substituents together with the atom(s) to which they are attached form an optionally substituted 3 to 10 membered ring; optionally R¹³ and R¹⁴, or R¹⁴ and R¹⁵ together with the atom(s) to which they are attached form an optionally substituted 3 to 10 membered ring.

In one embodiment, R³ is hydrogen, (C₁-C₁₀)alkyl, (C₂-C₁₀)alkenyl, (C₃-C₁₀)cycloalkyl, (C₆-C₁₀)aralkyl, (C₁-C₁₀)heteroalkyl, (3 to 10 membered)heterocyclyl, (6 to 10 membered) aryl, or (5 to 10 membered)heteroaryl, each of which may be optionally substituted with one or more R¹⁰. In one embodiment, R³ is (C₃-C₇) cycloalkyl optionally substituted with one or more R¹⁰. In one embodiment, R³ is cyclobutyl optionally substituted with one or more R¹⁰. In one embodiment, R³ is cyclobutyl.

In one embodiment, R⁴, R⁵, and R⁷ are hydrogen.

In one embodiment, R⁶ is (C₁-C₆)alkyl optionally substituted with one or more R¹⁰, (C₃-C₇)cycloalkyl optionally substituted with one or more R¹⁰, (3 to 10 membered)heterocyclyl optionally substituted with one or more R¹⁰, (6 to 10 membered)aryl optionally substituted with one or more R¹⁰, (5 to 10 membered) heteroaryl optionally substituted with one or more R¹⁰, —OR¹⁰, —N(R¹⁰)₂, —NR¹⁰C(O)R¹⁰, or —C(O)N(R¹⁰)₂; with the proviso that (i) R⁶ is not 6-membered heteroaryl substituted with oxo, hydroxyl, or halo; and (ii) R⁶ is not phenyl substituted with amido or sulfonyl.

In one embodiment, R⁶ is optionally substituted 9-membered heteroaryl, optionally substituted phenyl, optionally substituted pyrazolyl, optionally substituted imidazolyl, optionally substituted pyrimidinyl, optionally substituted pyrazinyl, optionally substituted pyridyl, optionally substituted indolyl, optionally substituted benzimidazolyl, optionally substituted imidazopyridinyl, optionally substituted piperidinyl, optionally substituted piperazinyl, or —CH₂R¹⁰; with the proviso that (i) R⁶ is not 6-membered heteroaryl substituted with oxo, hydroxyl, or halo; and (ii) R⁶ is not phenyl substituted with amido or sulfonyl.

In one embodiment, R⁶ is (C₁-C₄)alkyl optionally substituted with one or more R¹⁰, phenyl optionally substituted with one or more R¹⁰, (3 to 10 membered)heterocyclyl optionally substituted with one or more R¹⁰, (5 to 10 membered)heteroaryl optionally substituted with one or more R¹⁰; with the proviso that (i) R⁶ is not 6-membered heteroaryl substituted with oxo, hydroxyl, or halo; and (ii) R⁶ is not phenyl substituted with amido or sulfonyl.

In one embodiment, R⁶ is (C₁-C₄)alkyl optionally substituted with one or more R¹⁰. In one embodiment, R¹⁰ is (3 to 10 membered)heterocyclyl.

In one embodiment, R⁶ is phenyl optionally substituted with one or more halo, cyano, or alkyl. In one embodiment, R⁶ is unsubstituted phenyl.

In one embodiment, R⁶ is 5-membered heteroaryl optionally substituted with one or more R¹⁰. In one embodiment, R⁶ is unsubstituted 5-membered heteroaryl.

In one embodiment, R⁶ is 9-membered heteroaryl optionally substituted with one or more R¹⁰. In one embodiment, R⁶ is unsubstituted 9-membered heteroaryl.

In one embodiment, R⁶ is 6-membered heteroaryl optionally substituted with one or more R¹⁰, wherein R¹⁰ is not oxo, hydroxyl, or halo. In one embodiment, R⁶ is 6-membered heteroaryl optionally substituted with one or more alkyl or cyano. In one embodiment, R⁶ is unsubstituted 6-membered heteroaryl.

In one embodiment, specific examples include, but are not limited to, the following compounds:

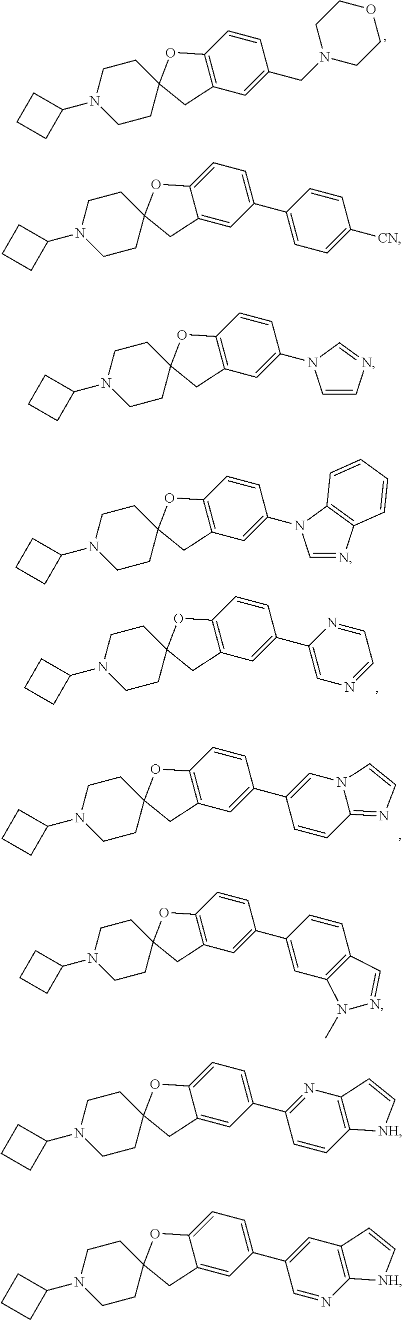

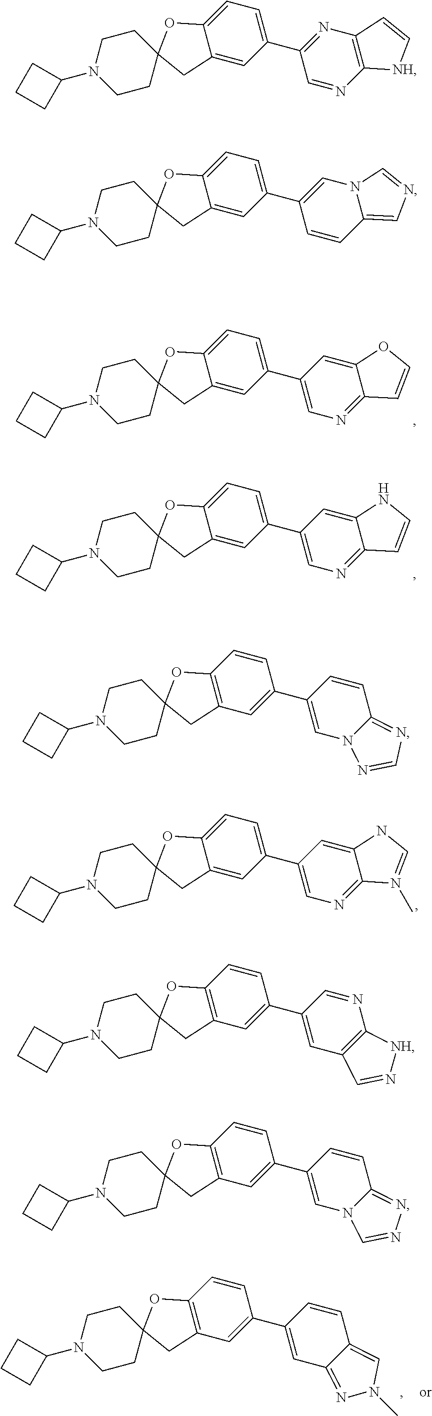

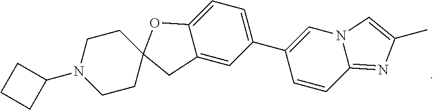

In one embodiment, provided herein is a compound of formula (Ma),

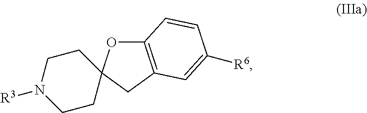

or a pharmaceutically acceptable salt or stereoisomer thereof, wherein

R³ is cyclobutyl optionally substituted with one or more R¹⁰;

R⁶ is (i) (C₁-C₆)alkyl, (C₁-C₆)heteroalkyl, (3 to 10 membered)heterocyclyl, 10-membered aryl, 5-membered heteroaryl, or (9 to 10 membered)heteroaryl, each of which may be optionally substituted with one or more R¹⁰; or (ii) 6-membered aryl or 6-membered heteroaryl, each of which may be optionally substituted with one or more cyano or alkyl;

each occurrence of R¹⁰ is independently a bond, hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered) heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹¹; optionally two germinal or vicinal R¹⁰ substituents together with the atom(s) to which they are attached form an optionally substituted 3 to 10 membered ring;

each occurrence of R¹¹ is independently hydrogen, halo, cyano, (C₁-C₁₀)alkyl optionally substituted with one or more R¹², (C₂-C₁₀)alkenyl optionally substituted with one or more R¹², (C₃-C₁₀)cycloalkyl optionally substituted with one or more R¹², (C₁-C₁₀)heteroalkyl optionally substituted with one or more R¹², (3 to 10 membered) heterocyclyl optionally substituted with one or more R¹², (C₆-C₁₂)aralkyl optionally substituted with one or more R¹², (6 to 10 membered)aryl optionally substituted with one or more R¹², (5 to 10 membered)heteroaryl optionally substituted with one or more R¹², ═O, —R¹³, —OR¹³, —NR¹³R¹⁴, —N(R¹³)C(O)R¹⁴, —C(O)NR¹³R¹⁴, —C(O)R¹³, —C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹¹ substituents together with the atom(s) to which they are attached form an optionally substituted 3 to 10 membered ring;

each occurrence of R¹² is independently hydrogen, halo, cyano, (C₁-C₆)alkyl optionally substituted with one or more R¹³, (C₂-C₆)alkenyl optionally substituted with one or more R¹³, (C₃-C₇)cycloalkyl optionally substituted with one or more R¹³, (3 to 8 membered)heterocyclyl optionally substituted with one or more R¹³, (6 to 10 membered)aryl optionally substituted with one or more R¹³, (5 to 10 membered) heteroaryl optionally substituted with one or more R¹³, ═O, —R¹³, —OR¹³, —NR¹³R¹⁴, —N(R¹³)C(O)R¹⁴, —C(O)NR¹³R¹⁴, —C(O)R¹³, —OC(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹² substituents together with the atom(s) to which they are attached form an optionally substituted 3 to 10 membered ring; and

R¹³, R¹⁴, and R¹⁵ are independently hydrogen, halo, cyano, (C₁-C₆)alkyl, (C₂-C₆)alkenyl, (C₃-C₇)cycloalkyl, (C₇-C₁₀)aralkyl; (C₁-C₆)heteroalkyl, (3 to 8 membered) heterocyclyl, (6 to 10 membered)aryl, or (5 to 10 membered)heteroaryl; optionally two germinal or vicinal R¹³ substituents together with the atom(s) to which they are attached form an optionally substituted 3 to 10 membered ring; optionally R¹³ and R¹⁴, or R¹⁴ and R¹⁵ together with the atom(s) to which they are attached form an optionally substituted 3 to 10 membered ring.

In one embodiment, R⁶ is (i) (C₁-C₆)alkyl, (C₁-C₆)heteroalkyl, (3 to 10 membered)heterocyclyl, 10-membered aryl, 5-membered heteroaryl, or (9 to 10 membered)heteroaryl, each of which may be optionally substituted with one or more R¹⁰; or (ii) 6-membered aryl or 6-membered heteroaryl, each of which may be optionally substituted with one or more cyano or alkyl. In one embodiment, R⁶ is (i) (C₁-C₆)alkyl, (C₁-C₆)heteroalkyl, (3 to 10 membered)heterocyclyl, 10-membered aryl, 5-membered heteroaryl, or (9 to 10 membered)heteroaryl, each of which may be optionally substituted with one or more R¹⁰; or (ii) 6-membered heteroaryl optionally substituted with one or more cyano or alkyl. In one embodiment, R⁶ is (i) (C₁-C₆)alkyl, (C₁-C₆)heteroalkyl, (3 to 10 membered)heterocyclyl, 10-membered aryl, 5-membered heteroaryl, or (9 to 10 membered)heteroaryl, each of which may be optionally substituted with one or more R¹⁰; or (ii) 6-membered heteroaryl optionally substituted with one or more alkyl. In one embodiment, In one embodiment, R⁶ is (i) (C₁-C₆)alkyl, (C₁-C₆)heteroalkyl, (3 to 10 membered)heterocyclyl, 10-membered aryl, 5-membered heteroaryl, or (9 to 10 membered)heteroaryl, each of which may be optionally substituted with one or more R¹⁰; or (ii) unsubstituted 6-membered heteroaryl. In one embodiment, R⁶ is (C₁-C₆)alkyl, (C₁-C₆)heteroalkyl, (3 to 10 membered)heterocyclyl, 10-membered aryl, 5-membered heteroaryl, or (9 to 10 membered)heteroaryl, each of which may be optionally substituted with one or more R¹⁰. In one embodiment, R⁶ is (i) (C₁-C₄)alkyl, (3 to 10 membered)heterocyclyl, 10-membered aryl, 5-membered heteroaryl, or (9 to 10 membered)heteroaryl, each of which may be optionally substituted with one or more R¹⁰; or (ii) 6-membered aryl or 6-membered heteroaryl, each of which may be optionally substituted with one or more cyano or alkyl. In one embodiment, R⁶ is (i) (C₁-C₄)alkyl, (3 to 10 membered)heterocyclyl, 10-membered aryl, 5-membered heteroaryl, or (9 to 10 membered)heteroaryl, each of which may be optionally substituted with one or more R¹⁰; or (ii) 6-membered heteroaryl optionally substituted with one or more cyano or alkyl. In one embodiment, R⁶ is (i) (C₁-C₄)alkyl, (3 to 10 membered)heterocyclyl, 5-membered heteroaryl, or (9 to 10 membered) heteroaryl, each of which may be optionally substituted with one or more R¹⁰; or (ii) 6-membered heteroaryl optionally substituted with one or more cyano or alkyl. In one embodiment, R⁶ is (i) (C₁-C₄)alkyl, 5-membered heteroaryl, or (9 to 10 membered) heteroaryl, each of which may be optionally substituted with one or more R¹⁰; or (ii) 6-membered heteroaryl optionally substituted with one or more cyano or alkyl. In one embodiment, R⁶ is (i) (C₁-C₄)alkyl, 5-membered heteroaryl, or (9 to 10 membered) heteroaryl, each of which may be optionally substituted with one or more R¹⁰; or (ii) 6-membered heteroaryl optionally substituted with one or more alkyl. In one embodiment, R⁶ is (i) (C₁-C₄)alkyl, 5-membered heteroaryl, or (9 to 10 membered) heteroaryl, each of which may be optionally substituted with one or more R¹⁰; or (ii) unsubstituted 6-membered heteroaryl. In one embodiment, R⁶ is (C₁-C₄)alkyl, 5-membered heteroaryl, or (9 to 10 membered)heteroaryl, each of which may be optionally substituted with one or more R¹⁰. In one embodiment, R⁶ is —CH₂R¹⁰, wherein R¹⁰ is optionally substituted (3 to 10 membered)heterocyclyl or optionally substituted (5 to 10 membered)heteroaryl.

In one embodiment, R⁶ is a (5 to 10 membered)heteroaryl optionally substituted with one or more R¹⁰. In one embodiment, R⁶ is a (5 to 10 membered) heteroaryl optionally substituted with one or more R¹⁰, wherein the heteroaryl has one or two ring(s), each containing one, two, or three heteroatoms selected from N, O, and S. In one embodiment, two adjacent R¹⁰ substituents together with the atoms to which they are attached further form an optionally substituted ring. In one embodiment, two adjacent R¹⁰ substituents together with the atoms to which they are attached further form an optionally substituted (5 to 6 membered) aryl or heteroaryl, wherein the heteroaryl contains one, two, or three heteroatoms selected from N, O, and S. In one embodiment, R⁶ is a (5 to 10 membered)heteroaryl optionally substituted with one to five R¹⁰. In one embodiment, R⁶ is a (5 to 10 membered)heteroaryl optionally substituted with one to three R¹⁰. Specific examples of R⁶ include, but are not limited to, optionally substituted pyrazolyl, optionally substituted imidazolyl, optionally substituted pyrimidinyl, optionally substituted pyrazinyl, optionally substituted pyridyl, optionally substituted indolyl, optionally substituted benzimidazolyl, or optionally substituted imidazopyridinyl.

In one embodiment, R⁶ is —CH₂R¹⁰, wherein R¹⁰ is as defined herein elsewhere.

In certain embodiments, R⁶ (including its optional substituents) has an estimated pKa of less than about 8, less than about 7, less than about 6, less than about 5, or less than about 4. In certain embodiments, R⁶ has an estimated pKa of less than about 8. In certain embodiments, R⁶ has an estimated pKa of greater than about 4, greater than about 5, greater than about 6, greater than about 7, or greater than about 8. In certain embodiments, R⁶ has an estimated pKa of greater than about 4. In certain embodiments, R⁶ has an estimated pKa of between about 8 and about 4, between about 8 and about 5, between about 7 and about 4, between about 8 and about 6, between about 7 and about 5, between about 6 and about 4. In certain embodiments, R⁶ has an estimated pKa of about 8.0, about 7.5, about 7.0, about 6.5, about 6.0, about 5.5, about 5.0, about 4.5, or about 4.0. It is understood that certain substituents on R⁶ may adjust the pKa value of R⁶. In one embodiment, the pKa value may be measured by an experimental methods known in the art. In one embodiment, the pKa value may be calculated using, e.g., a commercially available software program (e.g., ACD Lab). In one embodiment, the pKa value may be estimated based on known pKa values of analogous molecules.

In one embodiment, the compounds provided herein with R⁶ having certain pKa values as provided herein elsewhere have certain pharmaceutical properties, such as, e.g., high brain penetration and low brain accumulation.

In one embodiment, specific examples include, but are not limited to, the following compounds, where the pKa values of the R⁶ moiety were calculated using ACD Lab (Version 12.01, released 9 Feb., 2010, Advanced Chemistry Development, Toronto, Ontario, CA):

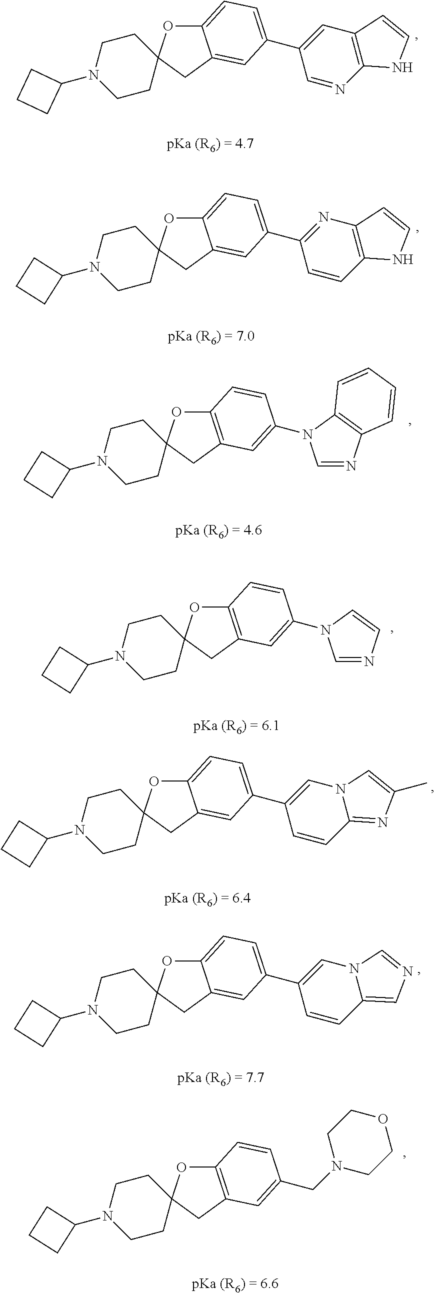

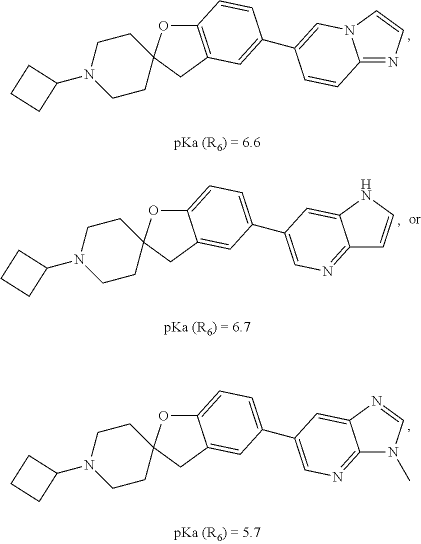

In one embodiment, provided herein is a compound of formula (IVa):

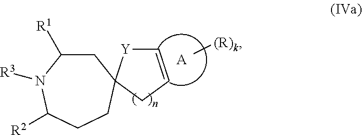

or a pharmaceutically acceptable salt or stereoisomer thereof, wherein

ring A is optionally substituted 5- or 6-membered aryl or heteroaryl;

Y is O, S, NH, or CH₂;

k is 0, 1, 2, 3, or 4;

n is 1, 2, or 3;

R¹, R², and R³ are independently hydrogen, ═O, (C₁-C₁₀)alkyl, (C₂-C₁₀)alkenyl, (C₃-C₁₀)cycloalkyl, (C₆-C₁₀)aralkyl, (C₁-C₁₀)heteroalkyl, (3 to 10 membered) heterocyclyl, (6 to 10 membered)aryl, or (5 to 10 membered)heteroaryl, each of which may be optionally substituted with one or more R¹⁰; optionally R¹ and R², or R¹ and R³, or R² and R³ together with the atoms to which they are attached form an optionally substituted 3 to 10 membered cycloalkyl or heterocyclyl ring;

each occurrence of R is independently hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹⁰; optionally two adjacent R substituents together with the atoms to which they are attached form an optionally substituted 3 to 10 membered cycloalkyl, heterocyclyl, aryl, or heteroaryl ring;

each occurrence of R¹⁰ is independently a bond, hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered) heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹¹; optionally two germinal or vicinal R¹⁰ substituents together with the atom(s) to which they are attached form an optionally substituted 3 to 10 membered ring;

each occurrence of R¹¹ is independently hydrogen, halo, cyano, (C₁-C₁₀)alkyl optionally substituted with one or more R¹², (C₂-C₁₀)alkenyl optionally substituted with one or more R¹², (C₃-C₁₀)cycloalkyl optionally substituted with one or more R¹², (C₁-C₁₀)heteroalkyl optionally substituted with one or more R¹², (3 to 10 membered) heterocyclyl optionally substituted with one or more R¹², (C₆-C₁₂)aralkyl optionally substituted with one or more R¹², (6 to 10 membered)aryl optionally substituted with one or more R¹², (5 to 10 membered)heteroaryl optionally substituted with one or more R¹², ═O, —R¹³, —OR¹³, —NR¹³R¹⁴, —N(R¹³)C(O)R¹⁴, —C(O)NR¹³R¹⁴, —C(O)R¹³, —C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹¹ substituents together with the atom(s) to which they are attached form an optionally substituted 3 to 10 membered ring;

each occurrence of R¹² is independently hydrogen, halo, cyano, (C₁-C₆)alkyl optionally substituted with one or more R¹³, (C₂-C₆)alkenyl optionally substituted with one or more R¹³, (C₃-C₇)cycloalkyl optionally substituted with one or more R¹³, (3 to 8 membered)heterocyclyl optionally substituted with one or more R¹³, (6 to 10 membered)aryl optionally substituted with one or more R¹³, (5 to 10 membered) heteroaryl optionally substituted with one or more R¹³, ═O, —R¹³, —OR¹³, —NR¹³R¹⁴, —N(R¹³)C(O)R¹⁴, —C(O)NR¹³R¹⁴, —C(O)R¹³, —C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹² substituents together with the atom(s) to which they are attached form an optionally substituted 3 to 10 membered ring; and

R¹³, R¹⁴, and R¹⁵ are independently hydrogen, halo, cyano, (C₁-C₆)alkyl, (C₂-C₆)alkenyl, (C₃-C₇)cycloalkyl, (C₇-C₁₀)aralkyl; (C₁-C₆)heteroalkyl, (3 to 8 membered) heterocyclyl, (6 to 10 membered)aryl, or (5 to 10 membered)heteroaryl; optionally two germinal or vicinal R¹³ substituents together with the atom(s) to which they are attached form an optionally substituted 3 to 10 membered ring; optionally R¹³ and R¹⁴, or R¹⁴ and R¹⁵ together with the atom(s) to which they are attached form an optionally substituted 3 to 10 membered ring.

In one embodiment, provided herein is a compound of formula (IVb):

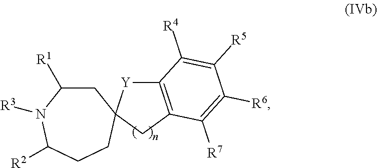

or a pharmaceutically acceptable salt or stereoisomer thereof, wherein R¹, R², R³, R⁴, R⁵, R⁶, R⁷, Y, and n are defined herein elsewhere. In one embodiment, R⁴, R⁵, R⁶, and R⁷ are each independently R; optionally R⁴ and R⁵, or R⁵ and R⁶, or R⁶ and R⁷ together with the atoms to which they are attached form an optionally substituted 3 to 10 membered cycloalkyl, heterocyclyl, aryl, or heteroaryl ring.

In one embodiment, provided herein is a compound of formula (IVc):

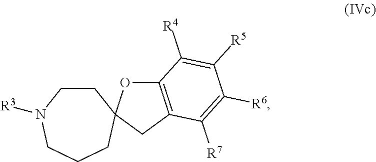

or a pharmaceutically acceptable salt or stereoisomer thereof, wherein R³, R⁴, R⁵, R⁶, and R⁷ are defined herein elsewhere. In one embodiment, R³ is hydrogen, (C₁-C₁₀)alkyl, (C₂-C₁₀)alkenyl, (C₃-C₁₀)cycloalkyl, (C₆-C₁₀)aralkyl, (C₁-C₁₀)heteroalkyl, (3 to 10 membered)heterocyclyl, (6 to 10 membered) aryl, or (5 to 10 membered)heteroaryl, each of which may be optionally substituted with one or more R¹⁰.

In one embodiment, R⁴, R⁵, and R⁷ are hydrogen, and R⁶ is defined herein elsewhere. In one embodiment, specific examples include, but are not limited to, the following compounds:

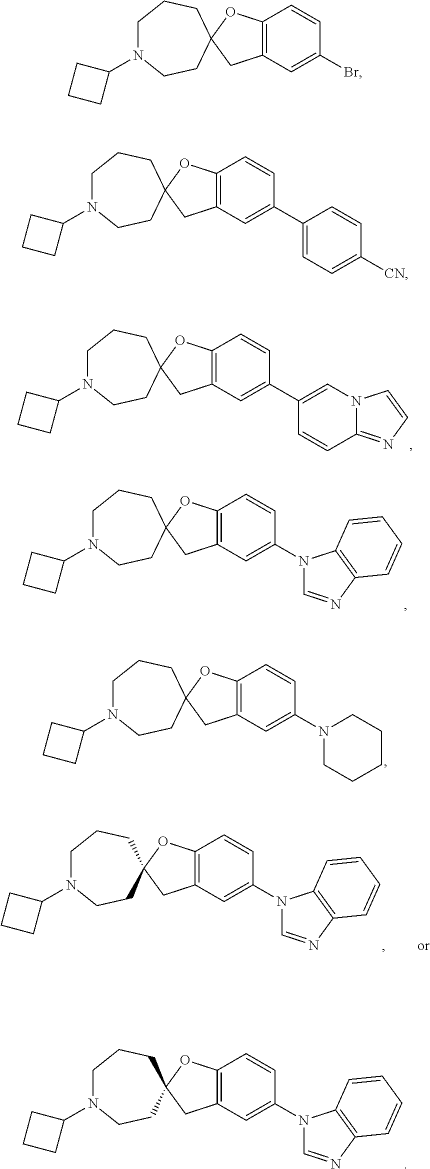

In one embodiment, R⁴, R⁶, and R⁷ are hydrogen, and R⁵ is defined herein elsewhere. In one embodiment, specific examples include, but are not limited to, the following compounds:

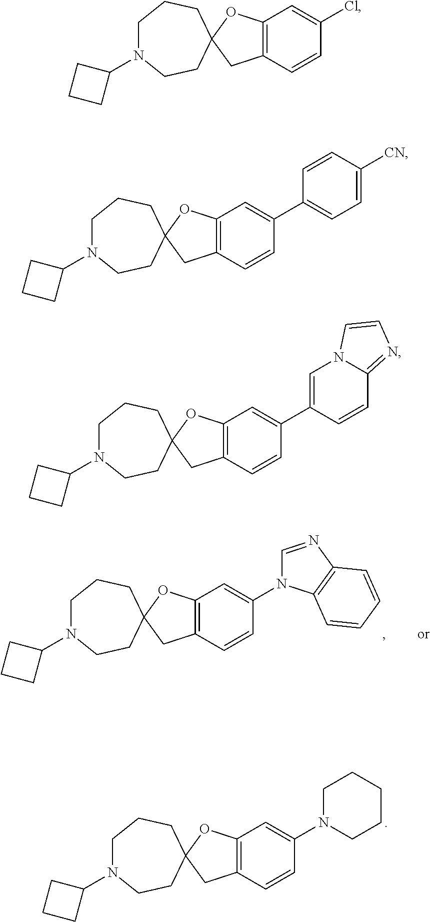

In one embodiment, provided herein is a compound of formula (Va) or (Vb):

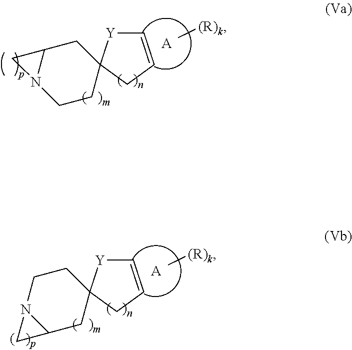

or a pharmaceutically acceptable salt or stereoisomer thereof, wherein R, Y, m, k, and ring A are defined herein elsewhere; n is 1, 2, or 3; and p is 1, 2, 3, 4, 5, 6, 7, or 8.

In one embodiment, provided herein is a compound of formula (VIa) or (VIb):

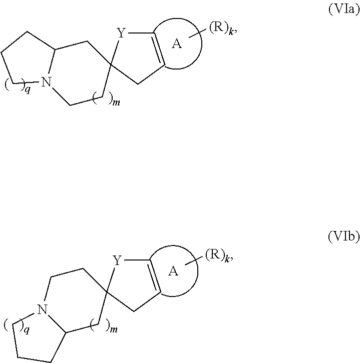

or a pharmaceutically acceptable salt or stereoisomer thereof, wherein R, Y, m, k, and ring A are defined herein elsewhere; and q is 1 or 2.

In one embodiment, provided herein is a compound of formula (VIIa) or (VIIb):

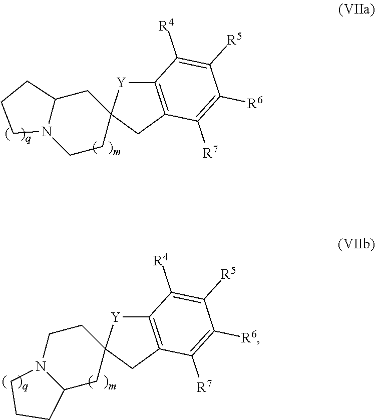

or a pharmaceutically acceptable salt or stereoisomer thereof, wherein R⁴, R⁵, R⁶, R⁷, Y, and m are defined herein elsewhere; and q is 1 or 2. In one embodiment, R⁴, R⁵, R⁶, and R⁷ are each independently R; optionally R⁴ and R⁵, or R⁵ and R⁶, or R⁶ and R⁷ together with the atoms to which they are attached form an optionally substituted 3 to 10 membered cycloalkyl, heterocyclyl, aryl, or heteroaryl ring.

In one embodiment, provided herein is a compound of formula (VIIIa) or (VIIIb):

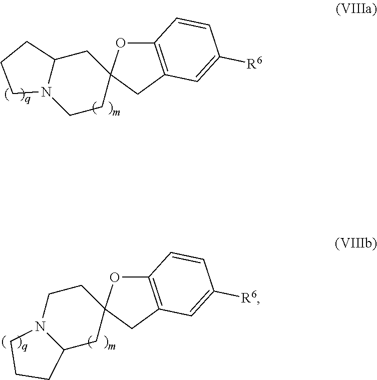

or a pharmaceutically acceptable salt or stereoisomer thereof, wherein R⁶ and m are defined herein elsewhere; and q is 1 or 2.

In one embodiment, q is 1 and m is 1. In one embodiment, specific examples include, but are not limited to, the following compounds:

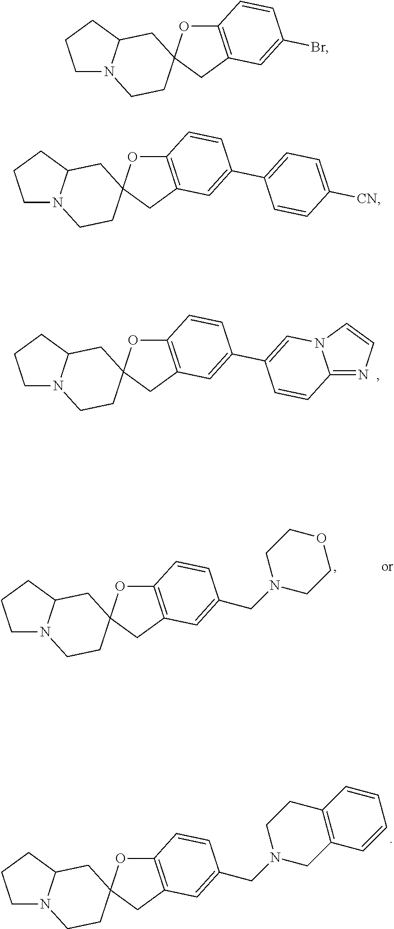

In one embodiment, q is 2 and m is 1. In one embodiment, specific examples include, but are not limited to, the following compounds:

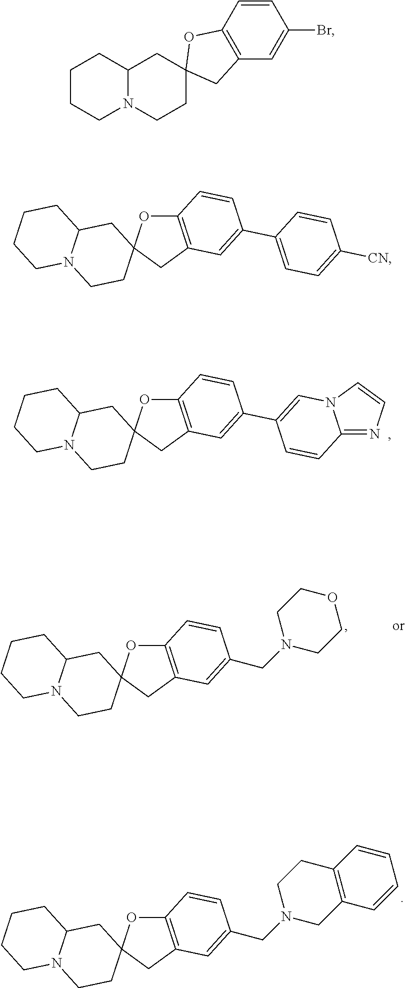

In one embodiment, specific examples include, but are not limited to, the following compounds:

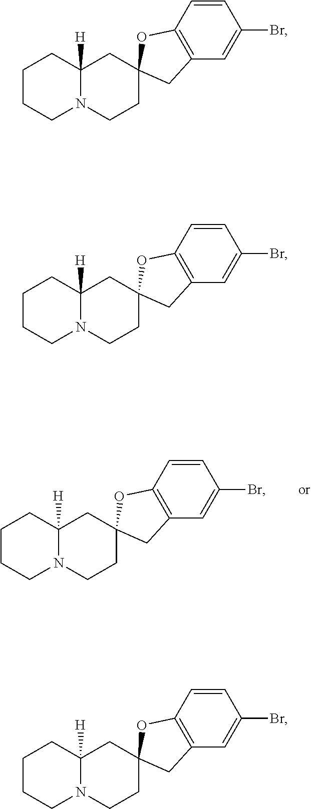

or a mixture of two or more thereof, such as, e.g., a mixture of two enantiomers of a certain diastereomer (e.g., syn or anti diastereomer).

In one embodiment, provided herein is a racemic mixture or an enantio-enriched mixture of:

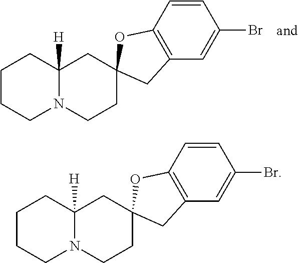

In one embodiment, provided herein is a racemic mixture or an enantio-enriched mixture of:

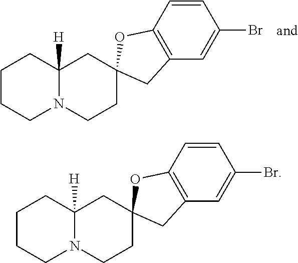

In one embodiment, specific examples include, but are not limited to, the following compounds:

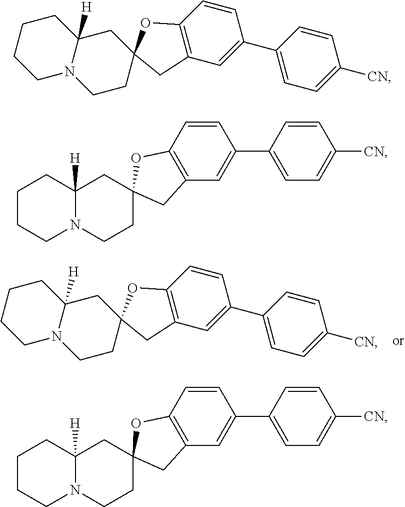

or a mixture of two or more thereof, such as, e.g., a mixture of two enantiomers of a certain diastereomer (e.g., syn or anti diastereomer).

In one embodiment, provided herein is a racemic mixture or an enantio-enriched mixture of:

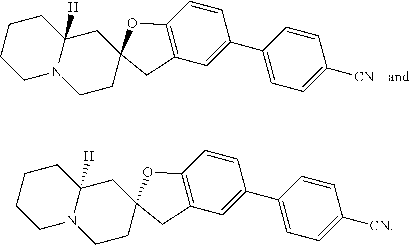

In one embodiment, provided herein is a racemic mixture or an enantio-enriched mixture of:

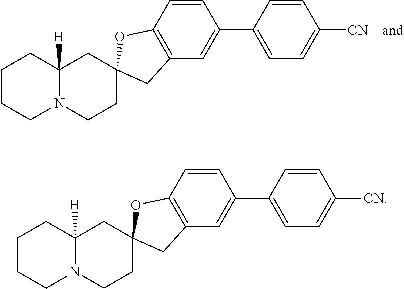

In one embodiment, specific examples include, but are not limited to, the following compounds:

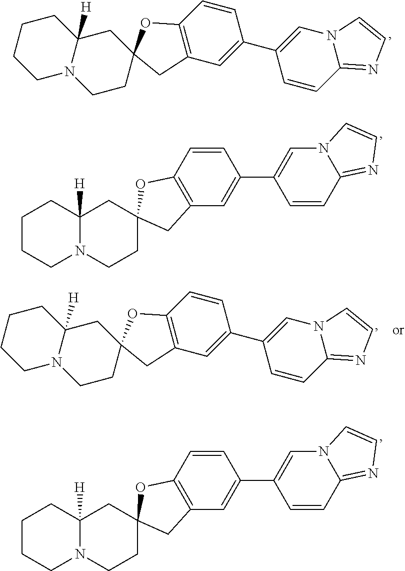

or a mixture of two or more thereof, such as, e.g., a mixture of two enantiomers of a certain diastereomer (e.g., syn or anti diastereomer).

In one embodiment, provided herein is a racemic mixture or an enantio-enriched mixture of:

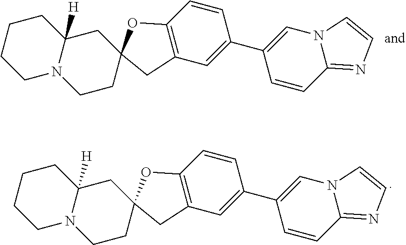

In one embodiment, provided herein is a racemic mixture or an enantio-enriched mixture of:

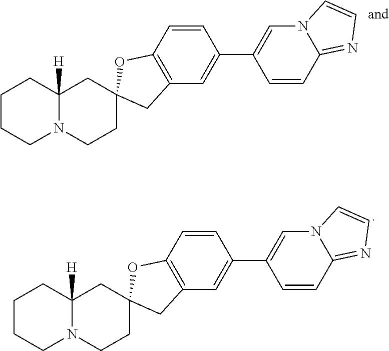

In one embodiment, specific examples include, but are not limited to, the following compounds:

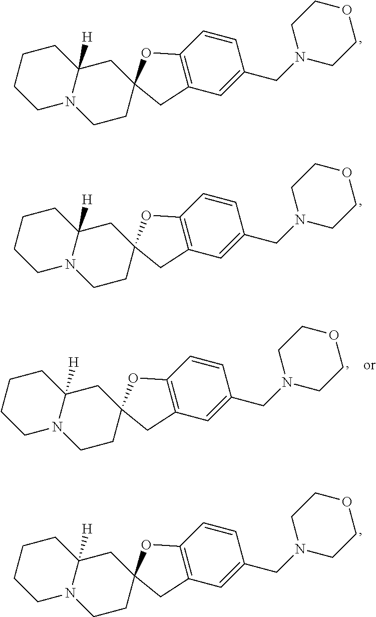

or a mixture of two or more thereof, such as, e.g., a mixture of two enantiomers of a certain diastereomer (e.g., syn or anti diastereomer).

In one embodiment, provided herein is a racemic mixture or an enantio-enriched mixture of:

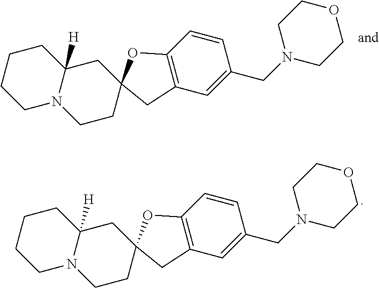

In one embodiment, provided herein is a racemic mixture or an enantio-enriched mixture of:

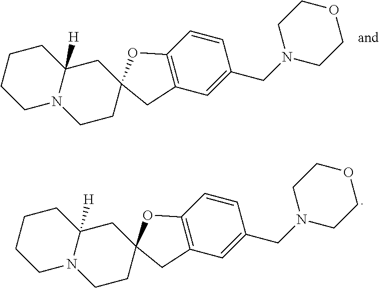

In one embodiment, specific examples include, but are not limited to, the following compounds:

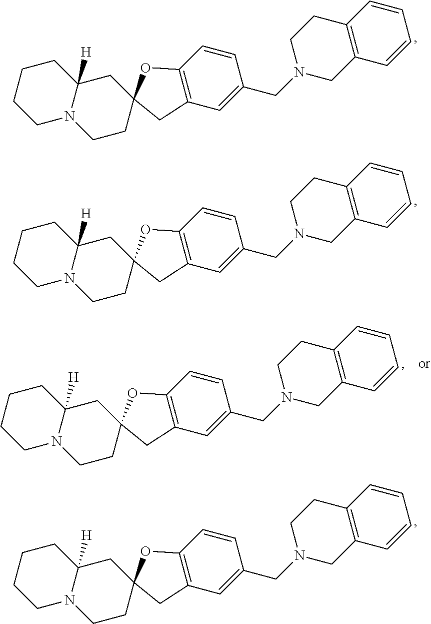

or a mixture of two or more thereof, such as, e.g., a mixture of two enantiomers of a certain diastereomer (e.g., syn or anti diastereomer).

In one embodiment, provided herein is a racemic mixture or an enantio-enriched mixture of:

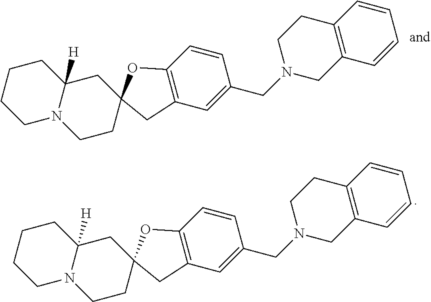

In one embodiment, provided herein is a racemic mixture or an enantio-enriched mixture of:

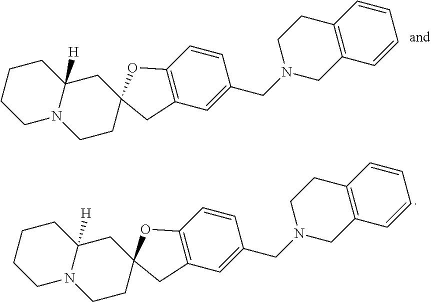

In one embodiment, provided herein is a compound of formula (VIIIa):

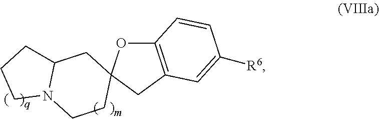

or a pharmaceutically acceptable salt or stereoisomer thereof, wherein R⁶ and m are defined herein elsewhere; and q is 1 or 2.

In one embodiment, q is 1 and m is 2. Specific examples include, but are not limited to, the following compounds:

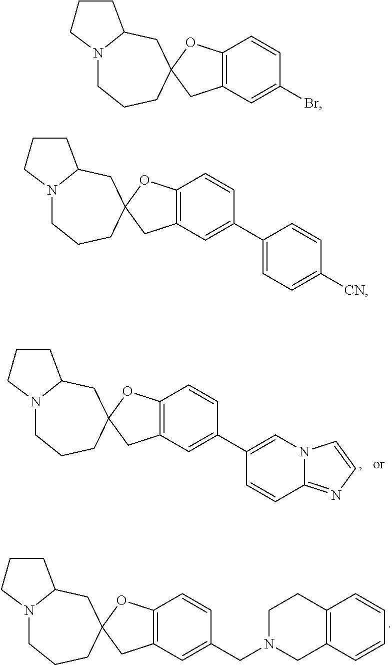

In one embodiment, q is 2 and m is 2. Specific examples include, but are not limited to, the following compounds:

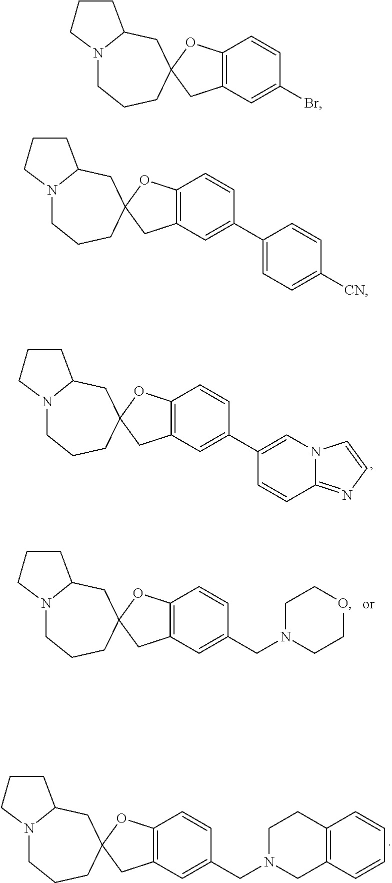

In one embodiment, provided herein is a compound of formula (VIIIb):

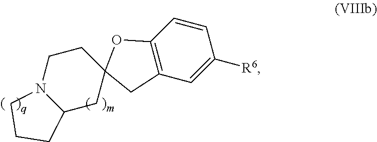

or a pharmaceutically acceptable salt or stereoisomer thereof, wherein R⁶ and m are defined herein elsewhere; and q is 1 or 2.

In one embodiment, q is 1 and m is 2. Specific examples include, but are not limited to, the following compounds:

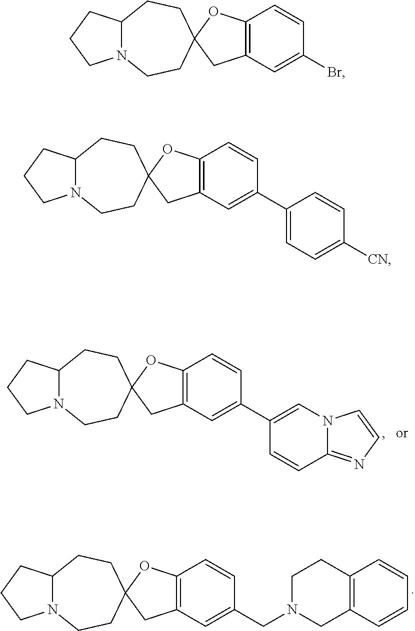

In one embodiment, q is 2 and m is 2. Specific examples include, but are not limited to, the following compounds:

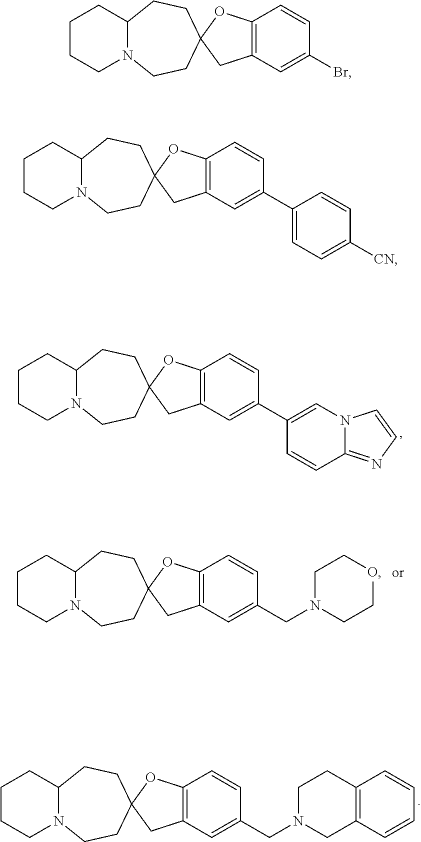

Any of the combinations of R, R¹, R², R³, R⁴, R⁵, R⁶, R⁷, R¹⁰, R¹¹, R¹², R¹³, R¹⁴, R¹⁵, R¹⁶, R¹⁷, R¹⁸, k, m, n, p, q, Y, and ring A are encompassed by this disclosure and specifically provided herein.

It should be noted that if there is a discrepancy between a depicted structure and a chemical name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it and mixtures of two or more stereoisomers of it. Where the compound provided herein contains an alkenyl or alkenylene group, the compound may exist as one or mixture of geometric cis/trans (or Z/E) isomers. Where structural isomers are inter-convertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

The compounds provided herein may be enantiomerically pure, such as a single enantiomer or a single diastereomer, or be stereoisomeric mixtures, such as a mixture of enantiomers, e.g., a racemic mixture of two enantiomers, an enantio-enriched mixture of two enantiomers; or a mixture of two or more diastereomers. In one embodiment, for compounds that undergo epimerization in vivo, one of skill in the art will recognize that administration of a compound in its (R) form is equivalent to administration of the compound in its (S) form, and vice versa. Conventional techniques for the preparation/isolation of individual enantiomers include synthesis from a suitable optically pure precursor, asymmetric synthesis from achiral starting materials, or resolution of an enantiomeric mixture, for example, by chiral chromatography, recrystallization, resolution, diastereomeric salt formation, or derivatization into diastereomeric adducts followed by separation.

When the compound provided herein contains an acidic or basic moiety, it may also be provided as a pharmaceutically acceptable salt (See, e.g., Berge et al., J. Pharm. Sci. 1977, 66, 1-19; and Handbook of Pharmaceutical Salts, Properties, and Use, Stahl and Wermuth, ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In certain embodiments, the compounds provided herein are pharmacologically acceptable salts of the compounds with one or more of hydrochloric, sulfuric, phosphoric, acetic, citric, oxalic, malonic, salicylic, malic, fumaric, succinic, ascorbic, maleic, methanesulfonic, and isoethonic acids; or with one or more of potassium carbonate, sodium or potassium hydroxide, ammonia, triethylamine, and triethanolamine.

The compound provided herein may also be provided as a prodrug, which is a functional derivative of the compound, for example, of Formula I and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See, e.g., Harper, Progress in Drug Research 1962, 4, 221-294; Morozowich et al. in Design of Biopharmaceutical Properties through Prodrugs and Analogs, Roche ed., APHA Acad. Pharm. Sci. 1977; Bioreversible Carriers in Drug in Drug Design, Theory and Application, Roche ed., APHA Acad. Pharm. Sci. 1987; Design of Prodrugs, Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in Transport Processes in Pharmaceutical Systems, Amidon et al., ed., Marcell Dekker, 185-218, 2000; Balant et al., Eur. J. Drug Metab. Pharmacokinet. 1990, 15, 143-53; Balimane & Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; and Waller et al., Br. J. Clin. Pharmac. 1989, 28, 497-507.

### C. SYNTHETIC SCHEMES

Schemes below provide exemplary synthetic methods for the preparation of the compounds provided herein. One of ordinary skills in the art will understand that similar methods may be employed to prepare the compounds provided herein. In other words, one of ordinary skills in the art will recognize that suitable adjustments to reagents, protecting groups, reaction conditions, and reaction sequences may be employed to prepare a desired embodiment. The reactions may be scaled upwards or downwards to suit the amount of material to be prepared.

Specific schemes for preparing compounds provided herein are shown below. Detailed reaction conditions are provided for various specific examples herein below. One of ordinary skills of the art will understand that the following schemes may be modified with appropriate reagents, protecting groups, conditions, starting materials, or reaction sequences to suit the preparation of other embodiments provided herein.

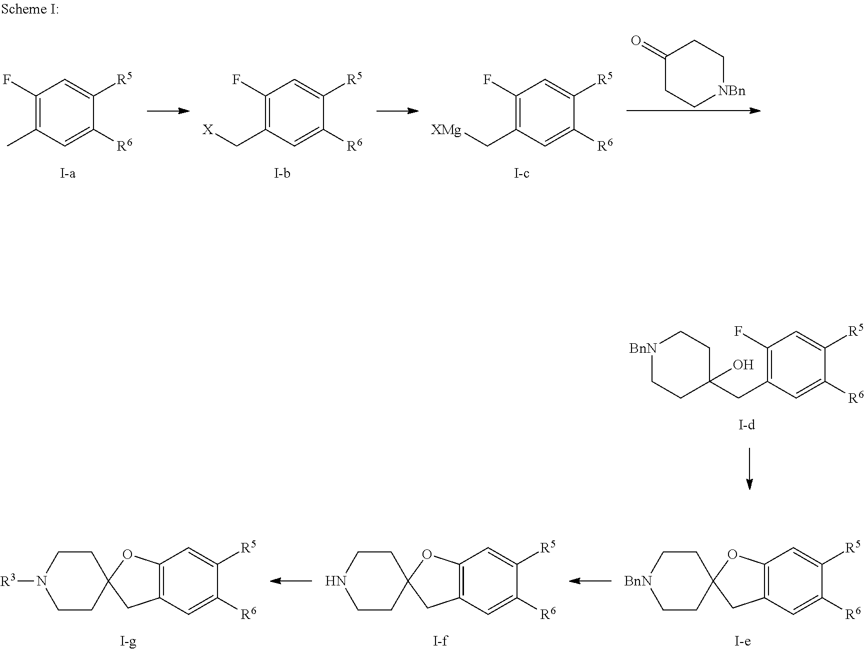

In one embodiment, a compound of formula (III) may be prepared following Scheme I. Compound I-a may be purchased from a commercial source or prepared following literature procedures. I-a is treated with a brominating reagent, such as NBS, to yield compound I-b, where X is bromo. Alternatively, I-b, where X is halo, such as bromo, chloro, or iodo, may be obtained from a commercial source or prepared following known procedures. Compound I-b is treated with magnesium, in a solvent such as ether, to generate the corresponding Grignard reagent I-c, which is reacted with 1-benzylpiperidin-4-one, to render compound I-d. Treating I-d with a base, such as NaH, yields cyclization product I-e. The benzyl protecting group is removed, for example via catalytic hydrogenation to render compound I-f. I-f may be alkylated via reductive hydrogenation or alkylation, to render compound I-g. Optionally, further organic transformations may convert R³, R⁵, and R⁶ to other suitable embodiments of R³, R⁵, and R⁶.

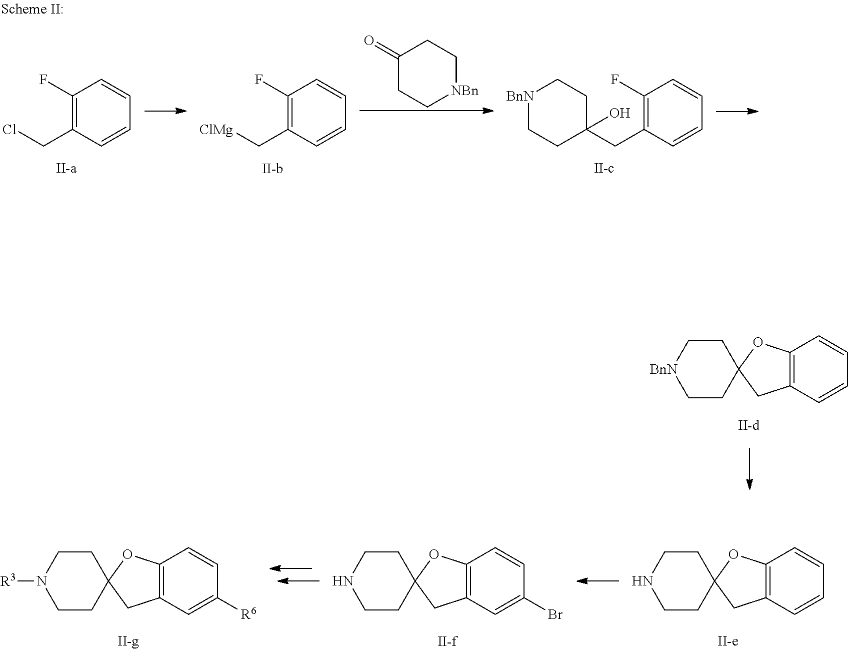

In one embodiment, a compound of formula (III) may also be prepared following Scheme II. Compound II-a is treated with magnesium, in a solvent such as ether, to generate the corresponding Grignard reagent II-b, which is reacted with 1-benzylpiperidin-4-one, to render compound II-c. Treating II-c with a base, such as NaH, yields cyclization product II-d. The benzyl protecting group is removed, for example via catalytic hydrogenation to render compound II-e. Treatment of II-e with brominating reagent, such as NBS, yields compound II-f. II-f may be alkylated via reductive hydrogenation or alkylation, to render compound II-g. Aryl bromide may be converted to suitable R⁶ substituents via known reaction. Optionally, further organic transformations may convert R³ to other suitable embodiments of R³. Optionally, further organic transformations may convert R⁶ to other suitable embodiments of R⁶.

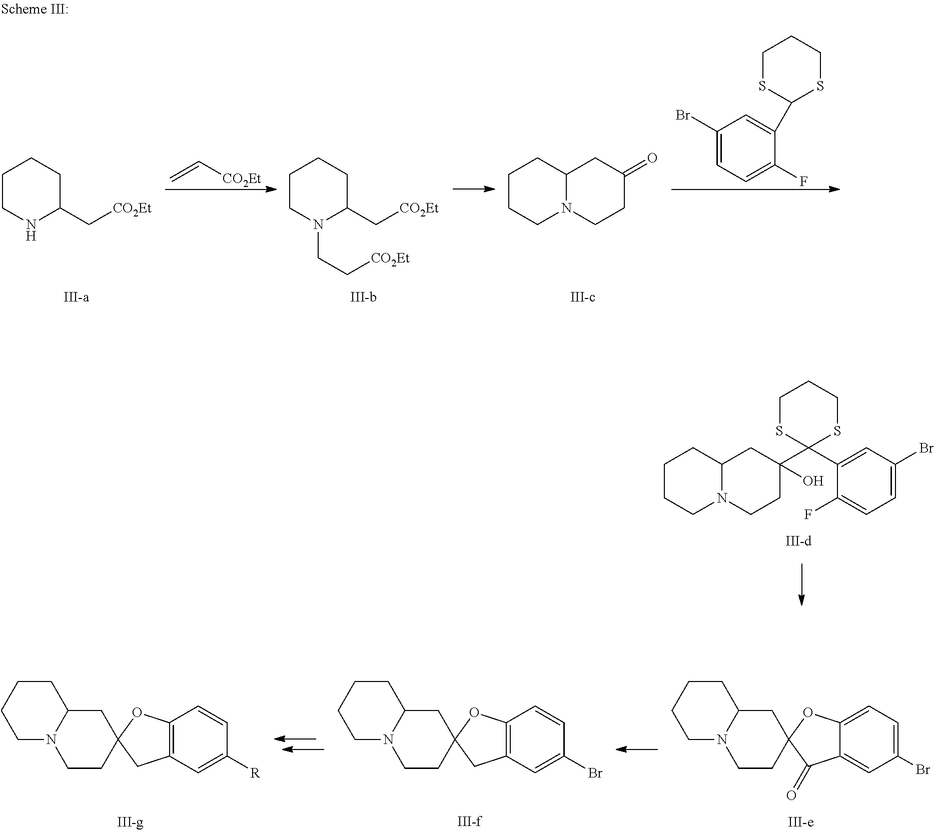

In one embodiment, a compound of formula (Va) or (Vb), specifically a compound of formula (VIIIa) or (VIIIb), may be prepared following Scheme III using suitable starting material. Compound III-a is treated with ethyl acrylate to generate compound III-b, which is treated with a base, such as LiHMDS, to render a cyclic β-ketoester intermediate which is decarboxylated by treatment with a strong acid, such as aqueous HCl, to prepare compound III-c. Treating compound III-c with the lithium salt of 2-(5-bromo-2-fluorophenyl)-1,3-dithiane yields the tertiary alcohol III-d. The dithiane protecting group is removed oxidatively, such as by treatment with pyridine tribromide, and the resulting ketone intermediate is cyclized by treatment with a base, such as KOH, to form the spiro-cyclic ketone III-e. The keto group of III-e is converted to a methylene group by treatment with a reducing agent, such as NaBH₄, followed by treatment with another reducing agent, such as Et₃SiH, to render III-f. The aryl bromide III-f may be converted to a suitable R substituents via known reaction. Optionally, further organic transformations may convert R to other suitable embodiments of R.

### D. METHODS OF TREATMENT, PREVENTION, AND/OR MANAGEMENT

1. Binding to Histamine Receptor

In various embodiments, provided herein is a method of binding a compound provided herein to a histamine receptor, such as, a histamine H3 receptor. The method comprises contacting the histamine receptor with a compound provided herein.

In other embodiments, provided herein is a method of inhibiting the binding of a histamine receptor ligand to a histamine receptor, such as, a histamine H3 receptor. The method comprises contacting the histamine receptor with a compound provided herein. In one embodiment, the histamine receptor ligand is an endogenous ligand. In another embodiment, the ligand is a drug molecule or another small molecule known to have binding affinity to the histamine receptor. In another embodiment, the histamine receptor ligand is a radioactively labeled compound, known to bind to the histamine receptor. In another embodiment, the ligand is an agonist, partial agonist, antagonist, or inverse agonist of the histamine receptor.

In one embodiment, inhibition of ligand binding is assessed using an in vitro binding assay, such as those described herein. In another embodiment, the compound provided herein inhibits mean binding by about 1%, about 5%, about 10%, about 20%, about 30%, about 40%, about 50%, about 60%, about 70%, about 80%, about 90%, about 95%, about 99%, or more, as compared to vehicle. In one embodiment, the inhibition of mean binding is dose dependent.

2. Inhibition of Histamine Receptor Activity

In various embodiments, provided herein is a method of modulating (e.g., inhibiting or augmenting) the activity of a histamine receptor, such as a histamine H3 receptor. The method comprises contacting the histamine receptor, such as histamine H3 receptor, with a compound provided herein, in vitro or in vivo. In one embodiment, the histamine receptor, such as histamine H3 receptor, is contacted with a compound provided herein by administering to a subject a therapeutically effective amount of the compound provided herein, or a pharmaceutically acceptable salt or stereoisomer thereof. The subject may be a human. In another embodiment, the histamine receptor is histamine H3 receptor.

In other embodiments, the compound provided herein inhibits or reduces the activity of a histamine receptor, such as histamine H3 receptor. Inhibition of histamine receptor activity may be measured using assays known in the art. In some embodiments, the activity of a histamine receptor is inhibited or reduced by about 1%, about 5%, about 10%, about 20%, about 30%, about 40%, about 50%, about 60%, about 70%, about 80%, about 90%, about 95%, about 99% or more, as compared with the activity obtained without contacting with the compounds provided herein. In one embodiment, the inhibition or reduction of receptor activity is dose dependent. Exemplary assay methods include, but are not limited to, in vitro functional assays. In one embodiment, the functional assay utilizes an appropriate cell-line expression a desired histamine receptor. In other embodiments, the functional assay utilizes synaptosomes isolated from brain tissue of an appropriate organism. In other embodiments, inhibition of histamine receptor activity may be assessed using receptor binding experiments know in the art, e.g. utilizing appropriate membrane preparations. In one embodiment, the assay involves treatment of a test subject (e.g., a rat) with a compound provided herein as well as a reference compound, followed by isolation of brain tissue and ex vivo analysis of receptor occupancy.

In certain embodiments, provided herein are methods of inhibiting or reducing the activity of a histamine receptor, e.g., H3 receptor, in a subject (e.g., human) comprising administering to the subject an effective amount of a compound provided herein. In some embodiments, the activity of histamine receptor is inhibited or reduced by about 1%, about 5%, about 10%, about 20%, about 30%, about 40%, about 50%, about 60%, about 70%, about 80%, about 90%, about 95%, about 99% or more, when measured using an assay described herein elsewhere.

In one embodiment, provided herein is a method of inhibiting or reducing the activity of a histamine receptor, such as a histamine H3 receptor, by a histamine receptor ligand. In one embodiment, the method comprises contacting the histamine receptor with an antagonist or an inverse agonist of the histamine receptor. In another embodiment, an antagonist or an inverse agonist of the histamine receptor is a compound provided herein.

3. Modulation of Histamine Release

In some embodiments, provided herein is a method of inhibiting a histamine receptor to increase the histamine release by a cell. The method includes contacting the cell with a compound provided herein. In one embodiment, the cell is a brain cell, such as a neuron or a glial cell. In one embodiment, the histamine release occurs in vivo. Thus, in certain embodiments, provided herein are methods of increasing the level of histamine release comprising administering to a subject (e.g., human) an effective amount of a compound provided herein. In an organism, the histamine release may occur, for example, at the synapse. Thus, in one embodiment, the neuronal cell is in contact with the synapse of a mammal. In another embodiment, the histamine release occurs in vitro. In some embodiments, the cell may be a brain cell, such as a neuronal cell or a cell type which expresses a histamine receptor, such as a histamine H3 receptor.

Stimulation of histamine release can be shown, for example, by performing various in vitro functional assays utilizing a cell type which expresses a certain type of histamine receptor, such as a histamine H3 receptor, together with an appropriate labeled histamine receptor ligand. In some embodiments, inhibition of the histamine receptor is demonstrated when an antagonist or inverse agonist (e.g., a compound provided herein) has an IC₅₀ of, for example, between about 0.1 nM and about 10 μM, between about 1 nM and about 1 μM, between about 1 nM and about 500 nM, and between about 1 nM and about 100 nM, in a functional histamine receptor assay, such as those described herein.

4. Treatment, Prevention, and/or Management of H3 Receptor Related Disorders

In one embodiment, provided herein are methods for the treatment, prevention, and/or management of a disorder provided herein, such as, e.g., a disorder related to histamine H3 receptor, such as, e.g., a neurological disorder provided herein. In one embodiment, provided herein are methods for the treatment, prevention, and/or management of one or more symptoms of a disorder provided herein, such as, e.g., a disorder related to histamine H3 receptor, such as, e.g., a neurological disorder provided herein. In one embodiment, the method provided herein comprises administering a compound provided herein. In one embodiment, the method provided herein comprises administering a compound provided herein, or a pharmaceutically acceptable salt or stereoisomer thereof. In one embodiment, the method provided herein comprises administering a composition provided herein. In one embodiment, the method provided herein comprises administering a pharmaceutical composition provided herein. In one embodiment, the method provided herein comprises administering a therapeutically effective amount of a compound provided herein. In one embodiment, the method provided herein comprises administering a prophylactically effective amount of a compound provided herein. In one embodiment, the method provided herein comprises administering a therapeutically effective or prophylactically effective amount of a compound provided herein, or a pharmaceutically acceptable salt or stereoisomer thereof.

In one embodiment, provided herein are uses of a compound provided herein in the manufacture of a medicament for the treatment, prevention, and/or management of a disorder provided herein, such as, e.g., a disorder related to histamine H3 receptor, such as, e.g., a neurological disorder. In one embodiment, provided herein are uses of a compound provided herein, or a pharmaceutically acceptable salt or stereoisomer thereof in the manufacture of a medicament for the treatment, prevention, and/or management of a disorder provided herein, such as, e.g., a disorder related to histamine H3 receptor, such as, e.g., a neurological disorder. In one embodiment, provided herein are uses of a composition provided herein in the manufacture of a medicament for the treatment, prevention, and/or management of a disorder provided herein, such as, e.g., a disorder related to histamine H3 receptor, such as, e.g., a neurological disorder. In one embodiment, provided herein are uses of a pharmaceutical composition provided herein in the manufacture of a medicament for the treatment, prevention, and/or management of a disorder provided herein, such as, e.g., a disorder related to histamine H3 receptor, such as, e.g., a neurological disorder.

In one embodiment, provided herein is a compound for use in the treatment, prevention, and/or management of a disorder provided herein, such as, e.g., a disorder related to histamine H3 receptor, such as, e.g., a neurological disorder provided herein. In one embodiment, provided herein is a compound, or a pharmaceutically acceptable salt or stereoisomer thereof, for use in the treatment, prevention, and/or management of a disorder provided herein, such as, e.g., a disorder related to histamine H3 receptor, such as, e.g., a neurological disorder provided herein. In one embodiment, provided herein is a composition for use in the treatment, prevention, and/or management of a disorder provided herein, such as, e.g., a disorder related to histamine H3 receptor, such as, e.g., a neurological disorder provided herein. In one embodiment, provided herein is a pharmaceutical composition for use in the treatment, prevention, and/or management of a disorder provided herein, such as, e.g., a disorder related to histamine H3 receptor, such as, e.g., a neurological disorder provided herein.

In one embodiment, provided herein are compounds, or pharmaceutically acceptable salts or stereoisomers thereof, for use in the treatment, prevention, and/or management of a disorder provided herein. In one embodiment, provided herein are compositions for use in the treatment, prevention, and/or management of a disorder provided herein. In one embodiment, provided herein are pharmaceutical compositions for use in the treatment, prevention, and/or management of a disorder provided herein. In one embodiment, provided herein are kits for use in the treatment, prevention, and/or management of a disorder provided herein.

In some embodiments, provided herein is a method of treating, preventing, and/or managing a disorder related to histamine H3 receptor, such as a neurological disorder. Without being limited by a particular theory, the treatment, prevention, and/or management is done by inhibiting or reducing the activity of histamine H3 receptor. Histamine H3 receptors modulate the release of neurotransmitters, including but not limited to, histamine, acetylcholine, norepinephrine, and dopamine, implicating a wide range of therapeutic indications. See, e.g., Haas et al., Physio. Rev. 88: 1183-241 (2008); Brown et al., Prog. Neurobio. 63: 637-72 (2001); Esbenshade et al., Mol. Interven. 6(2): 77-88 (2006); Esbenshade et al., British J. Pharmacol. 154(6): 1166-81 (2008); Sander et al., Bio. Pharm. Bull. 21: 2163-81 (2008).

In one embodiment, the method comprises administering to a subject (e.g., human) a therapeutically or prophylactically effective amount of a composition or compound provided herein. In one embodiment, the subject is a human. In another embodiment, the compound provided herein inhibits the activity of a histamine receptor. In another embodiment, the compound provided herein inhibits the activity of histamine H3 receptor. In certain embodiments, the compounds provided herein are inverse agonists of histamine H3 receptor. In other embodiments, the compounds provided herein are antagonists of histamine H3 receptors. In certain embodiments, the compounds provided herein are selective for histamine H3 receptor over other CNS-related targets. In one embodiment, the compounds provided herein are highly brain penetrable in animals, such as rodents, and human. In some embodiments, inhibition of the histamine receptor activity may be assessed by functional assays as described herein elsewhere. In certain embodiments, the efficacious concentration of the compounds provided herein is less than 10 nM, less than 100 nM, less than 1 μM, less than 10 μM, less than 100 μM, or less than 1 mM. In other embodiments, compound's activity may be assessed in various art-recognized animal models as described herein elsewhere.

In some embodiments, provided herein is a method of treating, preventing, and/or managing a disorder associated with excessive daytime sleepiness, such as narcolepsy, Parkinson's disease, Multiple Sclerosis, shift workers, jet lag, relief of side effects of other medications, and the like, comprising administering to a subject an effective amount of a compound provided herein. For example, without being limited by a particular theory, H3 antagonists or inverse agonists may have wake promoting effects. See, e.g., Lin et al., Br. Res. 523: 325-30 (1990); Barbier et al., Br. J. Pharm. 143: 649-61 (2004); Lin et al., Neurobiol. Dis. 30(1): 74-83 (2008).

In another embodiment, provided herein is a method of treating, preventing, and/or managing a sleeping disorder, such as insomnia, comprising administering to a subject an effective amount of a compound provided herein. For example, without being limited by a particular theory, H3 antagonists or inverse agonists may improve wakefulness and lead to an improved sleep pattern, and therefore H3 antagonists or inverse agonists may be useful in treating insomnia.

In another embodiment, provided herein is a method of treating, preventing, and/or managing substance abuse, comprising administering to a subject an effective amount of a compound provided herein. For example, without being limited by a particular theory, H3 antagonists can alter methamphetamine self-administration in rats, and therefore H3 antagonists may ameliorate the craving for addictive drugs. See, e.g., Munzar et al, Neuropsychopharmacology 29:705-17 (2004).

In another embodiment, provided herein is a method of treating, preventing, and/or managing a disorder related to cognitive impairments, impairments of learning, impairments of memory, and/or impairments of attention, vigilance and/or speed of response, such as those associated with Alzheimer's disease, Parkinson's disease, schizophrenia, mild cognitive impairment (MCI), and attention deficit hyperactivity disorder (ADHD), and the like, comprising administering to a subject an effective amount of a compound provided herein. For example, without being limited by a particular theory, H3 antagonists or inverse agonists may have pro-cognitive effects, such as, e.g., those measured by passive avoidance, novel object recognition, social recognition, and attention-set shifting. See, e.g., Medhurst et al., JPET 321: 1032-45 (2007); Medhurst et al., Biochem. Pharmcol. 73: 1182-94 (2007); Fox et al., JPET 313:176-190 (2005); Fox et al., JPET 305: 897-908 (2003). Further, without being limited by a particular theory, H3 receptor antagonists or inverse agonists may improve social memory, increase the acquisition of a test paradigm, and reverse scopolamine-induced deficits. H3 antagonists or inverse agonists may also reverse scopolamine-induced deficits in a passive avoidance memory test.

In another embodiment, provided herein is a method of treating, preventing, and/or managing a disorder related to psychosis, schizophrenia, ADHD, and/or mood disorders, such as depression and/or anxiety, comprising administering to a subject an effective amount of a compound provided herein. For example, without being limited by a particular theory, H3 antagonists or inverse agonists may improve the gating deficits of DBA/2 mice seen in the pre-pulse inhibition (PPI) test and reverse the methamphe-tamine-induced hyperlocomotor activity. See, e.g., Fox et al., JPET 313:176-190 (2005). Without being limited to a particular theory, H3 antagonists or inverse agonists may: 1) reverse the amphetamine-induced hyper-locomotor activity (See, e.g., Clapham et al., Eur. J. Pharmacol. 259: 107-14 (1994)); 2) be useful as antipsychotic agents and dosed sparing (See, e.g., Zhang et al., Br. Res. 1045: 142-49 (2005)); 3) improve attention and modulate impulsivity (See, e.g., Day et al., Biochem. Pharmacol. 73:1123-34 (2007)); 4) improve learning parameters in ADHD (See, e.g., Fox et al., JPET 313:176-90 (2005); Fox et al., JPET 305: 897-908 (2003); Fox et al., Behav. Br. Res. 131: 151-61 (2002); Komater et al., Psychopharm. 167: 363-72 (2003); Esbenshade et al., Biochem. Pharmacol. 68: 933-45 (2004)); 5) enhance learning ability and reduce anxiety in behavioral tests (See, e.g., Rizk et al., Eur. J. Neurosci. 19: 1992-96 (2004)); and 6) have an anti-depressant effect (See, e.g., Pérez-García et al., Psychopharm. 142(2): 215-20 (1999)).

In another embodiment, provided herein is a method of using the compounds provided herein as psycho-stimulants, which may lack the abuse liabilities generally associated with other classes of psycho-stimulants. Without being limited by a particular theory, H3 antagonists or inverse agonists increase the levels of histamine, dopamine, norepinephrine, and acetylcholine in the prefrontal cortical area, which is consistent with their pro-cognitive effects and their wake promoting effects seen in animal models. For example, H3 antagonists or inverse agonists may increase dopamine in the frontal cortex but not the striatum. H3 antagonists or inverse agonists may not induce increased locomotor activity or sensitization that is associated with other psycho-stimulus. See, e.g., Komater et al., Psychopharm. 167: 363-72 (2003).

In another embodiment, provided herein is a method of treating, preventing, and/or managing a disorder such as convulsion (e.g. epilepsy), seizures, vertigo, and pain, comprising administering to a subject an effective amount of a compound provided herein. For example, without being limited by a particular theory, H3 antagonists or inverse agonists may be protective against pentylenetetrazole (PTZ) and electrical-induced seizures. See, e.g., Vohora et al., Life Sci. 22: 297-301 (2000); Vohora et al., Pharmacol. Biochem. Behay. 68(4): 735-41 (2001); Zhang et al., Eur. J. Pharmacol. 15(581): 169-75 (2003). H3 antagonists or inverse agonists may increase the seizure threshold in humans. See, e.g., WO 2006/084833. H3 antagonists or inverse agonists may decrease electrical discharge from afferent neurons in an inner ear preparation. See, e.g., Chavez et al., Brain Res. 1064(1-2): 1-9 (2005). Further, H3 receptors are localized on neurons in the dorsal horn of the spinal cord, an area important for the transmission of nociceptive information in humans, and have shown efficacy in preclinical pain models. Thus, without being limited by a particular theory, H3 receptor antagonists or inverse agonists may increase the threshold for neuropathic pain, which was shown in models such as the chronic constriction injure (CCI) model, herpes virus-induced model, and capsaicin-induced allodynia model. See, e.g., Medhurst et al., Pain 138: 61-69 (2008); Medhurst et al., Biochem. Pharmacol. 73: 1182-94 (2007). Therefore, in some embodiments, the compounds provided herein are employed for their analgesic effects to treat, prevent, and/or manage disorders involving pain and the sensitization that accompanies many neuropathic pain disorders.

In yet another embodiment, provided herein is a method of treating, preventing, and/or managing a disorder related to satiety, gastric activity, irritable bowel syndrome (IBS), chronic constipation (CC), and/or metabolic disorders such as diabetes and obesity, comprising administering to a subject an effective amount of a compound provided herein. In other embodiments, provided herein is a method of mitigating the weight gain associated with other therapeutic agents, comprising administering to a subject an effective amount of a compound provided herein. For example, without being limited to a particular theory, H3 receptor plays a role in satiety. See, e.g., Masaki et al., Curr. Diabetes Rev. 3: 212-16 (2007); Ishizuka et al., Behav. Br. Res. 188: 250-54 (2008). H3 antagonists or inverse agonists may decrease food intake, reduce weight gain, reduce plasma triglyceride levels, modulate energy expenditure, reduce body weight and body fat, and normalize insulin tolerance. See, e.g., Malmlof et al., Obesity 14: 2154-62 (2006); Hancock et al., Eur J. Pharm. 487: 183-97 (2004). H3 antagonists or inverse agonists may also block olanzepine-induced decrease in satiety. See, e.g., WO 2006/084833.

In another embodiment, provided herein is a method of treating, preventing, and/or managing a disorder of enteric system and/or exocrine pancreatic system, such as acid secretion, digestion, and gut motility, comprising administering to a subject an effective amount of a compound provided herein. See, e.g., Breunig et al., J. Physiol. 583(2): 731-42 (2007); Singh et al., Inflamm. Res. 46: 159-65 (1997); Bertaccini et al., Dig. Dis. Sci. 40: 2052-63 (1995).

In another embodiment, provided herein is a method of treating, preventing, and/or managing movement disorders, such as Parkinson's disease, restless leg syndrome (RLS), and Huntington's disease, comprising administering to a subject an effective amount of a compound provided herein. For example, without being limited by a particular theory, an increased expression of H3 receptors have been found in the postmortem brain of subjects with Parkinson's disease. See, e.g., Anichtchik et al., Neurobiol. Dis. 8: 707-16 (2001); Anichtchik et al., Eur. J. Pharm. 12: 3823-32 (2000). Further, it was reported that a polymorphism in the primary enzyme that metabolizes histamine in the brain, the Thr105Ile polymorphism, results in a functional alteration in activity of the enzyme. This polymorphism has been associated with movement disorders such as Parkinson's disease and essential tremor. See, e.g., Preuss et al., JPET 53: 708-17 (1998); Agundez et al., Neuromol. Med. 10(1): 10-16 (2008); Ledesma et al., Neuromol. Med. 10(4): 356-61 (2008). Thus, H3 antagonists or inverse agonists may be useful in the treatment of Parkinson's disease. See, e.g., Gomez-Ramirez et al., Mov. Disord. 21: 839-46 (2006).

In some embodiments, the compounds provided herein are active in at least one model, which can be used to measure the activity of the compounds and estimate their efficacy in treating a neurological disorder. For example, when the model is for depression (e.g., mean immobility), the compounds are active when they inhibit mean immobility of a test subject by about 5%, about 10%, about 20%, about 30%, about 40%, about 50%, about 60%, about 70%, about 80%, about 90%, about 95%, about 99%, or more, when compared to vehicle. In some embodiments, the compounds provided herein produce a similar disparity in measured endpoint between treated animals and animals administered vehicle.

In other embodiments, provided herein is a method of effecting a therapeutic effect as described herein elsewhere. The method comprises administering to a subject (e.g., a mammal) a therapeutically effective amount of a compound or composition provided herein. The particular therapeutic effects may be measured using any model system known in the art and described herein, such as those involving an animal model of a disease.

In some embodiments, the neurological disorder is: depression (e.g., major depressive disorder, bipolar disorder, unipolar disorder, dysthymia and seasonal affective disorder); cognitive deficits; fibromyalgia; pain (e.g., neuropathic pain); sleep related disorders (e.g., sleep apnea, insomnia, narcolepsy, cataplexy) including those sleep disorders which are produced by psychiatric conditions; chronic fatigue syndrome; attention deficit disorder (ADD); attention deficit hyperactivity disorder (ADHD); restless leg syndrome; schizophrenia; anxieties (e.g., general anxiety disorder, social anxiety disorder, panic disorder); obsessive compulsive disorder; posttraumatic stress disorder; seasonal affective disorder (SAD); premenstrual dysphoria; post-menopausal vasomotor symptoms (e.g., hot flashes, night sweats); neurodegenerative disease (e.g., Parkinson's disease, Alzheimer's disease and amyotrophic lateral sclerosis); manic conditions; dysthymic disorder; cyclothymic disorder; obesity; or substance abuse or dependency (e.g., cocaine addiction, nicotine addiction). In another embodiment, the compounds provided herein are useful to treat, prevent, and/or manage two or more conditions/disorders, which are co-morbid, such as cognitive deficit and depression.

Neurological disorders include cerebral function disorders, including without limitation, senile dementia, Alzheimer's type dementia, cognition, memory loss, amnesia/amnestic syndrome, epilepsy, disturbances of consciousness, coma, lowering of attention, speech disorders, Lennox syndrome, autism, and hyperkinetic syndrome.

Neuropathic pain includes without limitation post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use).

Other exemplary diseases and conditions that may be treated, prevented, and/or managed using the methods, compounds, and/or compositions provided herein include, but are not limited to: obesity; migraine or migraine headache; urinary incontinence, including without limitation involuntary voiding of urine, dribbling or leakage of urine, stress urinary incontinence (SUI), urge incontinence, urinary exertional incontinence, reflex incontinence, passive incontinence, and overflow incontinence; and sexual dysfunction, in men or women, including without limitation sexual dysfunction caused by psychological and/or physiological factors, erectile dysfunction, premature ejaculation, vaginal dryness, lack of sexual excitement, inability to obtain orgasm, and psycho-sexual dysfunction, including without limitation, inhibited sexual desire, inhibited sexual excitement, inhibited female orgasm, inhibited male orgasm, functional dyspareunia, functional vaginismus, and atypical psychosexual dysfunction.

In one embodiment, the neurological disorder is excessive daytime sleepiness. In another embodiment, the neurological disorder is cognitive impairment. In another embodiment, the neurological disorder is mood disorders. In another embodiment, the neurological disorder is movement disorders. In another embodiment, the neurological disorder is schizophrenia. In another embodiment, the neurological disorder is attention disorders. In another embodiment, the neurological disorder is anxiety disorder. In another embodiment, the neurological disorder is seizure. In another embodiment, the neurological disorder is epilepsy. In another embodiment, the neurological disorder is vertigo. In another embodiment, the neurological disorder is pain. In another embodiment, the neurological disorder is neuropathic pain. In another embodiment, the neuropathic pain is diabetic neuropathy. In another embodiment, the neurological disorder is sleeping disorder. In another embodiment, the neurological disorder is insomnia. In another embodiment, the neurological disorder is substance abuse.

In one embodiment, the neurological disorder is a neurodegenerative disease. In one embodiment, the neurodegenerative disease is Parkinson's disease. In another embodiment, the neurodegenerative disorder is Alzheimer's disease.

In one embodiment, the disorder is obesity, and the therapeutically effective amount of compound to supply to a patient is sufficient so that said patient feels satiated. In another embodiment, the disorder is diabetes. In another embodiment, the disorder is metabolic diseases. In another embodiment, the disorder is a disease effecting the enteric system.

In one embodiment, the compounds described herein treat, prevent, and/or manage a central nervous disorder, without causing addiction to said compounds.

Any suitable route of administration can be employed for providing the patient with a therapeutically or prophylactically effective dose of an active ingredient. For example, oral, mucosal (e.g., nasal, sublingual, buccal, rectal, vaginal), parenteral (e.g., intravenous, intramuscular), transdermal, and subcutaneous routes can be employed. Exemplary routes of administration include oral, transdermal, and mucosal. Suitable dosage forms for such routes include, but are not limited to, transdermal patches, ophthalmic solutions, sprays, and aerosols. Transdermal compositions can also take the form of creams, lotions, and/or emulsions, which can be included in an appropriate adhesive for application to the skin or can be included in a transdermal patch of the matrix or reservoir type as are conventional in the art for this purpose. An exemplary transdermal dosage form is a “reservoir type” or “matrix type” patch, which is applied to the skin and worn for a specific period of time to permit the penetration of a desired amount of active ingredient. The patch can be replaced with a fresh patch when necessary to provide constant administration of the active ingredient to the patient.

The amount to be administered to a patient to treat, prevent, and/or manage the disorders described herein will depend upon a variety of factors including the activity of the particular compound employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount required. For example, the physician or veterinarian could start doses of the compounds employed at levels lower than that required in order to achieve the desired therapeutic effect and gradually increase the dosage until the desired effect is achieved.

In general, a suitable daily dose of a compound provided herein will be that amount of the compound which is the lowest dose effective to produce a therapeutic or prophylactic effect. Such an effective dose will generally depend upon the factors described above. Generally, oral, intravenous, intracerebroventricular and subcutaneous doses of the compounds provided herein for a patient will range from about 0.005 mg per kilogram to about 5 mg per kilogram of body weight per day. In one embodiment, the oral dose of a compound provided herein will range from about 10 mg to about 300 mg per day. In another embodiment, the oral dose of a compound provided herein will range from about 20 mg to about 250 mg per day. In another embodiment, the oral dose of a compound provided herein will range from about 100 mg to about 300 mg per day. In another embodiment, the oral dose of a compound provided herein will range from about 10 mg to about 100 mg per day. In another embodiment, the oral dose of a compound provided herein will range from about 25 mg to about 50 mg per day. In another embodiment, the oral dose of a compound provided herein will range from about 50 mg to about 200 mg per day. Each of the above-recited dosage ranges may be formulated as a single or multiple unit dosage formulations.

In some embodiments, the compound disclosed herein may be used in combination with one or more second active agent(s) to treat, prevent, and/or manage a disorder described herein. In one embodiment, the second active agent is known in the art, such as, e.g., those described in http://www.fda.gov/; The Merck Manual, 18th ed. 2006; and PDR: Physician Desk Reference 2010, 64th ed. 2009; the contents of each of which are hereby incorporated by reference in their entireties. In one embodiment, the second active agent is lurasidone, olanzapine, risperidone, aripiprazole, donepezil, rivastigmine, memantine, amphetamine, methylphenidate, atomoxetine, modafinil, sertraline, fluoxetine, or L-DOPA. In one embodiment, the second active agent includes, but is not limited to, lurasidone, olanzapine, risperidone, aripiprazole, donepezil, rivastigmine, memantine, amphetamine, methylphenidate, atomoxetine, modafinil, sertraline, fluoxetine, or L-DOPA.

5. Pharmaceutical Compositions and Dosage Forms

Pharmaceutical compositions can be used in the preparation of individual, single unit dosage forms. Pharmaceutical compositions and dosage forms provided herein comprise a compound provided herein, or a pharmaceutically acceptable salt or stereoisomer thereof, or a clathrate or prodrug thereof. Pharmaceutical compositions and dosage forms can further comprise one or more excipients.

Pharmaceutical compositions and dosage forms provided herein can also comprise one or more additional active ingredients. Examples of optional second, or additional, active ingredients are also disclosed herein.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms are used will vary from one another and will be readily apparent to those skilled in the art. See, e.g., Remington's Pharmaceutical Sciences, 18th ed., Mack Publishing, Easton Pa. (1990).

In one embodiment, pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided are pharmaceutical compositions and dosage forms that contain little, if any, lactose other mono- or disaccharides. As used herein, the term “lactose-free” means that the amount of lactose present, if any, is insufficient to substantially increase the degradation rate of an active ingredient.

Lactose-free compositions can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. In one embodiment, lactose-free dosage forms comprise active ingredients, microcrystalline cellulose, pre-gelatinized starch, and magnesium stearate.

Also provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are, in one embodiment, packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Also provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. In one embodiment, dosage forms comprise a compound provided herein in an amount of from about 0.10 to about 500 mg. In other embodiments, dosage forms comprise a compound provided herein in an amount of about 0.1, 1, 2, 5, 7.5, 10, 12.5, 15, 17.5, 20, 25, 50, 100, 150, 200, 250, 300, 350, 400, 450, or 500 mg.

In other embodiments, dosage forms comprise the second active ingredient in an amount of 1 to about 1000 mg, from about 5 to about 500 mg, from about 10 to about 350 mg, or from about 50 to about 200 mg. Of course, the specific amount of the second active agent will depend on the specific agent used, the diseases or disorders being treated or managed, and the amount(s) of a compound provided herein, and any optional additional active agents concurrently administered to the patient.

5.1 Oral Dosage Forms

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's The Science and Practice of Pharmacy, 21st ed., Lippincott Williams & Wilkins (2005).

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In one embodiment, oral dosage forms are tablets or capsules, in which case solid excipients are employed. In another embodiment, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in one embodiment, present in from about 50 to about 99 weight percent of the pharmaceutical composition or dosage form.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In one embodiment, pharmaceutical compositions comprise from about 0.5 to about 15 weight percent of disintegrant, or from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants may be used in an amount of less than about 1 weight percent of the pharmaceutical compositions or dosage forms into which they are incorporated.

In one embodiment, a solid oral dosage form comprises a compound provided herein, and optional excipients, such as anhydrous lactose, microcrystalline cellulose, polyvinylpyrrolidone, stearic acid, colloidal anhydrous silica, and gelatin.

5.2 Controlled Release Dosage Forms

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents provided herein. In one embodiment, provided are single unit dosage forms suitable for oral administration such as, but not limited to, tablets, capsules, gelcaps, and caplets that are adapted for controlled-release.

In one embodiment, controlled-release pharmaceutical products improve drug therapy over that achieved by their non-controlled counterparts. In another embodiment, the use of a controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

In another embodiment, the controlled-release formulations are designed to initially release an amount of drug (active ingredient) that promptly produces the desired therapeutic or prophylactic effect, and gradually and continually release of other amounts of drug to maintain this level of therapeutic or prophylactic effect over an extended period of time. In one embodiment, in order to maintain a constant level of drug in the body, the drug can be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.3 Parenteral Dosage Forms

Parenteral dosage forms can be administered to patients by various routes including, but not limited to, subcutaneous, intravenous (including bolus injection), intramuscular, and intra-arterial. In some embodiments, administration of a parenteral dosage form bypasses patients' natural defenses against contaminants, and thus, in these embodiments, parenteral dosage forms are sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms. For example, cyclodextrin and its derivatives can be used to increase the solubility of a compound provided herein. See, e.g., U.S. Pat. No. 5,134,127, which is incorporated herein by reference.

5.4 Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein are well known to those skilled in the pharmaceutical arts, and depend on the particular tissue to which a given pharmaceutical composition or dosage form will be applied. In one embodiment, excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms. Examples of additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Also, the polarity of a solvent carrier, its ionic strength, or tonicity can be adjusted to improve delivery. Compounds such as stearates can also be added to pharmaceutical compositions or dosage forms to alter the hydrophilicity or lipophilicity of one or more active ingredients so as to improve delivery. In other embodiments, stearates can serve as a lipid vehicle for the formulation, as an emulsifying agent or surfactant, or as a delivery-enhancing or penetration-enhancing agent. In other embodiments, salts, stereoisomers, solvates, prodrugs, or clathrates of the active ingredients can be used to further adjust the properties of the resulting composition.

6. Kits

In one embodiment, active ingredients provided herein are not administered to a patient at the same time or by the same route of administration. In another embodiment, provided are kits which can simplify the administration of appropriate amounts of active ingredients.

In one embodiment, a kit comprises a dosage form of a compound provided herein. Kits can further comprise one or more second active ingredients as described herein, or a pharmacologically active mutant or derivative thereof, or a combination thereof.

In other embodiments, kits can further comprise devices that are used to administer the active ingredients. Examples of such devices include, but are not limited to, syringes, drip bags, patches, and inhalers.

Kits can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### V. EXAMPLES

Certain embodiments are illustrated by the following non-limiting examples.

### A. Synthesis of Compounds

In the examples below, unless otherwise indicated, all temperatures are set forth in degrees Celsius and all parts and percentages are by weight. Reagents may be purchased from commercial suppliers, such as Sigma-Aldrich Chemical Company, and may be used without further purification unless otherwise indicated. Reagents may also be prepared following standard literature procedures known to those skilled in the art. Solvents may be purchased from Aldrich in Sure-Seal bottles and used as received. All solvents may be purified using standard methods known to those skilled in the art, unless otherwise indicated.

The reactions set forth below were done generally at ambient temperature, unless otherwise indicated. The reaction flasks were fitted with rubber septa for introduction of substrates and reagents via syringe. Analytical thin layer chromatography (TLC) was performed using glass-backed silica gel pre-coated plates (Merck Art 5719) and eluted with appropriate solvent ratios (v/v). Reactions were assayed by TLC or LCMS, and terminated as judged by the consumption of starting material. Visualization of the TLC plates was done with UV light (254 wavelength) or with an appropriate TLC visualizing solvent, such as basic aqueous KMnO₄ solution activated with heat. Flash column chromatography (See, e.g., Still et al., J. Org. Chem., 43: 2923 (1978)) was performed using silica gel 60 (Merck Art 9385) or various MPLC systems.

The compound structures in the examples below were confirmed by one or more of the following methods: proton magnetic resonance spectroscopy, mass spectroscopy, elemental microanalysis, and melting point. Proton magnetic resonance (¹H NMR) spectra were determined using a NMR spectrometer operating at a certain field strength. Chemical shifts are reported in parts per million (ppm, δ) downfield from an internal standard, such as TMS. Alternatively, ¹H NMR spectra were referenced to signals from residual protons in deuterated solvents as follows: CDCl₃=7.25 ppm; DMSO-d₆=2.49 ppm; C₆D₆=7.16 ppm; CD₃OD=3.30 ppm. Peak multiplicities are designated as follows: s, singlet; d, doublet; dd, doublet of doublets; t, triplet; dt, doublet of triplets; q, quartet; br, broadened; and m, multiplet. Coupling constants are given in Hertz (Hz). Mass spectra (MS) data were obtained using a mass spectrometer with APCI or ESI ionization.

### 1. Compound 1: 1′-Cyclobutyl-3H-spiro[benzofuran-2,4′-piperidine]

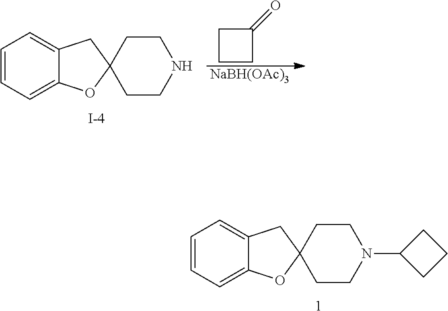

To a solution of I-4 (80 mg, 0.42 mmol) in dichloromethane was added acetic acid (25 mg, 0.42 mmol) and cyclobutanone (60 mg, 0.85 mmol) and the resulting mixture was stirred at room temperature for 1 hour. Solid NaBH(OAc)₃ (180 mg, 0.85 mmol) was added and the reaction mixture was stirred at room temperature overnight. The reaction mixture was diluted with additional dichloromethane and washed with saturated NaHCO₃. The combined organic layers were dried over Na₂SO₄, the solids were filtered and the filtrate was concentrated. The crude reaction mixture was purified by preparative TLC to give Compound 1 (40 mg, 40%). ¹H NMR (400 MHz, CDCl₃) δ: 1.67-2.09 (m, 10H), 2.44 (brs, 4H), 2.79 (m, 1H), 2.97 (s, 2H), 6.74-6.79 (dd, 1H), 6.80-6.82 (dd, 1H), 7.08-7.13 (m, 2H). MS (ESI): m/z 244.2 (M+H)⁺.

### 2. Compound 2: 1′-(1-Methylpiperidin-4-yl)-3H-spiro[benzofuran-2,4′-piperidine]

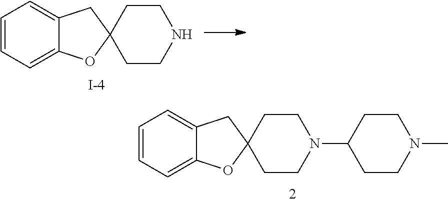

This compound was prepared in 72% yield (45 mg) as described for Compound 1 but using 1-methylpiperidin-4-one as the starting material. ¹H NMR (400 MHz, CDCl₃) δ: 1.60-1.69 (m, 2H), 1.75-1.82 (m, 4H), 1.91-1.99 (m, 4H), 2.26 (s, 3H), 2.30-2.36 (m, 1H), 2.60-2.63 (m, 2H), 2.72-2.77 (m, 2H), 2.90-2.96 (m, 4H), 6.73-6.81 (m, 2H), 7.07-7.28 (m, 2H). MS (ESI): m/z 287.2 (M+H)⁺.

### 3. Compound 3: 1′-((1H-Imidazol-4-yl)methyl)-3H-spiro [benzofuran-2,4′-piperidine]

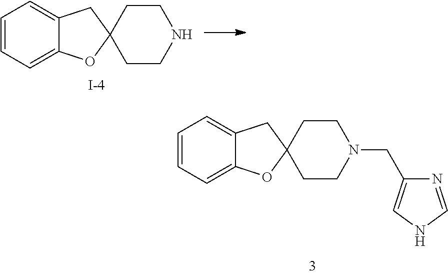

This compound was prepared in 70% yield (70 mg) as described for Compound 1 but using 1H-imidazole-4-carbaldehyde as the starting material. ¹H NMR (400 MHz, CDCl₃) δ: 1.78-1.85 (m, 2H), 1.95-2.00 (m, 2H), 2.61-2.70 (m, 4H), 2.98 (s, 2H), 3.61 (s, 2H), 6.75 (1H), 6.79-6.83 (m, 1H), 7.08-7.14 (m, 2H), 7.61 (1H). MS (ESI): m/z 270.1 (M+H)⁺.

### 4. Compound 4: 1′-(1-Cyclobutylethyl)-3H-spiro[benzofuran-2,4′-piperidine]

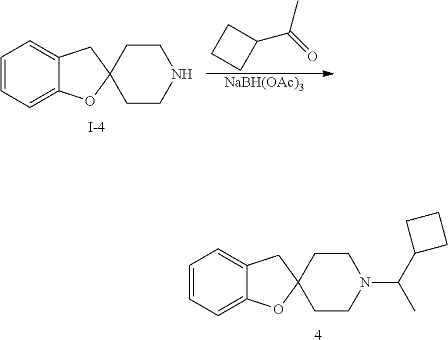

To a solution of I-4 (600 mg, 2.2 mmol) and 1-cyclobutylethanone (440 mg, 4.5 mmol) in 1,2-dichloroethane was added solid NaBH(OAc)₃ (950 mg, 4.5 mmol) and the resulting mixture was refluxed overnight. The reaction mixture was diluted with additional 1,2-dichloroethane and washed with saturated NaHCO₃. The combined organic layers were dried over Na₂SO₄, the solids were filtered and the filtrate was concentrated. The crude reaction mixture was purified by silica gel chromatography to give Compound 4 as a white solid (200 mg, 25%). ¹H NMR (400 MHz, CD₃OD) δ: 1.26 (d, 3H), 1.78-2.17 (m, 10H), 2.67-2.73 (m, 1H), 3.05 (s, 2H), 3.31-3.42 (m, 5H), 6.74 (1H), 6.80-6.84 (m, 1H), 7.06-7.16 (m, 2H). MS (ESI): m/z 272.2 (M+H)⁺.

### 5. Compound 5: 4-(1′-Cyclobutyl-3H-spiro[benzofuran-2,4′-piperidine]-5-yl)benzonitrile

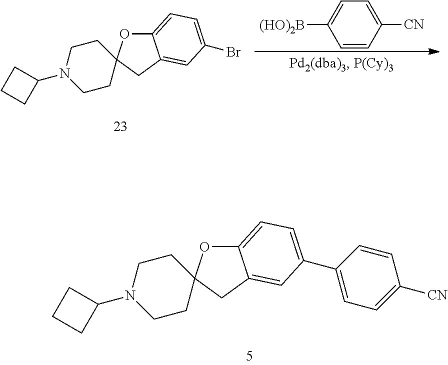

Compound 23 (80 mg, 25 mmol), Pd₂(dba)₃ (10 mg), P(Cy)₃ (10 mg) and KF (35 mg) were suspended in a mixture of dioxane (4.5 mL) and water (1.5 mL) and the reaction mixture was heated under microwave irradiation for 1 hour at 100° C. Solids were filtered, the filtrate was concentrated and the residue was purified by preparative TLC to give Compound 5 (20 mg). ¹H NMR (400 MHz, CDCl₃) δ: 1.70-1.74 (m, 2H), 1.80-2.08 (m, 9H), 2.46 (br s, 3H), 2.78-2.82 (m, 1H), 3.04 (s, 2H), 6.84 (d, J=8.0 Hz, 1H), 7.34-7.37 (m, 2H), 7.59 (d, J=8.4 Hz, 2H), 7.66 (d, J=8.4 Hz, 2H). MS (ESI): m/z 345.2 (M+H)⁺.

### 6. Compound 6: N-(4-(1′-Cyclobutyl-3H-spiro[benzofuran-2,4′-piperidine]-5-yl)phenyl)acetamide

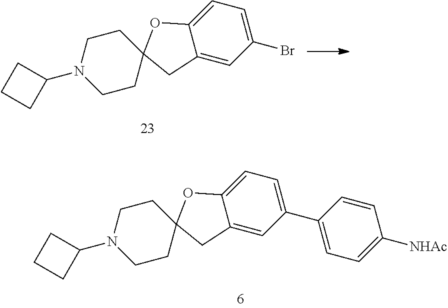

This compound was prepared in 16% yield (13 mg) as described for Compound 5 but using 4-acetamidophenylboronic acid as the starting material.

¹H NMR (400 MHz, CDCl₃) δ: 1.69-2.07 (m, 11H), 2.17 (s, 3H), 2.45 (br s, 3H), 2.81 (m, 1H), 3.01 (s, 2H), 6.79 (d, J=8.0 Hz, 1H), 7.28-7.30 (m, 2H), 7.44, (d, J=8.8 Hz, 2H), 7.52 (d, J=8.8 Hz, 1H). MS (ESI): m/z 377.2 (M+H)⁺.

### 7. Compound 7: 1′-Cyclobutyl-5-(1H-indol-5-yl)-3H-spiro[benzofuran-2,4′-piperidine]

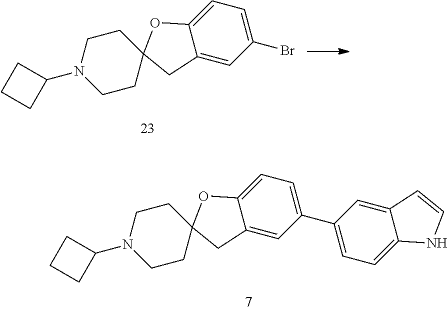

This compound was prepared in 10% yield (7 mg) as described for Compound 5 but using 1H-indol-5-ylboronic acid as the starting material. ¹H NMR (400 MHz, CDCl₃, D₂O exchange) δ: 1.70-1.75 (m, 2H), 1.82-1.88 (m, 4H), 1.91-1.95 (m, 4H), 2.48 (br s, 3H), 2.81 (m, 1H), 3.04 (s, 2H), 6.57 (m, 1H), 6.83 (dd, 1H), 7.21-7.27 (m, 1.5H), 7.37-7.41 (m, 3.5H), 7.77 (1H). MS (ESI): m/z 359.2 (M+H)⁺.

### 8. Compound 8: 4-(1′-(1-Methylpiperidin-4-yl)-3H-spiro[benzofuran-2,4′-piperidine]-5-yl)benzonitrile

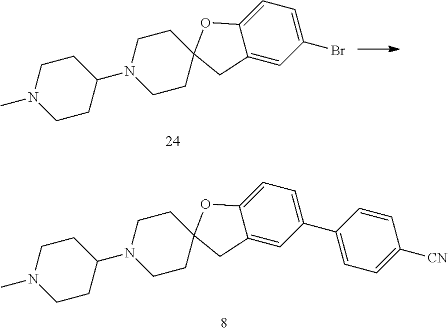

This compound was prepared in 16% yield (15 mg) as described for compound 5 but using Compound 24 as the starting material. ¹H NMR (400 MHz, CD₃OD) δ: 2.19-2.27 (m, 6H), 2.48 (d, 2H), 2.93 (s, 3H), 3.15-3.22 (m, 4H), 3.50 (br s, 2H), 3.63-3.75 (m, 5H), 6.89 (d, J=8.4 Hz, 1H), 7.48 (dd, J₁=8.4 Hz, J₂=1.6 Hz, 1H), 7.56 (d, J₂=1.6 Hz, 1H), 7.74 (s, 4H). MS (ESI): m/z 388.2 (M+H)⁺.

### 9. Compound 9: N-(4-(1′-(1-Methylpiperidin-4-yl)-3H-spiro[benzofuran-2,4′-piperidine]-5-yl)phenyl)acetamide

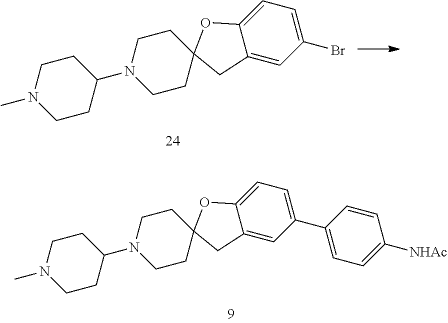

This compound was prepared in 21% yield (23 mg) as described for Compound 5 but using compound 24 and 4-acetamidophenylboronic acid as the starting material. ¹H NMR (400 MHz, CD₃OD) δ: 2.14-2.25 (m, 9H), 2.45-2.48 (m, 2H), 2.91 (s, 3H), 3.15-3.21 (m, 4H), 3.44 (br s, 2H), 3.60-3.72 (m, 5H), 6.81 (d, J=8.4 Hz, 1H), 7.37 (d, J=8.4 Hz, 1H), 7.43 (s, 1H), 7.49 (d, J=8.8 Hz, 2H), 7.58 (d, J=8.8 Hz, 2H). MS (ESI): m/z 420.2 (M+H)⁺.

### 10. Compound 10: 5-(1H-Indol-5-yl)-1′-(1-methylpiperidin-4-yl)-3H-spiro[benzofuran-2,4′-piperidine]

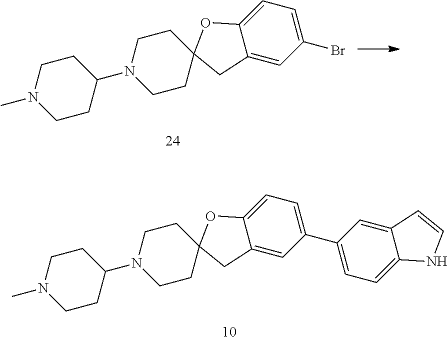

This compound was prepared in 11% yield (10 mg) as described for Compound 5 but using Compound 24 and 1H-indol-5-ylboronic acid as the starting material. ¹H NMR (400 MHz, CDCl₃, D₂O exchange) δ: 1.63-1.71 (m, 2H), 1.83-1.85 (m, 4H), 1.94-2.04 (m, 4H), 2.27 (s, 3H), 2.34-2.39 (m, 1H), 2.67 (br s, 2H), 2.76-2.81 (m, 2H), 2.93-2.95 (m, 2H), 3.03 (s, 2H), 6.56 (1H), 6.81 (1H), 7.21 (1H), 7.35-7.41 (m, 4H), 7.76 (s, 1H). MS (ESI): m/z 402.2 (M+H)⁺.

### 11. Compound 11: 4-(1′-(1-Cyclobutylethyl)-3H-spiro[benzofuran-2,4′-piperidine]-5-yl)benzonitrile

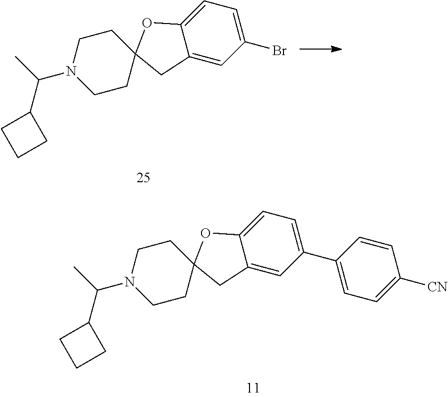

This compound was prepared in 16% yield (13 mg) as described for compound 5 but using compound 25 as the starting material. ¹H NMR (400 MHz, CDCl₃) δ: 0.92 (d, J=6.8 Hz, 3H), 1.64-1.88 (m, 6H), 1.94-2.02 (m, 4H), 2.40-2.42 (m, 1H), 2.49-2.57 (m, 3H), 2.66-2.75 (m, 2H), 3.01 (s, 2H), 6.84 (d, J=8.4 Hz, 1H), 7.33-7.36 (m, 2H), 7.59 (d, J=8.0 Hz, 2H), 7.66 (d, J=8.0 Hz, 2H). MS (ESI): m/z 373.3 (M+H)⁺.

### 12. Compound 12: N-(4-(1′-(1-Cyclobutylethyl)-3H-spiro[benzofuran-2,4′-piperidine]-5-yl)phenyl)acetamide

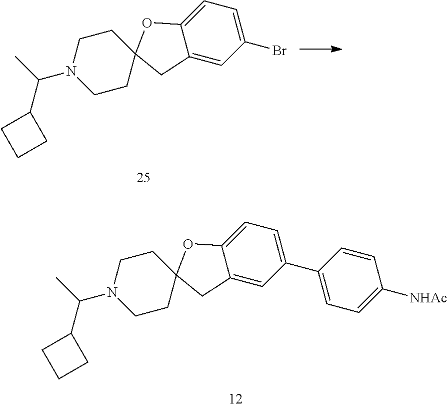

This compound was prepared in 19% yield (22 mg) as described for compound 5 but using compound 25 and 4-acetamidophenylboronic acid as the starting material. ¹H NMR (400 MHz, CDCl₃, D₂O exchange) δ: 0.92 (d, J=6.8 Hz, 3H), 1.65-1.86 (m, 6H), 1.94-1.98 (m, 4H), 2.18 (s, 3H), 2.43 (m, 1H), 2.54-2.58 (m, 3H), 2.65-2.80 (m, 2H), 2.99 (s, 2H), 6.79 (d, J=8.4 Hz, 1H), 7.28-7.31 (m, 2H), 7.45 (d, J=8.4 Hz, 2H), 7.52 (d, J=8.4 Hz, 2H). MS (ESI): m/z 405.3 (M+H)⁺.

### 13. Compound 13: 1′-(1-Cyclobutylethyl)-5-(1H-indol-5-yl)-3H-spiro[benzofuran-2,4′-piperidine]

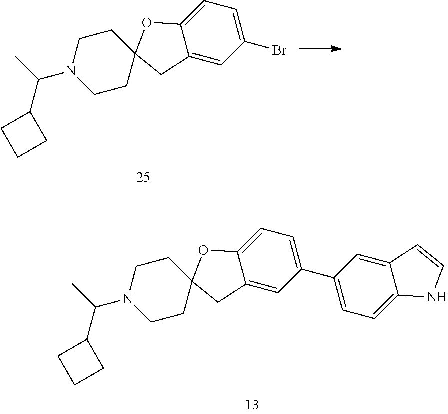

This compound was prepared in 8% yield (5.5 mg) as described for compound 5 but using compound 25 and 1H-indol-5-ylboronic acid as the starting material. ¹H NMR (400 MHz, CDCl₃, D₂O exchange) δ: 0.96 (3H), 1.66-1.69 (m, 2H), 1.80-1.93 (m, 4H), 1.95-2.10 (m, 4H), 2.46 (m, 1H), 2.60 (m, 3H), 2.77 (m, 2H), 3.02 (s, 2H), 6.57 (d, J=3.2 Hz, 1H), 6.81 (d, J=8.0 Hz, 1H), 7.22 (d, J=3.2 Hz, 1H), 7.35-7.45 (m, 4H), 7.76 (s, 1H). MS (ESI): m/z 387.2 (M+H)⁺.

### 14. Compound 14: (4-(1′-Cyclobutyl-3H-spiro[benzofuran-2,4′-piperidine]-5-yl)phenyl)methanamine

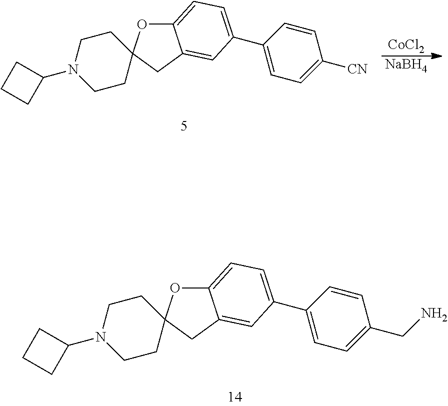

Compound 5 (40 mg, 0.12 mmol) was dissolved in a mixture of THF (6 mL) and H₂O (3 mL) and cobalt (II) chloride hexahydrate (20 mg) was added followed by NaBH₄ (20 mg, 0.53 mmol). The mixture was stirred until all starting material disappeared (monitored by TLC). The reaction mixture was concentrated and the residue was diluted with water and dichloromethane. The organic layer was separated, dried over sodium sulfate, the solids were filtered and the filtrate was concentrated. The crude reaction mixture was purified by preparative TLC to give 14 (18 mg, 44%). ¹H NMR (400 MHz, CDCl₃, D₂O exchange) δ: 1.67-2.09 (m, 11H), 2.45 (brs, 3H), 2.81 (m, 1H), 3.03 (s, 1H), 3.87 (s, 2H), 6.81 (d, J=8.0 Hz, 1H), 7.32-7.35 (m, 4H), 7.48 (d, J=8.0 Hz, 2H). MS (ESI): m/z 349.3 (M+H)⁺.

### 15. Compound 15: (4-(1′-(1-Methylpiperidin-4-yl)-3H-spiro[benzofuran-2,4′-piperidine]-5-yl)phenyl)methanamine

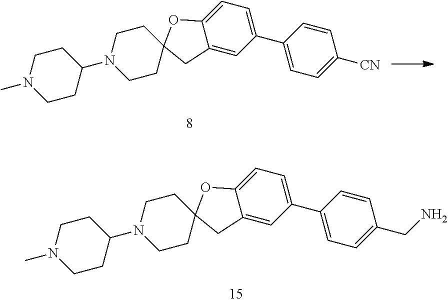

This compound was prepared in 48% yield (15 mg) as described for compound 14 but using compound 8 as the starting material. ¹H NMR (400 MHz, CDCl₃) δ: 1.60-2.10 (m, 10H), 2.31 (s, 3H), 2.48 (m, 1H), 2.70-2.90 (m, 4H), 2.97-3.00 (m, 2H), 3.04 (s, 2H), 3.89 (s, 2H), 6.81 (d, J₂=8.0 Hz, 1H), 7.33-7.35 (m, 4H), 7.48 (d, J₂=8.0 Hz, 12H). MS (ESI): m/z 392.3 (M+H)⁺.

### 16. Compound 16: (4-(1′-(1-Cyclobutylethyl)-3H-spiro[benzofuran-2,4′-piperidine]-5-yl)phenyl)methanamine

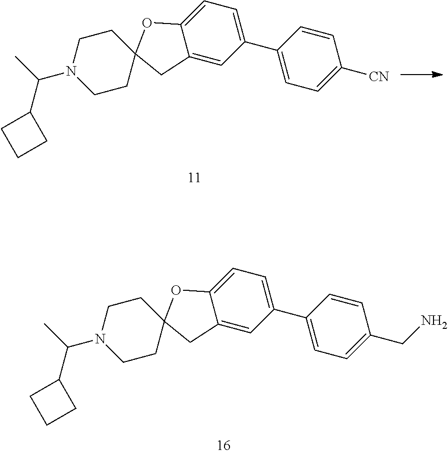

This compound was prepared in 30% yield (12 mg) as described for compound 14 but using compound 11 as the starting material. ¹H NMR (400 MHz, CDCl₃, D₂O exchange) δ: 0.92 (d, J=6.8 Hz, 3H), 1.65-1.86 (m, 6H), 1.94-1.98 (m, 4H), 2.42 (m, 1H), 2.54-2.58 (m, 3H), 2.65-2.80 (m, 2H), 3.00 (s, 2H), 3.86 (s, 2H), 6.80 (d, J=8.4 Hz, 1H), 7.31-7.34 (m, 4H), 7.48 (d, J=8.4 Hz, 2H). MS (ESI): m/z 377.1 (M+H)⁺.

### 17. Compound 17: 4-(1′-Cyclopentyl-3H-spiro[benzofuran-2,4′-piperidine]-5-yl)benzonitrile

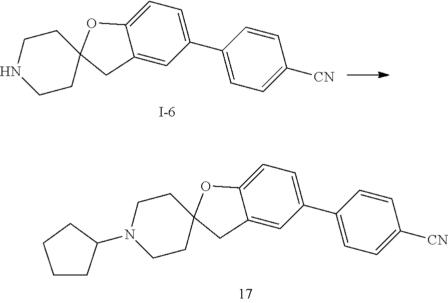

This compound was prepared in 18% yield (11 mg) as described for compound 1 but using cyclopentanone and intermediate I-6 as the starting material. ¹H NMR (400 MHz, CD₃OD) δ: 7.69-7.67 (m, 2H), 7.62-7.59 (m, 2H), 7.38-7.35 (m, 2H), 6.85 (d, J=8.4 Hz, 1H), 3.06 (s, 2H), 2.69-2.61 (m, 4H), 2.05-2.02 (m, 2H), 1.94-1.91 (m, 4H), 1.73-1.42 (m, 7H). MS (ESI): m/z 359.1 (M+H)⁺.

### 18. Compound 18: 4-(1′42-Methylcyclopentyl)-3H-spiro[benzofuran-2,4′-piperidine]-5-yl)benzonitrile

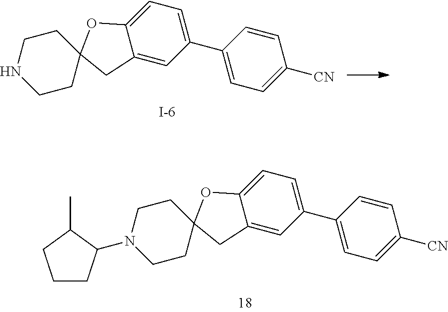

This compound was prepared in 15% yield (9 mg) as described for compound 1 but using 2-methylcyclopentanone and intermediate I-6 as the starting material. ¹H NMR (400 MHz, CD₃OD) δ: 7.64 (s, 4H), 7.42 (s, 1H), 7.34 (d, J=8.0 Hz, 1H), 6.70 (d, J=6.4 Hz, 1H), 2.99 (s, 2H), 2.71-2.38 (m, 4H), 2.17-2.04 (m, 1H), 1.95-1.68 (m, 8H), 1.56-1.42 (m, 3H), 0.79 (d, J=6.8 Hz, 3H). MS (ESI): m/z 373.1 (M+H)⁺.

### 19. Compound 19: 4-(1′-(Tetrahydro-2H-pyran-4-yl)-3H-spiro[benzofuran-2,4′-piperidine]-5-yl)benzonitrile

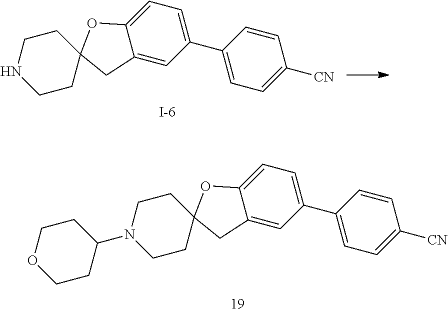

This compound was prepared in 17% yield (19 mg) as described for compound 1 but using dihydro-2H-pyran-4(3H)-one and intermediate I-6 as the starting material. ¹H NMR (400 MHz, CD₃OD) δ: 7.72-7.63 (m, 4H), 7.49 (s, 1H), 7.40 (d, J=8.4 Hz, 1H), 6.83 (d, J=8.0 Hz, 1H), 4.05-4.01 (m, 2H), 3.63-3.38 (m, 7 H), 3.13 (s, 2H), 2.27-2.04 (m, 6H), 1.84-1.76 (m, 2H). MS (ESI): m/z 375.1 (M+H)⁺.

### 20. Compound 20: 4-(1′-Cyclohexyl-3H-spiro[benzofuran-2,4′-piperidine]-5-yl)benzonitrile

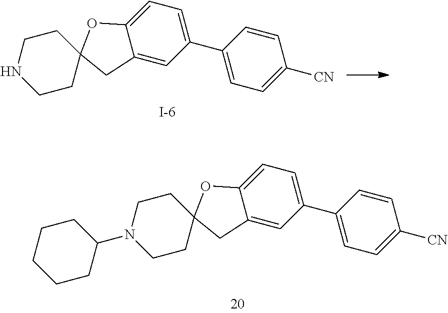

This compound was prepared in 20% yield (25 mg) as described for compound 1 but using cyclohexanone and intermediate I-6 as the starting material. ¹H NMR (400 MHz, CD₃OD) δ: 7.78-7.76 (m, 4H), 7.58 (s, 1H), 7.50 (d, J=7.8 Hz, 1H), 6.71 (d, J=8.4 Hz, 1H), 3.55-3.41 (m, 4H), 3.22 (s, 2H), 2.31-2.13 (m, 7 H), 2.01-1.97 (m, 2H), 1.78-1.74 (m, 1H), 1.63-1.42 (m, 5H). MS (ESI): m/z 373.1 (M+H)⁺.

### 21. Compound 21: 4-(1′-(Cyclobutylmethyl)-3H-spiro[benzofuran-2,4′-piperidine]-5-yl)benzonitrile

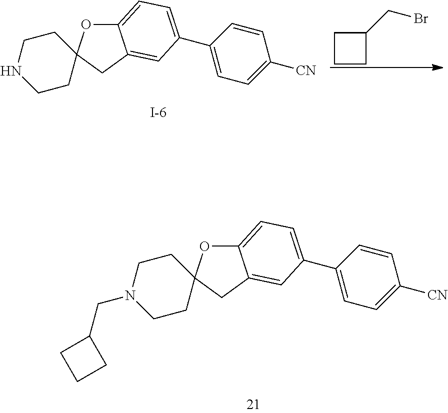

Intermediate I-6 (100 mg, 0.34 mmol), (bromomethyl)cyclobutane (72 mg, 0.68 mmol) and K₂CO₃ (140 mg, 1.02 mmol) were suspended in DMF (5 mL) and the reaction mixture was heated at 80° C. overnight. The reaction mixture was extracted with ethyl acetate and the organic layer was washed with H₂O. The combined organic layers were washed with H₂O and brine, dried with anhydrous Na₂SO₄, the solids were filtered and the filtrated was concentrated. The crude reaction mixture was purified by preparative reverse phase HPLC to give compound 21 (20 mg, 20%). ¹H NMR (400 MHz, CD₃OD) δ: 7.75-7.71 (m, 4H), 7.56 (s, 1H), 7.49 (d, J=8.4 Hz, 1H), 6.90-6.86 (m, 1H), 3.57-3.43 (m, 2H), 3.41-3.18 (m, 6H), 3.86-3.82 (m, 1H), 2.30-2.18 (m, 6H), 2.14-1.86 (m, 4H). MS (ESI): m/z 359.2 (M+H)⁺.

### 22. Compound 22: 4-(1′-Isopropyl-3H-spiro[benzofuran-2,4′-piperidine]-5-yl)benzonitrile

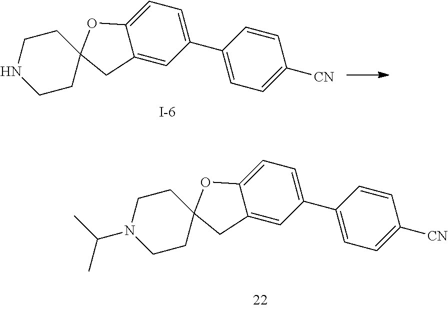

This compound was prepared in 23% yield (30 mg) as described for compound 21 but using 2-bromopropane as the starting material. ¹H NMR (400 MHz, CD₃OD) δ: 7.78-7.72 (m, 4H), 7.56 (s, 1H), 7.48 (d, J=9.6 Hz, 1H), 6.90 (d, J=8.4 Hz, 1H), 3.65-3.57 (m, 1H), 3.50-3.38 (m, 4H), 3.20 (s, 2H), 2.29-2.14 (m, 4H), 1.43 (d, J=6.8 Hz, 6H). MS (ESI): m/z 333.1 (M+H)⁺.

### 23. Compound 23: 5-Bromo-1′-cyclobutyl-3H-spiro[benzofuran-2,4′-piperidine]

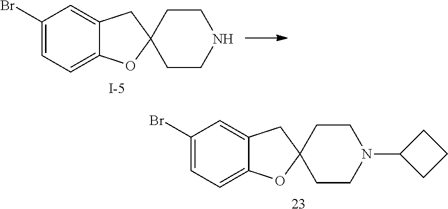

This compound was prepared in 80% yield (26 mg) as described for compound 1 but using I-5 as the starting material. ¹H NMR (400 MHz, CD₃OD) δ: 1.70-1.72 (m, 2H), 1.91-2.00 (m, 4H), 2.14-2.20 (m, 4H), 2.95-2.97 (m, 4H), 3.27-3.30 (m, 2H), 3.54-3.40 (m, 1H), 6.53 (d, J=8.4 Hz, 1H), 7.06-7.08 (m, 1H), 7.16 (1H). MS (ESI): m/z 322.1 (M+H)⁺.

### 24. Compound 24: 5-Bromo-1′-(1-methylpiperidin-4-yl)-3H-spiro[benzofuran-2,4′-piperidine]

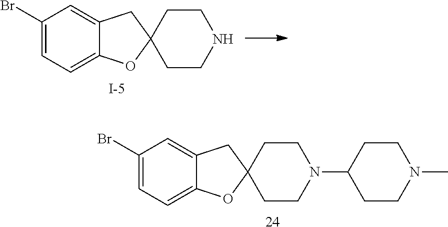

This compound was prepared in 75% yield (26 mg) as described for compound 2 but using I-5 as the starting material. ¹H NMR (400 MHz, CDCl₃) δ: 1.61-1.71 (m, 2H), 1.76-1.83 (m, 4H), 1.93-2.01 (m, 4H), 2.28 (s, 3H), 2.33-2.40 (m, 1H), 2.63 (m, 2H), 2.71-2.77 (m, 2H), 2.93-2.95 (m, 4H), 6.62 (d, J=8.4 Hz, 1H), 7.18 (dd, J₁=8.4 Hz, J₂=2.4 Hz, 1H), 7.21 (d, J=2.4 Hz, 1H). MS (ESI): m/z 367.1 (M+H)⁺.

### 25. Compound 25: 5-Bromo-1′-(1-cyclobutylethyl)-3H-spiro[benzofuran-2,4′-piperidine]

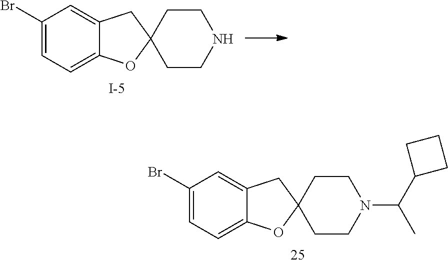

This compound was prepared in 40% yield (46 mg) as described for compound 4 but using I-5 as the starting material. ¹H-NMR (400 MHz, CD₃OD) δ: 1.30 (d, J=6.8 Hz, 3H), 1.81-2.22 (m, 10H), 2.70-2.77 (m, 1H), 3.11 (s, 2H), 3.35-3.44 (m, 4H), 6.72 (d, J=8.4 Hz, 1H), 7.25 (d, J=8.4 Hz, 1H), 7.33 (s, 1H). MS (ESI): m/z 352.1 (M+H)⁺.

### 26. Compound 26: 1′-((1H-Imidazol-4-yl)methyl)-5-bromo-3H-spiro[benzofuran-2,4′-piperidine]

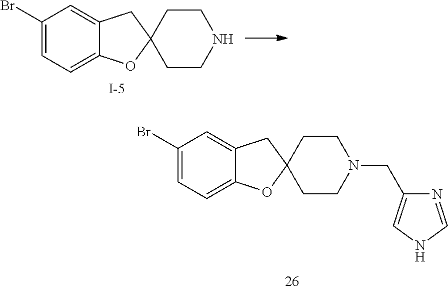

This compound was prepared in 70% yield (29 mg) as described for compound 3 but using I-5 as the starting material. ¹H NMR (400 MHz, CD₃OD) δ: 2.08-2.25 (m, 4H), 3.13 (s, 2H), 3.40-3.53 (m, 4H), 4.50 (s, 2H), 6.68 (d, J=8.4 Hz, 1H), 7.23-7.25 (dd, 1H), 7.32 (1H), 7.68 (s, 1H), 8.62 (s, 1H). MS (ESI): m/z 350.1 (M+H)⁺.

### 27. Compound 27: 1′-Cyclobutyl-5-(1H-pyrazol-4-yl)-3H-spiro[benzofuran-2,4′-piperidine]

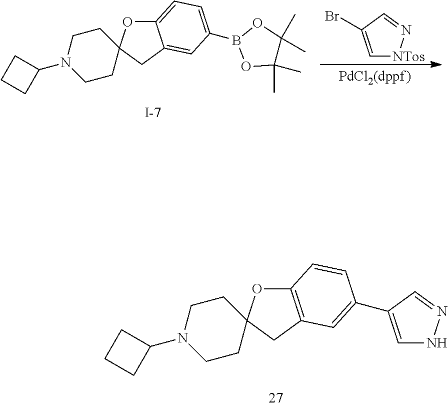

Intermediate I-7 (100 mg, 0.27 mmol), 4-bromo-1-tosyl-1H-pyrazole (98 mg, 0.33 mmol), PdCl₂(dppf) (22 mg, 0.03 mmol) and NaOH (33 mg, 0.81 mmol) was dissolved in DMF (4 mL) and the mixture was degassed by bubbling N₂. The reaction mixture was stirred at 150° C. under microwave for 1 hr. The solids were filtered, ethyl acetate was added to the filtrate and the filtrate was washed with water. The combined organic layers were dried over sodium sulfate, the solids were filtered and the filtrate was concentrated. The crude reaction mixture was purified silica gel chromatography to give 27 (42 mg, 50%). ¹H NMR (400 MHz, CD₃OD) δ: 1.79-1.85 (m, 2H), 2.05-2.18 (m, 4H), 2.26-2.37 (m, 4H), 3.01-3.17 (m, 4H), 3.37-3.47 (m, 2H), 3.62-3.74 (m, 1H), 6.78 (d, J=8.0 Hz, 1H), 7.41 (d, J=8.0 Hz, 1H), 7.50 (s, 1H), 8.52 (s, 1H). MS (ESI): m/z 310.1 (M+H)⁺.

### 28. Compound 28: 1′-Cyclobutyl-5-(4-methoxypyrimidin-2-yl)-3H-spiro[benzofuran-2,4′-piperidine]

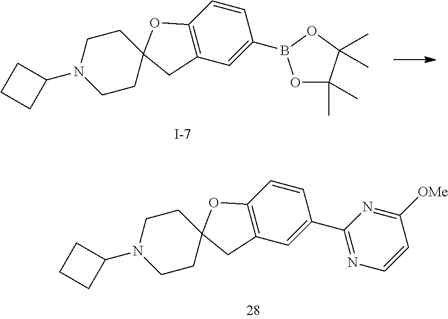

This compound was prepared in 36% yield (34 mg) as described for compound 27 but using 2-chloro-4-methoxypyrimidine as the starting material. ¹H NMR (400 MHz, CD₃OD) δ: 1.63-1.96 (m, 8H), 2.00-2.10 (m, 2H), 2.20-2.62 (m, 4H), 2.75-2.83 (m, 1H), 3.03 (s, 2H), 4.01 (s, 3H), 6.60 (d, J=5.6 Hz, 1H), 6.74 (d, J=8.8 Hz, 1H), 8.16 (m, 2H), 8.36 (d, J=6.0 Hz, 1H). MS (ESI): m/z 352.1 (M+H)⁺.

### 29. Compound 29: 1′-Cyclobutyl-5-(pyrazin-2-yl)-3H-spiro[benzofuran-2,4′-piperidine]

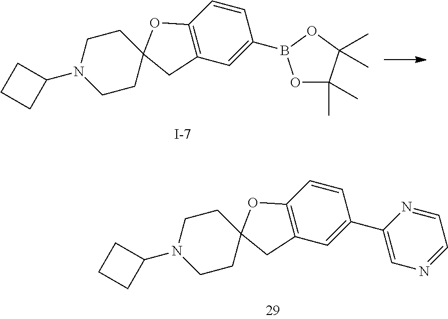

This compound was prepared in 50% yield (43 mg) as described for compound 27 but using 2-chloropyrazine as the starting material. ¹H NMR (400 MHz, CD₃OD) δ: 1.75-1.84 (m, 2H), 2.02-2.18 (m, 4H), 2.14-2.32 (m, 4H), 3.06-3.17 (m, 4H), 3.37-3.42 (m, 2H), 3.62-3.72 (m, 1H), 6.82 (d, J=8.4 Hz, 1H), 7.80 (d, J=8.0 Hz, 1H), 7.87 (s, 1H), 8.35 (s, 1H), 8.50 (s, 1H), 8.92 (s, 1H). MS (ESI): m/z 322.1 (M+H)⁺.

### 30. Compound 30: 1′-Cyclobutyl-5-(imidazo[1,2-a]pyridin-6-yl)-3H-spiro[benzofuran-2,4′-piperidine]

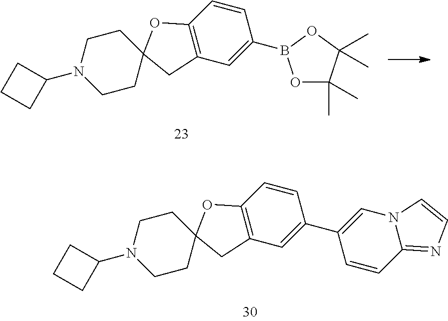

This compound was prepared in 80% yield (78 mg) as described for compound 27 but using 6-bromoimidazo[1,2-a]pyridine as the starting material. ¹H-NMR (400 MHz, CD₃OD) δ: 1.67-1.94 (m, 8H), 2.02-2.08 (m, 2H), 2.20-2.70 (m, 4H), 2.76-2.83 (m, 1H), 3.02 (s, 2H), 6.74 (d, J=8.4 Hz, 1H), 7.31 (m, 1H), 7.40 (s, 1H), 7.51 (m, 3H), 7.80 (s, 1H), 8.53 (s, 1H). MS (ESI): m/z 360.1 (M+H)⁺.

### 31. Compound 31: 1′-Cyclobutyl-5-(1H-imidazol-1-yl)-3H-spiro[benzofuran-2,4′-piperidine]

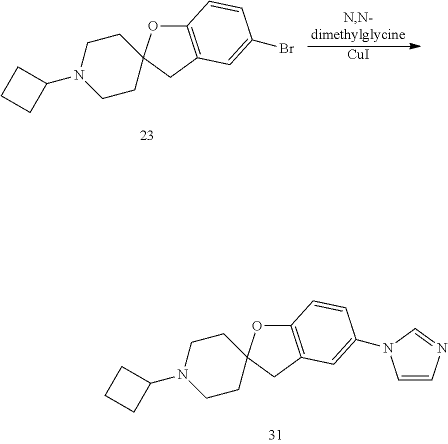

Compound 23 (40 mg, 0.12 mmol), N,N-dimethylglycine (8 mg), imidazole (15 mg, 0.22 mmol), CuI (5 mg) and K₃PO₄ (60 mg) were suspended in dry DMSO and the mixture was stirred at 110° C. overnight. Water was added and the reaction mixture was extracted with ethyl acetate. The combined organic phase were dried over sodium sulfate, the solids were filtered and the filtrate was concentrated. The crude reaction mixture was purified by preparative TLC to give compound 31 (20 mg, 52%). ¹H NMR (400 MHz, CDCl₃) δ: 1.69-1.76 (m, 2H), 1.90-2.15 (m, 8H), 2.40-2.60 (m, 4H), 2.90 (m, 1H), 3.05 (s, 2H), 6.78-6.81 (d, J=8.4 Hz, 1H), 7.09-7.26 (m, 4H), 7.74 (brs, 1H). MS (ESI): m/z 310.0 (M+H)⁺.

### 32. Compound 32: 5-(1H-Benzo[d]imidazol-1-yl)-1′-cyclobutyl-3H-spiro[benzofuran-2,4′-piperidine]

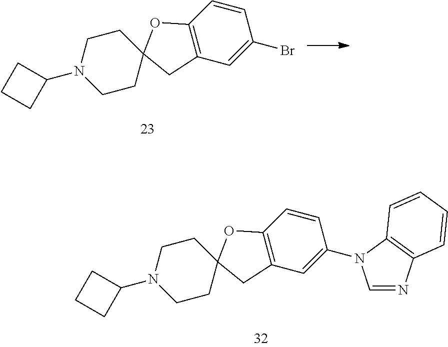

This compound was prepared in 45% yield (25 mg) as described for compound 31 but using 1H-benzo[d]imidazole as the starting material. ¹H NMR (400 MHz, CD₃OD, hydrochloric acid salt) δ: 1.80-2.00 (m, 2H), 2.20-2.45 (m, 8H), 3.05-3.15 (m, 2H), 3.50-3.55 (d, 2H), 3.59 (s, 2H), 3.75-3.85 (m, 1H), 7.08-7.10 (d, J=8.4 Hz, 1H), 7.52-7.57 (d, 1H), 7.60-7.80 (m, 4H), 7.94-7.96 (d, J=8.4 Hz, 1H), 9.70 (s, 1H). MS (ESI): m/z 360.1 (M+H)⁺.

### 33. Compound 33: 1′-Cyclobutyl-5-(4-methylpiperazin-1-yl)-3H-spiro[benzofuran-2,4′-piperidine]

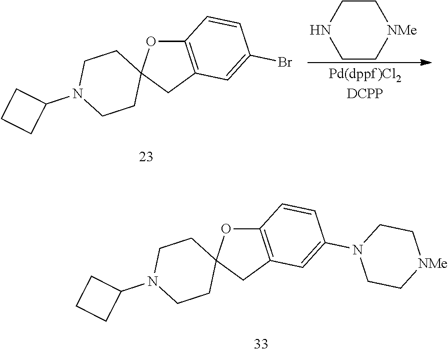

Compound 23 (70 mg, 0.22 mmol), 1-methylpiperazine (50 mg, 0.50 mmol), Pd(dppf)Cl₂ (15 mg), t-BuOK (55 mg) and dicyclohexyl(2′,4′,6′-triisopropylbiphenyl-2-yl)phosphine (DCPP) (18 mg) were suspended in toluene and the mixture was degassed by bubbling N₂. The reaction mixture was stirred at 100° C. under microwave for 1 hr. Water and methylene chloride were added, the organic layer was dried over sodium sulfate, the solids were filtered and the filtrate was concentrated. The crude reaction mixture was purified by preparative TLC to give compound 33 (25 mg, 34%). ¹H NMR (400 MHz, CD₃OD, hydrochloric acid salt) δ: 1.88 (brs, 2H), 2.18 (s, 2H), 2.36 (s, 2H), 3.03 (s, 3H), 3.10-3.20 (m, 3H), 3.40-3.90 (m, 12H), 6.86 (d, 1H), 7.31 (d, 1H), 7.43 (s, 1H). MS (ESI): m/z 342.2 (M+H)⁺.

### 34. Compound 34: 1′-Cyclobutyl-5-(piperidin-1-yl)-3H-spiro[benzofuran-2,4′-piperidine]

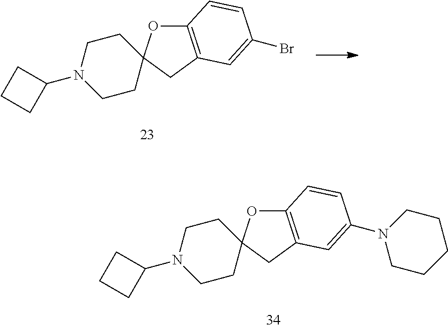

This compound was prepared in 40% yield (30 mg) as described for compound 33 but using piperidine as the starting material. ¹H NMR (400 MHz, CDCl₃) δ: 1.52-1.53 (m, 2H), 1.65-1.85 (m, 8H), 1.85-2.00 (m, 4H), 2.00-2.10 (m, 2H), 2.43 (brs, 4H), 2.74-2.80 (m, 1H), 2.93-2.98 (m, 6H), 6.64-6.66 (d, 1H), 6.71-6.72 (dd, 1H), 6.81 (s, 1H). MS (ESI): m/z 327.2 (M+H)⁺.

### 35. Compound 35: 4-(1′-Cyclobutyl-3H-spiro[benzofuran-2,4′-piperidine]-6-yl)benzonitrile

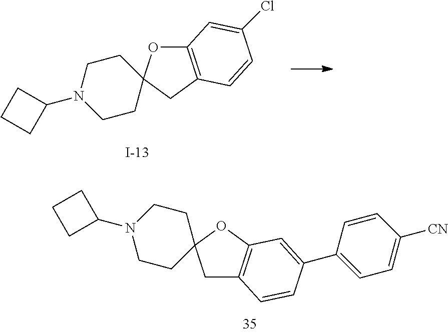

This compound was prepared in 65% yield (40 mg) as described for compound 5 but using intermediate I-13 as the starting material. ¹H NMR (400 MHz, CDCl₃) δ: 1.65-1.75 (m, 2H), 1.80-1.95 (m, 4H), 2.00-2.15 (m, 4H), 2.45 (brs, 4H), 2.80 (m, 1H), 3.03 (s, 2H), 6.98 (s, 1H), 7.03-7.05 (d, J=7.2 Hz, 1H), 7.21-7.23 (d, J=7.2 Hz, 1H), 7.62-7.64, (d, J=8.0 Hz, 2H), 7.68-7.70 (d, J=8.0 Hz, 2H). MS (ESI): m/z 345.1 (M+H)⁺.

### 36. Compound 36: 1′-Cyclobutyl-6-(4-methoxypyrimidin-2-yl)-3H-spiro[benzofuran-2,4′-piperidine]

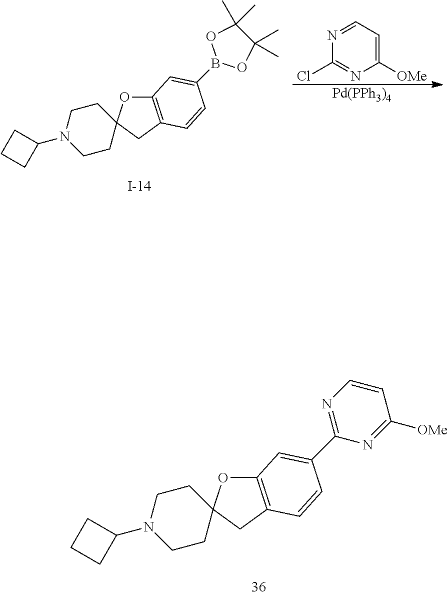

Intermediate I-14 (116 mg, 0.32 mmol), 2-chloro-4-methoxypyrimidine (70 mg, 0.48 mmol), Pd(PPh₃)₄ (36 mg, 0.032 mmol) and Na₂CO₃ (100 mg, 0.95 mmol) was suspended in a mixture of dioxane (3 mL) and H₂O (3 mL) and the mixture was degassed by bubbling N₂. The reaction mixture was heated at 100° C. under microwave irradiation for 1 hr. The solids were filtered and the filtrate was extracted with ethyl acetate. The combined organic layers were dried over sodium sulfate and the filtrate was evaporated. The crude reaction mixture was purified by silica gel chromatography to give Compound 36 (50 mg, 45%). ¹H NMR (400 MHz, CD₃OD) δ: 1.74-1.87 (m, 2H), 2.09-2.37 (m, 8H), 3.07-3.18 (m, 4H), 3.00-3.21 (m, 4H), 3.31-3.50 (m, 2H), 3.66-3.73 (m, 1H), 4.24 (s, 3H), 7.20 (d, J=7.2 Hz, 1H), 7.45 (d, J=7.6 Hz, 1H), 7.62-7.67 (m, 1H), 7.82 (d, J=7.6 Hz, 1H), 8.61 (d, J=6.8 Hz, 1H). MS (ESI): m/z 352.2 (M+H)⁺.

### 37. Compound 37: 1′-Cyclobutyl-6-(pyrazin-2-yl)-3H-spiro[benzofuran-2,4′-piperidine]

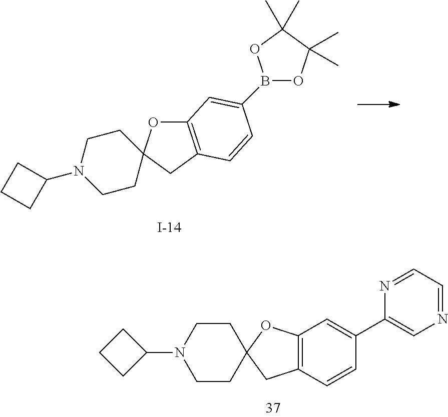

This compound was prepared in 40% yield (35 mg) as described for compound 36 but using 2-chloropyrazine as the starting material. ¹H NMR (400 MHz, CD₃OD) δ: 1.72-1.89 (m, 2H), 2.06-2.15 (m, 4H), 2.22-2.38 (m, 4H), 3.07-3.18 (m, 4H), 3.38-3.50 (m, 2H), 3.67-3.72 (m, 1H), 7.32 (d, J=7.6 Hz, 1H), 7.46-7.50 (m, 1 H), 7.60 (d, J=7.2 Hz, 1H), 8.62 (s, 1H), 8.94 (s, 1H), 9.21 (s, 1H). MS (ESI): m/z 322.2 (M+H)⁺.

### 38. Compound 38: 1′-Cyclobutyl-6-(4-methylpiperazin-1-yl)-3H-spiro[benzofuran-2,4′-piperidine]

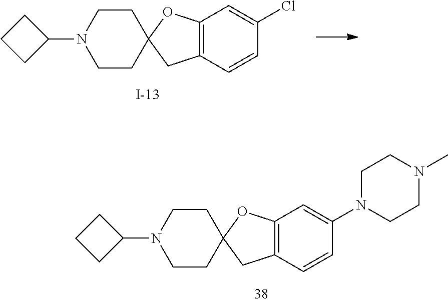

This compound was prepared in 50% yield (30 mg) as described for compound 33 but using intermediate I-13 as the starting material. ¹H NMR (400 MHz, CDCl₃) δ: 1.60-2.10 (m, 10H), 2.33 (s, 4H), 2.33-2.41 (brs, 3H), 2.55 (4H), 2.76-2.80 (m, 1H), 2.89 (s, 2H), 3.14-3.16 (m, 4H), 6.40-6.42 (m, 2H), 6.98-7.00 (d, 1H). MS (ESI): m/z 342.2 (M+H)⁺.

### 39. Compound 39: 1-(1′-Cyclobutyl-3H-spiro[benzofuran-2,4′-piperidine]-5-yl)-N,N-dimethylmethanamine

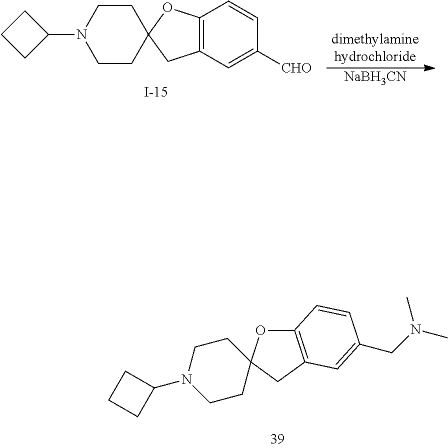

To a solution of intermediate I-15 (120 mg, 0.44 mmol) in a mixture of methanol/THF (2:3) was added dimethylamine hydrochloride (215 mg, 2.65 mmol), sodium bicarbonate (150 mg, 1.78 mmol) and sodium cyanoborohydride (28 mg, 0.44 mmol). The reaction mixture was stirred at room temperature for 4 hrs and poured into an aqueous solution of NaHCO₃. The organic phase was separated and dried on Na₂SO₄, the solids were filtered and the filtrate was concentrated. The crude reaction mixture was purified by reverse-phase HPLC to give Compound 39 (15 mg, 11%). ¹H NMR (400 MHz, CD₃OD, hydrochloride acid salt) δ: 1.80-1.95 (m, 2H), 2.17-2.20 (m, 4H), 2.33-2.37 (m, 4H), 2.81 (s, 6H), 3.18 (brs, 4H), 3.43 (brs, 2H), 3.74 (brs, 1H), 4.23 (s, 2H), 6.85 (d, 1H), 7.29 (d, 1H), 7.38 (s, 1H). (ESI): m/z 301.2 (M+H)⁺.

### 40. Compound 40: 1′-Cyclobutyl-5-(morpholinomethyl)-3H-spiro[benzofuran-2,4′-piperidine]

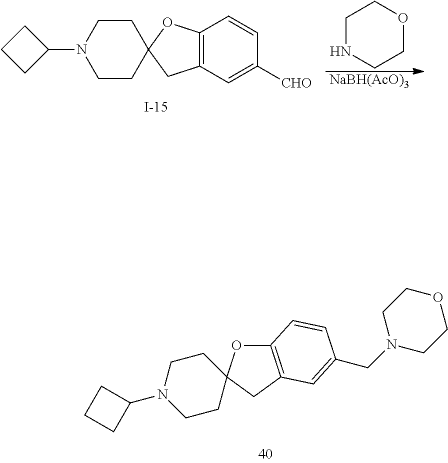

Intermediate I-15 (60 mg, 0.22 mmol), morpholine (60 mg, 0.69 mmol) and acetic acid (50 mg, 0.83 mmol) were dissolved in methylene chloride and stirred at room temperature for 1 hour. Solid NaBH(AcO)₃ was added and the mixture was stirred overnight. The reaction mixture was concentrated and saturated aqueous solution of NaHCO₃ was added followed by ethyl acetate. The organic phase was separated and dried over sodium sulfate, the solids were filtered and the filtrate was concentrated. The crude reaction mixture was purified by reverse-phase HPLC to give Compound 40 (23 mg, 26%). ¹H NMR (400 MHz, CDCl₃) δ: 1.65-1.75 (m, 2H), 1.75-1.85 (m, 2H), 1.85-2.01 (m, 4H), 2.02-2.06 (m, 2H), 2.42 (brs, 8H), 2.78 (m, 1H), 2.95 (s, 2H), 3.39 (s, 2H), 3.69 (t, 4H), 6.68 (d, J=6.4 Hz, 1H), 7.01 (d, J=6.4 Hz, 1H), 7.09 (s, 1H). (ESI): m/z 343.1 (M+H)⁺.

### 41. Compound 41: 1′-Cyclobutyl-5-(pyrrolidin-1-ylmethyl)-3H-spiro[benzofuran-2,4′-piperidine]

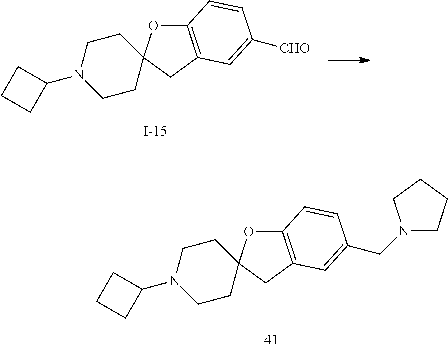

This compound was prepared in 28% yield (25 mg) as described for compound 40 but using pyrrolidine as the starting material. ¹H NMR (400 MHz, CDCl₃) δ: 1.66-1.72 (m, 2H), 1.76-1.81 (m, 6H), 1.85-1.91 (m, 2H), 1.93-2.00 (m, 2H), 2.03-2.08 (m, 2H), 2.48 (m, 8H), 2.78 (m, 1H), 2.94 (s, 2H), 3.51 (s, 2H), 6.67 (d, J=6.4 Hz, 1H), 7.01 (d, J=6.4 Hz, 1H), 7.11 (s, 1H). (ESI): m/z 327.1 (M+H)⁺.

### 42. Compound 42: 4-((1′-Cyclobutyl-3H-spiro[benzofuran-2,4′-piperidine]-5-yl)methylamino)benzonitrile

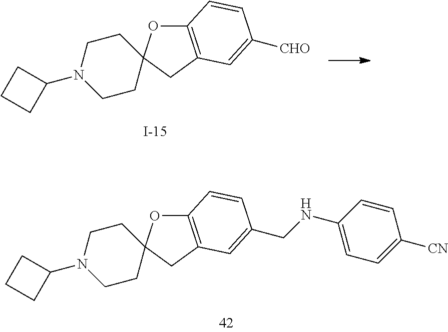

This compound was prepared in 30% yield (30 mg) as described for compound 40 but using 4-aminobenzonitrile as the starting material. ¹H NMR (400 MHz, CDCl₃) δ: 1.65-1.75 (m, 2H), 1.75-1.85 (m, 2H), 1.85-2.01 (m, 4H), 2.02-2.06 (m, 2H), 2.43 (brs, 3H), 2.77 (m, 1H), 2.96 (s, 2H), 4.24 (d, 2H), 4.46 (t, 4H), 6.58 (d, 2H), 6.73 (d, J=8.0 Hz, 1H), 7.05-7.10 (m, 2H), 7.42 (d, 2H). (ESI): m/z 374.1 (M+H)⁺.

### 43. Compound 43: 1-(1′-Cyclobutyl-3H-spiro[benzofuran-2,4′-piperidine]-6-yl)-N,N-dimethylmethanamine

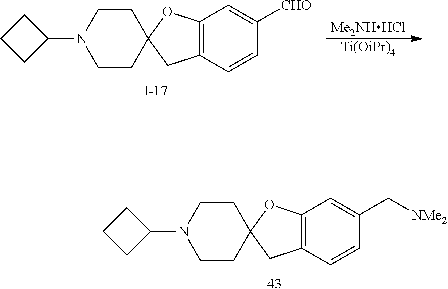

To a solution of triethylamine (0.13 mL, 0.89 mmol) in absolute ethanol (8 mL) were added dimethylamine hydrochloride (72 mg, 0.89 mmol), titanium isopropoxide (252 mg, 0.89 mmol), and intermediate I-17 (120 mg, 0.44 mmol). The reaction mixture was stirred at room temperature overnight, sodium borohydride (34 mg, 0.89 mmol) was added and the reaction mixture was stirred for additional 10 hrs at room temperature. The reaction was poured into an aqueous saturated solution of ammonia, the solids were filtered and the aqueous filtrate was extracted with dichloromethane. The combined organic layers were dried over sodium sulfate, the solids were filtered and the filtrate was concentrated. The crude reaction mixture was purified by silica gel chromatography to give compound 43 (26 mg, 20%). ¹H-NMR (400 MHz, CD₃OD): δ 7.33 (m, 1H), 7.04 (m, 2H), 4.28 (s, 2H), 3.78 (m, 1H), 3.48 (m, 2H), 3.12 (m, 4H), 2.86 (s, 6H), 2.52-2.06 (m, 8H), 1.92 (m, 2H). (ESI): m/z 301 (M+H⁺).

### 44. Compound 44: 1′-Cyclobutyl-6-(morpholinomethyl)-3H-spiro[benzofuran-2,4′-piperidine]

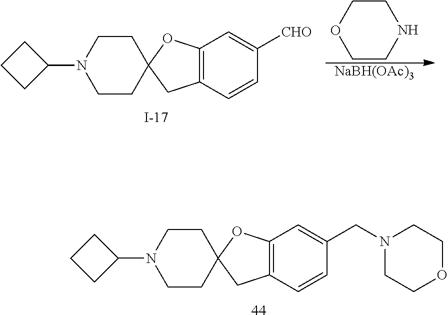

Intermediate I-17 (100 mg, 0.37 mmol), morpholine (64 mg, 0.74 mmol) and acetic acid (22 mg, 0.37 mmol) were dissolved in dry dichloromethane (10 mL) and the reaction mixture was stirred for 1 hr. 4 Å molecular sieves were added and the reaction mixture was stirred for an additional hour. Solid NaBH(OAc)₃ (117 mg, 0.56 mmol) was added and the reaction mixture was stirred overnight at room temperature. The solid were filtered and the filtrate was concentrated. The crude reaction mixture was purified by silica gel chromatography to give compound 44 (38 mg, 30%). ¹H-NMR (400 MHz, CD₃OD): δ 7.33 (m, 1H), 7.04 (m, 2H), 4.21 (s, 2H), 3.93 (m, 2H), 3.70 (m, 3H), 3.48 (m, 4H), 3.16 (s, 6H), 2.28 (m, 4H), 2.10 (m, 4H), 1.82 (m, 2H). (ESI): m/z 343 (M+H⁺).

### 45. Compound 45: 1′-Cyclobutyl-6-(pyrrolidin-1-ylmethyl)-3H-spiro[benzofuran-2,4′-piperidine]

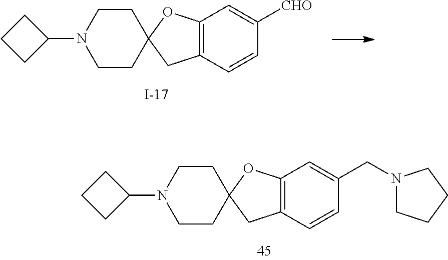

This compound was prepared in 41% yield (50 mg) as described for compound 44 but using pyrrolidine as the starting material. ¹H-NMR (400 MHz, CD₃OD): δ 7.31 (m, 1H), 7.00 (m, 2H), 4.32 (s, 2H), 3.78 (m, 1H), 3.48 (m, 4H), 3.17 (m, 6H), 2.38 (m, 4H), 2.19 (m, 6H), 2.02 (m, 2H), 1.91 (m, 2H). (ESI): m/z 327 (M+H⁺).

### 46. Compound 46: 4-((1′-Cyclobutyl-3H-spiro[benzofuran-2,4′-piperidine]-6-yl)methylamino)benzonitrile

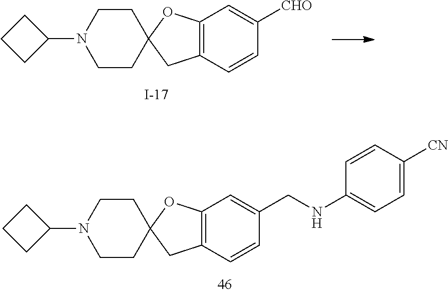

This compound was prepared in 37% yield (51 mg) as described for compound 44 but using 4-aminobenzonitrile as the starting material. ¹H-NMR (400 MHz, CD₃OD): δ 7.38 (d, 2H, J=8.8 Hz), 7.16 (d, 1H, J=7.6 Hz), 6.88 (d, 1H, J=7.6 Hz), 6.75 (s, 1H), 6.67 (m, 2H), 4.34 (s, 2H), 3.75 (m, 1H), 3.44 (m, 2H), 3.17-3.10 (m, 4H), 2.41-2.22 (m, 4H), 2.22-1.84 (m, 6H). (ESI): m/z 374 (M+H⁺).

### 47. Compound 47: 5′-Bromo-1-cyclobutyl-3′H-spiro[azepane-4,2′-benzofuran]

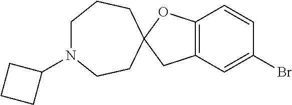

This compound was prepared in 78% yield as described for compound 1 but using intermediate I-23 as the starting material. ¹H NMR (400 MHz, CDCl₃) δ: 7.16-7.26 (m, 2H), 6.58 (d, J=8.4 Hz, 1H), 2.99 (s, 2H), 2.89-2.98 (m, 1H), 2.62-2.72 (m, 1H), 2.44-2.62 (m, 3H), 2.09-2.15 (m, 1H), 1.95-2.08 (m, 4H), 1.80-1.95 (m, 4H), 1.54-1.72 (m, 3H). MS (ESI): m/z 336.1 (M+H⁺).

### 48. Compound 48: 4-(1-Cyclobutyl-3′H-spiro[azepane-4,2′-benzofuran]-5′-yl)benzonitrile

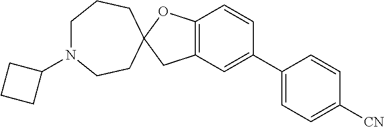

This compound was prepared in 35% yield as described for compound 5 but using compound 47 as the starting material. ¹H NMR (400 MHz, CDCl₃) δ: 7.64-7.66 (m, 2H), 7.57-7.60 (m, 2H), 7.32-7.35 (m, 2H), 6.80 (d, J=8.4 Hz, 1H), 3.07 (s, 2H), 2.90-3.00 (m, 1H), 2.45-2.75 (m, 4H), 2.13-2.22 (m, 1H), 1.95-2.12 (m, 5H), 1.80-1.93 (m, 3H), 1.55-1.72 (m, 3H). MS (ESI): m/z 359.2 (M+H⁺).

### 49. Compound 49: 1-Cyclobutyl-5′-(imidazo[1,2-a]pyridin-6-yl)-3′H-spiro[azepane-4,2′-benzofuran]

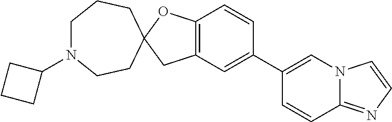

This compound was prepared in 62% yield as described for compound 30 but using intermediate I-24 as the starting material. ¹H NMR (400 MHz, CDCl₃) δ: 8.21 (m, 1H), 7.60-7.65 (m, 3H), 7.27-7.38 (m, 3H), 6.81 (d, J=8.4 Hz, 1H), 3.09 (s, 2H), 2.90-3.00 (m, 1H), 2.50-2.75 (m, 4H), 2.15-2.25 (m, 2H), 2.00-2.15 (m, 4H), 1.85-1.98 (m, 3H), 1.58-1.78 (m, 3H). MS (ESI): m/z 374.2 (M+H⁺).

### 50. Compound 50: 5′-(1H-Benzo[d]imidazol-1-yl)-1-cyclobutyl-3′H-spiro[azepane-4,2′-benzofuran]

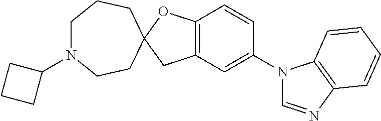

This compound was prepared in 23% yield as described for compound 32 but using compound 47 as the starting material. ¹H NMR (400 MHz, CDCl₃) δ: 8.02 (s, 1H), 7.83-7.88 (m, 1H), 7.42-7.48 (m, 1H), 7.28-7.35 (m, 2H), 7.18-7.23 (m, 2H), 6.86 (d, J=8.4 Hz, 1H), 3.12 (s, 2H), 2.95-3.05 (m, 1H), 2.50-2.80 (m, 4H), 2.19-2.28 (m, 1H), 2.00-2.18 (m, 4H), 1.75-1.98 (m, 4H), 1.60-1.75 (m, 3H). MS (ESI): m/z 374.2 (M+H⁺).

### 51. Compound 51: 1-Cyclobutyl-5′-(piperidin-1-yl)-3′H-spiro[azepane-4,2′-benzofuran]

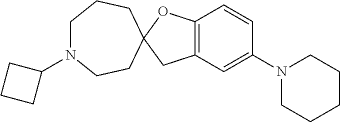

This compound was prepared in 20% yield as described for compound 34 but using compound 47 as the starting material. ¹H NMR (400 MHz, CDCl₃) δ: 6.80 (d, J=2.4 Hz, 1H), 6.71 (dd, J₁=8.4 Hz, J₂=2.4 Hz, 1H), 6.61 (d, J=8.4 Hz, 1H), 2.90-3.00 (m, 7H), 2.40-2.75 (m, 4H), 2.08-2.18 (m, 1H), 1.85-2.08 (m, 5H), 1.75-1.85 (m, 3H), 1.65-1.75 (m, 5H), 1.55-1.65 (m, 2H), 1.48-1.55 (m, 2H). MS (ESI): m/z 341.1 (M+H⁺).

### 52. Compound 52

### 53. Compound 53

### (S)-5′-(1H-Benzo[d]imidazol-1-yl)-1-cyclobutyl-3′H-spiro[azepane-4,2′-benzofuran] and (R)-5′-(1H-benzo[d]imidazol-1-yl)-1-cyclobutyl-3′H-spiro[azepane-4,2′-benzofuran]

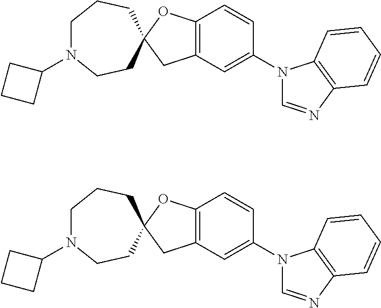

Enantiomers of compound 50 were separated by chiral chromatography. Compound 50 (15 mg) was dissolved in methanol and the solution was filtered through a 0.5μ filter cartridge. The isocratic SFC method used a mixture of 60% methanol with 1% isopropylamine in CO₂. The column was Chiracel AD-H™ (Chiral Technologies™) in a 3.0×25.0 cm format with a mobile phase flow of 80 g/minute. The enantiomers of compound 50 were isolated as two separate peaks during chiral separation. The faster eluting peak was designated as compound 52 (4.8 mg). The more slowly eluting peak was designated as compound 53 (2.6 mg).

### 54. Compound 54: 1′-Cyclobutyl-5-(2-methylimidazo[1,2-a]pyridin-6-yl)-3H-spiro[benzofuran-2,4′-piperidine]

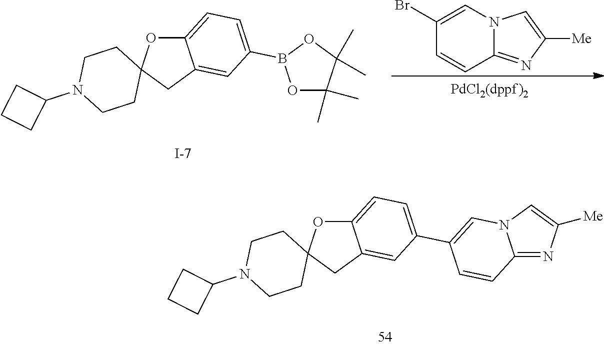

A microwave vial was charged with intermediate I-7 (57 mg, 0.15 mmol, 1.0 eq), 6-bromo-2-methylimidazo[1,2-a]pyridine (32 mg, 0.15 mmol, 1.0 eq), Pd(dppf)₂Cl₂ (10 mg, 0.012 mmol, 0.08 eq), Na₂CO₃ (48 mg, 0.45 mmol, 3.0 eq) and a mixture of DMF/water (2 mL/0.5 mL). The vial was sealed, evacuated and purged three times with nitrogen. The reaction mixture was heated under microwave irradiation at 110° C. for 2 hrs, the solids were removed by filtration and washed with ethyl acetate. Water was added, and the crude reaction mixture was extracted with ethyl acetate. The combined organic layers were washed with water and brine, and dried with sodium sulfate. The solids were removed by filtration, the filtrate was concentrated and the residue was purified by preparative TLC to give compound 54 (34 mg, 61%). ¹H NMR (400 MHz, CD₃OD) δ: 8.99 (s, 1H), 8.19 (d, J=7.2 Hz, 1H), 8.04 (s, 1H), 7.91 (d, J=8.4 Hz, 1H), 7.63 (s, 1H), 7.55 (d, J=6.8 Hz, 1H), 6.90-7.00 (m, 1H), 3.75-3.85 (m, 1H), 3.48-3.69 (m, 2H), 3.12-3.31 (m, 4H), 2.60 (s, 3H), 2.36-2.52 (m, 4H), 2.20-2.36 (m, 4H), 1.73-2.00 (m, 2H). MS (ESI): m/z 374.1 (M+H⁺).

### 55. Compound 55: 1′-Cyclobutyl-5-(1H-pyrrolo[2,3-b]pyridin-5-yl)-3H-spiro[benzofuran-2,4′-piperidine]

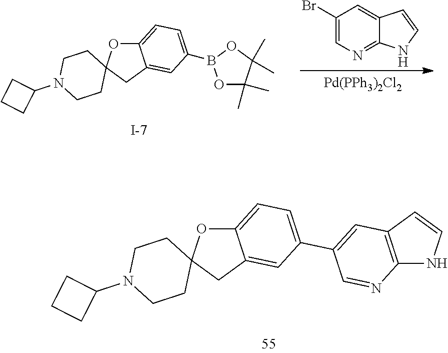

A microwave vial was charged with intermediate I-7 (110 mg, 0.31 mmol, 1.0 eq), 5-bromo-1H-pyrrolo[2,3-b]pyridine (60 mg, 0.31 mmol, 1.0 eq), Pd(PPh₃)₂Cl₂ (21 mg, 0.031 mmol, 0.1 eq), MeCN (2 mL), and aqueous sodium carbonate (2 mL, 2.0 M in water). The vial was sealed, evacuated and purged three times with nitrogen. The reaction mixture was heated under microwave irradiation at 150° C. for 30 minutes, and the solids were removed by filtration and washed with ethyl acetate. Water was added, and the crude reaction mixture was extracted with ethyl acetate. The combined organic layers were washed with water and brine, and dried with sodium sulfate. The solids were removed by filtration, the filtrate was concentrated and the residue was purified by preparative TLC to give compound 55 (66 mg, 60%). ¹H NMR (400 MHz, CD₃OD) δ: 8.96 (s, 1H), 8.69 (s, 1H), 7.82 (s, 1H), 7.71 (s, 1H), 7.55-7.62 (m, 1H), 6.90-7.07 (m, 2H), 3.75-3.87 (m, 1H), 3.48-3.70 (m, 2H), 3.17-3.31 (m, 4H), 2.36-2.52 (m, 4H), 2.20-2.36 (m, 4H), 1.80-2.04 (m, 2H). MS (ESI): m/z 360.1 (M+H⁺).

### 56. Compound 56: 1′-Cyclobutyl-5-(1H-pyrrolo[3,2-b]pyridin-5-yl)-3H-spiro[benzofuran-2,4′-piperidine]

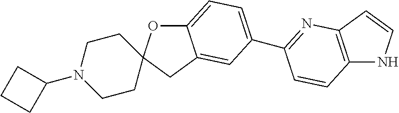

This compound was prepared in 59% yield as described for compound 55 but using 5-chloro-1H-pyrrolo[3,2-b]pyridine as the starting material. ¹H NMR (400 MHz, CD₃OD) δ: 8.55 (d, J=8.0 Hz, 1H), 8.09 (d, J=3.2 Hz, 1H), 7.85-7.89 (m, 2H), 7.78 (d, J=8.4 Hz, 1H), 7.07 (d, J=8.4 Hz, 1H), 6.90 (d, J=3.2 Hz, 1H), 3.77-3.81 (m, 1H), 3.49-3.65 (m, 2H), 3.18-3.32 (m, 4H), 2.20-2.42 (m, 8H), 1.88-1.99 (m, 2H). MS (ESI): m/z 360.2 (M+H⁺).

### 57. Compound 57: 1′-Cyclobutyl-5-(imidazo[1,5-a]pyridin-6-yl)-3H-spiro[benzofuran-2,4′-piperidine]

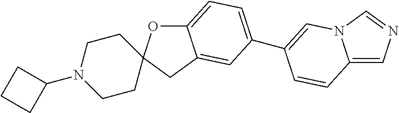

This compound was prepared in 62% yield as described for compound 54 but using 6-bromoimidazo[1,5-a]pyridine as the starting material. ¹H NMR (400 MHz, CD₃OD) δ: 9.53 (s, 1H), 8.74 (s, 1H), 8.02 (s, 1H), 7.88 (d, J=8.8 Hz, 1H), 7.50-7.60 (m, 3H), 6.90-6.92 (m, 1H), 3.74-3.80 (m, 1H), 3.42-3.61 (m, 2H), 3.10-3.22 (m, 4H), 2.17-2.50 (m, 8H), 1.80-1.96 (m, 2H). MS (ESI): m/z 360.2 (M+H⁺).

### 58. Compound 58: 1′-Cyclobutyl-5-(5H-pyrrolo[2,3-b]pyrazin-2-yl)-3H-spiro[benzofuran-2,4′-piperidine]

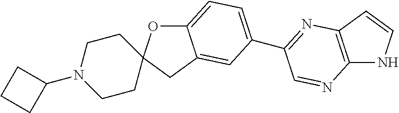

This compound was prepared in 66% yield as described for compound 55 but using 2-bromo-5H-pyrrolo[2,3-b]pyrazine as the starting material. ¹H NMR (400 MHz, CD₃OD) δ: 9.07 (s, 1H), 8.38 (s, 1H), 7.80-7.91 (m, 2H), 7.10 (d, J=8.4 Hz, 1H), 6.92-6.95 (m, 1H), 3.75-3.83 (m, 1H), 3.49-3.66 (m, 2H), 3.11-3.33 (m, 4H), 2.17-2.48 (m, 8H), 1.82-2.02 (m, 2H). MS (ESI): m/z 361.2 (M+H⁺).

### 59. Compound 59: 1′-Cyclobutyl-5-(1H-pyrrolo[3,2-b]pyridin-6-yl)-3H-spiro[benzofuran-2,4′-piperidine]

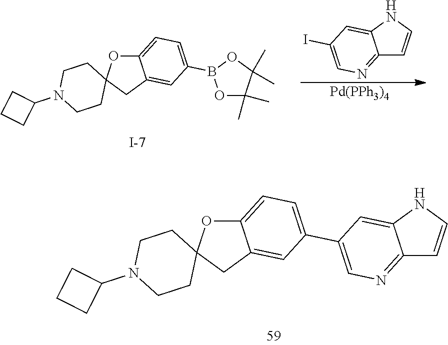

A microwave vial was charged with intermediate I-7 (100 mg, 0.27 mmol, 1.0 eq), 6-iodo-1H-pyrrolo[3,2-b]pyridine (66 mg, 0.27 mmol, 1.0 eq), Pd(PPh₃)₄ (31 mg, 0.027 mmol, 0.1 eq), dimethoxyethane (3 mL) and aqueous sodium carbonate (1 mL, 2.0 M in water). The vial was sealed, evacuated and purged three times with nitrogen. The reaction mixture was heated under microwave irradiation at 110° C. for 2 hrs, and the solids were removed by filtration and washed with ethyl acetate. Water was added, and the crude reaction mixture was extracted with ethyl acetate. The combined organic layers were washed with water and brine, and dried with sodium sulfate. The solids were removed by filtration, the filtrate was concentrated and the residue was purified by preparative TLC to give compound 59 (64 mg, 66%). ¹H NMR (400 MHz, CD₃OD) δ: 8.75 (s, 1H), 8.69 (s, 1H), 8.09 (s, 1H), 7.51-7.63 (m, 2H), 6.82-6.96 (m, 2H), 3.71-3.82 (m, 1H), 3.42-3.60 (m, 2H), 3.05-3.26 (m, 4H), 2.30-2.46 (m, 4H), 2.14-2.30 (m, 4H), 1.77-1.96 (m, 2H). MS (ESI): m/z 360.2 (M+H⁺).

### 60. Compound 60: 1′-Cyclobutyl-5-(furo[3,2-b]pyridin-6-yl)-3H-spiro[benzofuran-2,4′-piperidine]

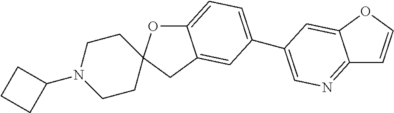

This compound was prepared in 66% yield as described for compound 59 but using 6-iodofuro[3,2-b]pyridine as the starting material. ¹H NMR (400 MHz, CD₃OD) δ: 8.82 (s, 1H), 8.58 (s, 1H), 8.32 (s, 1H), 7.58 (s, 1H), 7.49 (J=8.4 Hz, 1H), 7.15 (s, 1H), 6.87 (J=8.4 Hz, 1H), 3.64-3.73 (m, 1H), 3.38-3.56 (m, 2H), 3.08-3.16 (m, 4H), 1.99-2.31 (m, 8H), 1.77-1.86 (m, 2H). MS (ESI): m/z 361.2 (M+H⁺).

### 61. Compound 61: 1′-Cyclobutyl-5-(3-methyl-3H-imidazo[4,5-b]pyridin-6-yl)-3H-spiro[benzofuran-2,4′-piperidine]

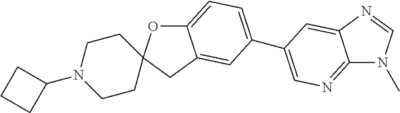

This compound was prepared in 55% yield as described for compound 59 but using 6-iodo-3-methyl-3H-imidazo[4,5-b]pyridine as the starting material. ¹H NMR (400 MHz, CD₃OD) δ: 9.60 (s, 1H), 8.93 (s, 1H), 8.40 (s, 1H), 7.61 (s, 1H), 7.52 (J=7.6 Hz, 1H), 6.93 (J=8.4 Hz, 1H), 4.16 (s, 3H), 3.75-3.85 (m, 1H), 3.46-3.51 (m, 2H), 3.16-3.22 (m, 4H), 2.36-2.41 (m, 4H), 2.17-2.30 (m, 4H), 1.86-1.96 (m, 2H). MS (ESI): m/z 375.2 (M+H⁺).

### 62. Compound 62: 5-([1,2,4]Triazolo[1,5-a]pyridin-6-yl)-1′-cyclobutyl-3H-spiro[benzofuran-2,4′-piperidine]

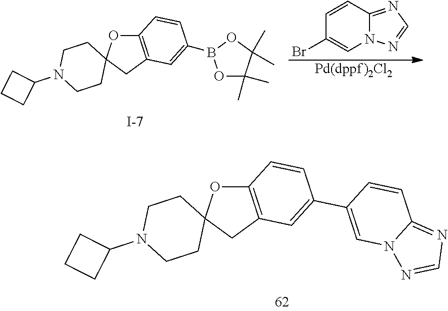

A microwave vial was charged with intermediate I-7 (100 mg, 0.27 mmol, 1.0 eq), 6-bromo-[1,2,4]triazolo[1,5-a]pyridine (54 mg, 0.27 mmol, 1.0 eq), Pd(dppf)₂Cl₂ (22 mg, 0.027 mmol, 0.1 eq), EtOH (1.5 mL), toluene (1.5 mL) and aqueous sodium carbonate (0.75 mL, 2.0 M in water). The vial was sealed, evacuated and purged three times with nitrogen. The reaction mixture was heated under microwave irradiation at 110° C. for 2 hrs, and the solids were removed by filtration and washed with ethyl acetate. Water was added, and the crude reaction mixture was extracted with ethyl acetate. The combined organic layers were washed with water and brine, and dried with sodium sulfate. The solids were removed by filtration, the filtrate was concentrated and the residue was purified by preparative TLC to give compound 62 (66 mg, 68%). ¹H NMR (400 MHz, CD₃OD) δ: 9.32 (s, 1H), 9.12 (d, J=3.2 Hz, 1H), 8.45 (d, J=8.8 Hz, 1H), 8.10 (d, J=8.8 Hz, 1H), 7.68 (s, 1H), 7.60 (J=7.2 Hz, 1H), 6.96 (J=8.0 Hz, 1H), 3.72-3.81 (m, 1H), 3.49-3.61 (m, 2H), 3.10-3.27 (m, 4H), 2.31-2.44 (m, 4H), 2.13-2.30 (m, 4H), 1.81-1.97 (m, 2H). MS (ESI): m/z 375.2 (M+H⁺).

### 63. Compound 63: 5-([1,2,4]Triazolo[1,5-a]pyridin-6-yl)-1′-cyclobutyl-3H-spiro[benzofuran-2,4′-piperidine]

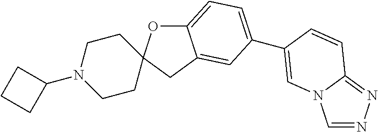

This compound was prepared in 70% yield as described for compound 62 but using 6-bromo-[1,2,4]triazolo[4,3-a]pyridine as the starting material. ¹H NMR (400 MHz, CD₃OD) δ: 9.54 (s, 1H), 9.10 (s, 1H), 8.47 (d, J=9.6 Hz, 1H), 8.15 (d, J=9.6 Hz, 1H), 7.69 (s, 1H), 7.60 (J=8.4 Hz, 1H), 6.99 (J=8.0 Hz, 1H), 3.74-3.81 (m, 1H), 3.49-3.61 (m, 2H), 3.10-3.30 (m, 4H), 2.30-2.44 (m, 4H), 2.19-2.30 (m, 4H), 1.85-2.00 (m, 2H). MS (ESI): m/z 361.2 (M+H⁺).

### 64. Compound 64: 1′-Cyclobutyl-5-(1H-pyrazolo[3,4-b]pyridin-5-yl)-3H-spiro[benzofuran-2,4′-piperidine]

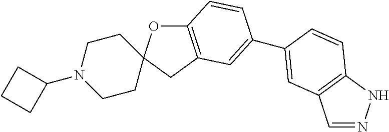

This compound was prepared in 60% yield as described for compound 62 but using 5-bromo-1H-pyrazolo[3,4-b]pyridine as the starting material. ¹H NMR (400 MHz, CD₃OD) δ: 9.01 (s, 1H), 8.93 (s, 1H), 8.58 (s, 1H), 7.65 (s, 1H), 7.56 (J=8.4 Hz, 1H), 6.97 (J=8.8 Hz, 1H), 3.74-3.81 (m, 1H), 3.49-3.61 (m, 2H), 3.10-3.29 (m, 4H), 2.14-2.44 (m, 8H), 1.85-2.00 (m, 2H). MS (ESI): m/z 361.2 (M+H⁺).

### 65. Compound 65: 1′-Cyclobutyl-5-(1-methyl-1H-indazol-6-yl)-3H-spiro[benzofuran-2,4′-piperidine]

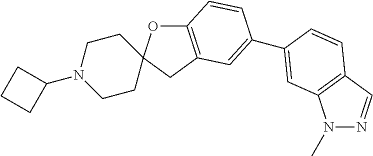

This compound was prepared in 34% yield as described for compound 62 but using 6-bromo-1-methyl-1H-indazole as the starting material. ¹H NMR (400 MHz, CD₃OD) δ: 8.07 (s, 1H), 7.79 (J=8.4 Hz, 1H), 7.68 (s, 1H), 7.59 (s, 1H), 7.51 (J=8.4 Hz, 1H), 7.41 (J=8.4 Hz, 1H), 6.87 (J=8.4 Hz, 1H), 4.10 (s, 3H), 3.71-3.79 (m, 1H), 3.43-3.60 (m, 2H), 3.10-3.20 (m, 4H), 2.30-2.41 (m, 4H), 2.14-2.27 (m, 4H), 1.85-1.95 (m, 2H). MS (ESI): m/z 374.2 (M+H⁺).

### 66. Compound 66: 1′-Cyclobutyl-5-(2-methyl-2H-indazol-6-yl)-3H-spiro[benzofuran-2,4′-piperidine]

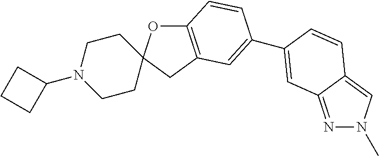

This compound was prepared in 63% yield as described for compound 62 but using 6-bromo-2-methyl-2H-indazole as the starting material. ¹H NMR (400 MHz, CD₃OD) δ: 8.78 (s, 1H), 7.99 (J=8.8 Hz, 1H), 7.80 (s, 1H), 7.69 (J=8.8 Hz, 1H), 7.64 (s, 1H), 7.56 (J=9.6 Hz, 1H), 6.92 (J=8.4 Hz, 1H), 4.39 (s, 3H), 3.76-3.81 (m, 1H), 3.47-3.61 (m, 2H), 3.10-3.24 (m, 4H), 2.30-2.41 (m, 4H), 2.12-2.30 (m, 4H), 1.82-1.97 (m, 2H). MS (ESI): m/z 374.2 (M+H⁺).

### 67. Compound 67: 6′-Chloro-1-cyclobutyl-3′H-spiro[azepane-4,2′-benzofuran]

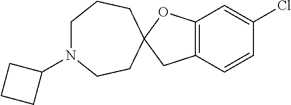

This compound was prepared in 81% yield as described for compound 1 but using intermediate I-29 as the starting material. ¹HNMR (400 MHz, CDCl3): 7.00-6.98 (m, 1H), 6.77-6.74 (m, 1H), 6.70 (s, 1H), 2.95 (m, 3H), 2.64 (m, 1H), 2.53 (m, 1H), 2.46 (m, 2H), 2.04-1.81 (m, 10H), 1.66-1.57 (m, 2H). MS (ESI): m/z 292.1 (M+H⁺).

### 68. Compound 68: 4-(1-Cyclobutyl-3′H-spiro[azepane-4,2′-benzofuran]-6′-yl)benzonitrile

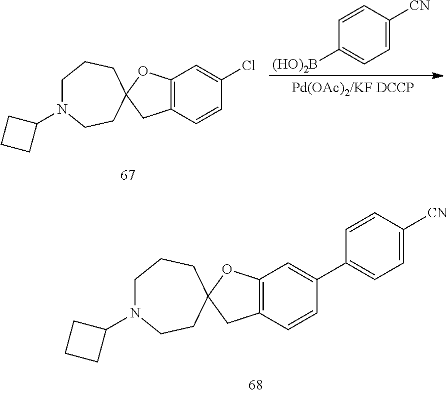

A microwave vial was charged with compound 67 (50 mg, 0.17 mmol, 1.0 eq), 4-cyanophenylboronic acid (28 mg, 0.19 mmol, 1.1 eq), Pd(OAc)₂ (10 mg, 0.045 mmol, 0.4 eq), KF (30 mg, 0.52 mmol, 3.0 eq), dicyclohexyl(2′,4′,6′-triisopropyl-biphenyl-2-yl)phosphine (DCCP) (12 mg, 0.025 mmol, 0.15 eq) and dioxane. The vial was sealed, evacuated and purged three times with nitrogen. The reaction mixture was heated under microwave irradiation at 110° C. for 90 minutes, and the solids were removed by filtration and the filtrate was concentrated by evaporation. The crude reaction product was purified preparative HPLC to give compound 68 (4 mg, 7%). ¹HNMR (400 MHz, CDCl3): 7.70-7.62 (m, 4H), 7.21 (m, 1H), 7.06 (1H, s), 6.93 (s, 1H), 3.07 (m, 3H), 3.01-2.55 (m, 4H), 2.21-1.99 (m, 10H), 1.76 (m, 2H). MS (ESI): m/z 359.1 (M+H⁺).

### 69. Compound 69: 1-Cyclobutyl-6′-(imidazo[1,2-a]pyridin-6-yl)-3′H-spiro[azepane-4,2′-benzofuran]

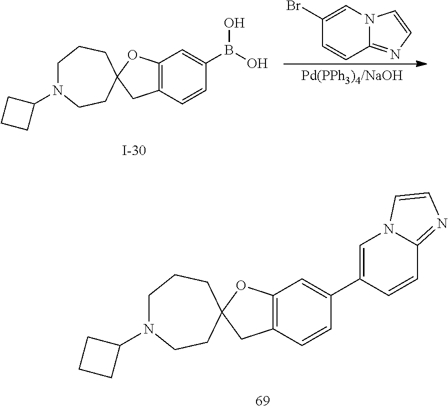

A microwave vial was charged with intermediate I-30 (150 mg, 0.50 mmol, 1.0 eq), 6-bromoimidazo[1,2-a]pyridine (100 mg, 0.50 mmol, 1.0 eq), Pd(PPh₃)₄ (58 mg, 0.05 mmol, 0.1 eq), sodium hydroxide (60 mg, 1.5 mmol, 3.0 eq) and DME (5 mL). The vial was sealed, evacuated and purged three times with nitrogen. The reaction mixture was heated under microwave irradiation at 120° C. for 2 hrs, the solids were removed by filtration and the filter cake was washed with ethyl acetate. The filtrate was concentrated, and the crude reaction product was purified by preparative TLC to give compound 69 (100 mg, 55%). ¹H NMR (400 MHz, CD₃OD) δ: 9.04 (s, 1H), 8.20-8.24 (m, 2H), 7.98-8.05 (m, 2H), 7.35 (d, J=8.0 Hz, 1H), 7.23 (d, J=8.0 Hz, 1H), 7.13 (s, 1H), 3.80-3.93 (m, 1H), 3.37-3.80 (m, 2H), 2.93-3.30 (m, 4H), 1.75-2.41 (m, 12H). MS (ESI): m/z 374.2 (M+H⁺).

### 70. Compound 70: 6′-(1H-Benzo[d]imidazol-1-yl)-1-cyclobutyl-3′H-spiro[azepane-4,2′-benzofuran]

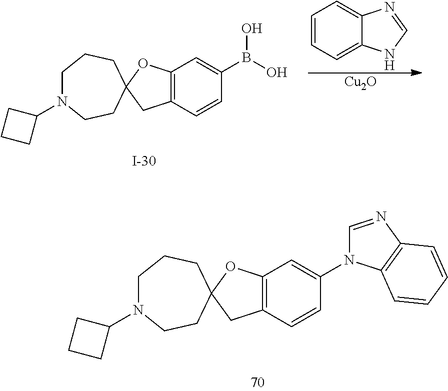

A mixture of intermediate I-30 (189 mg, 0.63 mmol, 1.0 eq), 1H-benzo[d]imidazole (82 mg, 0.69 mmol, 1.1 eq) and Cu₂O (27 mg, 0.19 mmol, 0.3 eq) in methanol (10 mL) was stirred at room temperature for 8 hrs and then at 85° C. for additional 8 hrs. Solids were removed by filtration, and the filtrate was concentrated. The crude reaction product was purified by preparative HPLC to give compound 70 (93 mg, 40%). ¹H NMR (400 MHz, CD₃OD) δ: 9.73 (s, 1H), 7.97 (d, J=7.2 Hz, 1H), 7.68-7.85 (m, 3H), 7.52 (d, J=7.2 Hz, 1H), 7.25 (d, J=7.2 Hz, 1H), 7.19 (s, 1H), 3.82-3.96 (m, 1H), 3.58-3.71 (m, 1H), 3.35-3.58 (m, 2H), 2.95-3.30 (m, 3H), 1.75-2.52 (m, 12H). MS (ESI): m/z 374.2 (M+H⁺).

### 71. Compound 71: 1-Cyclobutyl-6′-(piperidin-1-yl)-3′H-spiro[azepane-4,2′-benzofuran]

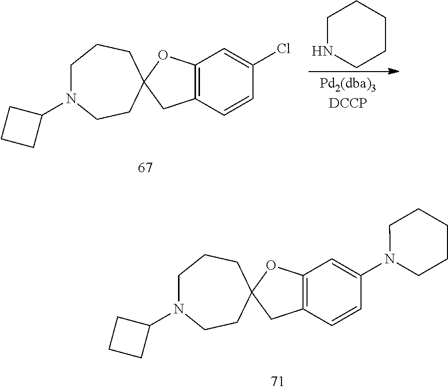

A microwave vial was charged with compound 67 (100 mg, 0.34 mmol, 1.0 eq), piperidine (35 mg, 0.41 mmol, 1.2 eq), Pd₂(dba)₃ (25 mg, 0.027 mmol, 0.1 eq), DCCP (30 mg, 0.064 mmol, 0.2 eq), ^{t}BuONa (98 mg, 1.0 mmol, 3.0 eq) and toluene. The vial was sealed, evacuated and purged three times with nitrogen. The reaction mixture was heated under microwave irradiation at 100° C. for 2 hrs, the solids were removed by filtration and the filter cake was washed with ethyl acetate. The crude reaction product was purified by preparative HPLC to give compound 71 (30 mg, 26%). ¹HNMR (400 MHz, CDCl3): 6.96-6.94 (m, 1H), 6.40-6.36 (m, 2H), 3.08 (m, 4H), 2.91 (m, 3H), 2.46 (m, 4H), 2.04-1.84 (m, 9H), 1.67-1.53 (m, 9H). MS (ESI): m/z 341.2 (M+H⁺).

### 72. Compound 72

### 73. Compound 73

### 5-Bromo-1′,3′,4′,6′,7′,8′,9′,9a′-octahydro-3H-spiro[benzofuran-2,2′-quinolizine]

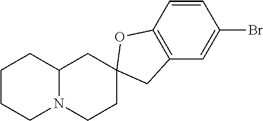

Intermediate I-40 (5 g) were subjected to separation by chiral column chromatography. Intermediate I-40 was dissolved in methanol, and the solution was filtered through a 0.5μ filter cartridge. The isocratic SFC method used a mixture of 26% methanol with 0.1% diethylamine in CO₂. The column was Daicel in a 2×25 cm format with a mobile phase flow of 50 g/minute. The diastereomers of intermediate I-40 were isolated as two separate peaks during chiral separation. The faster eluting peak was designated as compound 72 (2 g) and its optical rotation was determined as [α]_{D}²⁰=1.119°. The more slowly eluting peak was designated as compound 73 (2 g) and its optical rotation was determined as [α]_{D}²⁰=2.254°.

### Compound 72

¹H NMR (400 MHz, CDCl₃) δ: 7.23 (s, 1H), 7.20 (d, 1H, J=8.0 Hz,), 6.63 (d, 1H, J=8.0 Hz,), 2.92-2.96 (m, 3H), 2.78-2.81 (m, 1H), 2.56-2.63 (m, 1H), 2.32-2.37 (m, 1H), 2.17-2.21 (m, 1H), 1.88-1.96 (m, 3H), 1.70-1.77 (m, 3H), 1.56-1.63 (m, 2H), 1.30-1.34 (m, 2H). MS (ESI): m/z 322, 324 (M+H⁺).

### Compound 73

¹H NMR (400 MHz, CDCl₃) δ: 7.22 (s, 1H), 7.20 (d, 1H, J=8.0 Hz,), 6.63 (d, 1H, J=8.0 Hz,), 2.92-2.96 (m, 3H), 2.59-2.73 (m, 1H), 2.52-2.58 (m, 1H), 2.32-2.37 (m, 1H), 2.17-2.21 (m, 1H), 1.88-1.96 (m, 3H), 1.70-1.77 (m, 3H), 1.56-1.63 (m, 2H), 1.30-1.34 (m, 2H). MS (ESI): m/z 322, 324 (M+H⁺).

### 74. Compound 74: 4-(-1′,3′,4′,6′,7′,8′,9′,9a′-Octahydro-3H-spiro[benzofuran-2,2′-quinolizin]-5-yl)benzonitrile

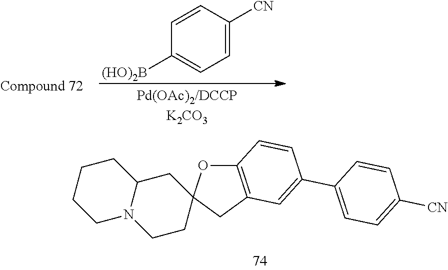

A microwave vial was charged with compound 72 (60 mg, 0.19 mmol, 1.0 eq), 4-cyanophenylboronic acid (41 mg, 0.28 mmol, 1.5 eq), Pd(OAc)₂ (14 mg, 0.03 mmol, 0.05 eq), dicyclohexyl(2′,4′,6′-triisopropylbiphenyl-2-yl)phosphine (DCCP) (14 mg, 0.03 mmol, 0.15 eq), K₂CO₃ (78 mg, 0.57 mmol, 3.0 eq) and dioxane (3 mL). The reaction mixture was heated under microwave irradiation at 100° C. for 1 hr, solids were removed by filtration through a short plug of Celite and the filtrate was washed with brine. The organic layer was dried over anhydrous Na₂SO₄, the solids were removed by filtration and the filtrated was concentrated by evaporation. The crude product was purified by preparative TLC to give compound 74 (18 mg, 28%). ¹H NMR (400 MHz, CDCl₃, δ): 7.66-7.69 (m, 2H), 7.59-7.62 (m, 2H), 7.35-7.37 (m, 2H), 6.85 (d, 1H, J=8.0 Hz), 3.03 (s, 2H), 2.89-2.92 (m, 1H), 2.73-2.77 (m, 1H), 2.53-2.60 (m, 1H), 2.25-2.30 (m, 1H), 2.13-2.20 (m, 1H), 1.87-2.02 (m, 3H), 1.74-1.77 (m, 1H), 1.62-1.68 (m, 2H), 1.52-1.58 (m, 2H), 1.29-1.39 (m, 2H). MS (ESI): m/z 345 (M+H⁺).

### 75. Compound 75: 4-(-1′,3′,4′,6′,7′,8′,9′,9a′-Octahydro-3H-spiro[benzofuran-2,2′-quinolizin]-5-yl)benzonitrile

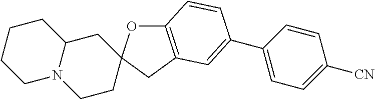

This compound was prepared in 8% yield as described for compound 74 but using compound 73 as the starting material. ¹H NMR (400 MHz, CDCl₃) δ: 7.67 (dd, 2H, J₁=6.8 Hz, J₂=2.0 Hz), 7.60 (dd, 2H, J₁=6.8 Hz, J₂=2.0 Hz,), 7.36 (m, 2H,), 6.86 (d, 1H, J=8.4 Hz), 3.03 (s, 2H), 2.90-2.93 (s, 1H), 2.74-2.78 (m, 1H), 2.54-2.61 (m, 1H), 2.27-2.32 (m, 1H), 2.14-2.20 (m, 1H), 1.88-2.02 (m, 3H), 1.53-1.77 (m, 5H), 1.21-1.36 (m, 2H). MS (ESI): m/z 345 (M+H⁺).

### 76. Compound 76: 5-(Imidazo[1,2-a]pyridin-6-yl)-1′,3′,4′,6′,7′,8′,9′,9a′-octahydro-3H-spiro[benzofuran-2,2′-quinolizine]

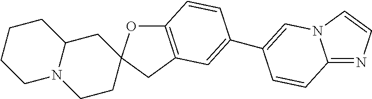

This compound was prepared in 31% yield as described for compound 30 but using intermediate I-41 as the starting material. ¹H NMR (400 MHz, CDCl₃) δ: 8.22 (s, 1H), 7.64 (m, 2H), 7.60 (s, 1H), 7.37 (dd, 1H, J₁=1.6 Hz, J₂=9.6 Hz), 7.33 (s, 1H), 6.85 (dd, 1H, J₁=1.6 Hz, J₂=8.0 Hz), 6.85 (d, 1H, J=8.0 Hz), 3.05 (s, 2H), 2.99-3.01 (m, 1H), 2.83-2.86 (m, 1H), 2.64-2.71 (m, 1H), 2.42-2.45 (m, 1H), 2.22-2.29 (m, 1H), 1.95-2.03 (m, 3H), 1.60-1.80 (m, 5H), 1.33-1.43 (m, 2H). MS (ESI): 360 (M+H⁺).

### 77. Compound 77: 5-(Imidazo[1,2-a]pyridin-6-yl)-1′,3′,4′,6′,7′,8′,9′,9a′-octahydro-3H-spiro[benzofuran-2,2′-quinolizine]

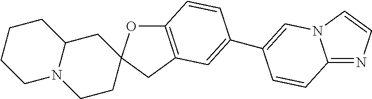

This compound was prepared in 12 mg quantity as described for compound 76 but using the diastereomer of intermediate I-41 derived from compound 73 as the starting material. ¹H NMR (400 MHz, CDCl₃) δ: 8.21 (s, 1H), 7.64 (m, 2H), 7.60 (s, 1H), 7.37 (d, 1H, J=9.2 Hz), 7.31 (m, 2H), 6.85 (d, 1H, J=8.8 Hz), 3.03 (s, 2H), 2.91-293 (m, 1H), 2.74-2.78 (m, 1H), 2.56-2.63 (m, 1H), 2.29-2.34 (m, 1H), 2.15-2.22 (m, 1H), 1.90-2.03 (m, 3H), 1.65-1.78 (m, 3H), 1.54-1.60 (m, 2H), 1.29-1.37 (m, 2H). MS (ESI): 360 (M+H⁺).

### 78. Compound 78: 5-(Morpholinomethyl)-1′,3′,4′,6′,7′,8′,9′,9a′-octahydro-3H-spiro[benzofuran-2,2′-quinolizine]

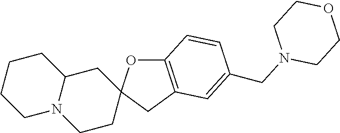

This compound was prepared in 11% yield as described for compound 44 but using intermediate I-43 as the starting material. ¹H NMR (400 MHz, CDCl₃) δ: 7.40 (s, 1H), 7.33 (d, 1H, J=8.0 Hz,), 7.00 (d, 1H, J=8.0 Hz,), 4.00 (t, 4H, J=4.8 Hz), 3.70 (s, 2H), 3.21-3.25 (m, 3H), 3.04-3.07 (m, 1H), 2.84-2.92 (m, 1H), 2.72 (br, 4H), 2.68 (br, 1H), 2.19-2.26 (m, 3H), 2.40-2.45 (m, 1H), 1.99-2.07 (m, 3H), 1.85-1.87 (m, 2H), 1.55-1.59 (m, 2H). MS (ESI): 343 (M+H⁺).

### 79. Compound 79: 5-(Morpholinomethyl)-1′,3′,4′,6′,7′,8′,9′,9a′-octahydro-3H-spiro[benzofuran-2,2′-quinolizine]

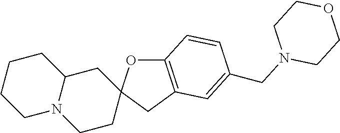

This compound was prepared in 40% yield as described for compound 78 but using the diastereomer of intermediate I-43 derived from compound 73 as the starting material. ¹H NMR (400 MHz, CDCl₃) δ: 7.10 (s, 1H), 7.02 (d, 1H, J=8.0 Hz,), 6.69 (d, 1H, J=8.0 Hz,), 3.70 (t, 4H, J=4.8 Hz), 3.40 (s, 2H), 2.92-2.95 (m, 3H), 2.75-2.79 (m, 1H), 2.57-2.64 (m, 1H), 2.42 (br, 4H), 2.32-2.38 (m, 1H), 2.16-2.23 (m, 1H), 1.89-1.97 (m, 3H), 1.67-1.77 (m, 3H), 1.55-1.61 (m, 2H), 1.29-1.36 (m, 2H). MS (ESI): 343 (M+H⁺).

### 80. Compound 80: 5-((3,4-Dihydroisoquinolin-2(1H)-yl)methyl)-1′,3′,4′,6′,7′,8′,9′,9a′-octahydro-3H-spiro[benzofuran-2,2′-quinolizine]

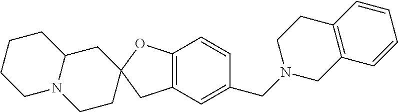

This compound was prepared in 30% yield as described for compound 78 but using 1,2,3,4-tetrahydroisoquinoline as the starting material. ¹H NMR (400 MHz, CDCl₃) δ: 7.18 (s, 1H), 7.07-7.12 (m, 4H), 6.95-6.98 (m, 1H), 6.71 (d, 1H, J=8.0 Hz), 3.60 (s, 2H), 3.58 (s, 2H), 2.96-3.00 (s, 3H), 2.87-2.90 (m, 2H), 2.81-2.84 (m, 1H), 2.69-2.75 (m, 2H), 2.62-2.67 (m, 1H), 2.41-2.46 (m, 1H), 2.21-2.27 (m, 1H), 1.92-2.02 (m, 3H), 1.58-1.78 (m, 5H), 1.35-1.40 (m, 2H). MS (ESI): 389 (M+H⁺),

### 81. Compound 81: 5-((3,4-Dihydroisoquinolin-2(1H)-yl)methyl)-1′,3′,4′,6′,7′,8′,9′,9a′-octahydro-3H-spiro[benzofuran-2,2′-quinolizine]

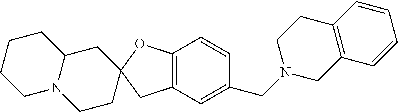

This compound was prepared in 46% yield as described for compound 79 but using 1,2,3,4-tetrahydroisoquinoline as the starting material. ¹H NMR (400 MHz, CDCl₃, δ): 7.18 (s, 1H), 7.06-7.12 (m, 4H), 6.96-6.99 (m, 1H), 6.70 (d, 1H, J=8.0 Hz), 3.60 (s, 2H), 3.58 (s, 2H), 2.96-2.99 (s, 3H), 2.89 (t, 2H, J=5.6 Hz), 2.79-2.83 (m, 1H), 2.73 (t, 2H, J=5.6 Hz), 2.61-2.68 (m, 1H), 2.39-2.44 (m, 1H), 2.21-2.26 (m, 1H), 1.91-2.00 (m, 3H), 1.57-1.77 (m, 5H), 1.36-1.40 (m, 2H). MS (ESI): 389 (M+H⁺).

### 82. Compounds 82 and 82a: 5-Bromo-2′,3′,4′,6′,7′,9′,10′,10a′-octahydro-1′H,3H-spiro[benzofuran-2,8′-pyrido[1,2-a]azepine] and 5-bromo-2′,3′,4′,6′,7′,8′,10′,10a′-octahydro-1′H,3H-spiro[benzofuran-2,9′-pyrido[1,2-a]azepine]

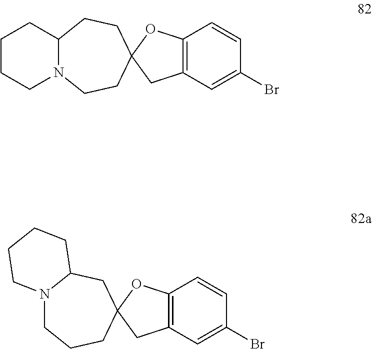

A mixture of these compounds was prepared in 40% yield as described for intermediate I-40 but using the mixture of intermediates I-47 and I-47a as the starting material. ¹H NMR (400 MHz, CDCl₃) δ: 7.16-7.20 (m, 2H), 6.58-6.60 (m, 1H), 2.98 (m, 2H), 2.83-2.88 (m, 2H), 2.38-2.41 (m, 2H), 2.23-2.04 (m, 4H), 1.81-1.88 (m, 2H), 1.70-1.73 (m, 1H), 1.53-1.57 (m, 3H), 1.40-1.43 (m, 1H), 1.29-1.39 (m, 2H). MS (ESI): m/z 336 (M+H⁺).

### 83. Compound 83

### 84. Compound 84 5-(Imidazo[1,2-a]pyridin-6-yl)-2′,3′,4′,6′,7′,9′,10′,10a′-octahydro-1′H,3H-spiro[benzofuran-2,8′-pyrido[1,2-a]azepine] and 5-(imidazo[1,2-a]pyridin-6-yl)-2′,3′,4′,6′,7′,8′,10′,10a′-octahydro-1′H,3H-spiro[benzofuran-2,9′-pyrido[1,2-a]azepine]

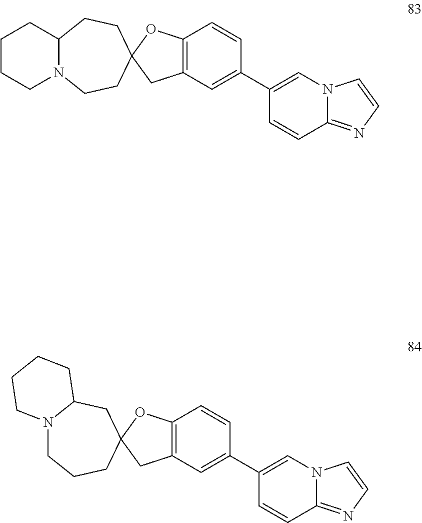

A mixture of these compounds was prepared in 57% yield as described for compound 76 but using the mixture of intermediates I-48 and I-48a as the starting material. The compounds were separated further by preparative TLC using CH₂Cl₂/MeOH (vol/vol=25/1) as the eluent. Chemical structures were assigned on the basis of ¹H-NMR spectra.

Compound 83: (9 mg) ¹H NMR (400 MHz, CDCl₃) δ: 8.22 (s, 1H), 7.64 (m, 2H), 7.60 (s, 1H), 7.37 (dd, 1H, J₁=1.6 Hz, J₂=9.6 Hz), 7.27-7.30 (m, 2H), 6.85 (d, 1H, J₂=8.0 Hz), 3.06 (d, 2H, J=3.2 Hz), 2.86-2.89 (m, 2H), 2.32-2.45 (m, 1H), 2.21-2.31 (m, 3H), 2.10-2.20 (m, 2H), 1.81-2.05 (m, 3H), 1.72-1.80 (m, 1H), 1.54-1.61 (m, 3H), 1.41-1.48 (m, 1H), 1.29-1.38 (m, 1H). MS (ESI): m/z 374 (M+H⁺).

### Compound 84

(4 mg) ¹H NMR (400 MHz, CDCl₃) δ: 8.21 (s, 1H), 7.64 (m, 2H), 7.60 (s, 1H), 7.37 (dd, 1H, J₁=1.6 Hz, J₂=9.6 Hz), 7.27-7.30 (m, 2H), 6.79-6.81 (d, 1H, J₂=8.0), 3.16-319 (m, 2H), 2.92-3.06 (m, 1H), 2.35-2.48 (m, 1H), 2.23-2.35 (m, 3H), 1.87-1.96 (m, 2H), 1.80-1.87 (m, 3H), 1.72-1.80 (m, 2H), 1.54-1.61 (m, 3H), 1.41-1.48 (m, 1H), 1.29-1.38 (m, 1H). MS (ESI): m/z 374 (M+H⁺).

### 85. Compound 85

### 86. Compound 86: 4-(2′,3′,4′,6′,7′,9′,10′,10a′-Octahydro-1′H,3H-spiro[benzofuran-2,8′-pyrido[1,2-a]azepin]-5-yl)benzonitrile and 4-(2′,3′,4′,6′,7′,8′,10′,10a′-octahydro-1′H,3H-spiro[benzofuran-2,9′-pyrido[1,2-a]azepin]-5-yl)benzonitrile

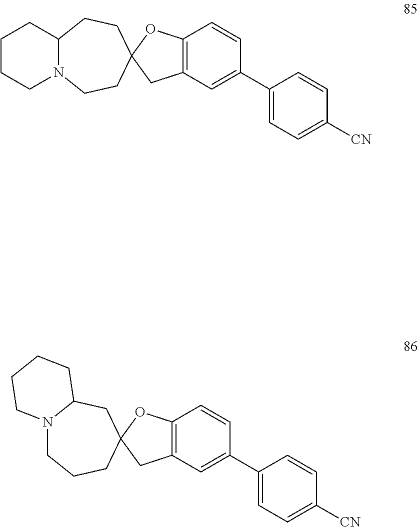

A microwave vial was charged with the mixture of compounds 82 and 82a (103 mg, 0.30 mmol, 1.0 eq), 4-cyanophenylboronic acid (70 mg, 0.60 mmol, 2.0 eq), Pd(dppf)Cl2 (20 mg, 0.03 mmol, 0.1 eq), K₂CO₃ (127 mg, 0.90 mmol, 3.0 eq) and dioxane (3 mL). The reaction mixture was heated under microwave irradiation at 100° C. for 2 hrs, filtered through a short plug of Celite. The filtrate was washed with brine, the combined organic layers were dried over anhydrous Na₂SO₄, the solids were removed by filtration, and the filtrate was concentrated by evaporation. The crude reaction product was purified by preparative TLC using CH₂Cl₂/MeOH (vol/vol=30/1) as the eluent to give compounds 85 and 86. Chemical structures were assigned on the basis of ¹H-NMR spectra.

### Compound 85

(10 mg, 8%) ¹H NMR (400 MHz, CDCl₃) δ: 7.66-7.68 (m, 2H), 7.58-7.62 (m, 2H), 7.35-7.37 (m, 2H), 6.81 (d, 1H, J=8.0 Hz), 3.06 (s, 2H), 2.82-2.87 (m, 2H), 2.21-2.44 (m, 4H), 2.04-2.20 (m, 2H), 1.86-1.91 (m, 2H), 1.70-1.75 (m, 1H), 1.52-1.58 (m, 4H), 1.41-1.48 (m, 1H), 1.29-1.39 (m, 1H). MS (ESI): m/z 359 (M+H⁺).

### Compound 86

(5 mg, 4%) ¹H NMR (400 MHz, CDCl₃) δ: 7.66-7.70 (m, 2H), 7.59-7.63 (m, 2H), 7.35-7.37 (m, 2H), 6.83-6.86 (m, 1H), 3.05-3.11 (m, 3H), 2.86-2.89 (m, 1H), 2.63-2.66 (m, 2H), 2.32 (m, 1H), 2.04-2.20 (m, 2H), 1.85-1.98 (m, 3H), 1.53-1.69 (m, 5H), 1.52-1.58 (m, 5H), 1.29-1.39 (m, 2H). MS (ESI): m/z 359 (M+H⁺).

### 87. Compounds 87 and 87a: 5-(Morpholinomethyl)-2′,3′,4′,6′,7′,9′,10′,10a′-octahydro-1′H,3H-spiro[benzofuran-2,8′-pyrido[1,2-a]azepine] and 5-(morpholinomethyl)-2′,3′,4′,6′,7′,8′,10′,10a′-octahydro-1′H,3H-spiro[benzofuran-2,9′-pyrido[1,2-a]azepine]

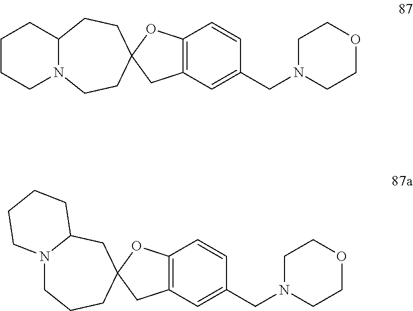

A mixture of these compounds was prepared in 3% yield as described for compound 78 but using the mixture of intermediates I-50 and I-50a as the starting material. ¹H NMR (400 MHz, CDCl₃) δ: 7.05 (s, 1H), 6.93-6.95 (d, 1H, J=8.4 Hz), 6.59-6.61 (d, 1H, J=8.0 Hz,), 3.62-3.64 (m, 4H), 3.32 (s, 2H), 3.03 (m, 1H), 2.89-2.95 (m, 2H), 2.80-2.85 (m, 1H), 2.56-2.61 (m, 2H), 2.35 (m, 4H), 2.13-2.22 (m, 2H), 1.85-2.05 (m, 4H), 1.81-1.85 (m, 3H), 1.45-1.48 (m, 2H), 1.29-1.39 (m, 2H). MS (ESI): m/z 357 (M+H⁺).

### 88. Compounds 88 and 88a: 5-((3,4-Dihydroisoquinolin-2(1H)-yl)methyl)-2′,3′,4′,6′,7′,9′,10′,10a′-octahydro-1′H,3H-spiro[benzofuran-2,8′-pyrido[1,2-a]azepine] and 5-((3,4-dihydroisoquinolin-2(1H)-yl)methyl)-2′,3′,4′,6′,7′,8′,10′,10a′-octahydro-1′H,3H-spiro[benzofuran-2,9′-pyrido[1,2-a]azepine]

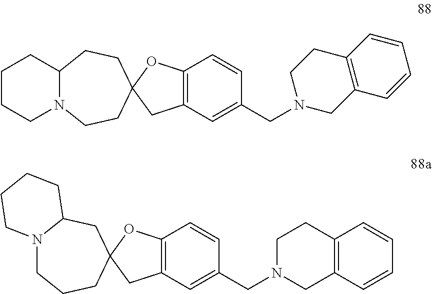

A mixture of these compounds was prepared in 5% yield as described for compound 80 but using the mixture of intermediates I-50 and I-50a as the starting material. ¹H NMR (400 MHz, CDCl₃, δ): 7.18 (s, 1H), 7.06-7.10 (m, 4H), 6.97-6.99 (m, 1H), 6.68-6.70 (m, 1H), 3.60 (s, 2H), 3.58 (s, 2H), 3.01-3.16 (m, 1H), 2.78-2.95 (m, 6H), 2.60-2.73 (m, 4H), 2.31-2.41 (m, 1H), 2.20-2.25 (m, 1H), 2.0-2.26 (m, 4H), 1.55-1.59 (m, 1H), 1.29-1.39 (m, 1H). MS (ESI): m/z 403 (M+H⁺).

### 89. Compound 89: 5-Bromo-2′,3′,5′,6′,8′,8a′-hexahydro-1′H,3H-spiro[benzofuran-2,7′-indolizine]

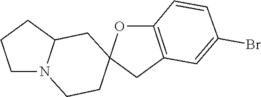

This compound was prepared in 80% yield as described for intermediate I-40 but using intermediate I-60 as the starting material. ¹H NMR (400 MHz, CDCl₃) δ: 7.26 (m, 1H), 7.21 (dd, J₁=8.4 Hz J₂=2.4 Hz, 1H), 6.61 (d, J=8.4 Hz, 1H), 3.46 (m, 1H), 3.35 (m, 1H), 3.05 (m, 1H), 2.86 (m, 1H), 2.77 (dd, J₁=11.2 Hz J₂=4.8 Hz, 1H), 2.51 (t, J=9.6 Hz, 1H), 2.23 (m, 1H), 2.10-1.97 (m, 4H), 1.96-1.80 (m, 2H), 1.75-1.63 (m, 1H). MS (ESI): m/z 308 (M+H⁺).

### 90. Compound 90: 4-(2′,3′,5′,6′,8′,8a′-Hexahydro-1′H,3H-spiro[benzofuran-2,7′-indolizin]-5-yl)benzonitrile

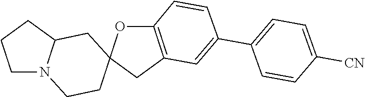

This compound was prepared in 19% yield as described for compound 74 but using compound 89 as the starting material. ¹H NMR (400 MHz, CDCl₃) δ 7.64 (m, 4H), 7.39-7.35 (m, 2H), 6.84 (d, J=8.4 Hz, 1H), 3.17-3.00 (m, 4H), 2.05 (m, 1H), 2.44-2.34 (m, 1H), 2.29-2.18 (m, 1H), 2.07-1.99 (m, 1H), 1.93-1.83 (m, 1H), 1.81-1.72 (m, 1H), 1.55-1.37 (m, 1H). MS (ESI): m/z 331 (M+H⁺).

### 91. Compound 91: 5-(Imidazo[1,2-a]pyridin-6-yl)-2′,3′,5′,6′,8′,8a′-hexahydro-1′H,3H-spiro[benzofuran-2,7′-indolizine]

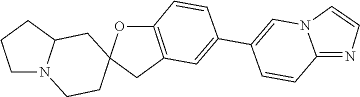

This compound was prepared in 21% yield as described for compound 76 but using intermediate I-61 as the starting material. ¹H NMR (400 MHz, CDCl₃-d₁) δ 8.22 (s, 1H), 7.38-7.28 (m, 3H), 7.63 (m, 3H), 6.83 (d, J=8.0 Hz, 1H), 3.14-3.00 (m, 4H), 2.53 (m, 1H), 2.44 (m, 1H), 2.24 (m, 2H), 2.03 (m, 1H), 1.95-1.71 (m, 4H), 1.54 (t, J=12.4 Hz, 1H), 1.44 (m, 1H). MS (ESI): m/z 346 (M+H⁺).

### 92. Compound 92: 5-(Morpholinomethyl)-2′,3′,5′,6′,8′,8a′-hexahydro-1′H,3H-spiro[benzofuran-2,7′-indolizine]

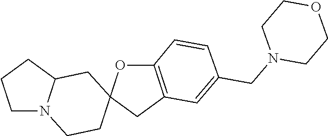

This compound was prepared in 29% yield as described for compound 44 but using intermediate I-63 as the starting material. ¹H NMR (400 MHz, CDCl₃) δ: 7.10 (s, 1H), 7.02 (d, J=8.0 Hz, 1H), 6.68 (d, J=7.6 Hz, 1H), 3.70 (t, J=4.4 Hz, 4H), 3.40 (s, 1H), 3.11 (m, 1H), 3.05-2.97 (m, 3H), 2.53-2.33 (m, 6H), 2.27-2.14 (m, 2H), 2.00 (m, 1H), 1.90-1.70 (m, 4H), 1.50-1.35 (m, 2H). MS (ESI): m/z 329 (M+H⁺).

### 93. Compound 93: 5-(Morpholinomethyl)-2′,3′,5′,6′,8′,8a′-hexahydro-1′H,3H-spiro[benzofuran-2,7′-indolizine]

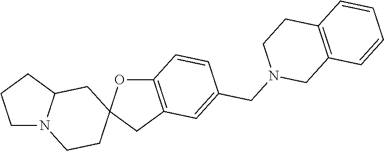

This compound was prepared in 19% yield as described for compound 80 but using intermediate I-63 as the starting material. ¹H NMR (400 MHz, CDCl₃) δ 7.18 (s, 1H), 7.08 (m, 4H), 6.98 (m, 1H), 6.69 (d, J=8.4 Hz, 1H), 3.59 (d, J=8.0 Hz, 4H), 3.11 (m, 1H), 3.05-2.96 (m, 3H), 2.89 (t, J=6.0 Hz, 2H), 2.73 (t, J=6.0 Hz, 2H), 2.50 (m, 1H), 2.39 (m, 1H), 2.28-2.15 (m, 2H), 2.00 (m, 1H), 1.90-1.70 (m, 4H) 1.50-1.37 (m, 2H). MS (ESI): m/z 375 (M+H⁺).

### 94. Compounds 94 and 94a: 5-Bromo-1′,2′,3′,5′,6′,7′,9′,9a′-octahydro-3H-spiro[benzofuran-2,8′-pyrrolo[1,2-a]azepine] and 5-bromo-1′,2′,3′,5′,6′,8′,9′,9a′-octahydro-3H-spiro[benzofuran-2,7′-pyrrolo[1,2-a]azepine]

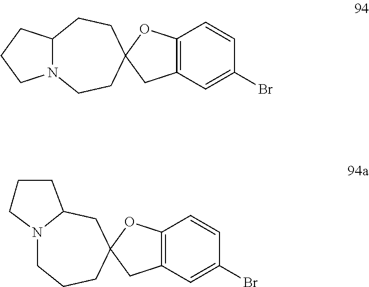

A mixture of these compounds was prepared in 60% yield as described for intermediate I-40 but using the mixture of intermediates I-67 and I-67a as the starting material. ¹HNMR (400 MHz, CDCl₃) δ: 7.16-7.22 (m, 2H), 6.58-6.63 (m, 1H), 2.63-3.22 (m, 5H), 2.16-2.50 (m, 3H), 1.92-2.08 (m, 3H), 1.59-1.88 (m, 5H), 1.40-1.52 (m, 1H). MS (ESI): m/z 322.0 (M+H⁺).

### 95. Compounds 95 and 95a: 4-(1′,2′,3′,5′,6′,7′,9′,9a′-Octahydro-3H-spiro[benzofuran-2,8′-pyrrolo[1,2-a]azepin]-5-yl)benzonitrile and 4-(1′,2′,3′,5′,6′,8′,9′,9a′-octahydro-3H-spiro[benzofuran-2,7′-pyrrolo[1,2-a]azepin]-5-yl)benzonitrile

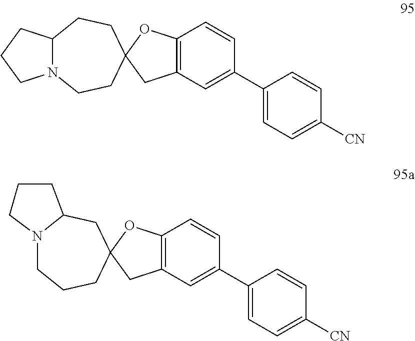

A mixture of these compounds was prepared in 10% yield as described for compounds 85 and 86 but using the mixture of compounds 94 and 94a as the starting material. ¹HNMR (400 MHz, CDCl₃) δ: 7.66-7.68 (d, J=8.0 Hz, 2H), 7.59-7.61 (d, J=8.0 Hz, 2H), 7.36 (m, 2H), 6.82-6.84 (d, J=8.0 Hz, 1H), 3.18-3.23 (m, 1H), 2.88-3.09 (m, 5H), 2.63-2.69 (m, 1H), 2.10-2.29 (m, 4H), 1.93-1.99 (m, 1H), 1.80-1.90 (m, 3H), 1.67-1.73 (m, 1H), 1.54-1.59 (m 1H). MS (ESI): m/z 345.0 (M+H⁺).

### 96. Compounds 96 and 96a: 5-(Imidazo[1,2-a]pyridin-6-yl)-1′,2′,3′,5′,6′,7′,9′,9a′-octahydro-3H-spiro[benzofuran-2,8′-pyrrolo[1,2-a]azepine] and 5-(imidazo[1,2-a]pyridin-6-yl)-1′,2′,3′,5′,6′,8′,9′,9a′-octahydro-3H-spiro[benzofuran-2,7′-pyrrolo[1,2-a]azepine]

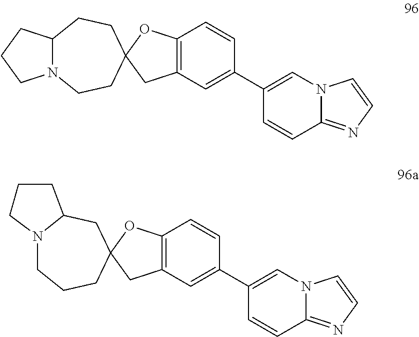

A mixture of these compounds was prepared in 10% yield as described for compounds 83 and 84 but using the mixture of intermediates I-68 and I-68a as the starting material. ¹HNMR (400 MHz, CDCl₃) δ: 8.22 (s, 1H), 7.60-7.65 (m, 3H), 7.28-7.38 (m, 3H), 6.81-6.83 (d, J=8.0 Hz, 1H), 2.82-3.25 (m, 5H), 2.20-2.36 (m, 3H) 2.15 (m, 1H), 1.97-2.07 (m, 3H), 1.84-1.93 (m, 2H), 1.65-1.76 (m, 2H), 1.51-1.60 (m, 1H). MS (ESI): m/z 360.0 (M+H⁺).

### 97. Compounds 97 and 97a: 5-((3,4-Dihydroisoquinolin-2(1H)-yl)methyl)-1′,2′,3′,5′,6′,7′,9′,9a′-octahydro-3H-spiro[benzofuran-2,8′-pyrrolo[1,2-a]azepine] and 5-((3,4-dihydroisoquinolin-2(1H)-yl)methyl)-1′,2′,3′,5′,6′,8′,9′,9a′-octahydro-3H-spiro[benzofuran-2,7′-pyrrolo[1,2-a]azepine]

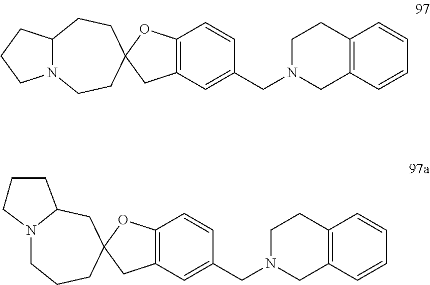

A mixture of these compounds was prepared in 35% yield as described for compound 80 but using the mixture of intermediates I-70 and I-70a as the starting material. ¹H NMR (400 MHz, CDCl₃, δ): 7.19 (s, 1H), 7.06-7.12 (m, 4H), 6.98 (m, 1H), 6.69-6.89 (d, J=8.0 Hz, 1H), 3.60 (s, 2H), 3.58 (s, 2H), 3.09-3.23 (m, 2H), 3.00-3.07 (m, 2H), 2.89 (t, J=6.0 Hz, 2H), 2.74 (t, J=6.0 Hz, 2H), 2.42-2.60 (m, 2H), 2.22-2.32 (m, 2H), 2.11-2.19 (m, 2H), 1.97-2.08 (m, 2H), 1.81-1.93 (m, 2H), 1.67-1.78 (m, 2H), 1.48-1.58 (m, 1H). MS (ESI): m/z 389.0 (M+H⁺).

### 98. Intermediate I-2: 1-Benzyl-4-(2-fluorobenzyl)piperidin-4-ol

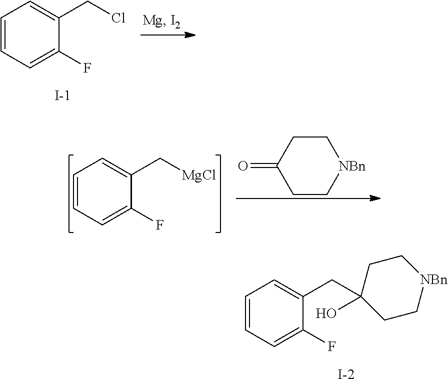

To a stirring suspension of Mg turnings (9.6 g, 0.4 mol) in anhydrous ether (10 mL) was added crystalline iodine (100 mg), the flask was filled with nitrogen and equipped with a reflux condenser. A solution of I-1 (28.8 g, 0.2 mol, Aldrich) in anhydrous ether (200 mL) was prepared and 2-3 mL of this solution were added to the suspension of Mg turnings. The reaction mixture was heated gently to initiate the reaction and the remaining solution of I-1 in ether was added slowly at such a rate that reaction mixture kept refluxing. After the addition was complete, the reaction mixture was refluxed for additional 2 hrs. The mixture was cooled to 0° C., and a solution of 1-benzylpiperidin-4-one (27.9 g, 0.14 mol, Aldrich) in anhydrous ether (150 mL) was added. The reaction mixture was allowed to warm to room temperature and stirred overnight. Saturated aqueous solution of NH₄Cl was added. The aqueous layer was extracted with ether, the combined organic layers were dried over Na₂SO₄, the solids were removed by filtration and the filtrate was concentrated. The crude reaction mixture was purified by silica gel chromatography give 1-2 as a colorless liquid (14 g, 33%). MS (ESI): m/z 300.2 (M+H)⁺.

### 99. Intermediate I-3: 1′-Benzyl-3H-spiro[benzofuran-2,4′-piperidine]

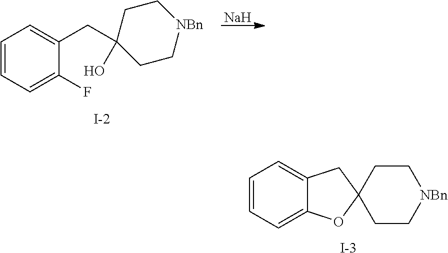

To a stirring suspension of sodium hydride (60% in mineral oil, 10.7 g, 0.27 mol) in a mixture of dry DMF (80 mL) and benzene (16 mL) under nitrogen atmosphere was added a solution of I-2 (16 g, 54 mmol) in a mixture of dry DMF (70 mL) and dry benzene (28 mL). The reaction mixture was refluxed overnight and cooled to room temperature. The reaction mixture was poured into ice water, the aqueous layer was extracted with ether, the combined organic layers were dried over MgSO₄ and concentrated. The crude reaction mixture was purified by silica gel chromatography to give I-3 as a light yellow solid (5.67 g, 38%). ¹H NMR (400 MHz, CDCl₃) δ: 1.77-1.84 (m, 2H), 1.95-1.98 (m, 2H), 2.50-2.67 (m, 4H), 2.98 (s, 2H), 3.57 (s, 2H), 6.74-6.76 (d, 1H), 6.80-6.82 (t, 1H), 7.08-7.13 (m, 2H), 7.25-7.27 (m, 1H), 7.30-7.35 (m, 4H). MS (ESI): m/z 280.2 (M+H)⁺.

### 100. Intermediate I-4: 3H-Spiro[benzofuran-2,4′-piperidine]

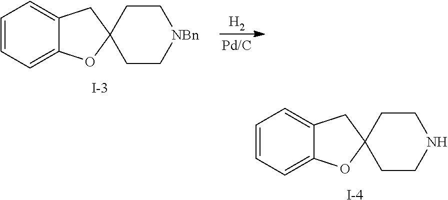

To a solution of I-3 (5.67 g, 20.3 mmol) in i-PrOH (50 mL) was added Pd catalyst (10% wt/wt Pd on carbon, 560 mg) and the mixture was stirred under the atmosphere of hydrogen (4 atm) at 70° C. for 8 hours. The solids were filtered and the filtrate was concentrated to give 1-4 (3.5 g, 92%). MS (ESI): m/z 190.2 (M+H)⁺. This material was used in the next step without purification.

### 101. Intermediate I-5: 5-Bromo-3H-spiro[benzofuran-2,4′-piperidine]

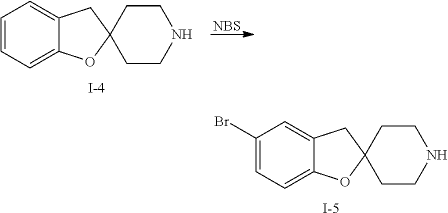

To a solution of I-4 (7.44 g, 39.4 mmol) in methanol at 0° C. was added N-bromosuccinimide (13.9 g, 78.7 mmol) and the mixture was stirred at 0° C. for 6 hours. The reaction mixture was concentrated under reduced pressure and the residue was diluted with water and ethyl acetate. The organic layer was separated and the aqueous phase was basified to pH 8 with saturated solution of sodium bicarbonate. The aqueous layer was extracted with ethyl acetate, the combined organic phase were dried over sodium sulfate, the solids were filtered and the filtrate was concentrated to give 1-5 as a brown solid (6 g, 62%). ¹H NMR (400 MHz, CD₃OD) δ: 7.39 (s, 1H), 7.29 (d, J=8.4 Hz, 1H), 6.75 (d, J=8.4 Hz, 1H), 3.43-3.40 (m, 4H), 3.19 (s, 2H), 2.22-2.19 (m, 2H), 2.12-2.04 (m, 2H). MS (ESI): MS (ESI): m/z 268.2, 270 (M+H)⁺.

### 102. Intermediate I-6: 4-(3H-Spiro[benzofuran-2,4′-piperidine]-5-yl)benzonitrile

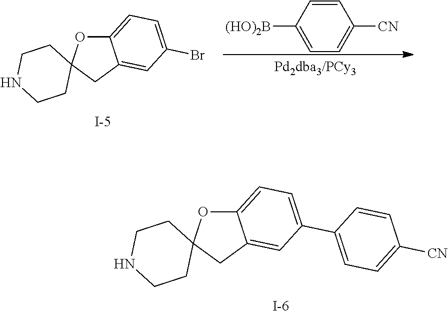

Intermediate I-5 (634 mg, 2.37 mmol), 4-cyanophenylboronic acid (524 mg, 3.57 mmol), Pd₂(dba)₃ (109 mg, 0.12 mmol), PCy₃ (109 mg, 0.40 mmol) and KF (412 mg, 7.11 mmol) were dissolved in a mixture of dioxane (10 mL) and H₂O (3 mL) and the reaction mixture was heated at 100° C. under microwave irradiation for 1 hour. The solids were filtered, the filtrate was concentrated and the crude reaction mixture was purified by flash chromatography to give 1-6 (58% yield).

### 103. Intermediate I-7: 1′-Cyclobutyl-5-(4,4,5,5-tetramethyl-1,3,2-dioxaborolan-2-yl)-3H-spiro[benzofuran-2,4′-piperidine]

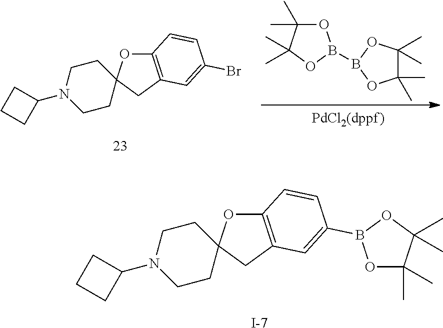

Compound 23 (1.0 g, 3.1 mmol), bis(pinacolato)diboron (1.0 g, 4.1 mmol), PdCl₂(dppf) (260 mg, 0.31 mmol) and KOAc (920 mg, 9.4 mmol) were dissolved in DMF (10 mL) and the mixture was degassed by bubbling N₂. The reaction mixture was stirred at 110° C. under microwave for 2 hrs. The solids were filtered, ethyl acetate was added to the filtrate and the filtrate was washed with water. The combined organic layers were dried over sodium sulfate, the solids were filtered and the filtrate was concentrated. The crude reaction mixture was purified silica gel chromatography to give I-7 (980 mg, 85%).

### 104. Intermediate I-9: 1-(Bromomethyl)-4-chloro-2-fluorobenzene

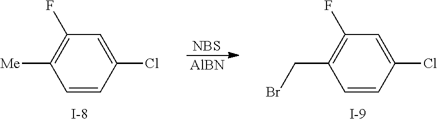

Intermediate I-8 (30 g, 0.21 mol, Aldrich) was dissolved in dry CCl₄ and NBS (37 g, 0.21 mol) was added followed by AIBN (0.5 g). The mixture was refluxed overnight, the solids were filtered and the filtrate was concentrated to give intermediate I-9 as colorless oil (30 g). This crude product was used in the following step without further purification.

### 105. Intermediate I-10: 1-Benzyl-4-(4-chloro-2-fluorobenzyl)piperidin-4-ol

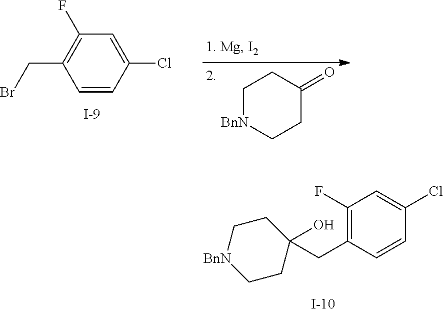

To a mixture of Mg (9.6 g, 0.4 mol) in anhydrous Et₂O (10 mL) under a nitrogen atmosphere was added I₂ (100 mg) followed by drop-wise addition of a solution of 1-9 (18 g, 81 mmol) in anhydrous Et₂O (250 mL). The reaction was initiated by gentle heating and the addition was kept at such a rate that the reaction mixture was refluxing gently. After the addition was completed, the reaction mixture was refluxed for additional 2 hrs. The reaction mixture was cooled to 0° C. and a solution of 1-benzylpiperidin-4-one (13.5 g, 71 mmol) in Et₂O (150 mL) was added. The reaction mixture was allowed to warm to room temperature and stirred at room temperature overnight. Saturated aqueous solution of NH₄Cl and Et₂O was evaporated. The residue was extracted with ethyl acetate, the combined organic layers were dried over sodium sulfate, the solids were filtered and the filtrate was concentrated. The residue was used in the following step without further purification. MS (ESI): m/z 300.2 (M+H)⁺.

### 106. Intermediate I-11: 1′-Benzyl-6-chloro-3H-spiro[benzofuran-2,4′-piperidine]

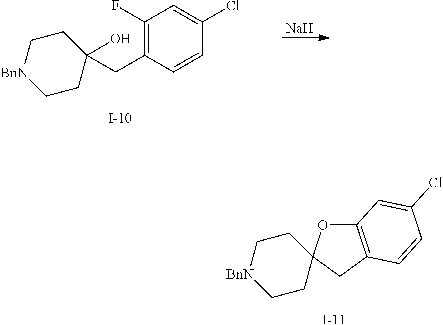

To a stirred suspension of sodium hydride (2.2 g, 55 mmol) in benzene (100 mL) was added a solution of intermediate I-10 (6.0 g, 18 mmol) in benzene (50 mL). The reaction was brought to reflux and DMF (15 mL) was added. The reaction was refluxed for 4 hours and water (100 mL) was added carefully. The organic layer was separated and dried over sodium sulfate, the solids were filtered and the filtrate was concentrated. The crude reaction mixture was purified by silica gel chromatography to give intermediate I-11 (2.1 g). ¹H NMR (400 MHz, CDCl₃) δ: 1.79-1.84 (m, 2H), 1.93-1.97 (m, 2H), 2.57 (brs, 4H), 2.92 (s, 2H), 3.56 (s, 2H), 6.74-6.78 (m, 2H), 7.00-7.02 (m, 2H), 7.25-7.34 (m, 5H). MS (ESI): m/z 314.1 (M+H)⁺.

### 107. Intermediate I-12: 6-Chloro-3H-spiro[benzofuran-2,4′-piperidine]

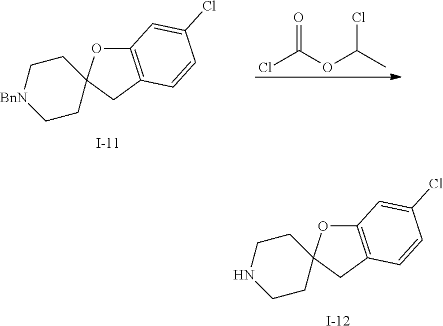

Intermediate I-11 (1.5 g, 19 mmol) and 1-chloroethyl carbonochloridate (2 mL) were dissolved in dichloroethane (20 mL) and the reaction mixture was refluxed overnight. Methanol (20 mL) was added drop-wise and the reaction mixture was stirred for 1 hour at room temperature. The crude reaction mixture was concentrated and saturated aqueous solution of NaHCO₃ was added followed by methylene chloride. The organic layer was separated, dried over sodium sulfate, the solids were filtered and the filtrate was concentrated to give intermediate I-12 (500 mg, 50%). MS (ESI): m/z 224.1 (M+H)⁺.

### 108. Intermediate I-13: 6-Chloro-1′-cyclobutyl-3H-spiro[benzofuran-2,4′-piperidine]

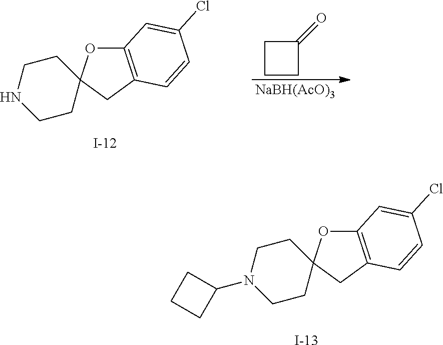

Intermediate I-12 (320 mg, 57 mmol), cyclobutanone (200 mg, 2.9 mmol) and acetic acid (100 mg) were dissolved in methylene chloride (20 mL) and the mixture was stirred at room temperature for 1 hr. Sodium triacetoxyborohydride (610 mg) was added portion wise and the reaction mixture was stirred overnight at room temperature. Saturated aqueous solution of NaHCO₃ was added, the organic layer was separated and dried over sodium sulfate, the solids were filtered and the filtrate was concentrated to give intermediate I-13 (250 mg). MS (ESI): m/z 278.1 (M+H)⁺.

### 109. Intermediate I-14: 1′-Cyclobutyl-6-(4,4,5,5-tetramethyl-1,3,2-dioxaborolan-2-yl)-3H-spiro[benzofuran-2,4′-piperidine]

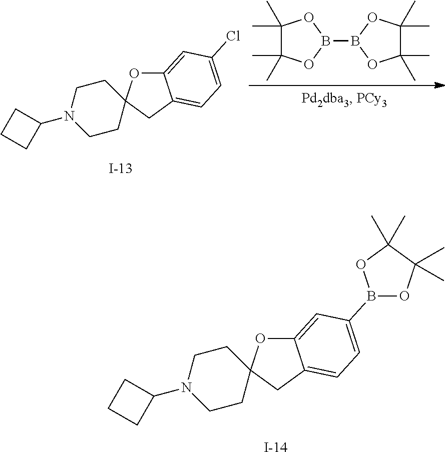

Intermediate I-13 (356 mg, 1.3 mmol), bis(pinacolato)diboron (424 mg, 1.7 mmol), Pd₂ dba₃ (120 mg, 0.13 mmol), PCy₃ (36 mg, 0.13 mmol) and KOAc (382 mg, 3.9 mmol) were dissolved in dioxane (10 mL) and the mixture was degassed by bubbling N₂. The reaction mixture was heated to 130° C. under microwave for 2 hrs and the solids were filtered. The filtrate was concentrated the crude reaction mixture was purified by silica gel chromatography to give intermediate I-14 (288 mg, 60%).

### 110. Intermediate I-15: 1′-Cyclobutyl-3H-spiro[benzofuran-2,4′-piperidine]-5-carbaldehyde

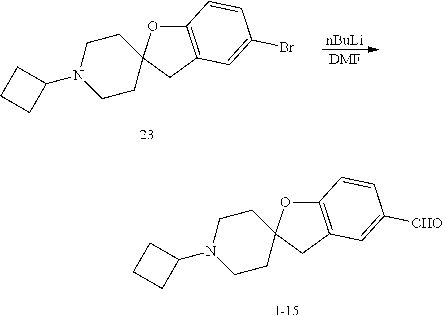

To a stirred solution of compound 23 (800 mg, 2.48 mmol) in THF at −78° C. under nitrogen was added n-butyl lithium (2.0 mL, 2.5 M solution in hexane). The reaction mixture was allowed to warm to −20° C. and stirred at that temperature for additional 30 minutes. The reaction mixture was cooled to −78° C. and dry DMF was added drop-wise. After the addition was complete the reaction mixture was allowed to warm to room temperature and stirred for additional 30 minutes. Methanol was added and the reaction mixture was concentrated. Water and ethyl acetate were added to the residue and the organic layer was separated. The combined organic layers were dried over sodium sulfate, the solids were filtered and the filtrate was concentrated. The crude reaction mixture was purified by silica gel chromatography to give intermediate I-15 (400 mg, 60%). MS (ESI): m/z 272.2 (M+H)⁺.

### 111. Intermediate I-16: 1′-Cyclobutyl-3H-spiro[benzofuran-2,4′-piperidine]-6-carbonitrile

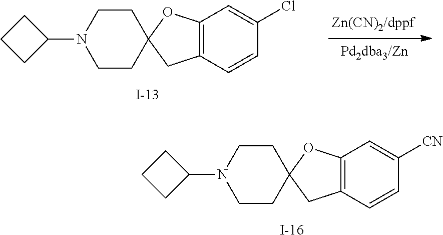

Intermediate I-13 (150 mg, 0.54 mmol), Zn(CN)₂ (126 mg, 1.1 mmol), Pd₂(dba)₃ (25 mg, 0.027 mmol), dppf (30 mg, 0.054 mmol) and Zn powder (26 mg, 0.41 mmol) were suspended in dimethylacetate (4 mL) and the mixture was degassed by bubbling N₂. The reaction mixture was stirred at 150° C. under microwave irradiation for 4 hrs. The solids were filtered and water (60 mL) was added to the filtrate followed by ethyl acetate. The organic layer was washed with water, dried over sodium sulfate, the solids were filtered and the filtrate was concentrated. The crude reaction mixture was purified by silica gel chromatography to give intermediate I-16 (44 mg, 30%). (ESI): m/z 268 (M+H⁺).

### 112. Intermediate I-17: 1′-Cyclobutyl-3H-spiro[benzofuran-2,4′-piperidine]-6-carbaldehyde

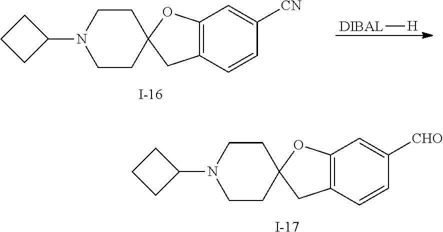

To a solution of intermediate I-16 (500 mg, 1.87 mmol) in dichloromethane (10 mL) cooled to −78° C. was added drop-wise DIBAL-H (5.6 mL, 5.6 mmol, 1.0M solution in dichloromethane) and the reaction mixture was stirred −78° C. for exactly 1 hr. Saturated aqueous solution of NH₄Cl (1.0 mL) was added and the reaction mixture was allowed to warm to room temperature. Additional dichloromethane was added and the organic layer was washed with water and brine, dried over sodium sulfate, the solids were filtered and the filtrate was concentrated. The crude reaction mixture was purified by silica gel chromatography to give intermediate I-17 (405 mg, 80%). (ESI): m/z 272 (M+H⁺).

### 113. Intermediate I-19: 1-benzylazepan-4-one

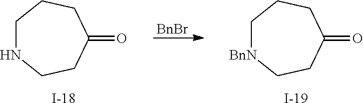

To a mixture of benzyl bromide (5.0 g, 29 mmol, 1.0 eq) and 1-18 hydrochloride salt (4.4 g, 29 mmol, 1.0 eq, Aldrich) in acetone (10 mL) was added solid K₂CO₃ (12 g, 88 mmol, 3.0 eq), and the mixture was stirred for 16 hrs at room temperature. The crude reaction mixture was concentrated by evaporation, the residue was dissolved in ethyl acetate and the organic layer was washed with water. The crude reaction product was purified by silica gel chromatography to give intermediate I-19 (5.6 g, 95%). MS (ESI): m/z 204.1 (M+H⁺).

### 114. Intermediate I-20: 1-benzyl-4-(2-fluorobenzyl)azepan-4-ol

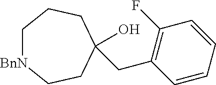

This intermediate was prepared in 55% crude yield as described for intermediate I-2 but using intermediate I-19 as the starting material. MS (ESI): m/z 314.2 (M+H⁺).

### 115. Intermediate I-21: 1-benzyl-3′H-spiro[azepane-4,2′-benzofuran]

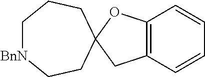

This intermediate was prepared in 61% yield as described for intermediate I-3 but using intermediate I-20 as the starting material. ¹H NMR (400 MHz, CDCl₃) δ: 7.24-7.34 (m, 6H), 7.08-7.12 (m, 2H), 6.79-6.81 (m, 1H), 6.71-6.73 (d, 1H), 3.65 (s, 2H), 3.03 (s, 2H), 2.62-2.75 (m, 3H), 2.55-2.60 (m, 1H), 2.09-2.20 (m, 2H), 1.94-1.98 (m, 3H), 1.62-1.68 (m, 1H). MS (ESI): m/z 294.2 (M+H⁺).

### 116. Intermediate I-22: 3′H-spiro[azepane-4,2′-benzofuran]

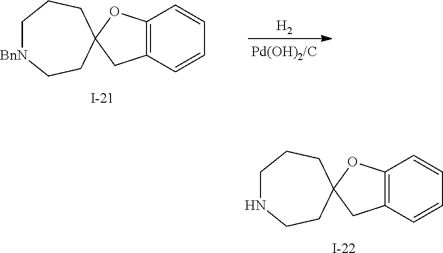

Intermediate I-21 (2.0 g, 6.8 mmol, 1.0 eq) was dissolved in methanol (10 mL), Pd(OH)₂/C (0.2 g, 10 wt/wt %) was added, and the mixture was stirred under H₂ (1 atm) for 2 hours. The solids were removed by filtration through a shot plug of Celite and the filtrate was concentrated to give intermediate I-22 (1.3 g, 95%). MS (ESI): m/z 204.2 (M+H⁺).

### 117. Intermediate I-23: 5′-bromo-3′H-spiro[azepane-4,2′-benzofuran]

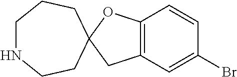

This intermediate was prepared in 80% yield as described for intermediate I-5 but using intermediate I-22 as the starting material. MS (ESI): m/z 282.0 (M+H⁺).

### 118. Intermediate I-24: 1-cyclobutyl-5′-(4,4,5,5-tetramethyl-1,3,2-dioxaborolan-2-yl)-3′H-spiro[azepane-4,2′-benzofuran]

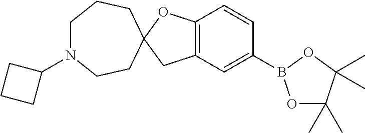

This intermediate was prepared in 38% yield as described for intermediate I-7 but using compound 47 as the starting material. MS (ESI): m/z 384.1 (M+H⁺).

### 119. Intermediate I-26: 1-(bromomethyl)-4-chloro-2-fluorobenzene

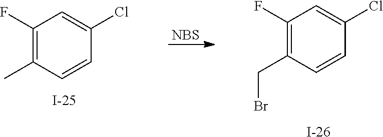

To a stirred solution of intermediate I-25 (15 g, 0.10 mol, 1.0 eq, Aldrich) in CCl₄ was added NBS (19 g, 0.10 mol, 1.0 eq) and AIBN (0.5 g, 3 mmol, 0.03 eq), and the reaction mixture was refluxed for 3 hrs. Solids were removed by filtration, and the volume of the filtrate was reduced by evaporation. The crude residue was filtered through a short plug of silica gel to give intermediate I-26 (22 g, 94%) as a colorless liquid that was used in the following step without further purification.

### 120. Intermediate I-27: benzyl 4-(4-chloro-2-fluorobenzyl)-4-hydroxyazepane-1-carboxylate

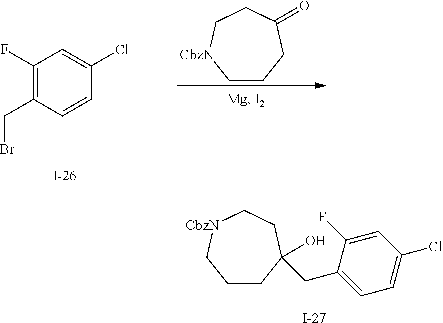

A three-necked flask was charged at room temperature with magnesium turnings (2.7 g, 0.11 mol, 2.3 eq) and I₂ (100 mg, 0.40 mmol, 0.008 eq) and dry ether was added. Intermediate I-26 (10 g, 49 mmol, 1.0 eq) was added via syringe, and the reaction mixture was stirred at room temperature until I₂ was consumed (judged by color disappearance). The reaction mixture was refluxed for 1 hour, additional ether (50 mL) was added, and the reaction mixture was allowed to cool to room temperature and was added to a solution of benzyl 4-oxoazepane-1-carboxylate (7.7 g, 31 mmol, 0.6 eq) in ether at room temperature. A white precipitate formed during the addition. The reaction mixture was stirred at room temperature for an additional hour, the solids were removed by filtration and the filtrate was extracted with ethyl acetate. The combined organic phases were dried over anhydrous MgSO₄, the solids were removed by filtration and the filtrate was concentrated. The crude residue was purified by flash silica gel chromatography to give intermediate I-27 (7.0 g, 40%). MS (ESI): m/z 392.1 (M+H⁺).

### 121. Intermediate I-28: benzyl 6′-chloro-3′H-spiro[azepane-4,2′-benzofuran]-1-carboxylate

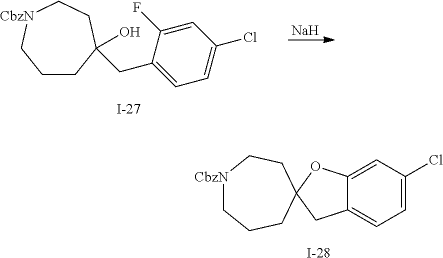

To a solution of intermediate I-27 (5.0 g, 13 mmol, 1.0 eq) in dry DMF at 0° C. was added sodium hydride (60% in mineral oil, 1.3 g, 33 mmol, 2.5 eq), and the reaction mixture was stirred at room temperature for 16 hrs. Water was added carefully, and the crude reaction mixture was extracted with ethyl acetate. The combined organic phases were dried over anhydrous MgSO₄, the solids were removed by filtration and the filtrate was concentrated. The crude residue was purified by flash silica gel chromatography to give intermediate I-28 (2.8 g, 59%). ¹H NMR (400 MHz, CDCl₃) δ: 7.30-7.40 (m, 5H), 7.00 (d, 1H, J=8.0 Hz), 6.78 (m, 1H), 6.72 (s, 1H), 5.15 (s, 2H), 3.65-3.85 (m, 2H), 3.35-3.45 (m, 2H), 2.90-2.98 (m, 2H), 2.00-2.20 (m, 3H), 1.80-1.98 (m, 1H), 1.65-1.80 (m, 2H). MS (ESI): m/z 372.1 (M+H⁺).

### 122. Intermediate I-29: 6′-chloro-3′H-spiro[azepane-4,2′-benzofuran]

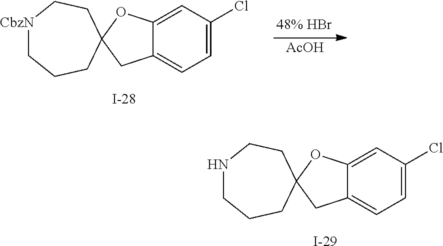

Intermediate I-28 (2.0 g, 5.4 mmol, 1.0 eq) was dissolved in acetic acid (5 mL) and a solution of hydrobromic acid (48% in AcOH, 5 mL) was added. The reaction mixture was stirred at room temperature for 16 hrs, water was added carefully and the reaction mixture was extracted with ether. The aqueous phase was dried by evaporation, and a concentrated aqueous solution of sodium hydroxide was added followed by dichloromethane. The crude reaction product was partitioned into dichloromethane. The combined dichloromethane phases were dried over anhydrous MgSO₄, the solids were removed by filtration and the filtrate was concentrated to give intermediate I-29 (1.1 g, 86%). MS (ESI): m/z 238.0 (M+H⁺).

### 123. Intermediate I-30: (1-cyclobutyl-3′H-spiro[azepane-4,2′-benzofuran]-6′-yl)boronic acid

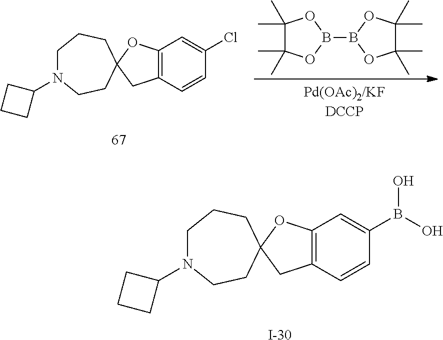

A microwave vial was charged with compound 67 (490 mg, 1.7 mmol, 1.0 eq), bis(pinacolato)diboron (560 mg, 2.2 mmol, 1.3 eq), Pd(OAc)₂ (38 mg, 0.17 mmol, 0.1 eq), DCCP (160 mg, 0.34 mmol, 0.2 eq), KF (150 mg, 2.5 mmol, 1.5 eq) and 1,4-dioxane (10 mL). The vial was sealed, evacuated and purged three times with nitrogen. The reaction mixture was heated under microwave irradiation at 130° C. for 2 hrs, and the solids were removed by filtration and washed with ethyl acetate. Water was added, and the crude reaction mixture was extracted with ethyl acetate. The combined organic layers were washed with water and brine, and dried with sodium sulfate. The solids were removed by filtration, the filtrate was concentrated and the residue was purified by reverse phase flash chromatography to give intermediate I-30 (320 mg, 65%). MS (ESI): m/z 302.2 (M+H⁺).

### 124. Intermediate I-32: ethyl 3-(2-(2-ethoxy-2-oxoethyl)piperidin-1-yl)propanoate

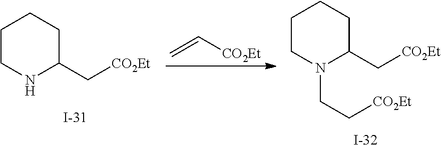

A mixture of intermediate I-31 (10 g, 59 mmol, 1.0 eq, Aldrich), ethyl acrylate (19 mL, 180 mmol, 3.0 eq) and triethylamine (50 mL, 140 mmol, 6.0 eq) in ethanol (100 mL) was stirred at room temperature under nitrogen for 90 hr. The crude reaction mixture was concentrated by evaporation to give intermediate I-32 (15 g, 97%) which was used in the following step without further purification. MS (ESI): m/z 272 (M+H⁺).

### 125. Intermediate I-33: ethyl 2-oxooctahydro-1H-quinolizine-3-carboxylate

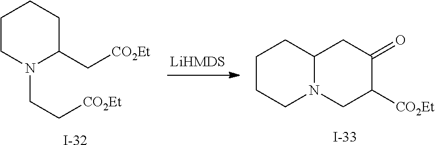

LiHMDS (80 mL, 1.0 M in THF, 80 mmol, 2.0 eq) was added drop-wise to a stirred solution of intermediate I-32 (10 g, 40 mmol, 1.0 eq) in anhydrous THF (30 mL) at −78° C. under nitrogen atmosphere and stirred at −78° C. for 2 hrs. A solution of hydrochloric acid (12 M in water, 6 mL) was added, and the solution was warmed to room temperature. Water (50 mL) was added, and the mixture was extracted with ether. The aqueous layer was basified to pH ˜10 by addition of saturated aqueous solution of potassium carbonate, and the aqueous layer was extracted with ether. The combined organic layers were dried over anhydrous Na₂SO₄, the solids were removed by filtration and the filtrate was concentrated by evaporation to give intermediate I-33 (7.8 g, 93%) which was used in the following step without further purification. MS (ESI): m/z 226 (M+H⁺).

### 126. Intermediate I-34: hexahydro-1H-quinolizin-2(6H)-one

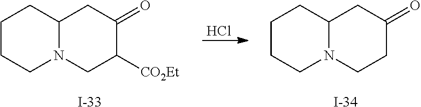

A mixture of intermediate I-33 (5.8 g, 27 mmol) in aqueous HCl (6 M in water, 100 mL) was refluxed for 20 hr, and the solution was neutralized carefully with solid potassium carbonate until gas evolution stopped and the aqueous solution was saturated. The solids were removed by filtration and washed with ether. The aqueous layer was extracted with ether, the combined organic layers were dried over anhydrous Na₂SO₄, the solids were removed by filtration, and the filtrate was concentrated by evaporation to give intermediate I-34 (3.4 g, 81%) which was used in the following step without further purification. ¹H NMR (400 MHz, CDCl₃) δ: 3.11 (m, 1H), 2.99 (d, 1H, J=11.2 Hz), 2.64-2.72 (m, 1H), 2.28-2.38 (m, 4H), 2.03-2.10 (m, 2H), 1.62-1.77 (m, 4H), 1.23-1.36 (m, 2H). MS (ESI): m/z 154 (M+H⁺).

### 127. Intermediate I-36: 2-(5-bromo-2-fluorophenyl)-1,3-dithiane

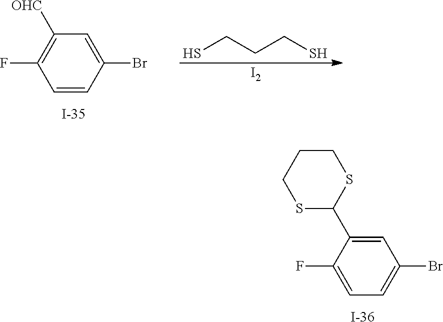

A mixture of intermediate I-35 (12 g, 58 mmol, 1.0 eq, Aldrich), propane-1,3-dithiol (5.7 mL, 57 mmol, 0.99 eq) and I₂ (0.43 g, 1.7 mmol, 0.03 eq) in CHCl₃ (100 mL) was stirred at room temperature for 20 hr. The orange solution was poured into a solution of Na₂SO₃ (0.8 M in water, 120 mL) and a solution of NaOH (40 wt % in water, 90 mL) was added. The organic layer was collected, and the aqueous layers was extracted with additional CHCl₃. The combined organic layers were washed with water and dried over anhydrous Na₂SO₄, the solids were removed by filtration and the filtrate was concentrated by evaporation. The crude reaction product was purified by silica gel flash chromatography to give intermediate I-36 (12 g, 71%). MS (ESI): m/z 293, 295 (M+H⁺).

### 128. Intermediate I-37: 2-(2-(5-bromo-2-fluorophenyl)-1,3-dithian-2-yl)octahydro-1H-quinolizin-2-ol

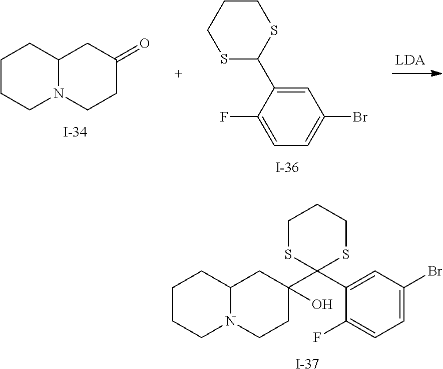

To a solution of intermediate I-36 (11 g, 38 mmol, 1.0 eq) in anhydrous THF (200 mL) at −78° C. under nitrogen atmosphere was added LDA (22 mL, 1.8 M in THF/Heptane, 39.6 mmol, 1.05 eq) drop-wise, and the reaction mixture was stirred at −78° C. for an additional 30 minutes. The reaction mixture was allowed to warm −20° C. and stirred at −20° C. for an additional 30 minutes. The reaction mixture was cooled to −78° C., a solution of intermediate I-34 (5.5 g, 36 mmol, 0.95 eq) in THF (50 mL) was added drop-wise and the reaction mixture was stirred at −78° C. for 2 hrs. A saturated aqueous solution of NH₄Cl (50 mL) was added, and the organic layer was collected. The aqueous layer was extracted with ethyl acetate, the combined organic layers were dried over anhydrous Na₂SO₄, the solids were removed by filtration, and the filtrate was evaporated. The crude reaction product was purified by silica gel flash chromatography to give intermediate I-37 (12.5 g, 78%). MS (ESI): m/z 446, 448 (M+H⁺).

### 129. Intermediate I-38: (5-bromo-2-fluorophenyl)(2-hydroxyoctahydro-1H-quinolizin-2-yl)methanone

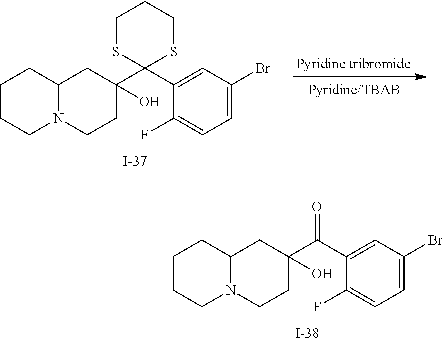

A mixture of intermediate I-37 (13 g, 29 mmol, 1.0 eq), pyridine tribromide (19 g, 58 mmol, 2.0 eq), TBAB (0.93 g, 2.9 mmol, 0.1 eq) and pyridine (3 mL, 44 mmol, 1.5 eq) in a mixture of CH₂Cl₂ (150 mL) and water (40 mL) was stirred at room temperature for 14 hrs. Solids were removed by filtration, the filtrate was washed with water, the organic layer concentrated by evaporation, the intermediate I-38 (7.5 g, 72%) was allowed to air-dry and used in the following step without further purification. MS (ESI): m/z 356, 358 (M+H⁺).

### 130. Intermediate I-39: 5-bromo-1′,3′,4′,6′,7′,8′,9′,9a′-octahydro-3H-spiro[benzofuran-2,2′-quinolizin]-3-one

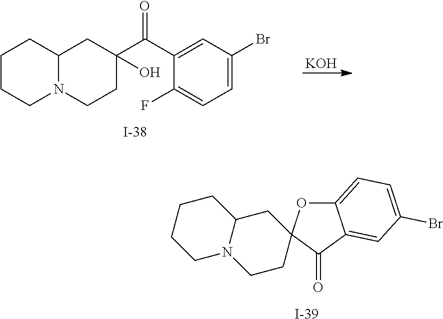

To a solution of intermediate I-38 (6.0 g, 17 mmol, 1.0 eq) in methanol (100 mL) at room temperature was added KOH (1.9 g, 34 mmol, 2.0 eq), and the reaction mixture was stirred at 60° C. for 2 hrs. Water (50 mL) was added, methanol was removed under reduced pressure and the crude reaction mixture was extracted with ethyl acetate. The combined organic layers were dried over anhydrous Na₂SO₄, the solids were removed by filtration and the filtrate was concentrated by evaporation to give intermediate I-39 (4.1 g, 72%) which was used in following step without further purification. MS (ESI): m/z 336,338 (M+H⁺).

### 131. Intermediate I-40: 5-bromo-1′,3′,4′,6′,7′,8′,9′,9a′-octahydro-3H-spiro[benzofuran-2,2′-quinolizine]

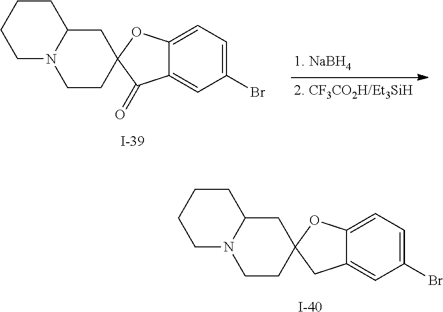

To a solution of intermediate I-39 (4.6 g, 14 mmol, 1.0 eq) in methanol (50 mL) was added NaBH₄ (1.1 g, 29 mmol, 2.0 eq), and the reaction mixture was stirred at 0° C. for 1 hr. Water (20 mL) was added, methanol was removed under reduced pressure and the aqueous residue was extracted with ethyl acetate. The combined organic layers were dried over anhydrous Na₂SO₄, the solids were removed by filtration and the filtrate was concentrated by evaporation. To the residue was added excess CF₃CO₂H (20 mL) and Et₃SiH (3.6 g, 31 mmol, 2.0 eq), and the reaction mixture was stirred at 50° C. for 2 hrs. Excess CF₃CO₂H was removed under reduced pressure, the residue was diluted with ethyl acetate (30 mL) and pH was adjusted to 7-8 by adding saturated aqueous solution of NaHCO₃. The crude reaction mixture was extracted with ethyl acetate, the combined organic layers were dried over anhydrous Na₂SO₄, the solids were removed by filtration and the filtrate was concentrated by evaporation. The crude reaction product was purified by silica gel flash chromatography to give intermediate I-40 (4.4 g, 88%). MS (ESI): m/z 322,324 (M+H⁺).

### 132. Intermediate I-41: 5-(4,4,5,5-tetramethyl-1,3,2-dioxaborolan-2-yl)-1′,3′,4′,6′,7′,8′,9′,9a′-octahydro-3H-spiro[benzofuran-2,2′-quinolizine]

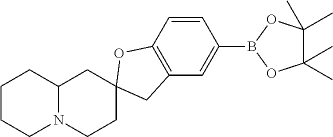

This intermediate was prepared in 48% yield as described for intermediate 1-7 but using compound 72 as the starting material. MS (ESI): m/z 370 (M+H⁺).

### 133. Intermediate I-42: 1′,3′,4′,6′,7′,8′,9′,9a′-octahydro-3H-spiro[benzofuran-2,2′-quinolizine]-5-carbonitrile

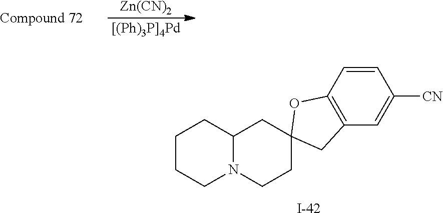

A microwave tube was charged with compound 72 (500 mg, 1.6 mmol, 1.0 eq), dicyanozinc (360 mg, 3.1 mmol, 2.0 eq), [(Ph₃)P]₄Pd (180 mg, 0.16 mmol, 0.1 eq) and DMF (3 mL). The reaction mixture was heated under microwave irradiation at 130° C. for 3 hrs. The solids were removed by filtration through a short plug of Celite, the filtrate was washed with ethyl acetate and the combiner organic layers were washed with brine, dried over anhydrous Na₂SO₄, the solids were removed by filtration and the filtrate was concentrated by evaporation. The crude product was purified by silica gel flash chromatography to give intermediate I-42 (100 mg, 24%). MS (ESI): m/z 269 (M+H⁺).

### 134. Intermediate I-43: 1′,3′,4′,6′,7′,8′,9′,9a′-octahydro-3H-spiro[benzofuran-2,2′-quinolizine]-5-carbaldehyde

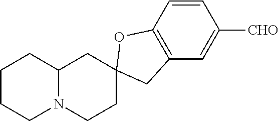

This intermediate was prepared in 69% yield as described for intermediate I-17 but using intermediate I-42 as the starting material. MS (ESI): m/z 272 (M+H⁺).

### 135. Intermediates I-44 and I-44a: octahydropyrido[1,2-a]azepin-8(2H)-one and octahydropyrido[1,2-a]azepin-9(6H)-one

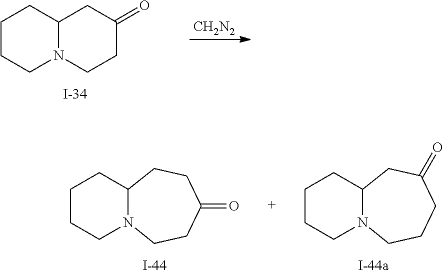

To a solution of intermediate I-34 (4.0 g, 26 mmol, 1.0 eq) in ethanol (25 mL) at 0° C. was added drop-wise a solution of diazomethane ethyl ether (100 mL, 56 mmol, 2.2 eq) over a period of 15 min. The reaction mixture was stirred at room temperature for 4 hrs. The reaction was stopped by drop-wise addition of a solution of acetic acid in ethyl ether. The crude reaction mixture was concentrated by evaporation to give a 1:1 mixture (based LC-MS analysis) of intermediates I-44 and I-44a (4.5 g, 85%) which was used in the following step without further purification. MS (ESI): m/z 168 (M+H⁺).

### 136. Intermediates I-45 and I-45a: 8-(2-(5-bromo-2-fluorophenyl)-1,3-dithian-2-yl)decahydropyrido[1,2-a]azepin-8-ol and 9-(2-(5-bromo-2-fluorophenyl)-1,3-dithian-2-yl)decahydropyrido[1,2-a]azepin-9-ol

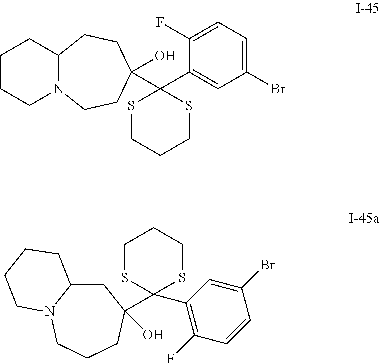

A mixture of these intermediates was prepared in 80% yield as described for intermediate I-37 but using the mixture of intermediates I-44 and I-44a as the starting material. MS (ESI): m/z 469 (M+H⁺).

### 137. Intermediates I-46 and I-46a: (5-bromo-2-fluorophenyl)(8-hydroxydecahydropyrido[1,2-a]azepin-8-yl)methanone and (5-bromo-2-fluorophenyl)(9-hydroxydecahydropyrido[1,2-a]azepin-9-yl)methanone

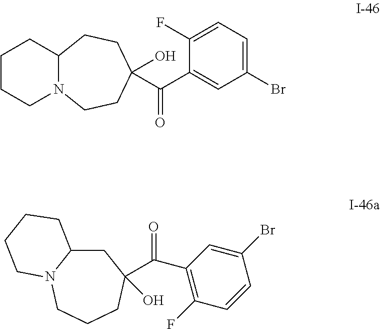

A mixture of these intermediates was prepared in 71% yield as described for intermediate I-38 but using the mixture of intermediates I-45 and I-45a as the starting material. MS (ESI): m/z 370 (M+H⁺).

### 138. Intermediates I-47 and I-47a: 5-bromo-2′,3′,4′,6′,7′,9′,10′,10a′-octahydro-1′H,3H-spiro[benzofuran-2,8′-pyrido[1,2-a]azepin]-3-one and 5-bromo-2′,3′,4′,6′,7′,8′,10′,10a′-octahydro-1′H,3H-spiro[benzofuran-2,9′-pyrido[1,2-a]azepin]-3-one

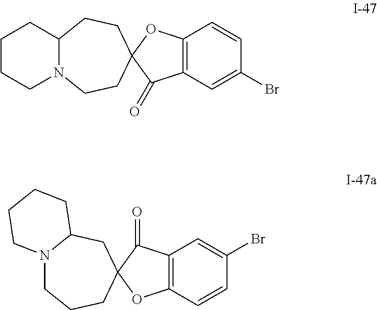

A mixture of these intermediates was prepared in 72% yield as described for intermediate I-39 but using the mixture of intermediates I-46 and I-46a as the starting material. MS (ESI): m/z 350 (M+H⁺).

### 139. Intermediates I-48 and I-48a: 5-(4,4,5,5-tetramethyl-1,3,2-dioxaborolan-2-yl)-2′,3′,4′,6′,7′,9′,10′,10a′-octahydro-1′H,3H-spiro[benzofuran-2,8′-pyrido[1,2-a]azepine] and 5-(4,4,5,5-tetramethyl-1,3,2-dioxaborolan-2-yl)-2′,3′,4′,6′,7′,8′,10′,10a′-octahydro-1′H,3H-spiro[benzofuran-2,9′-pyrido[1,2-a]azepine]

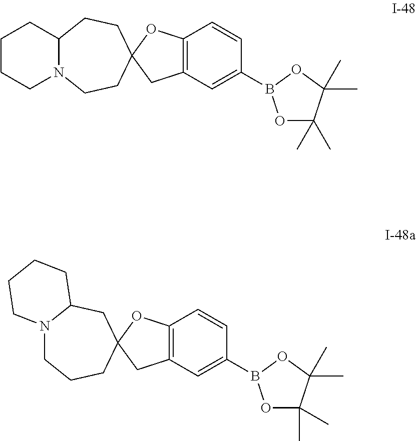

A mixture of these intermediates was prepared in 90% yield as described for intermediate I-7 but using the mixture of compounds 82 and 82a as the starting material. MS (ESI): m/z 383 (M+H⁺).

### 140. Intermediates I-49 and I-49a: 2′,3′,4′,6′,7′,9′,10′,10a′-octahydro-1′H,3H-spiro[benzofuran-2,8′-pyrido[1,2-a]azepine]-5-carbonitrile and 2′,3′,4′,6′,7′,8′,10′,10a′-octahydro-1′H,3H-spiro[benzofuran-2,9′-pyrido[1,2-a]azepine]-5-carbonitrile

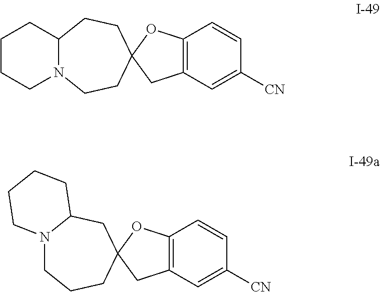

A mixture of these intermediates was prepared in 30% yield as described for intermediate I-42 but using the mixture of compounds 82 and 82a as the starting material. MS (ESI): m/z 283 (M+H⁺).

### 141. Intermediates I-50 and I-50a: 2′,3′,4′,6′,7′,9′,10′,10a′-octahydro-1′H,3H-spiro[benzofuran-2,8′-pyrido[1,2-a]azepine]-5-carbaldehyde and 2′,3′,4′,6′,7′,8′,10′,10a′-octahydro-1′H,3H-spiro[benzofuran-2,9′-pyrido[1,2-a]azepine]-5-carbaldehyde

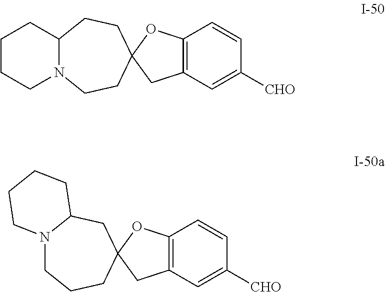

A mixture of these intermediates was prepared in 60% yield as described for intermediate I-17 but using the mixture of intermediates I-49 and I-49a as the starting material. MS (ESI): m/z 286 (M+H⁺).

### 142. Intermediate I-52: tert-butyl 2-(2-diazoacetyl)pyrrolidine-1-carboxylate

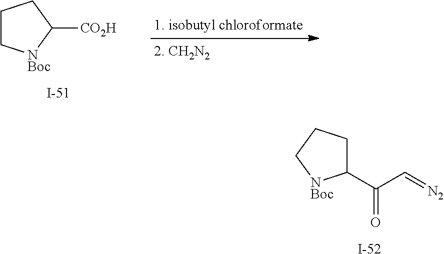

To a mixture of intermediate I-51 (35 g, 163 mmol, 1.0 eq, Aldrich) in dry ether (190 mL) at −15° C. was added triethylamine (23 g, 163 mmol, 1.0 eq) followed by the addition of isobutyl chloroformate (24 g, 179 mmol, 1.1 eq) at −20° C. After 25 minutes, diazomethane (326 mmol, 2.0 eq) in ether (580 mL) was added to the reaction mixture at 0° C., and the reaction mixture was stirred at 0° C. for 4 hrs. Excess diazomethane was quenched by addition of glacial acetic acid. Water was added and the organic layer was washed with saturated aqueous solution of NaHCO₃, water and brine. The combined organic layers were dried over anhydrous Na₂SO₄, solids were removed by filtration and the filtrate was concentrated. The crude reaction product was purified by silica gel flash chromatography to give intermediate I-52 (32 g, 82%).

### 143. Intermediate I-53: tert-butyl 2-(2-methoxy-2-oxoethyl)pyrrolidine-1-carboxylate

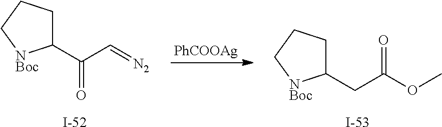

To a suspension of intermediate I-52 (32 g, 134 mmol, 1.0 eq) and silver benzoate (596 mg, 2.6 mmol, 0.02 eq) in CH₃OH (200 mL) at 0° C. was added triethylamine (1.4 g, 13.4 mmol, 0.1 eq) and the reaction mixture was stirred in the dark for 2 hrs. Activated carbon (200 mg) was added, and the reaction mixture was heated to near boiling for 10 mins. The solids were removed by filtration through a short plug of Celite, and the filtrate was concentrated. The residue was dissolved in ether and washed with a saturated aqueous solution of NaHCO₃ and brine, the combined organic layers were dried over anhydrous Na₂SO₄, the solids were removed by filtration and the filtrate was evaporated to give intermediate I-53 (30 g, 92%). MS (ESI): m/z 244 (M+H⁺).

### 144. Intermediate I-54: methyl 2-(pyrrolidin-2-yl)acetate

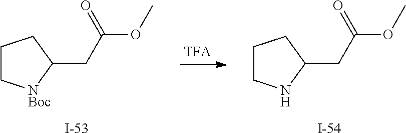

To a solution of intermediate I-53 (30 g, 123 mmol, 1.0 eq) in CH₂Cl₂ (50 mL) was added trifluoroacetic acid (36 g, 370 mmol, 3.0 eq) at 0° C., and the reaction mixture was stirred at room temperature for 2 hrs. The crude reaction mixture was concentrated by evaporation to give intermediate I-54 (28 g, 95%) which was used in the following step without purification. MS (ESI): m/z 144 (M+H⁺).

### 145. Intermediate I-55: methyl 3-(2-(2-methoxy-2-oxoethyl)pyrrolidin-1-yl)propanoate

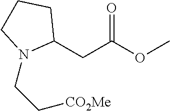

This intermediate was prepared in 56% yield as described for intermediate I-32 but using intermediate I-54 and methyl acrylate as the starting materials. MS (ESI): m/z 230 (M+H⁺).

### 146. Intermediate I-56: methyl 7-oxooctahydroindolizine-6-carboxylate

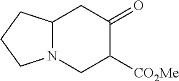

This intermediate was prepared in 78% yield as described for intermediate I-33 but using intermediate I-55 as the starting material. MS (ESI): m/z 198 (M+H⁺).

### 147. Intermediate I-57: hexahydroindolizin-7(1H)-one

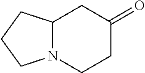

This intermediate was prepared in 43% yield as described for intermediate I-34 but using intermediate I-56 as the starting material. MS (ESI): m/z 140 (M+H⁺).

### 148. Intermediate I-58: 7-(2-(5-bromo-2-fluorophenyl)-1,3-dithian-2-yl)octahydroindolizin-7-ol

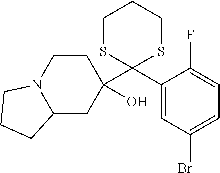

This intermediate was prepared in 63% yield as described for intermediate I-37 but using intermediate I-57 as the starting material. MS (ESI): m/z 432 (M+H⁺).

### 149. Intermediate I-59: (5-bromo-2-fluorophenyl)(7-hydroxyoctahydroindolizin-7-yl)methanone

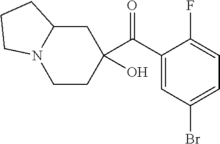

This intermediate was prepared in 88% yield as described for intermediate I-38 but using intermediate I-58 as the starting material. MS (ESI): m/z 342 (M+H⁺).

### 150. Intermediate I-60: 5-bromo-2′,3′,5′,6′,8′,8a′-hexahydro-1′H,3H-spiro[benzofuran-2,7′-indolizin]-3-one

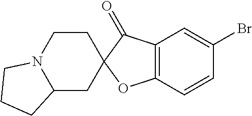

This intermediate was prepared in 84% yield as described for intermediate I-39 but using intermediate I-59 as the starting material. MS (ESI): m/z 322 (M+H⁺).

### 151. Intermediate I-61: 5-(4,4,5,5-tetramethyl-1,3,2-dioxaborolan-2-yl)-2′,3′,5′,6′,8′,8a′-hexahydro-1′H,3H-spiro[benzofuran-2,7′-indolizine]

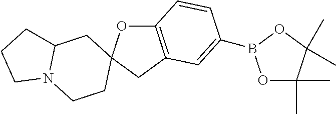

This intermediate was prepared in 87% yield as described for intermediate I-7 but using compound 89 as the starting material. MS (ESI): m/z 356 (M+H⁺).

### 152. Intermediate I-62: 2′,3′,5′,6′,8′,8a′-hexahydro-1′H,3H-spiro[benzofuran-2,7′-indolizine]-5-carbonitrile

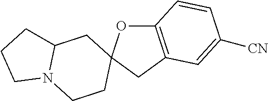

This intermediate was prepared in 43% yield as described for intermediate I-42 but using compound 89 as the starting material. MS (ESI): m/z 255 (M+H⁺).

### 153. Intermediate I-63: 2′,3′,5′,6′,8′,8a′-hexahydro-1′H,3H-spiro[benzofuran-2,7′-indolizine]-5-carbaldehyde

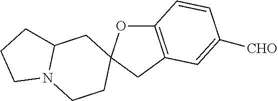

This intermediate was prepared in 90% yield as described for intermediate I-43 but using intermediate I-62 as the starting material. MS (ESI): m/z 258 (M+H⁺).

### 154. Intermediates I-64 and I-64a: hexahydro-1H-pyrrolo[1,2-a]azepin-7(8H)-one and hexahydro-1H-pyrrolo[1,2-a]azepin-8(5H)-one

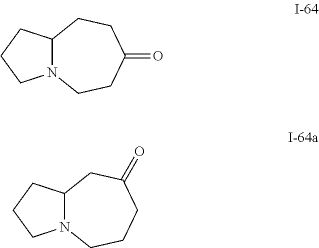

A mixture of these intermediates was prepared in 65% yield as described for the mixture of intermediates I-44 and I-44a but using intermediate I-57 as the starting material. MS (ESI): m/z 154.0 (M+H⁺).

### 155. Intermediates I-65 and I-65a: 7-(2-(5-bromo-2-fluorophenyl)-1,3-dithian-2-yl)octahydro-1H-pyrrolo[1,2-a]azepin-7-ol and 8(2-(5-bromo-2-fluorophenyl)-1,3-dithian-2-yl)octahydro-1H-pyrrolo[1,2-a]azepin-8-ol

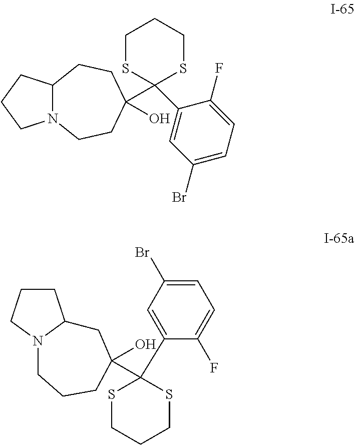

A mixture of these intermediates was prepared in 69% yield as described for the mixture of intermediates I-45 and I-45a but using a mixture of intermediates I-64 and I-64a as the starting material. MS (ESI): m/z 456.0 (M+H⁺).

### 156. Intermediates I-66 and I-66a: (5-bromo-2-fluorophenyl)(7-hydroxyoctahydro-1H-pyrrolo[1,2-a]azepin-7-yl)methanone and (5-bromo-2-fluorophenyl)(8-hydroxyoctahydro-1H-pyrrolo[1,2-a]azepin-8-yl)methanone

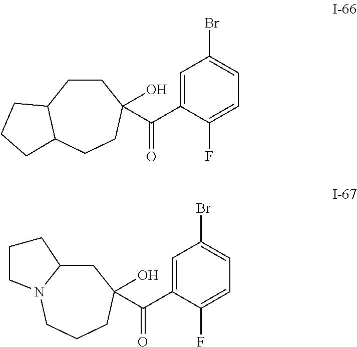

A mixture of these intermediates was prepared in 100% yield as described for the mixture of intermediates I-46 and I-46a but using a mixture of intermediates I-65 and I-65a as the starting material. MS (ESI): m/z 356.0 (M+H⁺).

### 157. Intermediates I-67 and I-67a: 5-bromo-1′,2′,3′,5′,6′,8′,9′,9a′-octahydro-3H-spiro[benzofuran-2,7′-pyrrolo[1,2-a]azepin]-3-one and 5-bromo-1′,2′,3′,5′,6′,7′,9′,9a′-octahydro-3H-spiro[benzofuran-2,8′-pyrrolo[1,2-a]azepin]-3-one

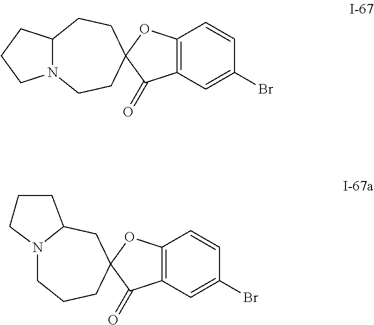

A mixture of these intermediates was prepared in 67% yield as described for the mixture of intermediates I-47 and I-47a but using a mixture of intermediates I-66 and I-66a as the starting material. MS (ESI): m/z 336.0 (M+H⁺).

### 158. Intermediates I-68 and I-68a: 5-(4,4,5,5-tetramethyl-1,3,2-dioxaborolan-2-yl)-1′,2′,3′,5′,6′,7′,9′,9a′-octahydro-3H-spiro[benzofuran-2,8′-pyrrolo[1,2-a]azepine] and 5-(4,4,5,5-tetramethyl-1,3,2-dioxaborolan-2-yl)-1′,2′,3′,5′,6′,8′,9′,9a′-octahydro-3H-spiro[benzofuran-2,7′-pyrrolo[1,2-a]azepine]

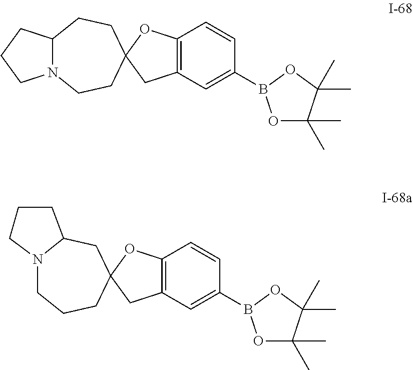

A mixture of these intermediates was prepared in 38% yield as described for intermediate I-7 but using a mixture of compounds 94 and 94a as the starting material. MS (ESI): m/z 370.0 (M+H⁺).

### 159. Intermediates I-69 and I-69a: 1′,2′,3′,5′,6′,7′,9′,9a′-octahydro-3H-spiro[benzofuran-2,8′-pyrrolo[1,2-a]azepine]-5-carbonitrile and 1′,2′,3′,5′,6′,8′,9′,9a′-octahydro-3H-spiro[benzofuran-2,7′-pyrrolo[1,2-a]azepine]-5-carbonitrile

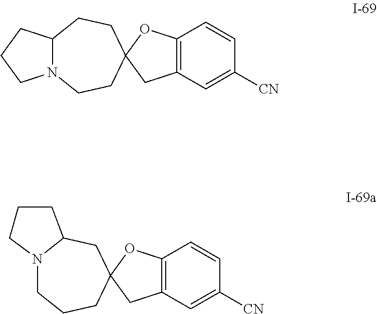

A mixture of these intermediates was prepared in 24% yield as described for intermediate I-42 but using the mixture of compounds 94 and 94a as the starting material. MS (ESI): m/z 269.0 (M+H⁺).

### 160. Intermediates I-70 and I-70a: 1′,2′,3′,5′,6′,7′,9′,9a′-octahydro-3H-spiro[benzofuran-2,8′-pyrrolo[1,2-a]azepine]-5-carbaldehyde and 1′,2′,3′,5′,6′,8′,9′,9a′-octahydro-3H-spiro[benzofuran-2,7′-pyrrolo[1,2-a]azepine]-5-carbaldehyde

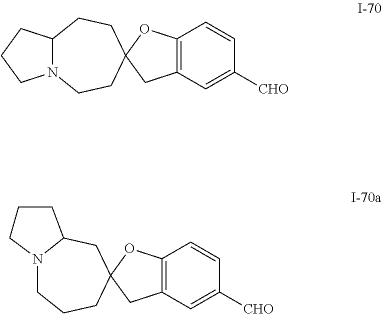

A mixture of these intermediates was prepared in 75% yield as described for intermediate I-17 but using the mixture of intermediates I-69 and I-69a as the starting material. MS (ESI): m/z 272.0 (M+H⁺).

### B. Histamine H3 In Vitro Assay

H3 GTPγS assay (SPA method) was performed at EuroScreen (Belgium, ES-392-C) using conventional methods. Briefly, cells expressing the human histamine H3 receptor were homogenized in 15 mM Tris-HCl pH 7.5, 2 mM MgCl₂, 0.3 mM EDTA, and 1 mM EGTA. Membranes were washed twice in the above tris buffer, collected by centrifugation (40,000×g, 25 min), and re-suspended in 75 mM Tris-HCl pH 7.5, 12.5 mM MgCl₂, 0.3 mM EDTA, 1 mM EGTA, and 250 mM sucrose. Membranes were frozen in liquid nitrogen until use. On the day of the assay, membranes were thawed and diluted in assay buffer (20 mM HEPES pH 7.4, 100 mM NaCl, 10 μg/ml saponin, 1 mM MgCl₂) to give 500 μg/ml and mixed (v/v) with GDP in assay buffer for a final GDP concentration of 10 μM and incubated on ice for at least 15 min. PVT-WGA beads (Amersham, RPNQ001) were diluted in assay buffer at 50 mg/mL and mixed with GTPγ[³⁵S] (Amersham, SJ1308) diluted in assay buffer to give ˜25,000 dpm/10 μL, and mixed vol/vol just before the start of the reaction. The reaction was started by adding 50 μL of test compound, 20 μL of the membranes:GDP mix, 10 μL of buffer, and 20 μL of the GTPγ[³⁵S]:beads mix in a 96 well plate Optiplate™ (PerkinElmer, 6005299) covered with topseal (TopCount™, PerkinElmer), mixed with an orbital shaker for 2 min, incubated for 1 hour at room temperature, centrifuged for 10 min at 2000 rpm, incubated for 1 h at room temperature, and counted for 1 min in a TopCount™ reader (PerkinElmer). Dose response curves and IC₅₀ values (concentration to inhibit the reaction by 50%) were calculated by nonlinear regression using XLfit software (IDBS).

For antagonists testing, 10 μL of a reference agonist (R-γ-Me-Histamine) instead of 10 μL buffer was added at a concentration corresponding to the EC₈₀ (30 nM). Control ligands were R-γ-Me-Histamine (Tocris, 0569), Imetit (Sigma, I-135), Thioperamide (Tocris, 0644), and Clobenpropit (Tocris, 0754) diluted in assay buffer.

The compounds provided herein were tested in the histamine H3 in vitro assay. In one embodiment, the respective HCl salts of the compounds provided herein were prepared using standard chemical procedures and tested in the histamine H3 in vitro assay. The functional potency of the compounds (as indicated by their IC₅₀s) are shown in Table 1.

TABLE 1

| Compound | Potency |
|---|---|
| 1 | (++) |
| 4 | (+) |
| 5 | (+++) |
| 6 | (+++) |
| 7 | (++) |
| 8 | (++) |
| 9 | (++) |
| 10 | (+) |
| 11 | (+++) |
| 12 | (+++) |
| 13 | (++) |
| 14 | (++) |
| 15 | (++) |
| 16 | (+++) |
| 17 | (++) |
| 18 | (++) |
| 19 | (++) |
| 20 | (++) |
| 21 | (+) |
| 22 | (+++) |
| 23 | (+) |
| 24 | (+) |
| 25 | (+) |
| 27 | (++) |
| 28 | (++) |
| 29 | (+++) |
| 30 | (+++) |
| 31 | (+++) |
| 32 | (+++) |
| 33 | (+++) |
| 34 | (++) |
| 35 | (++) |
| 36 | (++) |
| 37 | (++) |
| 38 | (++) |
| 39 | (+++) |
| 40 | (+++) |
| 41 | (+++) |
| 42 | (++) |
| 43 | (++) |
| 44 | (++) |
| 45 | (++) |
| 46 | (++) |
| 47 | (+) |
| 48 | (+++) |
| 49 | (++) |
| 50 | (+++) |
| 51 | (++) |
| 52 | (++) |
| 53 | (++) |
| 54 | (+++) |
| 55 | (++) |
| 56 | (+++) |
| 57 | (+++) |
| 58 | (+++) |
| 59 | (+++) |
| 60 | (+++) |
| 61 | (+++) |
| 62 | (+++) |
| 63 | (+++) |
| 64 | (+++) |
| 65 | (+++) |
| 66 | (+++) |
| 67 | (+) |
| 68 | (++) |
| 69 | (++) |
| 70 | (+++) |
| 71 | (++) |
| 72 | (+) |
| 73 | (++) |
| 74 | (+++) |
| 75 | (+++) |
| 76 | (+++) |
| 77 | (+++) |
| 78 | (+++) |
| 79 | (+++) |
| 80 | (+++) |
| 81 | (+++) |
| 82/82a | (+) |
| 83 | (++) |
| 84 | (++) |
| 85 | (+++) |
| 86 | (+++) |
| 87/87a | (++) |
| 88/88a | (++) |
| 89 | (++) |
| 90 | (+++) |
| 91 | (+++) |
| 92 | (+++) |
| 93 | (+++) |
| 94/94a | (+) |
| 95/95a | (++) |
| 96/96a | (+++) |
| 97/97a | (+++) |

(+++) ≦10 nM
(++) ≦100 nM
(+) ≦1,000 nM

In one embodiment, Compounds 2, 3, and 26 were tested in the histamine H3 in vitro assay and gave IC₅₀ values >1,000 nM.

C. In Vivo Rat PK—Brain Exposures

In vivo rat PK studies were performed using certain compounds provided herein. In one embodiment, a compound was dissolved in water and was administered to adult male rats (n=3 per time point) by oral gavage at a dose of 3 to 10 mg/kg. The plasma samples were collected and brain tissues were harvested at various time points after dosing, e.g., 15 min, 30 min, 1 hr, 2 hr, and 24 hr after dosing. The PK samples were analyzed; and the compound concentrations in these samples were quantified by LC/MS. The results of compound concentrations in the rat brain at 1 hr and 24 hr after dosing are summarized in Table 2. The pKa values of the R⁶ moiety of the compounds were calculated using ACD Lab (Version 12.01).

TABLE 3

| | | | Compound | |
|---|---|---|---|---|
| | | | Concentration | |
| Compound | Dose | Calculated | (ng/g rat brain) | |
| Number | (mg/kg) | pKa of R⁶ | 1 hr | 24 hr |
| 41 | 3 | 9.8 | <25 | 450 |
| 40 | 3 | 6.6 | 900 | 140 |
| 30 | 10 | 6.6 | 5200 | 250 |
| 5 | 5 | <0 | 3100 | 400 |

These studies indicated that compounds provided herein with an R⁶ moiety having certain pKa values (e.g., a pKa value as provided herein elsewhere) had high brain penetration and low brain accumulation in a subject (e.g., a rat) treated with the compounds.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the disclosure and are encompassed by the appended claims.

All of the patents, patent applications and publications referred to herein are incorporated herein in their entireties. Citation or identification of any reference in this application is not an admission that such reference is available as prior art to this application. The full scope of the disclosure is better understood with reference to the appended claims.

1. A compound of formula (I): 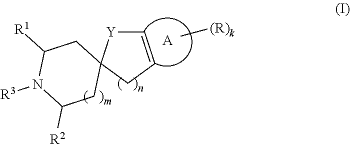
or a pharmaceutically acceptable salt or stereoisomer thereof, wherein
ring A is optionally substituted 5- or 6-membered aryl or heteroaryl;
Y is O, S, NH, or CH₂;
k is 0, 1, 2, 3, or 4;
m is 0, 1, 2, 3, or 4;
n is 1, 2, or 3;
(i) R¹ and R³ together with the atoms to which they are attached form a 3 to 10 membered heterocyclyl optionally substituted with one or more R¹⁰; and R² is hydrogen, ═O, (C₁-C₁₀)alkyl, (C₂-C₁₀)alkenyl, (C₃-C₁₀)cycloalkyl, (C₆-C₁₀)aralkyl, (C₁-C₁₀) heteroalkyl, (3 to 10 membered) heterocyclyl, (6 to 10 membered)aryl, or (5 to 10 membered)heteroaryl, each of which may be optionally substituted with one or more R¹⁰; or (ii) R² and R³ together with the atoms to which they are attached form a 3 to 10 membered heterocyclyl optionally substituted with one or more R¹⁰; and R¹ is hydrogen, ═O, (C₁-C₁₀)alkyl, (C₂-C₁₀)alkenyl, (C₃-C₁₀)cycloalkyl, (C₆-C₁₀)aralkyl, (C₁-C₁₀)heteroalkyl, (3 to 10 membered) heterocyclyl, (6 to 10 membered)aryl, or (5 to 10 membered)heteroaryl, each of which may be optionally substituted with one or more R¹⁰;
each occurrence of R is independently hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹⁰; optionally two adjacent R substituents together with the atoms to which they are attached form an optionally substituted 3 to 10 membered cycloalkyl, heterocyclyl, aryl, or heteroaryl ring;
each occurrence of R¹⁰ is independently a bond, hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered) heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹¹; optionally two germinal or vicinal R¹⁰ substituents together with the atom(s) to which they are attached form an optionally substituted 3 to 10 membered ring;
each occurrence of R¹¹ is independently hydrogen, halo, cyano, (C₁-C₁₀)alkyl optionally substituted with one or more R¹², (C₂-C₁₀)alkenyl optionally substituted with one or more R¹², (C₃-C₁₀)cycloalkyl optionally substituted with one or more R¹², (C₁-C₁₀)heteroalkyl optionally substituted with one or more R¹², (3 to 10 membered) heterocyclyl optionally substituted with one or more R¹², (C₆-C₁₂)aralkyl optionally substituted with one or more R¹², (6 to 10 membered)aryl optionally substituted with one or more R¹², (5 to 10 membered)heteroaryl optionally substituted with one or more R¹², ═O, —R¹³, —OR¹³, —NR¹³R¹⁴, —N(R¹³)C(O)R¹⁴, —C(O)NR¹³R¹⁴, —C(O)R, —C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹¹ substituents together with the atom(s) to which they are attached form an optionally substituted 3 to 10 membered ring;
each occurrence of R¹² is independently hydrogen, halo, cyano, (C₁-C₆)alkyl optionally substituted with one or more R¹³, (C₂-C₆)alkenyl optionally substituted with one or more R¹³, (C₃-C₇)cycloalkyl optionally substituted with one or more R¹³, (3 to 8 membered)heterocyclyl optionally substituted with one or more R¹³, (6 to 10 membered)aryl optionally substituted with one or more R¹³, (5 to 10 membered) heteroaryl optionally substituted with one or more R¹³, ═O, —R¹³, —OR¹³, —NR¹³R¹⁴, —N(R¹³)C(O)R¹⁴, —C(O)NR¹³R¹⁴, —C(O)R¹³, —C(O)OR¹³, —OC(O)R¹³, —OC(O)NR¹³R¹⁴, —NR¹³C(O)OR¹⁴, —SR¹³, —(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹² substituents together with the atom(s) to which they are attached form an optionally substituted 3 to 10 membered ring; and
R¹³, R¹⁴, and R¹⁵ are independently hydrogen, halo, cyano, (C₁-C₆)alkyl, (C₂-C₆)alkenyl, (C₃-C₇)cycloalkyl, (C₇-C₁₀)aralkyl; (C₁-C₆)heteroalkyl, (3 to 8 membered) heterocyclyl, (6 to 10 membered)aryl, or (5 to 10 membered)heteroaryl; optionally two germinal or vicinal R¹³ substituents together with the atom(s) to which they are attached form an optionally substituted 3 to 10 membered ring; optionally R¹³ and R¹⁴, or R¹⁴ and R¹⁵ together with the atom(s) to which they are attached form an optionally substituted 3 to 10 membered ring.

2. The compound of claim 1, or a pharmaceutically acceptable salt or stereoisomer thereof, having formula (II): 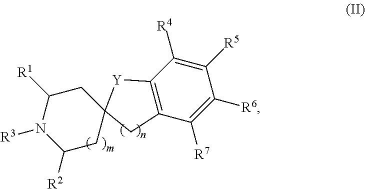
wherein R⁴, R⁵, R⁶, and R⁷ are each independently R; optionally R⁴ and R⁵, or R⁵ and R⁶, or R⁶ and R⁷ together with the atoms to which they are attached form an optionally substituted 3 to 10 membered cycloalkyl, heterocyclyl, aryl, or heteroaryl ring.

3. The compound of claim 1, or a pharmaceutically acceptable salt or stereoisomer thereof, having formula (Va) or (Vb): 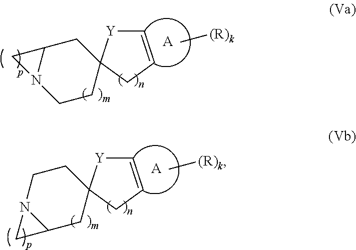
wherein p is 1, 2, 3, 4, 5, 6, 7, or 8.

4. The compound of claim 3, or a pharmaceutically acceptable salt or stereoisomer thereof, having formula (VIa) or (VIb): 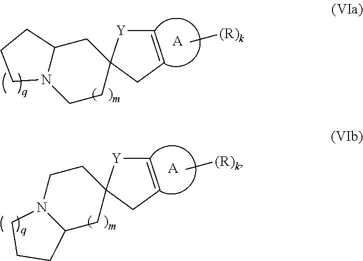
wherein q is 1 or 2.

5. The compound of claim 4, or a pharmaceutically acceptable salt or stereoisomer thereof, having formula (VIIa) or (VIIb): 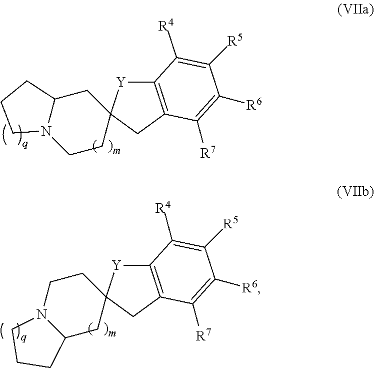
wherein R⁴, R⁵, R⁶, and R⁷ are each independently R; optionally R⁴ and R⁵, or R⁵ and R⁶, or R⁶ and R⁷ together with the atoms to which they are attached form an optionally substituted 3 to 10 membered cycloalkyl, heterocyclyl, aryl, or heteroaryl ring.

6. The compound of claim 5, wherein R⁴ and R⁷ are hydrogen; and R⁵ and R⁶ are each independently hydrogen, halo, cyano, (C₁-C₆)alkyl optionally substituted with one or more R¹⁰, (C₃-C₇)cycloalkyl optionally substituted with one or more R¹⁰, (3 to 10 membered)heterocyclyl optionally substituted with one or more R¹⁰, (6 to 10 membered) aryl optionally substituted with one or more R¹⁰, (5 to 10 membered)heteroaryl optionally substituted with one or more R¹⁰, —OR¹⁰, —N(R¹⁰)₂, —C(O)R¹⁰, —NR¹⁰C(O)R¹⁰, or —C(O)N(R¹⁰)₂.

7. The compound of claim 6, wherein one of R⁵ and R⁶ is hydrogen, and the other is hydrogen, halo, cyano, (C₁-C₆)alkyl optionally substituted with one or more R¹⁰, (C₃-C₇)cycloalkyl optionally substituted with one or more R¹⁰, (3 to 10 membered) heterocyclyl optionally substituted with one or more R¹⁰, (6 to 10 membered)aryl optionally substituted with one or more R¹⁰, (5 to 10 membered) heteroaryl optionally substituted with one or more R¹⁰, —OR¹⁰, —N(R¹⁰)₂, —C(O)R¹⁰, —NR¹⁰C(O)R¹⁰, or —C(O)N(R¹⁰)₂.

8. The compound of claim 7, wherein R⁵ is hydrogen, and R⁶ is hydrogen, halo, optionally substituted 9-membered heteroaryl, optionally substituted phenyl, optionally substituted pyrazolyl, optionally substituted imidazolyl, optionally substituted pyrimidinyl, optionally substituted pyrazinyl, optionally substituted pyridyl, optionally substituted indolyl, optionally substituted benzimidazolyl, optionally substituted imidazopyridinyl, optionally substituted piperidinyl, optionally substituted piperazinyl, or —CH₂R¹⁰.

9. The compound of claim 7, wherein R⁶ is hydrogen, and R⁵ is hydrogen, halo, optionally substituted 9-membered heteroaryl, optionally substituted phenyl, optionally substituted pyrimidinyl, optionally substituted pyrazinyl, optionally substituted pyridyl, optionally substituted piperidinyl, optionally substituted piperazinyl, or —CH₂R¹⁰.

10. The compound of claim 5 or a pharmaceutically acceptable salt or stereoisomer thereof, having formula (VIIIa) or (VIIIb):

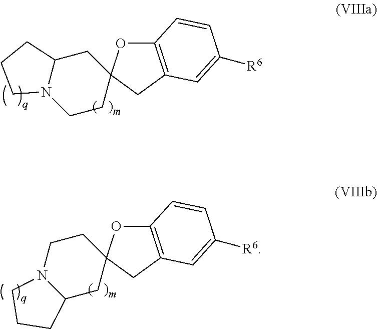

11. The compound of claim 10, wherein q is 1 and m is 1.

12. The compound of claim 11, selected from the group consisting of:

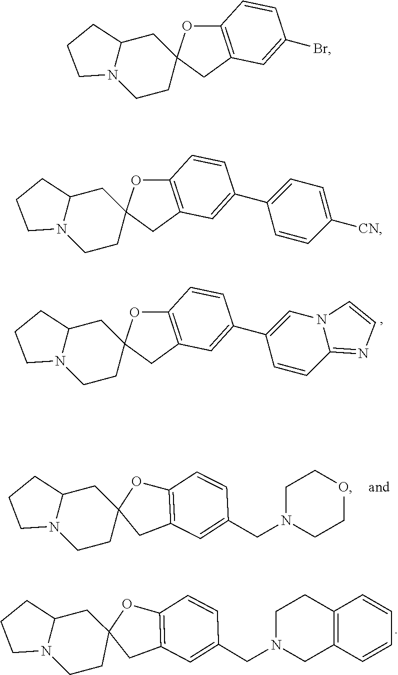

13. The compound of claim 10, wherein q is 2 and m is 1.

14. The compound of claim 13, selected from the group consisting of:

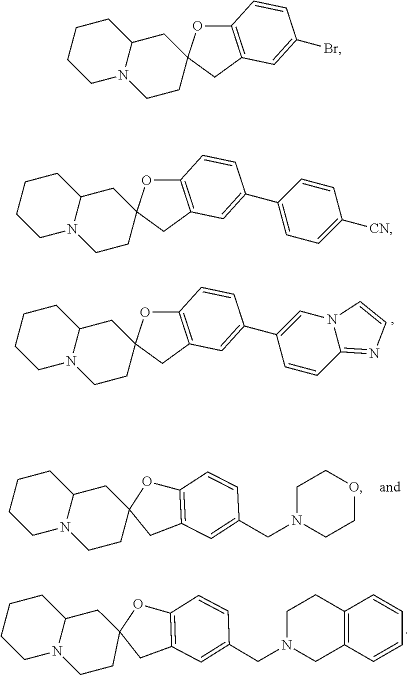

15. The compound of claim 10, or a pharmaceutically acceptable salt or stereoisomer thereof, having formula (VIIIa):

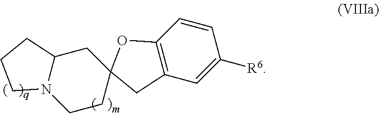

16. The compound of claim 15, wherein q is 1 and m is 2.

17. The compound of claim 16, selected from the group consisting of:

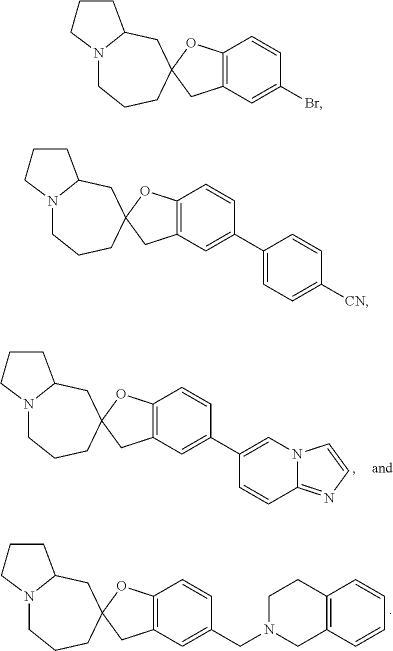

18. The compound of claim 15, wherein q is 2 and m is 2.

19. The compound of claim 18, selected from the group consisting of:

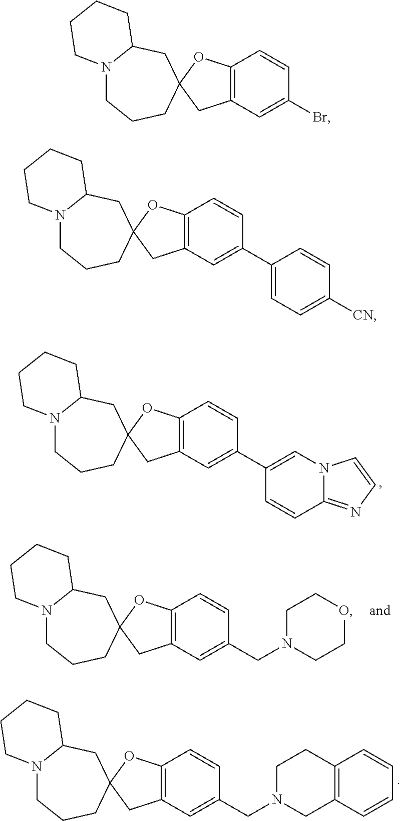

20. The compound of claim 10, or a pharmaceutically acceptable salt or stereoisomer thereof, having formula (VIIIb):

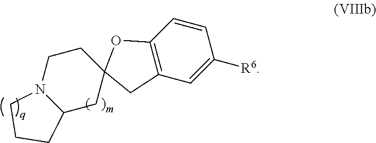

21. The compound of claim 20, wherein q is 1 and m is 2.

22. The compound of claim 21, selected from the group consisting of

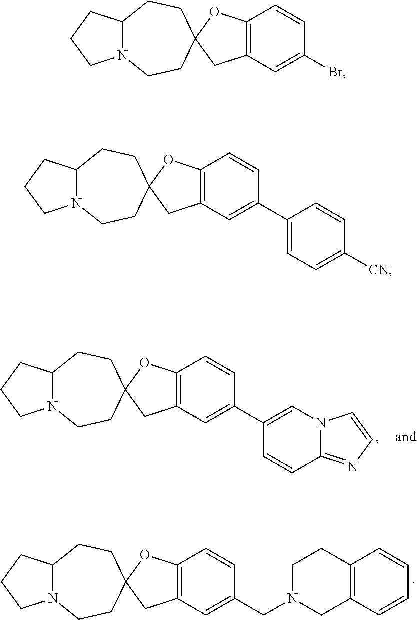

23. The compound of claim 20, wherein q is 2 and m is 2.

24. The compound of claim 23, selected from the group consisting of:

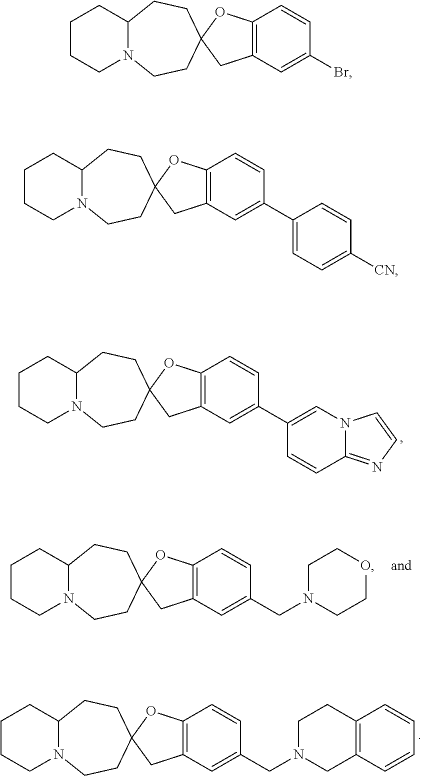

25. A compound of formula (IVa): 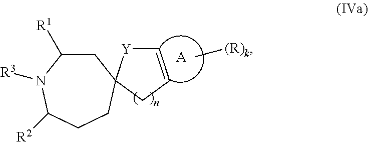
or a pharmaceutically acceptable salt or stereoisomer thereof, wherein
ring A is optionally substituted 5- or 6-membered aryl or heteroaryl;
Y is O, S, NH, or CH₂;
k is 0, 1, 2, 3, or 4;
n is 1, 2, or 3;
R¹, R², and R³ are independently hydrogen, ═O, (C₁-C₁₀)alkyl, (C₂-C₁₀)alkenyl, (C₃-C₁₀)cycloalkyl, (C₆-C₁₀)aralkyl, (C₁-C₁₀)heteroalkyl, (3 to 10 membered) heterocyclyl, (6 to 10 membered)aryl, or (5 to 10 membered)heteroaryl, each of which may be optionally substituted with one or more R¹⁰; optionally R¹ and R², or R¹ and R³, or R² and R³ together with the atoms to which they are attached form an optionally substituted 3 to 10 membered cycloalkyl or heterocyclyl ring;
each occurrence of R is independently hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹⁰; optionally two adjacent R substituents together with the atoms to which they are attached form an optionally substituted 3 to 10 membered cycloalkyl, heterocyclyl, aryl, or heteroaryl ring;
each occurrence of R¹⁰ is independently a bond, hydrogen, halo, cyano, (C₁-C₁₀)alkyl, (C₇-C₁₀)alkenyl, (C₃-C₁₀)cycloalkyl, (C₁-C₁₀)heteroalkyl, (3 to 10 membered) heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹¹; optionally two germinal or vicinal R¹⁰ substituents together with the atom(s) to which they are attached form an optionally substituted 3 to 10 membered ring;
each occurrence of R¹¹ is independently hydrogen, halo, cyano, (C₁-C₁₀)alkyl optionally substituted with one or more R¹², (C₂-C₁₀)alkenyl optionally substituted with one or more R¹², (C₃-C₁₀)cycloalkyl optionally substituted with one or more R¹², (C₁-C₁₀)heteroalkyl optionally substituted with one or more R¹², (3 to 10 membered) heterocyclyl optionally substituted with one or more R¹², (C₆-C₁₂)aralkyl optionally substituted with one or more R¹², (6 to 10 membered)aryl optionally substituted with one or more R¹², (5 to 10 membered)heteroaryl optionally substituted with one or more R¹², ═O, —R¹³, —OR¹³, —NR¹³R¹⁴, —N(R¹³)C(O)R¹⁴, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹¹ substituents together with the atom(s) to which they are attached form an optionally substituted 3 to 10 membered ring;
each occurrence of R¹² is independently hydrogen, halo, cyano, (C₁-C₆)alkyl optionally substituted with one or more R¹³, (C₂-C₆)alkenyl optionally substituted with one or more R¹³, (C₃-C₇)cycloalkyl optionally substituted with one or more R¹³, (3 to 8 membered)heterocyclyl optionally substituted with one or more R¹³, (6 to 10 membered)aryl optionally substituted with one or more R¹³, (5 to 10 membered) heteroaryl optionally substituted with one or more R¹³, ═O, —R¹³, —OR¹³, —NR¹³R¹⁴, —N(R¹³)C(O)R¹⁴, —C(O)NR¹³R¹⁴, —C(O)R¹³, —C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹² substituents together with the atom(s) to which they are attached form an optionally substituted 3 to 10 membered ring; and
R¹³, R¹⁴ and R¹⁵ are independently hydrogen, halo, cyano, (C₁-C₆)alkyl, (C₂-C₆)alkenyl, (C₃-C₇)cycloalkyl, (C₇-C₁₀)aralkyl; (C₁-C₆)heteroalkyl, (3 to 8 membered) heterocyclyl, (6 to 10 membered)aryl, or (5 to 10 membered)heteroaryl; optionally two germinal or vicinal R¹³ substituents together with the atom(s) to which they are attached form an optionally substituted 3 to 10 membered ring; optionally R¹³ and R¹⁴, or R¹⁴ and R¹⁵ together with the atom(s) to which they are attached form an optionally substituted 3 to 10 membered ring.

26-37. (canceled)

38. A compound of formula (III): 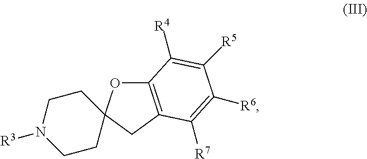
or a pharmaceutically acceptable salt or stereoisomer thereof, wherein
R³ is hydrogen, ═O, (C₁-C₁₀)alkyl, (C₂-C₁₀)alkenyl, (C₃-C₁₀)cycloalkyl, (C₆-C₁₀)aralkyl, (C₁-C₁₀)heteroalkyl, (3 to 10 membered)heterocyclyl, (6 to 10 membered) aryl, or (5 to 10 membered)heteroaryl, each of which may be optionally substituted with one or more R¹⁰;
R⁴, R⁵, and R⁷ are each independently hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹⁰;
R⁶ is (C₁-C₁₀)alkyl, (C₂-C₁₀)alkenyl, (C₃-C₁₀)cycloalkyl, (C₁-C₁₀)heteroalkyl, (3 to 10 membered)heterocyclyl, (6 to 10 membered)aryl, (5 to 10 membered)heteroaryl, alkoxyl, aminoalkyl, hydroxyl, amino, imino, amido, thiol, sulfinyl, or sulfonyl, each of which may be optionally substituted with one or more R¹⁰; with the proviso that (i) R⁶ is not 6-membered heteroaryl substituted with oxo, hydroxyl, or halo; and (ii) R⁶ is not phenyl substituted with amido or sulfonyl;
optionally R⁴ and R⁵, R⁵ and R⁶, or R⁶ and R⁷ together with the atoms to which they are attached form an optionally substituted 3 to 10 membered cycloalkyl, heterocyclyl, aryl, or heteroaryl ring;
each occurrence of R¹⁰ is independently a bond, hydrogen, halo, cyano, (C₁-C₁₀)alkyl, (C₂-C₁₀)alkenyl, (C₃-C₁₀)cycloalkyl, (C₁-C₁₀)heteroalkyl, (3 to 10 membered) heterocyclyl, (6 to 10 membered)aryl, (5 to 10 membered)heteroaryl, alkoxyl, aminoalkyl, hydroxyl, amino, imino, amido, carbonyl, thiol, sulfinyl, or sulfonyl, each of which may be optionally substituted with one or more R¹¹; optionally two germinal or vicinal R¹⁰ substituents together with the atom(s) to which they are attached form an optionally substituted 3 to 10 membered ring;
each occurrence of R¹¹ is independently hydrogen, halo, cyano, (C₁-C₁₀)alkyl optionally substituted with one or more R¹², (C₂-C₁₀)alkenyl optionally substituted with one or more R¹², (C₃-C₁₀)cycloalkyl optionally substituted with one or more R¹², (C₁-C₁₀)heteroalkyl optionally substituted with one or more R¹², (3 to 10 membered) heterocyclyl optionally substituted with one or more R¹², (C₆-C₁₂)aralkyl optionally substituted with one or more R¹², (6 to 10 membered)aryl optionally substituted with one or more R¹², (5 to 10 membered)heteroaryl optionally substituted with one or more R¹², ═0, —R¹³, —0R¹³, —NR¹³R¹⁴, —N(R¹³)C(O)R¹⁴, —C(O)NR¹³R¹⁴, —C(O)R¹³, —C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹¹ substituents together with the atom(s) to which they are attached form an optionally substituted 3 to 10 membered ring;
each occurrence of R¹² is independently hydrogen, halo, cyano, (C₁-C₆)alkyl optionally substituted with one or more R¹³, (C₂-C₆)alkenyl optionally substituted with one or more R¹³, (C₃-C₇)cycloalkyl optionally substituted with one or more R¹³, (3 to 8 membered)heterocyclyl optionally substituted with one or more R¹³, (6 to 10 membered)aryl optionally substituted with one or more R¹³, (5 to 10 membered) heteroaryl optionally substituted with one or more R¹³, ═O, —R¹³, —OR¹³, —NR¹³R¹⁴, —N(R¹³)C(O)R¹⁴, —C(O)NR¹³R¹⁴, —C(O)R¹³, —C(O)OR¹³, —OC(O)R¹³, —OC(O)NR¹³R¹⁴, —NR¹³C(O)OR¹⁴, —SR¹³, —S(O)R¹³, —S(O)₂R¹³, —S(O)₂NR¹³R¹⁴, —NR¹³S(O)₂R¹⁴, or —NR¹³C(O)NR¹⁴R¹⁵; optionally two germinal or vicinal R¹² substituents together with the atom(s) to which they are attached form an optionally substituted 3 to 10 membered ring; and
R¹³, R¹⁴, and R¹⁵ are independently hydrogen, halo, cyano, (C₁-C₆)alkyl, (C₂-C₆)alkenyl, (C₃-C₇)cycloalkyl, (C₇-C₁₀)aralkyl; (C₁-C₆)heteroalkyl, (3 to 8 membered) heterocyclyl, (6 to 10 membered)aryl, or (5 to 10 membered)heteroaryl; optionally two germinal or vicinal R¹³ substituents together with the atom(s) to which they are attached form an optionally substituted 3 to 10 membered ring; optionally R¹³ and R¹⁴, or R¹⁴ and R¹⁵ together with the atom(s) to which they are attached form an optionally substituted 3 to 10 membered ring.

39-54. (canceled)

55. A pharmaceutical composition comprising a compound of claim 1.

56. The pharmaceutical composition of claim 55, which further comprises one or more additional active agents.

57. A method of reducing the activity of a histamine receptor, said method comprising contacting said histamine receptor and a compound of claim 1, or a pharmaceutically acceptable salt or stereoisomer thereof.

58. (canceled)

59. A method of treating, preventing, or managing a disorder related to histamine H3 receptor comprising administering to a subject a therapeutically or prophylactically effective amount of a compound of claim 1, or a pharmaceutically acceptable salt or stereoisomer thereof.

60. (canceled)

61. The method of claim 59, wherein said disorder is neurological disorder, neurodegenerative disease, schizophrenia, Alzheimer's disease, Parkinson's disease, affective disorder, attention deficit hyperactivity disorder (ADHD), psychosis, convulsion, seizure, vertigo, epilepsy, narcolepsy, pain, neuropathic pain, sensitization accompanying neuropathic pain, psychosis, mood disorder, depression, anxiety, excessive daytime sleepiness, narcolepsy, multiple sclerosis, jet lag, drowsy side effect of medications, insomnia, substance abuse, cognitive impairment, impairment of learning, impairment of memory, impairment of attention, vigilance or speed of response, metabolic disorder, diabetes, obesity, disorder related to satiety, disorder of gastric activity, disorder of enteric system, disorder of exocrine pancreatic system, acid secretion, digestive disorder, disorder of gut motility; movement disorder, restless leg syndrome (RLS), or Huntington's disease.

62. A pharmaceutical composition comprising a compound of claim 25.

63. A pharmaceutical composition comprising a compound of claim 38.

64. A method of reducing the activity of a histamine receptor, said method comprising contacting said histamine receptor and a compound of claim 25, or a pharmaceutically acceptable salt or stereoisomer thereof.

65. A method of reducing the activity of a histamine receptor, said method comprising contacting said histamine receptor and a compound of claim 38, or a pharmaceutically acceptable salt or stereoisomer thereof.

66. A method of treating, preventing, or managing a disorder related to histamine H3 receptor comprising administering to a subject a therapeutically or prophylactically effective amount of a compound of claim 25, or a pharmaceutically acceptable salt or stereoisomer thereof.

67. A method of treating, preventing, or managing a disorder related to histamine H3 receptor comprising administering to a subject a therapeutically or prophylactically effective amount of a compound of claim 38, or a pharmaceutically acceptable salt or stereoisomer thereof.

68. A pharmaceutical composition comprising lurasidone, or a pharmaceutically acceptable salt thereof, and an H3 antagonist or inverse agonist.

69. A method for treating a patient comprising administering lurasidone, or a pharmaceutically acceptable salt thereof, and an H3 antagonist or inverse agonist to a patient in need thereof.

70. The method of claim 77, wherein the patient has a disorder selected from schizophrenia; Alzheimer's disease; Parkinson's disease; attention deficit hyperactivity disorder; excessive daytime sleepiness; and cognitive impairment associated with schizophrenia, Alzheimer's disease, Parkinson's disease, or attention deficit hyperactivity disorder.
